(19) United States
(12) Patent Application Publication
(10) Pub. No.: US 2025/0377570 A1
(43) Pub. Date: Dec. 11, 2025

(54) COMPOUND, LIQUID CRYSTAL COMPOSITION, AND LIQUID CRYSTAL DISPLAY ELEMENT, SENSOR, LIQUID CRYSTAL LENS, OPTICAL COMMUNICATION EQUIPMENT, AND ANTENNA USING SAME

(71) Applicant: DIC Corporation, Tokyo (JP)

(72) Inventors: Mika TAKASAKI; Masanao HAYASHI, Saitama (JP); Daiki NORO, Saitama (JP); Junichi MAMIYA, Saitama (JP); Takaya IKEUCHI, Saitama (JP)

(73) Assignee: DIC Corporation, Tokyo (JP)

(21) Appl. No.: 18/878,619

(22) PCT Filed: Jul. 13, 2023
(86) PCT No.: PCT/JP2023/025828

§ 371 Date: Dec. 23, 2024

(30) Foreign Application Priority Data
Jul. 28, 2022 (JP) 2022-120427

(51) Int. Cl. G02F 1/139; C09K 19/12; C09K 19/16; C09K 19/18; C09K 19/24; C09K 19/30; C09K 19/32; G02F 1/29; H01Q 1/38; H01Q 3/44

(52) U.S. Cl. CPC G02F 1/1393; C09K 19/12; C09K 19/16; C09K 19/18; C09K 19/24; C09K 19/3059; C09K 19/32; G02F 1/294; H01Q 1/38; H01Q 3/44; C09K 2019/122; C09K 2019/163; C09K 2019/183; C09K 2019/188; C09K 2019/3009; C09K 2019/301; C09K 2019/3063

(57) ABSTRACT

The present invention addresses the problem of providing a compound with which a liquid crystal composition having a high Tₙᵢ, a large Δn, a low Vₜₕ, a large Δεᵣ, and a small tan δᵢₛₒ and having good storability at low temperatures can be provided, a liquid crystal composition, and a liquid crystal display element, a sensor, a liquid crystal lens, optical communication equipment, and an antenna made using it. Specifically, the liquid crystal composition is one that contains one or two or more types of compounds represented by general formula (i), which have an indane structure and an isothiocyanate group (—NCS), and one or two or more types of compounds represented by general formula (ii), which have an isothiocyanate group (—NCS).

### TECHNICAL FIELD

The present invention relates to a compound, a liquid crystal composition, and a liquid crystal display element, a sensor, a liquid crystal lens, optical communication equipment, and an antenna using it.

### BACKGROUND ART

As a new application of liquid crystals, which are often used in display applications, antennas made using liquid crystals that exchange radio waves between moving entities, such as automobiles, and communication satellites are attracting attention. Known satellite communications use parabolic antennas, but when used with a moving entity, such as an automobile, the parabolic antenna needs to be continuously pointed toward a satellite, requiring a large movable section. An antenna made using liquid crystals, however, can change the direction of transmission and reception of radio waves through the operation of the liquid crystals within the panel, eliminating the need to move the antenna itself and allowing the antenna to be in a flat shape. In parallel, research on low Earth orbit satellite constellations, composed of numerous low Earth orbit satellites, is ongoing to achieve global high-capacity and high-speed communications. When it comes to tracking low Earth orbit satellites, which appear to be constantly moving from the ground, liquid crystal antennas, which can easily change the direction of transmission and reception of radio waves, are useful.

In general, automated driving, for example of automobiles, requires the downloading of massive data of highly accurate 3D map information. With an antenna made using liquid crystals, however, the downloading of massive data from communication satellites can be achieved without a mechanical movable section by integrating the antenna into the automobile. The frequency band used in satellite communications is an approximately 13 GHz band and is significantly different from the frequencies used in the existing liquid crystal display applications. The required characteristics of liquid crystals, therefore, are also greatly different: for liquid crystals for antennas, the Δn required is, for example, approximately 0.4, and the operating temperature range is, for example, −20° C. to 120° C.

Separately, infrared laser image recognition and distance measurement devices made using liquid crystals are also attracting attention as sensors for automated driving of automobiles and other moving entities. For liquid crystals used in this application, the Δn required is, for example, from 0.3 to 0.6, and the operating temperature range is, for example, 10° C. to 100° C.

In addition, it is known that many of liquid crystalline compounds that constitute liquid crystal compositions exhibiting a high Δn of 0.2 or higher have low compatibility. It is, therefore, also important to select liquid crystalline compounds with high compatibility.

To address these, an example of a liquid crystal technology for antennas is PTL 1.

In NPL 1, furthermore, the use of liquid crystal materials as constituent components of radio-frequency devices is advocated.

### CITATION LIST

### Patent Literature

PTL 1: Japanese Unexamined Patent Application Publication No. 2016-37607

### Non Patent Literature

NPL 1: D. Dolfi, “Electronics Letters,” (UK), 1993, Volume 29, Issue 10, p. 926-928

### SUMMARY OF INVENTION

### Technical Problem

The present invention addresses the problem of providing a compound with which a liquid crystal composition having a high Tₙᵢ, a large Δn, a low Vₜₕ, a large Δεᵣ, and a small tan δᵢₛₒ and having good storability at low temperatures can be provided, a liquid crystal composition, and a liquid crystal display element, a sensor, a liquid crystal lens, optical communication equipment, and an antenna made using it.

### Solution to Problem

After extensive research, the inventors found that a liquid crystal composition containing one or two or more types of compounds represented by general formula (i), which have an indane structure and an isothiocyanate group (—NCS), and one or two or more types of compounds represented by general formula (ii), which have an isothiocyanate group (—NCS), can address the problem described above, thereby completing the present invention.

An example of a configuration of the present invention that addresses the above problem is as follows.

Item 1. A liquid crystal composition containing one or two or more types of compounds represented by general formula (i) below

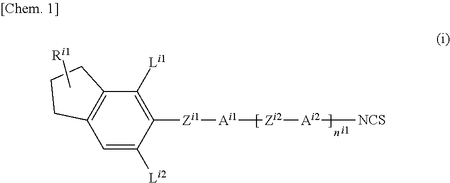

(In general formula (i),
Rⁱ¹ represents a hydrogen atom or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, has optionally been replaced with —CH═CH—CO—O—, —CH═CH—O—CO—, —CO—O—CH═CH—, or —O—CO—CH═CH—, and
one hydrogen atom in the alkyl group, or each of two or more independently, has optionally been replaced with a halogen atom, with the proviso that
an oxygen atom and an oxygen atom are not directly bound together,
Aⁱ¹ and Aⁱ² each independently represent any of a C3 to C16 hydrocarbon ring or a C3 to C16 heterocycle, where
one hydrogen atom in the Aⁱ¹ and Aⁱ², or each of two or more independently, has optionally been replaced by a substituent Sⁱ¹,
the substituent Sⁱ¹ represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, and/or —CO—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, is optionally replaced with —CH═CH—CO—O—, —CH═CH—O—CO—, —CO—O—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together, and
when there are multiple substituents Sⁱ¹, the substituents may be the same or may be different, Lⁱ¹ and Lⁱ² each independently represent any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, is optionally replaced with —O—CO—O—,
one —CH₂—CH₂—CH₂—CH₂— in the alkyl group, or each of two or more independently, has optionally been replaced with —CH═CH—CO—O—, —CH═CH—O—CO—, —COO—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together,
Zⁱ¹ and Zⁱ² each independently represent any of a single bond or a C1 to C20 alkylene group, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—, and
one —CH₂—CH₂—CH₂—CH₂— in the alkylene group, or each of two or more independently, has optionally been replaced with —CH═N—N═CH—, with the proviso that
an oxygen atom and an oxygen atom are not directly bound together, and
nⁱ¹ represents an integer of 0 to 3, with the proviso that
when multiple Aⁱ²s or Zⁱ²s are present, the Aⁱ²s may be the same or may be different, and the Zⁱ²s may be the same or may be different.) and
one or two or more types of compounds represented by general formula (ii) below

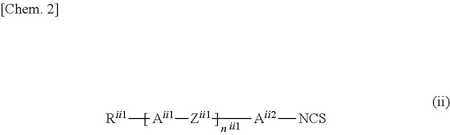

(In general formula (ii),
Rⁱⁱ¹ represents a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, is optionally replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced by a halogen atom, with the proviso that
an oxygen atom and an oxygen atom are not directly bound together,
Aⁱⁱ¹ and Aⁱⁱ² each independently represent a group selected from the group consisting of group (a), group (b), group (c), and group (d) below:
(a) a 1,4-cyclohexylene group (One —CH₂— or two or more nonadjacent —CH₂-s present in the group are optionally replaced by —O— and/or —S—.);
(b) a 1,4-phenylene group (One —CH═ or two or more —CH═s present in the group are optionally replaced by —N═.);
(c) a 1,4-cyclohexenylene group, a bicyclo[2.2.2]octan-1,4-diyl group, a naphthalen-2,6-diyl group, a naphthalen-1,4-diyl group, a 1,2,3,4-tetrahydronaphthalen-2,6-diyl group, a 5,6,7,8-tetrahydronaphthalen-1,4-diyl group, a decahydronaphthalen-2,6-diyl group, an anthracen-2,6-diyl group, an anthracen-1,4-diyl group, an anthracen-9,10-diyl group, or a phenanthren-2,7-diyl group (One —CH═ or two or more —CH═s present in the naphthalen-2,6-diyl group, naphthalen-1,4-diyl group, 1,2,3,4-tetrahydronaphthalen-2,6-diyl group, 5,6,7,8-tetrahydronaphthalen-1,4-diyl group, anthracen-2,6-diyl group, anthracen-1,4-diyl group, anthracen-9,10-diyl group, or phenanthren-2,7-diyl group are optionally replaced by —N═.);
(d) a thiophen-2,5-diyl group, a benzothiophen-2,5-diyl group, a benzothiophen-2,6-diyl group, a benzothiophen-3,7-diyl group, a dibenzothiophen-2,6-diyl group, a thieno[3,2-b]thiophen-2,5-diyl group, or a benzo[1,2-b:4,5-b′]dithiophen-2,6-diyl group (One —CH═ or two or more —CH═s present in the group are optionally replaced by —N═.), where
one hydrogen atom in the Aⁱⁱ¹ and Aⁱⁱ², or each of two or more independently, has optionally been replaced by a substituent Sⁱⁱ¹,
the substituent Sⁱⁱ¹ represents any of a halogen atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced by a halogen atom, with the proviso that
an oxygen atom and an oxygen atom are not directly bound together, and
when there are multiple substituents Sⁱⁱ¹, the substituents may be the same or may be different, Zⁱⁱ¹ represents any of a single bond or a C1 to C20 alkylene group, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—,
one —CH₂—CH₂—CH₂— in the alkylene group, or each of two or more independently, has optionally been replaced with —O—CO—O—, and
an oxygen atom and an oxygen atom are not directly bound together,
nⁱⁱ¹ represents an integer of 1 to 4, and
when multiple Aⁱⁱ¹s and multiple Zⁱⁱ¹s are present, the Aⁱⁱ¹s may be the same or may be different, and the Zⁱⁱ¹s may be the same or may be different.

Compounds represented by general formula (i), however, are excluded.).

Item 2. The liquid crystal composition described in item 1, wherein the compound or compounds represented by general formula (i) are selected from the group consisting of compounds represented by general formula (i-1) to (i-14) below

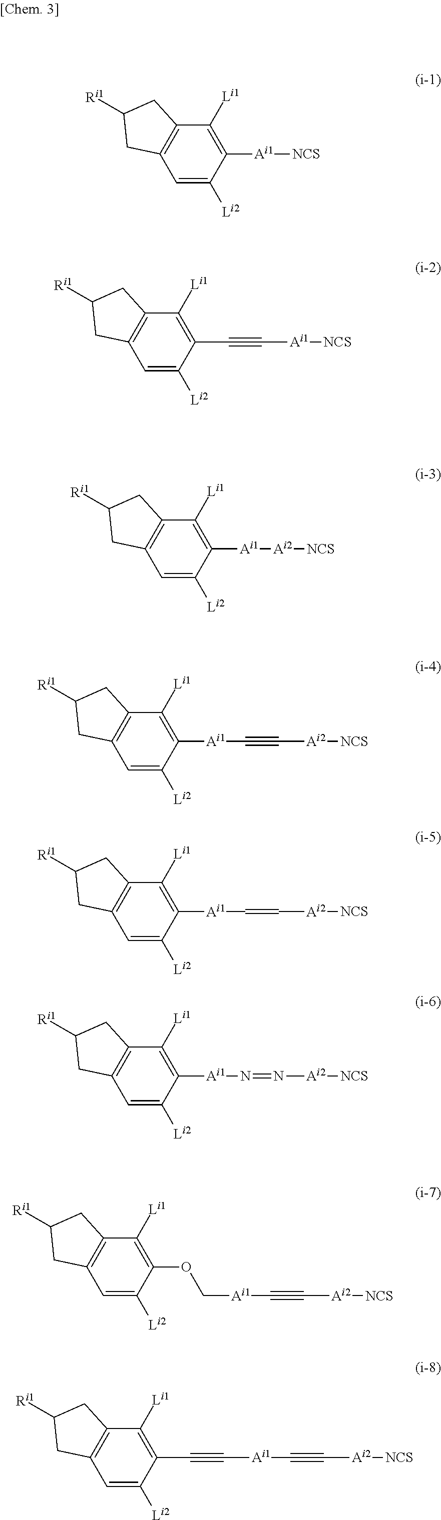

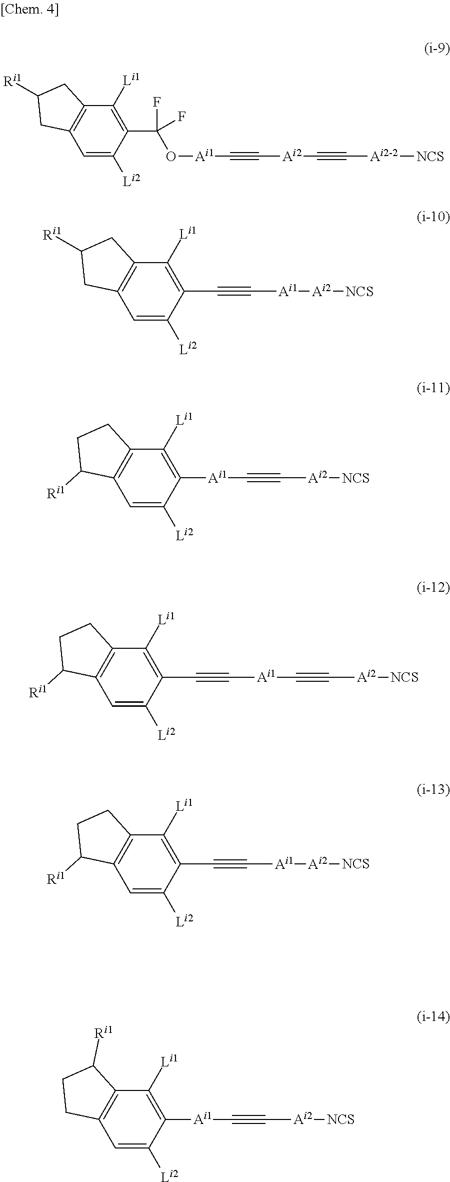

(In general formula (i-1) to (i-14),
Rⁱ¹, Aⁱ¹, Aⁱ², Lⁱ¹, and Lⁱ² have the same meanings as Rⁱ¹, Aⁱ¹, Aⁱ², Lⁱ¹, and Lⁱ², respectively, in general formula (i) above, and
in general formula (i-9), a definition of Aⁱ²⁻² is the same as a definition of Aⁱ² in general formula (i) above.)

Item 3. The liquid crystal composition described in item 1 or 2, wherein the compound or compounds represented by general formula (ii) are selected from the group consisting of compounds represented by general formula (ii-1) to (ii-8) below

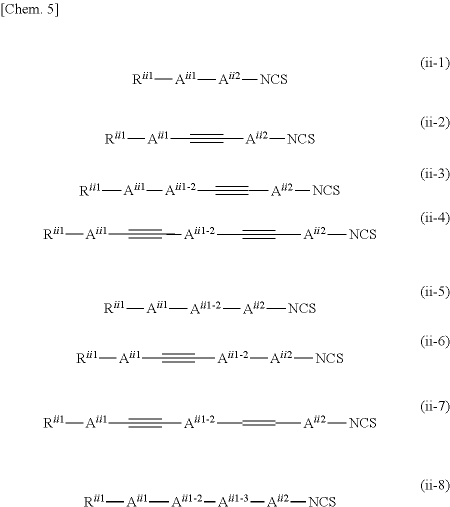

(In general formula (ii-1) to (ii-8),
Rⁱⁱ¹, Aⁱⁱ¹, and Aⁱⁱ² have the same meanings as Rⁱ¹, Aⁱⁱ¹, and Aⁱⁱ², respectively, in general formula (ii) above, and
in general formula (ii-3) to (ii-9), definitions of Aiii-2 and Aⁱⁱ¹⁻³ are each independently the same as a definition of Aⁱⁱ¹ in general formula (ii) above.)

Item 4. The liquid crystal composition described in any one of items 1 to 3, further containing one or two or more types of compounds represented by general formula (vt) below

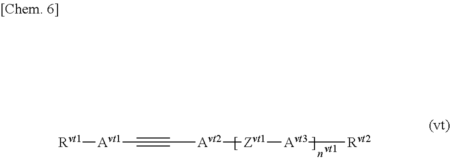

(In general formula (vt),
R^{vt1} represents a hydrogen atom or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced with a halogen atom, with the proviso that
an oxygen atom and an oxygen atom are not directly bound together,
R^{vt2} represents any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced with a halogen atom, with the proviso that
an oxygen atom and an oxygen atom are not directly bound together,
A^{vt1}, A^{vt2}, and A^{vt3} each independently represent any of a C3 to C16 hydrocarbon ring or a C3 to C16 heterocycle, where
one hydrogen atom in the A^{vt1}, A^{vt2}, and A^{vt3}, or each of two or more independently, has optionally been replaced by a substituent S^{vt1},
the substituent S^{vt1} represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, and/or —CO—,
one —CH₂—CH₂— in the alkyl group, or each of two or more independently, is optionally replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, is optionally replaced with —O—CO—O—, and
one hydrogen atom in the alkyl group, or each of two or more independently, has optionally been replaced with a halogen atom, with the proviso that
an oxygen atom and an oxygen atom are not directly bound together, and
when there are multiple substituents S^{vt1}, the substituents may be the same or may be different,
Z^{vt1} represents any of a single bond or a C1 to C20 alkylene group, independently at each occurrence, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—, and
one —CH₂—CH₂—CH₂— in the alkylene group, or each of two or more independently, has optionally been replaced with —O—CO—O—, with the proviso that
an oxygen atom and an oxygen atom are not directly bound together, and
n^{vt1} represents an integer of 1 to 3, with the proviso that
when multiple A^{vt3}s and multiple Z^{vt1}s are present, the A^{vt3}s may be the same or may be different, and the Z^{vt1}s may be the same or may be different.)

Item 5. The liquid crystal composition described in any one of items 1 to 4, wherein Δn at 25° C. and 589 nm is 0.38 or greater.

Item 6. A liquid crystal display element made using the liquid crystal composition described in any one of items 1 to 5.

Item 7. The liquid crystal display element described in item 6, wherein the element operates using an active matrix scheme or a passive matrix scheme.

Item 8. A liquid crystal display element that reversibly switches a dielectric constant by reversibly change a direction of orientation of liquid crystal molecules in the liquid crystal composition described in any one of items 1 to 5.

Item 9. A sensor made using the liquid crystal composition described in any one of items 1 to 5.

Item 10. A liquid crystal lens made using the liquid crystal composition described in any one of items 1 to 5.

Item 11. Optical communication equipment made using the liquid crystal composition described in any one of items 1 to 5.

Item 12. An antenna made using the liquid crystal composition described in any one of items 1 to 5.

Item 13. The antenna described in item 12, wherein:
the antenna includes a first substrate having multiple slots,
a second substrate facing the first substrate and provided with a power feed section,
a first dielectric layer disposed between the first substrate and the second substrate,
multiple patch electrodes positioned corresponding to the multiple slots,
a third substrate provided with the patch electrodes, and
a liquid crystal layer disposed between the first substrate and the third substrate; and
the liquid crystal layer contains the liquid crystal composition described in any one of items 1 to 5.

Item 14. A compound represented by general formula (i) below

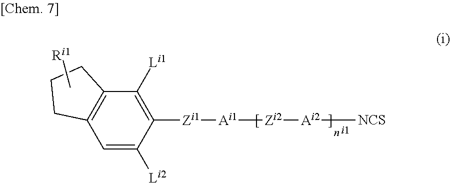

(In general formula (i),
Rⁱ¹ represents a hydrogen atom or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, has optionally been replaced with —CH═CH—CO—O—, —CH═CH—O—CO—, —CO—O—CH═CH—, or —O—CO—CH═CH—, and
one hydrogen atom in the alkyl group, or each of two or more independently, has optionally been replaced with a halogen atom, with the proviso that
an oxygen atom and an oxygen atom are not directly bound together,
Aⁱ¹ and Aⁱ² each independently represent any of a C3 to C16 hydrocarbon ring or a C3 to C16 heterocycle, where
one hydrogen atom in the Aⁱ¹ and Aⁱ², or each of two or more independently, has optionally been replaced by a substituent Sⁱ¹,
the substituent Sⁱ¹ represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, and/or —CO—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, is optionally replaced with —CH═CH—CO—O—, —CH═CH—O—CO—, —CO—O—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together, and
when there are multiple substituents Sⁱ¹, the substituents may be the same or may be different, Lⁱ¹ and Lⁱ² each independently represent any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, is optionally replaced with —O—CO—O—,
one —CH₂—CH₂—CH₂—CH₂— in the alkyl group, or each of two or more independently, has optionally been replaced with —CH═CH—CO—O—, —CH═CH—O—CO—, —COO—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together,
Zⁱ¹ and Zⁱ² each independently represent any of a single bond or a C1 to C20 alkylene group, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—, and
one —CH₂—CH₂—CH₂—CH₂— in the alkylene group, or each of two or more independently, has optionally been replaced with —CH═N—N═CH—, with the proviso that
an oxygen atom and an oxygen atom are not directly bound together, and
nⁱ¹ represents an integer of 0 to 3, with the proviso that
when multiple Aⁱ²s or Zⁱ²s are present, the Aⁱ²s may be the same or may be different, and the Zⁱ²s may be the same or may be different.)

### Advantageous Effects of Invention

According to the present invention, a liquid crystal composition having a high Tₙᵢ, a large Δn, a low Vₜₕ, a large Δεᵣ, and a small tan δᵢₛₒ and having good storability at low temperatures can be obtained by containing one or two or more types of compounds represented by general formula (i), which have an indane structure and an isothiocyanate group (—NCS), and one or two or more types of compounds represented by general formula (ii), which have an isothiocyanate group (—NCS), into a liquid crystal composition. This liquid crystal composition is useful for liquid crystal display elements, sensors, liquid crystal lenses, optical communication equipment, and antennas.

### DESCRIPTION OF EMBODIMENTS

### Compound(s) Represented by General Formula (i)

A liquid crystal composition according to the present invention contains one or two or more types of compounds represented by general formula (i), which have an indane structure and an isothiocyanate group (—NCS).

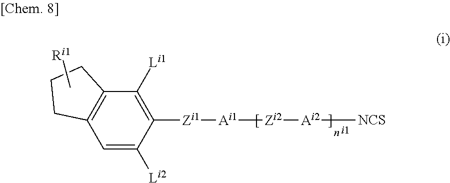

In general formula (i), Rⁱ¹ represents a hydrogen atom or a C1 to C20 alkyl group.

A C1 to C20 alkyl group is a linear-chain, branched, or cyclic alkyl group and preferably is a linear-chain alkyl group.

The number of carbon atoms in the C1 to C20 alkyl group is preferably from two to ten, preferably from two to six.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CF—, and/or —C≡C—.

Moreover, one —CH₂—CH₂—CH₂— in the alkyl group, or each of two or more independently, may have been replaced with —O—CO—O—.

One —CH₂—CH₂—CH₂—CH₂— in the alkyl group, or each of two or more independently, furthermore, may have been replaced with —CH═CH—CO—O—, —CH═CH—O—CO—, —CO—O—CH═CH—, or —O—CO—CH═CH—.

In addition, one hydrogen atom in the alkyl group, or each of two or more independently, may have been replaced with a halogen atom.

Examples of halogen atoms include a fluorine atom, a chlorine atom, and a bromine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

For example, Rⁱ¹ can represent a C1 to C19 alkoxy group as a result of the replacement of one —CH₂— in the alkyl group by —O—. The alkoxy group is a linear-chain, branched, or cyclic alkoxy group and preferably is a linear-chain alkoxy group.

The number of carbon atoms in the alkoxy group is preferably from two to ten, preferably from two to six.

Rⁱ¹, furthermore, can represent a C1 to C19 alkylsulfanyl group (alkylthio group) as a result of the replacement of one —CH₂— in the alkyl group by —S—.

The alkylsulfanyl group is a linear-chain, branched, or cyclic alkylsulfanyl group and preferably is a linear-chain alkylsulfanyl group.

The number of carbon atoms in the alkylsulfanyl group is preferably from two to ten, preferably from two to six.

Moreover, Rⁱ¹ can represent a C2 to C20 alkenyl group as a result of the replacement of one or two or more —CH₂—CH₂-s in the alkyl group by —CH═CH—.

The alkenyl group is a linear-chain, branched, or cyclic alkenyl group and preferably is a linear-chain alkenyl group.

The number of carbon atoms in the alkenyl group is preferably from two to ten, preferably from two to six.

Rⁱ¹, furthermore, can represent a C2 to C20 alkynyl group as a result of the replacement of one or two or more —CH₂—CH₂-s in the alkyl group by —C≡C—.

The alkynyl group is a linear-chain, branched, or cyclic alkynyl group and preferably is a linear-chain alkynyl group.

The number of carbon atoms in the alkynyl group is preferably from two to ten, preferably from two to six.

Moreover, Rⁱ¹ can represent a C2 to C19 alkenyloxy group as a result of the replacement of one —CH₂— in the alkyl group by —O— and the replacement of one or two or more —CH₂—CH₂-s by —CH═CH—.

The alkenyloxy group is a linear-chain, branched, or cyclic alkenyloxy group and preferably is a linear-chain alkenyloxy group.

The number of carbon atoms in the alkenyloxy group is preferably from two to ten, preferably from two to six.

Rⁱ¹, furthermore, can represent a C1 to C20 halogenated alkyl group as a result of the replacement of one or two or more hydrogen atoms in the alkyl group by a halogen atom.

The halogenated alkyl group is a linear-chain, branched, or cyclic halogenated alkyl group and preferably is a linear-chain halogenated alkyl group.

The number of carbon atoms in the halogenated alkyl group is preferably from two to ten, preferably from two to six.

Moreover, Rⁱ¹ can represent a C1 to C19 halogenated alkoxy group as a result of the replacement of one —CH₂— in the alkyl group by —O— and the replacement of one or two or more hydrogen atoms in the alkyl group by a halogen atom.

The halogenated alkoxy group is a linear-chain, branched, or cyclic halogenated alkoxy group and preferably is a linear-chain halogenated alkoxy group.

The number of carbon atoms in the halogenated alkoxy group is preferably from two to ten, preferably from two to six.

Specific examples of C1 to C20 alkyl groups (including substituted ones) at Rⁱ¹ include the groups represented by formula (Rⁱ¹-1) to (Rⁱ¹-36).

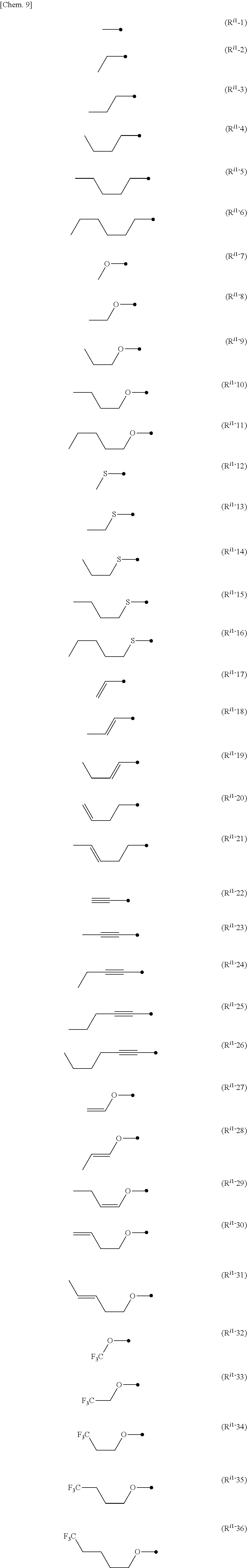

In formula (Rⁱ¹-1) to (Rⁱ¹-36), the black dot represents a bond to Aⁱ¹.

It should be noted that for use as Rⁱ¹, a C2 to C6 linear-chain alkyl group is preferred from the viewpoints of Δn and compatibility with other liquid crystal compounds.

The position in the indane structure at which it is substituted with Rⁱ¹ is preferably any of formula (Rⁱ¹—SP-1) to (Rⁱ¹—SP-3) below. From the viewpoint of the improvement of Δn, (Rⁱ¹—SP-2) or (Rⁱ¹—SP-3) is preferred.

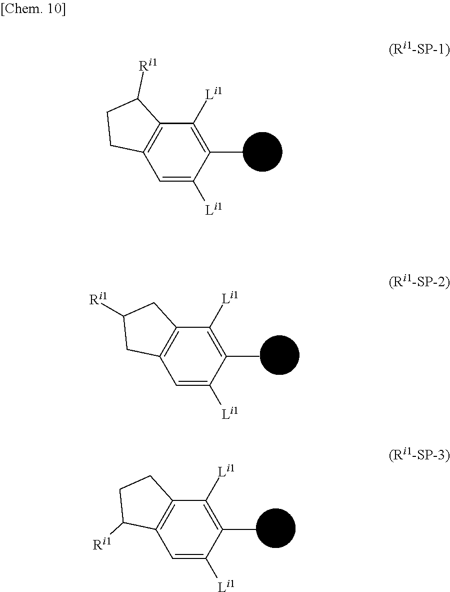

In formula (Rⁱ¹—SP-1) to (Rⁱ¹—SP-3), the black dot represents a bond to Zⁱ¹.

In general formula (i), Aⁱ¹ and Aⁱ² each independently represent any of a C3 to C16 hydrocarbon ring or a C3 to C16 heterocycle.

More specifically, it is preferred that the C3 to C16 hydrocarbon ring or C3 to C16 heterocycle represent a group selected from the group consisting of group (a), group (b), group (c), and group (d) below:
(a) a 1,4-cyclohexylene group (One —CH₂—, or two or more —CH₂-s not adjacent to each other, present in the group may be replaced by —O— or —S—.);
(b) a 1,4-phenylene group (One —CH═, or two or more —CH═s not adjacent to each other, present in the group may be replaced by —N═.);
(c) a 1,4-cyclohexenylene group, a bicyclo[2.2.2]octan-1,4-diyl group, a naphthalen-2,6-diyl group, a naphthalen-1,4-diyl group, a 1,2,3,4-tetrahydronaphthalen-2,6-diyl group, a 5,6,7,8-tetrahydronaphthalen-1,4-diyl group, a decahydronaphthalen-2,6-diyl group, an anthracen-2,6-diyl group, an anthracen-1,4-diyl group, an anthracen-9,10-diyl group, or a phenanthren-2,7-diyl group (One —CH═ or two or more —CH═s present in a naphthalen-2,6-diyl group, naphthalen-1,4-diyl group, 1,2,3,4-tetrahydronaphthalen-2,6-diyl group, 5,6,7,8-tetrahydronaphthalen-1,4-diyl group, anthracen-2,6-diyl group, anthracen-1,4-diyl group, anthracen-9,10-diyl group, or phenanthren-2,7-diyl group may be replaced by —N═.);
(d) a thiophen-2,5-diyl group, a benzothiophen-2,5-diyl group, a benzothiophen-2,6-diyl group, a benzothiophen-3,7-diyl group, a dibenzothiophen-2,6-diyl group, or a thieno[3,2-b]thiophen-2,5-diyl group (One —CH═, or two or more —CH═s not adjacent to each other, present in the group may be replaced by —N═.)

One hydrogen atom in Aⁱ¹ and Aⁱ², or each of two or more independently, may have been replaced by a substituent Sⁱ¹.

The substituent Sⁱ¹ represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group.

The alkyl group is a linear-chain, branched, or cyclic alkyl group and preferably is a linear-chain alkyl group.

The number of carbon atoms in the alkyl group is preferably from two to ten, preferably from three to six.

One —CH₂— in the alkyl group, or each of two or more independently, may have been replaced with —O—, —S—, and/or —CO—.

One —CH₂—CH₂— in the alkyl group, or each of two or more independently, furthermore, may be replaced with —CH═CH—, —CF═CF—, —C≡C—, —CO—O—, —O—CO—, —CO—S—, —S—CO—, —CO—NH—, and/or —NH—CO—.

Moreover, one —CH₂—CH₂—CH₂— in the alkyl group, or each of two or more independently, may be replaced with —O—CO—O—.

One —CH₂—CH₂—CH₂—CH₂— in the alkyl group, or each of two or more independently, furthermore, may be replaced with —CH═CH—CO—O—, —CH═CH—O—CO—, —CO—O—CH═CH—, and/or —O—CO—CH═CH—.

One hydrogen atom in the alkyl group, or each of two or more independently, may have been replaced with a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

The substituent Sⁱ¹ is preferably a fluorine atom.

It is, furthermore, preferred that at least one of Aⁱ¹ or Aⁱ² be substituted with at least one substituent Sⁱ¹.

Moreover, Aⁱ² is preferably substituted with at least one substituent Sⁱ¹.

It should be noted that when there are multiple Sⁱ¹s, they may be the same or may be different.

The position in Aⁱ¹ at which it is substituted with a substituent or substituents Sⁱ¹ is preferably any of formula (Aⁱ¹-SP-1) to (Aⁱ¹-SP-3) below.

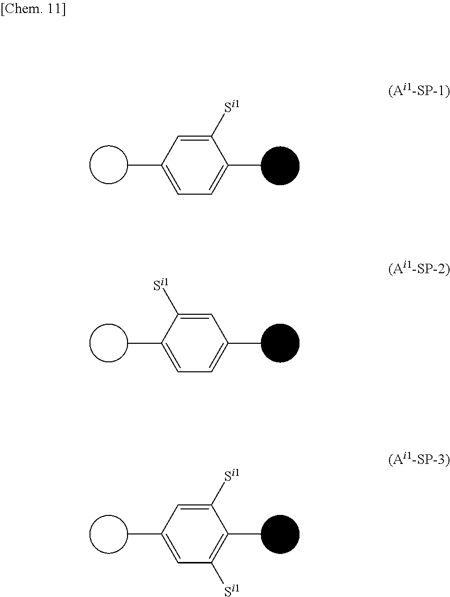

In formula (Aⁱ¹-SP-1) to (Aⁱ¹-SP-3), the white dot represents a bond to Zⁱ¹, and the black dot represents a bond to Zⁱ² or the isothiocyanate group (—NCS).

The position in Aⁱ² at which it is substituted with a substituent or substituents Sⁱ¹ is preferably any of formula (Aⁱ²-SP-1) to (Aⁱ²-SP-3) below.

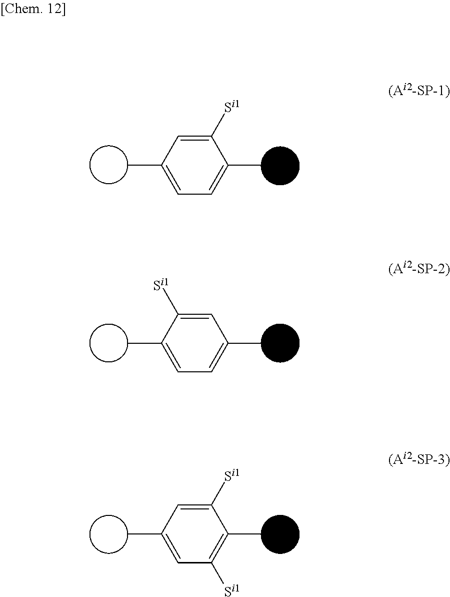

In formula (Aⁱ²-SP-1) to (Aⁱ²-SP-3), the white dot represents a bond to Zⁱ², and the black dot represents a bond to Zⁱ² or the isothiocyanate group (—NCS).

More specifically, it is preferred that Aⁱ¹ represent any of formula (Aⁱ¹-1) to (Aⁱ¹-8) below.

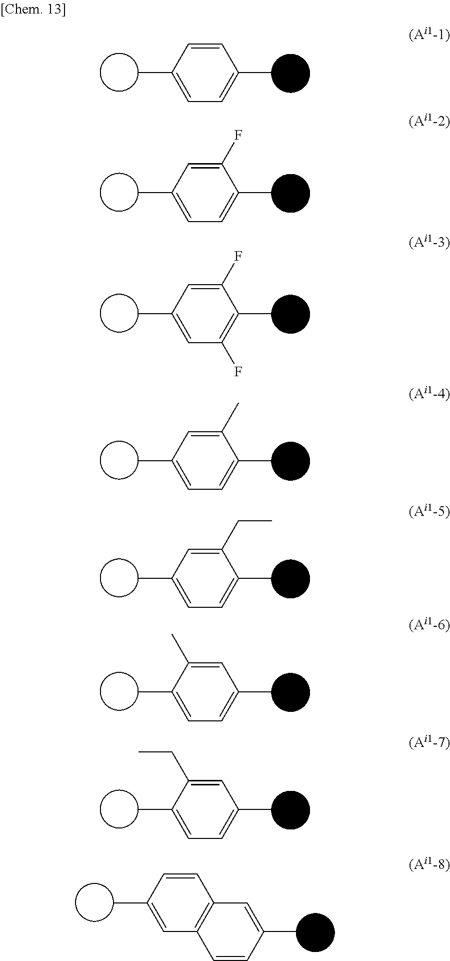

In formula (Aⁱ¹-1) to (Aⁱ¹-8), the white dot represents a bond to Zⁱ¹, and the black dot represents a bond to Zⁱ² or the isothiocyanate group (—NCS).

More specifically, it is preferred that Aⁱ² represent any of formula (Aⁱ²-1) to (Aⁱ²-5) below.

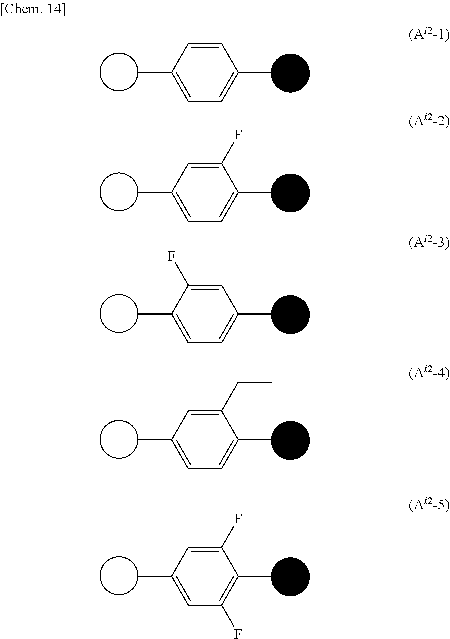

In formula (Aⁱ²-1) to (Aⁱ²-5), the white dot represents a bond to Zⁱ², and the black dot represents a bond to Zⁱ² or the isothiocyanate group (—NCS).

In general formula (i), Lⁱ¹ and Lⁱ² each independently represent any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group.

A C1 to C20 alkyl group is a linear-chain, branched, or cyclic alkyl group and preferably is a linear-chain alkyl group.

The number of carbon atoms in the C1 to C20 alkyl group is preferably from two to ten, preferably from two to six.

One —CH₂— in the alkyl group or each of two or more independently, may have been replaced with —O—, —S—, —CO—, and/or —CS—.

One —CH₂—CH₂— in the alkyl group, or each of two or more independently, furthermore, may be replaced with —CH═CH—, —CF═CF—, —C≡C—, —CO—O—, —O—CO—, —CO—S—, —S—CO—, —CO—NH—, and/or —NH—CO—.

Moreover, one —CH₂—CH₂—CH₂— in the alkyl group, or each of two or more independently, may be replaced with —O—CO—O—.

One —CH₂—CH₂—CH₂—CH₂— in the alkyl group, or each of two or more independently, furthermore, may be replaced with —CH═CH—CO—O—, —CH═CH—O—CO—, —CO—O—CH═CH—, and/or —O—CO—CH═CH—.

Moreover, one hydrogen atom in the alkyl group, or each of two or more independently, may have been replaced with a halogen atom.

Examples of halogen atoms include a fluorine atom, a chlorine atom, and a bromine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

For example, Lⁱ¹ and Lⁱ² can represent C1 to C19 alkoxy groups as a result of the replacement of one —CH₂— in the alkyl groups by —O—.

The alkoxy groups are linear-chain, branched, or cyclic alkoxy groups and preferably are linear-chain alkoxy groups.

The number of carbon atoms in the alkoxy groups is preferably from two to ten, preferably from two to six.

Lⁱ¹ and Lⁱ², furthermore, can represent C1 to C19 alkylsulfanyl groups (alkylthio groups) as a result of the replacement of one —CH₂— in the alkyl groups by —S—.

The alkylsulfanyl groups are linear-chain, branched, or cyclic alkylsulfanyl groups and preferably are linear-chain alkylsulfanyl groups.

The number of carbon atoms in the alkylsulfanyl groups is preferably from two to ten, preferably from two to six.

Moreover, Lⁱ¹ and Lⁱ² can represent C2 to C20 alkenyl groups as a result of the replacement of one or two or more —CH₂—CH₂-s in the alkyl groups by —CH═CH—.

The alkenyl groups are linear-chain, branched, or cyclic alkenyl groups and preferably are linear-chain alkenyl groups.

The number of carbon atoms in the alkenyl groups is preferably from two to ten, preferably from two to six.

Lⁱ¹ and Lⁱ², furthermore, can represent C2 to C20 alkynyl groups as a result of the replacement of one or two or more —CH₂—CH₂-s in the alkyl groups by —C≡C—.

The alkynyl group are linear-chain, branched, or cyclic alkynyl groups and preferably are linear-chain alkynyl groups.

The number of carbon atoms in the alkynyl groups is preferably from two to ten, preferably from two to six.

Moreover, Lⁱ¹ and Lⁱ² can represent C2 to C19 alkenyloxy groups as a result of the replacement of one —CH₂— in the alkyl groups by —O— and the replacement of one or two or more —CH₂—CH₂-s by —CH═CH—.

The alkenyloxy groups are linear-chain, branched, or cyclic alkenyloxy groups and preferably are linear-chain alkenyloxy groups.

The number of carbon atoms in the alkenyloxy groups is preferably from two to ten, preferably from two to six.

Lⁱ¹ and Lⁱ², furthermore, can represent C1 to C20 halogenated alkyl groups as a result of the replacement of one or two or more hydrogen atoms in the alkyl groups by a halogen atom.

The halogenated alkyl groups are linear-chain, branched, or cyclic halogenated alkyl groups and preferably are linear-chain halogenated alkyl groups.

The number of carbon atoms in the halogenated alkyl groups is preferably from two to ten, preferably from two to six.

Moreover, Lⁱ¹ and Lⁱ² can represent C1 to C19 halogenated alkoxy groups as a result of the replacement of one —CH₂— in the alkyl groups by —O— and the replacement of one or two or more hydrogen atoms in the alkyl groups by a halogen atom.

The halogenated alkoxy groups are linear-chain, branched, or cyclic halogenated alkoxy groups and preferably are linear-chain halogenated alkoxy groups.

The number of carbon atoms in the halogenated alkoxy groups is preferably from two to ten, preferably from two to six.

Specific examples of C1 to C20 alkyl groups (including substituted ones) at Lⁱ¹ and Lⁱ² include the groups represented by formula (L^{i1/2}-1) to (L^{i1/2}-36).

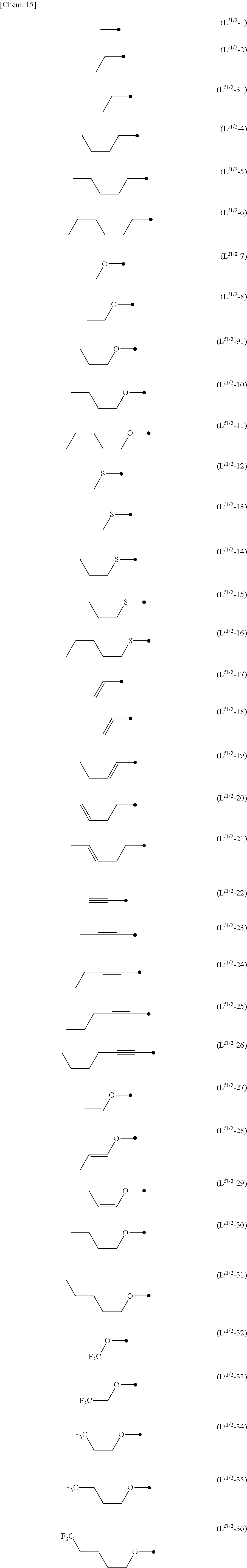

In formula (L^{i1/2}-1) to (L^{i1/2}-36), the black dot represents a bond to the indane structure.

From the viewpoint of compatibility with other liquid crystal compounds, it is preferred that at least one of Lⁱ¹ or Lⁱ² be a hydrogen atom or fluorine atom, and it is preferred that Lⁱ¹ and Lⁱ² be both hydrogen atoms or fluorine atoms.

Zⁱ¹ and Zⁱ² each independently represent any of a single bond or a C1 to C20 alkylene group.

The alkylene group is a linear-chain, branched, or cyclic alkylene group and preferably is a linear-chain alkylene group.

The number of carbon atoms in the alkylene group is preferably from two to ten, preferably from two to six.

One —CH₂— in the alkylene group, or each of two or more independently, may have been replaced with —O—, —CF₂—, and/or —CO—.

One —CH₂—CH₂— in the alkylene group, or each of two or more independently, furthermore, may be replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—.

Moreover, one —CH₂—CH₂—CH₂—CH₂— in the alkylene group, or each of two or more independently may have been replaced with —CH═N—N═CH—.

When the alkylene group is substituted with a predetermined group or groups, however, an oxygen atom and an oxygen atom are not directly bound together.

Specific examples of C1 to C20 alkylene groups (including substituted ones) include the groups represented by formula (Z^{i1/2}-1) to (Z^{i1/2}-24).

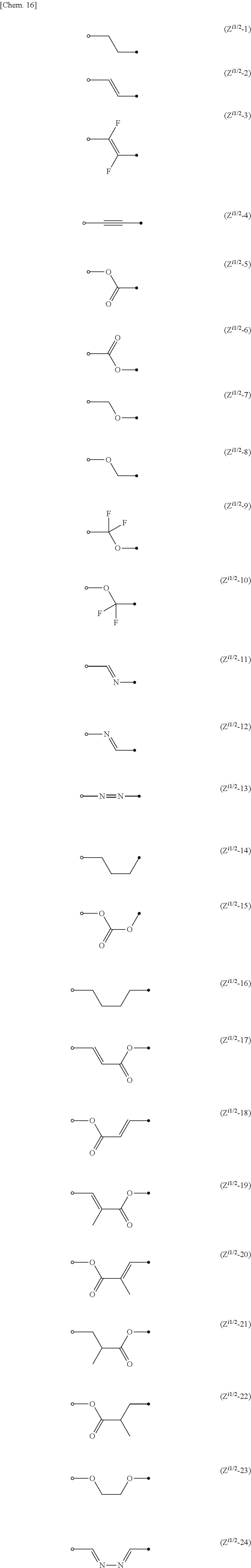

In formula (Z^{i1/2}-1) to (Z^{i1/2}-24), the white dot represents a bond to the indane structure, Aⁱ¹, or Aⁱ², and the black dot represents a bond to Aⁱ¹ or Aⁱ².

From the viewpoints of the improvement of Δn and low viscosity, it is preferred that at least one of Zⁱ¹ or Zⁱ² be formula (Z^{i1/2}-4) (—C≡C—), and it is preferred that Zⁱ¹ and Zⁱ² be both formula (Z^{i1/2}-4) (—C≡C—).

In general formula (i), nⁱ¹ represents an integer of 0 to 3.

From the viewpoints of compatibility with other liquid crystal compounds, phase transition temperatures, dielectric anisotropy, ease of synthesis, and availability of raw materials, it is preferred that nⁱ¹ be 1 or 2.

When multiple Aⁱ²s or Zⁱ²s are present, the Aⁱ²s may be the same or may be different, and the Zⁱ²s may be the same or may be different.

The compound or compounds represented by general formula (i) are preferably at least one compound represented by general formula (i-1) to (i-14) below.

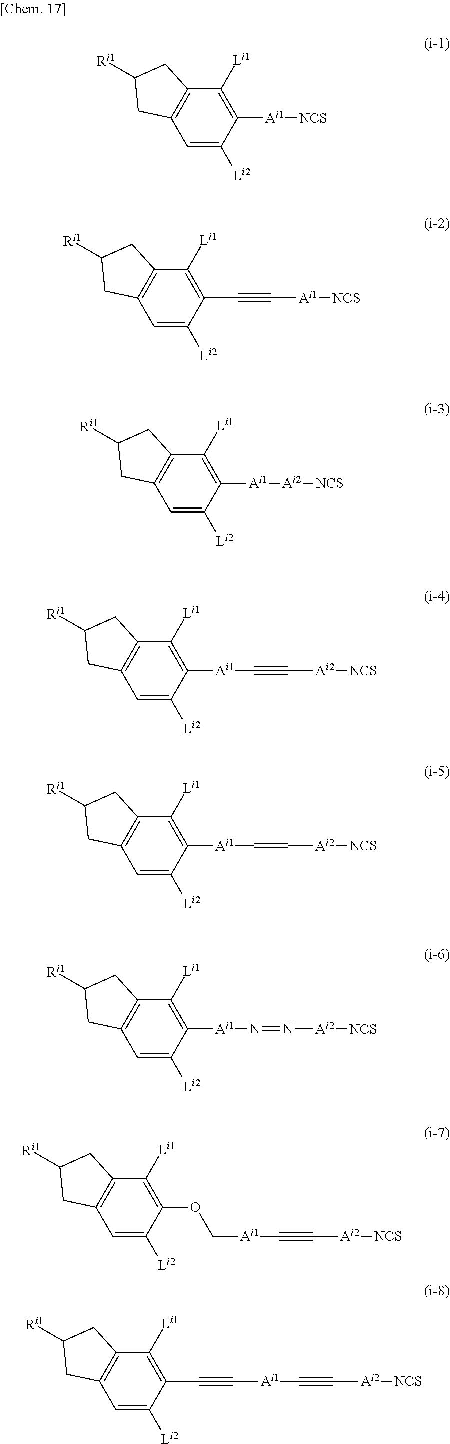

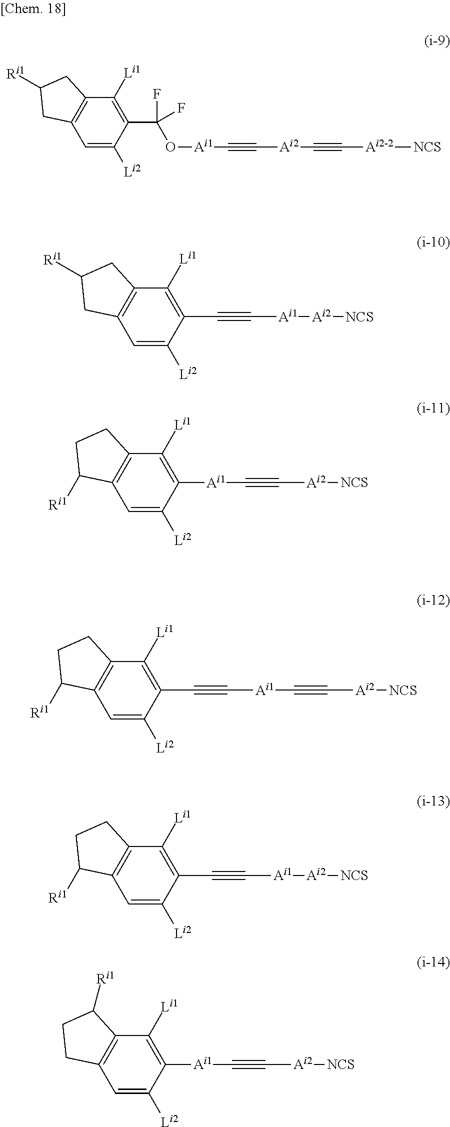

In general formula (i-1) to (i-14), Rⁱ¹, Aⁱ¹, Aⁱ², Lⁱ¹, and Lⁱ² each independently have the same meanings as Rⁱ¹, Aⁱ¹, Aⁱ², Lⁱ¹, and Lⁱ² in general formula (i) above.

In general formula (i-9), furthermore, the definition of Aⁱ²⁻² is the same as the definition of Aⁱ² in general formula (i) above.

Compounds represented by general formula (i-1) are preferably compounds represented by general formula (i-1-1) and (i-1-2) below.

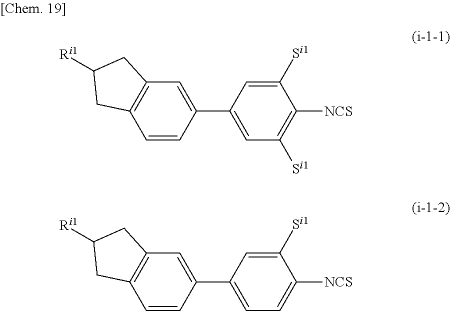

In general formula (i-1-1) and (i-1-2), Rⁱ¹ and Sⁱ¹ each independently have the same meanings as Rⁱ¹ and Sⁱ¹ in general formula (i) above.

Specific examples of compounds represented by general formula (i-1-1) include the compounds represented by structural formula (i-1-1.1) to (i-1-1.3) below.

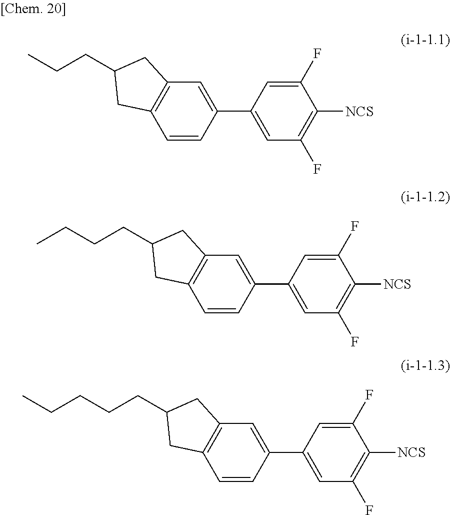

Specific examples of compounds represented by general formula (i-1-2) include the compounds represented by structural formula (i-1-2.1) to (i-1-2.3) below.

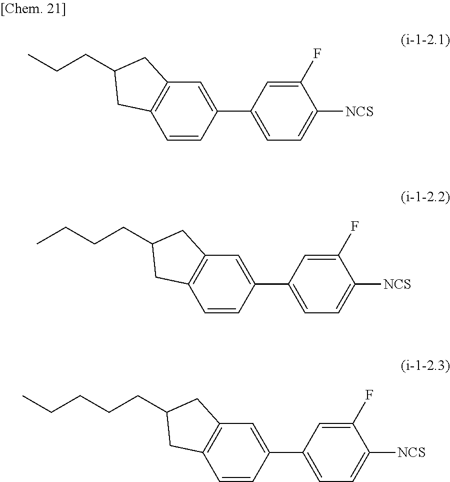

Compounds represented by general formula (i-2) are preferably compounds represented by general formula (i-2-1) to (i-2-4) below.

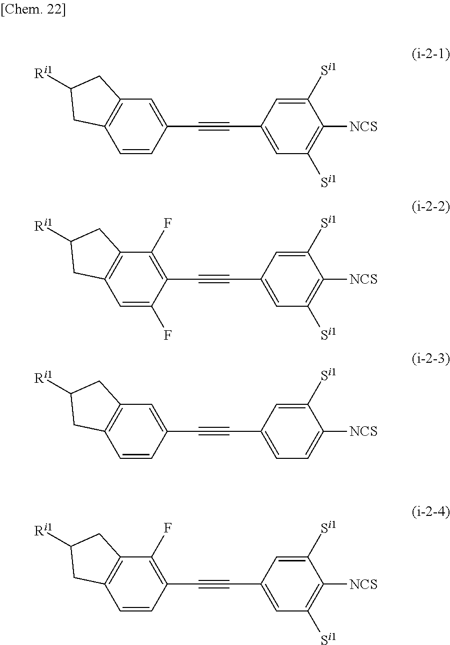

In general formula (i-2-1) to (i-2-4), Rⁱ¹ and Sⁱ¹ each independently have the same meanings as Rⁱ¹ and Sⁱ¹ in general formula (i) above.

Specific examples of compounds represented by general formula (i-2-1) include the compounds represented by structural formula (i-2-1.1) to (i-2-1.3) below.

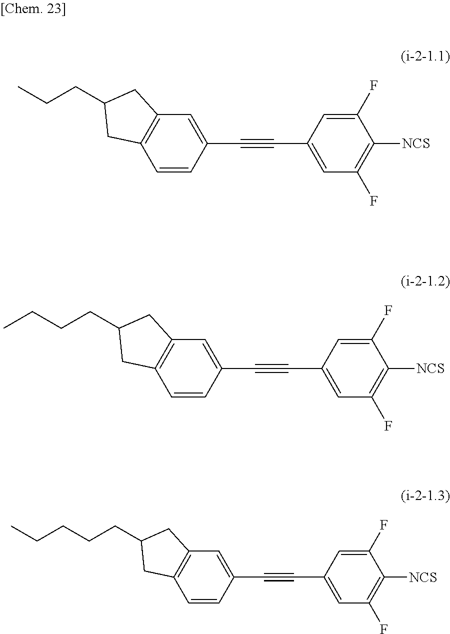

Specific examples of compounds represented by general formula (i-2-2) include the compounds represented by structural formula (i-2-2.1) to (i-2-2.3) below.

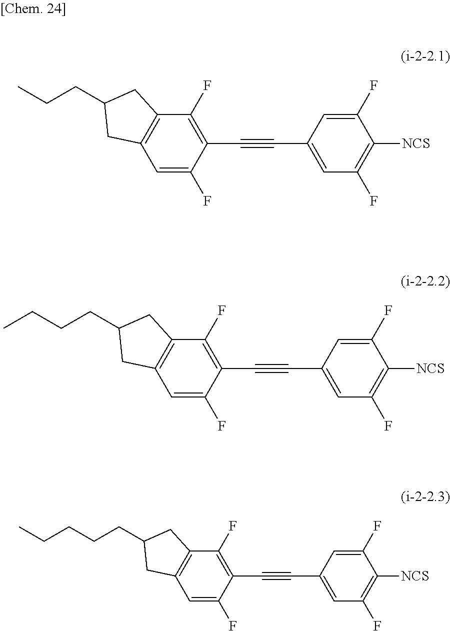

Specific examples of compounds represented by general formula (i-2-3) include the compounds represented by structural formula (i-2-3.1) to (i-2-3.3) below.

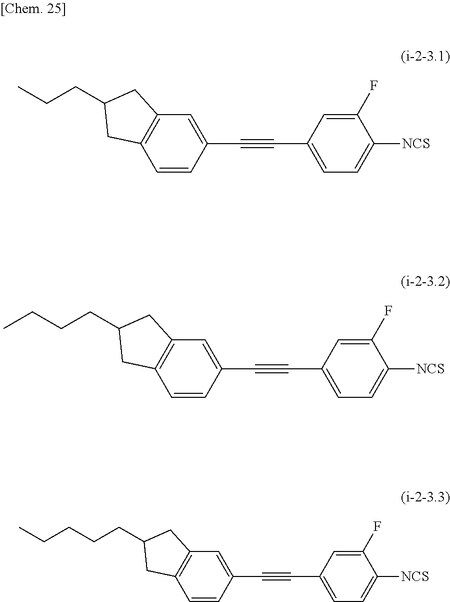

Specific examples of compounds represented by general formula (i-2-4) include the compound represented by structural formula (i-2-4.1) below.

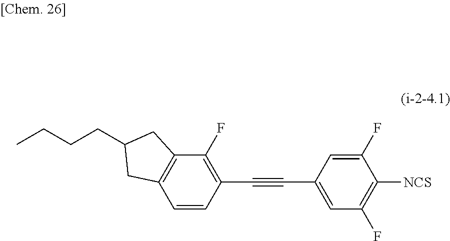

Compounds represented by general formula (i-3) are preferably compounds represented by general formula (i-3-1) to (i-3-3).

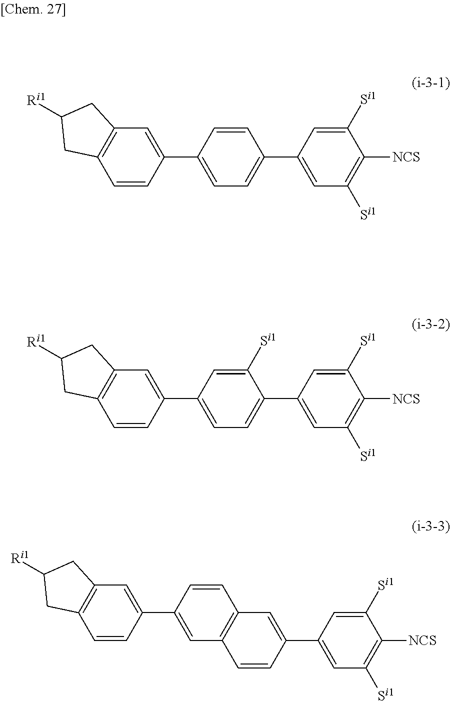

In general formula (i-3-1) to (i-3-3), Rⁱ¹ and Sⁱ¹ each independently have the same meanings as Rⁱ¹ and Sⁱ¹ in general formula (i) above.

Specific examples of compounds represented by general formula (i-3-1) include the compounds represented by structural formula (i-3-1.1) to (i-3-1.3) below.

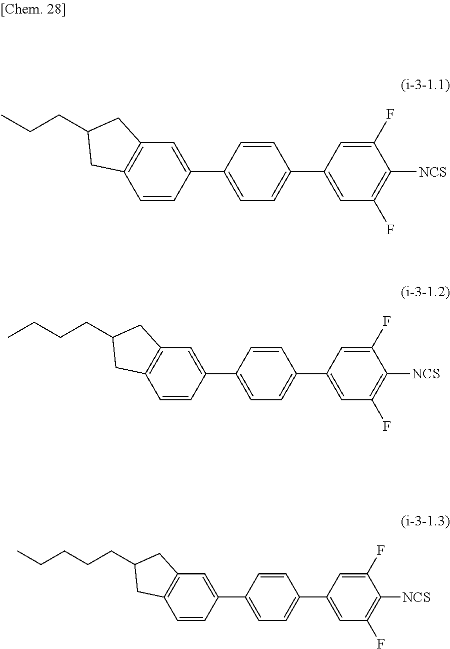

Specific examples of compounds represented by general formula (i-3-2) include the compounds represented by structural formula (i-3-2.1) to (i-3-2.3) below.

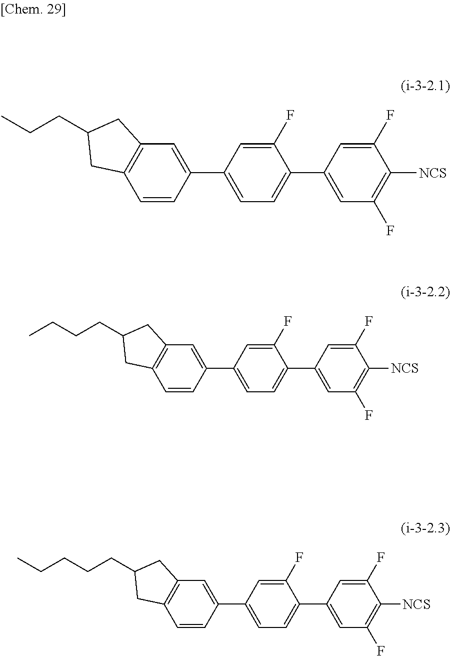

Specific examples of compounds represented by general formula (i-3-3) include the compounds represented by structural formula (i-3-3.1) to (i-3-3.3) below.

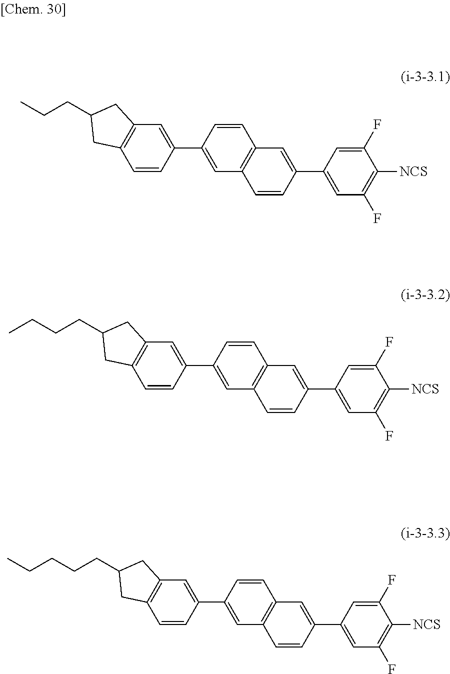

Compounds represented by general formula (i-4) are preferably compounds represented by general formula (i-4-1) to (i-4-6).

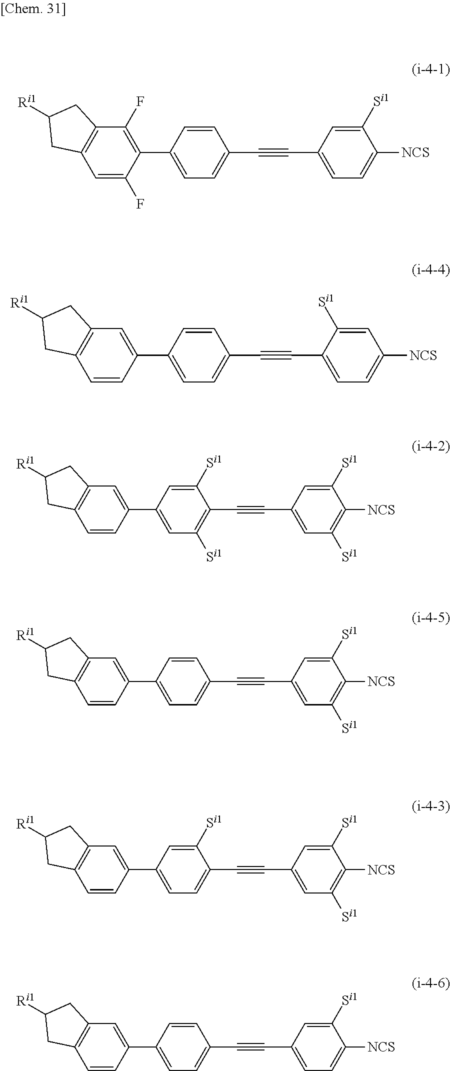

In general formula (i-4-1) to (i-4-6), Rⁱ¹ and Sⁱ¹ each independently have the same meanings as Rⁱ¹ and Sⁱ¹ in general formula (i) above.

Specific examples of compounds represented by general formula (i-4-1) include the compounds represented by structural formula (i-4-1.1) to (i-4-1.3) below.

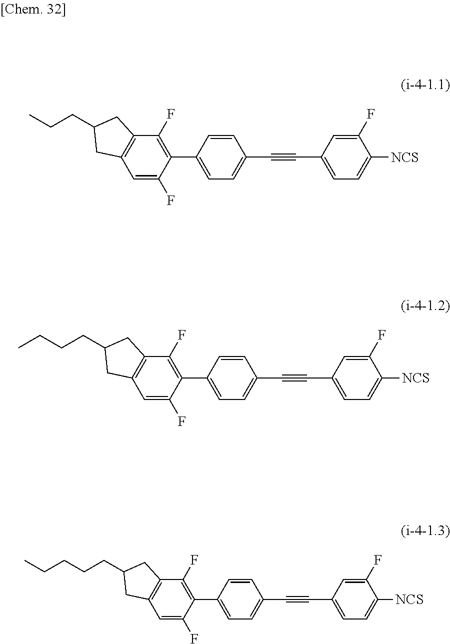

Specific examples of compounds represented by general formula (i-4-2) include the compounds represented by structural formula (i-4-2.1) to (i-4-2.3) below.

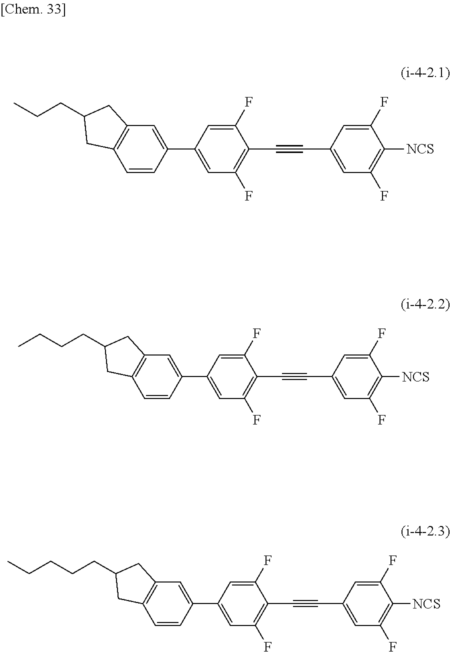

Specific examples of compounds represented by general formula (i-4-3) include the compounds represented by structural formula (i-4-3.1) to (i-4-3.3) below.

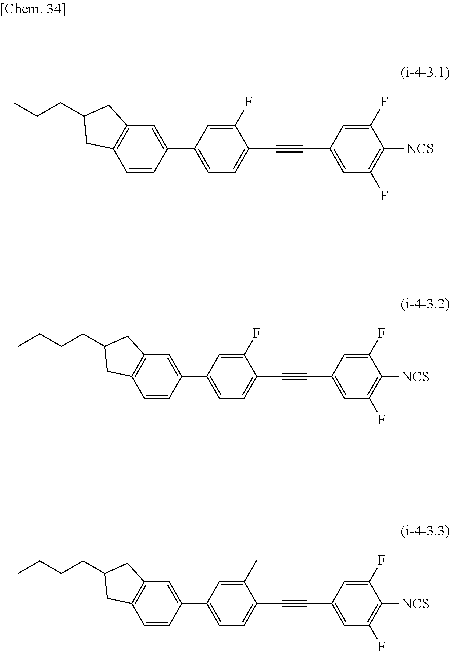

Specific examples of compounds represented by general formula (i-4-4) include the compounds represented by structural formula (i-4-4.1) to (i-4-4.3) below.

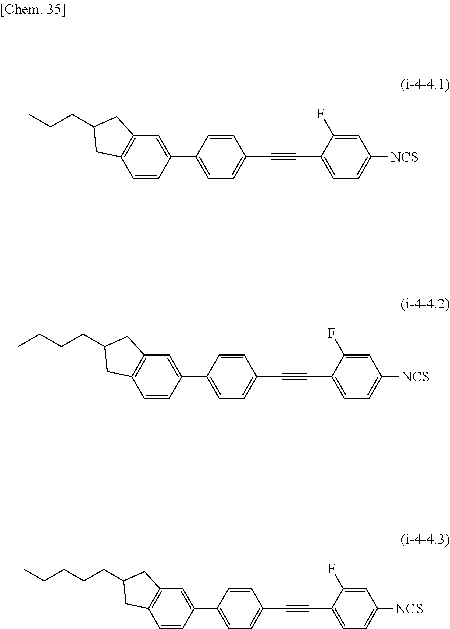

Specific examples of compounds represented by general formula (i-4-5) include the compounds represented by structural formula (i-4-5.1) to (i-4-5.4) below.

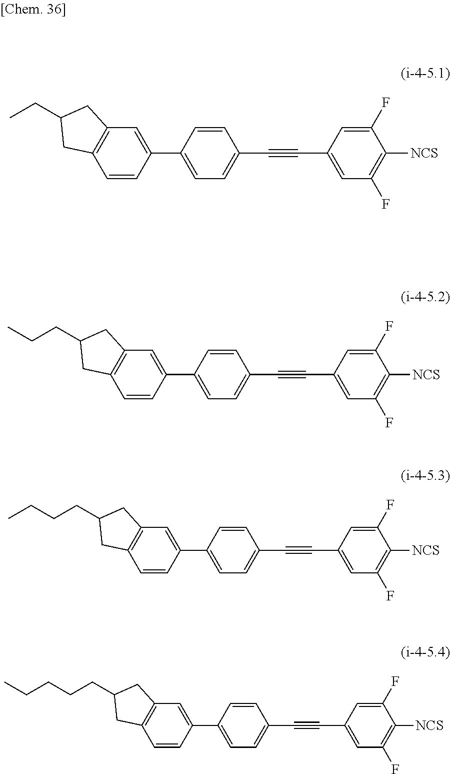

Specific examples of compounds represented by general formula (i-4-6) include the compounds represented by structural formula (i-4-6.1) to (i-4-6.3) below.

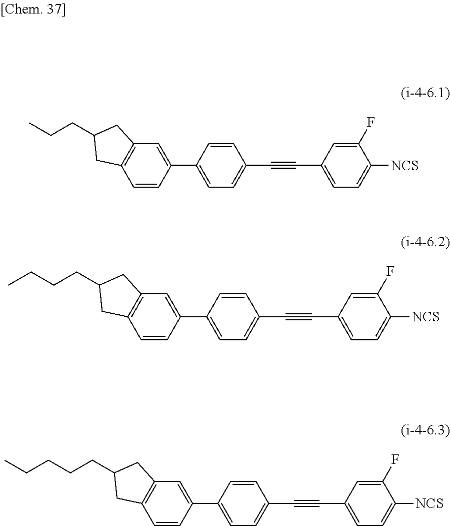

Compounds represented by general formula (i-5) are preferably compounds represented by general formula (i-5-1).

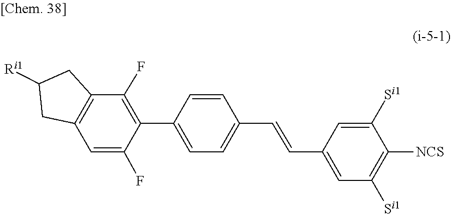

In general formula (i-5-1), Rⁱ¹ and Sⁱ¹ each independently have the same meanings as Rⁱ¹ and Sⁱ¹ in general formula (i) above.

Specific examples of compounds represented by general formula (i-5-1) include the compounds represented by structural formula (i-5-1.1) and (i-5-1.2) below.

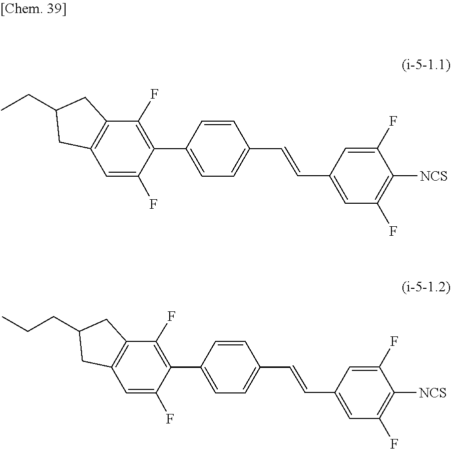

Compounds represented by general formula (i-6) are preferably compounds represented by general formula (i-6-1) and (i-6-2).

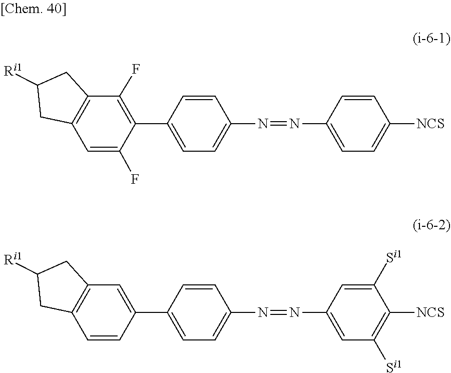

In general formula (i-6-1) and (i-6-2), Rⁱ¹ and Sⁱ¹ each independently have the same meanings as Rⁱ¹ and Sⁱ¹ in general formula (i) above.

Specific examples of compounds represented by general formula (i-6-1) include the compounds represented by structural formula (i-6-1.1) and (i-6-1.2) below.

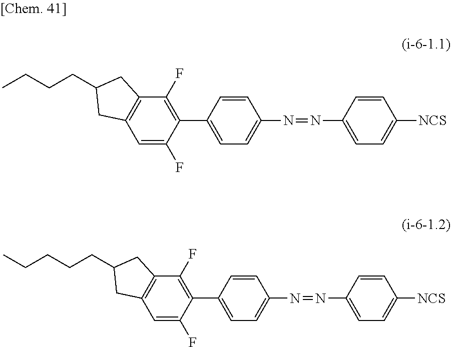

Specific examples of compounds represented by general formula (i-6-2) include the compounds represented by structural formula (i-6-2.1) and (i-6-2.2) below.

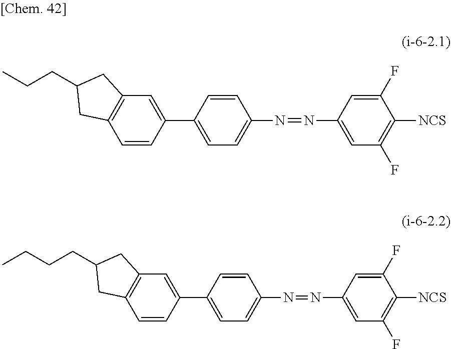

Compounds represented by general formula (i-7) are preferably compounds represented by general formula (i-7-1) and (i-7-2) below.

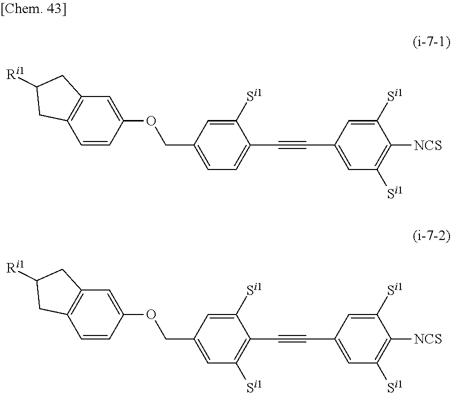

In general formula (i-7-1) and (i-7-2), Rⁱ¹ and Sⁱ¹ each independently have the same meanings as Rⁱ¹ and Sⁱ¹ in general formula (i) above.

Specific examples of compounds represented by general formula (i-7-1) include the compound represented by structural formula (i-7-1.1) below.

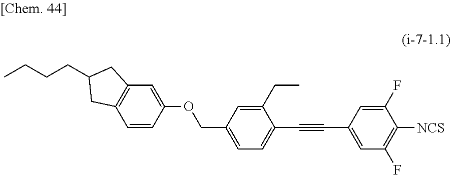

Specific examples of compounds represented by general formula (i-7-2) include the compound represented by structural formula (i-7-2.1) below.

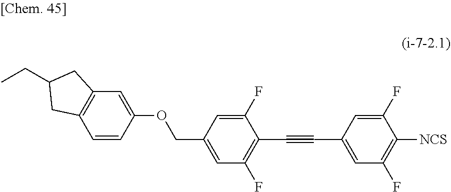

Compounds represented by general formula (i-8) are preferably compounds represented by general formula (i-8-1) to (i-8-5) below.

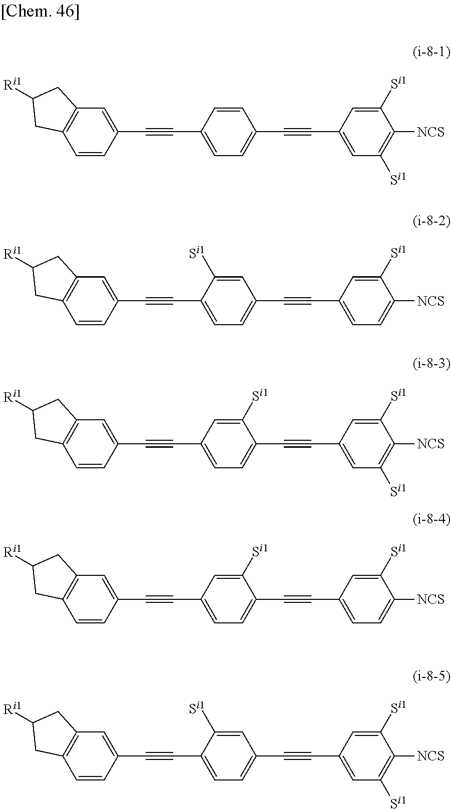

In general formula (i-8-1) to (i-8-5), Rⁱ¹ and Sⁱ¹ each independently have the same meanings as Rⁱ¹ and Sⁱ¹ in general formula (i) above.

Specific examples of compounds represented by general formula (i-8-1) include the compounds represented by structural formula (i-8-1.1) and (i-8-1.2) below.

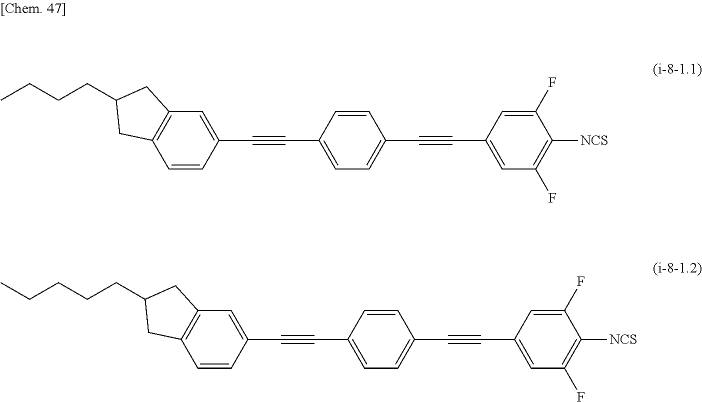

Specific examples of compounds represented by general formula (i-8-2) include the compounds represented by structural formula (i-8-2.1) and (i-8-2.2) below.

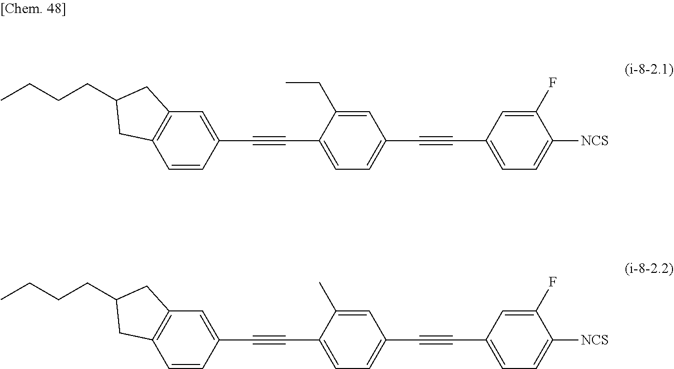

Specific examples of compounds represented by general formula (i-8-3) include the compounds represented by structural formula (i-8-3.1) to (i-8-3.4) below.

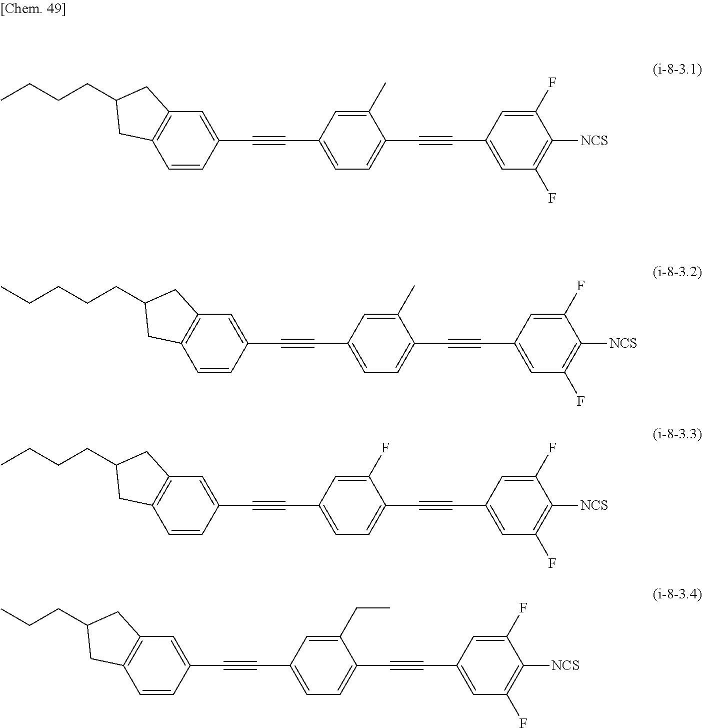

Specific examples of compounds represented by general formula (i-8-4) include the compounds represented by structural formula (i-8-4.1) and (i-8-4.2) below.

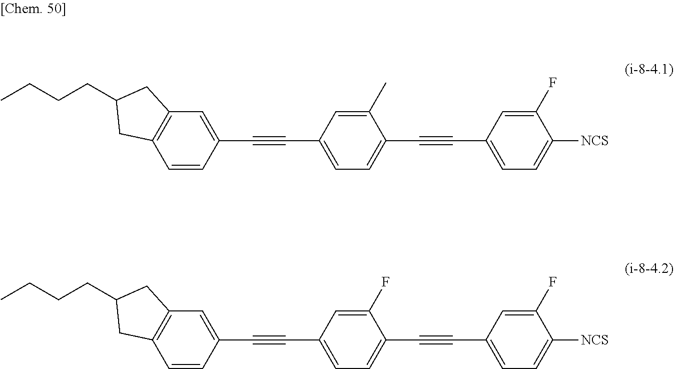

Specific examples of compounds represented by general formula (i-8-5) include the compounds represented by structural formula (i-8-5.1) and (i-8-5.2) below.

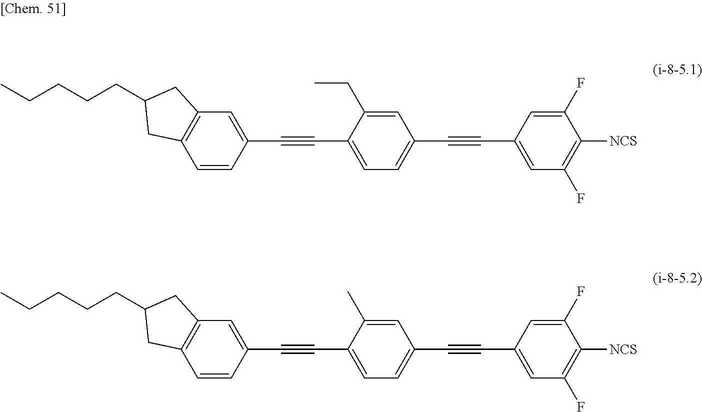

Compounds represented by general formula (i-9) are preferably compounds represented by general formula (i-9-1) below.

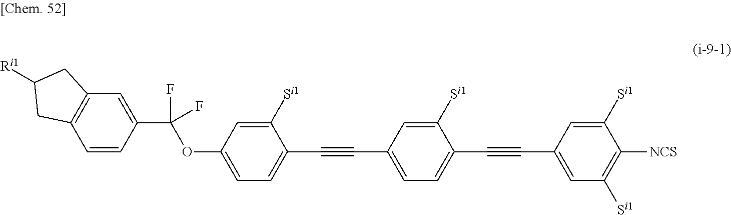

In general formula (i-9-1), Rⁱ¹ and Sⁱ¹ each independently have the same meanings as Rⁱ¹ and Sⁱ¹ in general formula (i) above.

Specific examples of compounds represented by general formula (i-9-1) include the compound represented by structural formula (i-9-1.1) below.

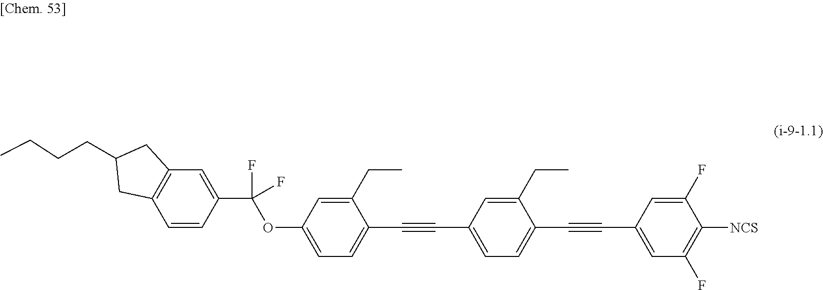

Compounds represented by general formula (i-10) are preferably compounds represented by general formula (i-10-1) to (i-10-3) below.

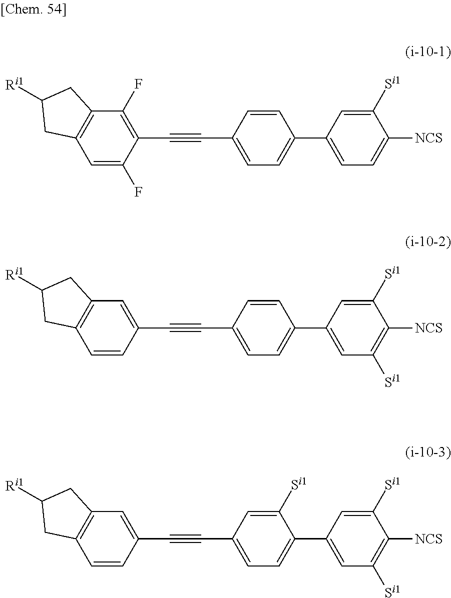

In general formula (i-10-1) to (i-10-3), Rⁱ¹ and Sⁱ¹ each independently have the same meanings as Rⁱ¹ and Sⁱ¹ in general formula (i) above.

Specific examples of compounds represented by general formula (i-10-1) include the compounds represented by structural formula (i-10-1.1) to (i-10-1.3) below.

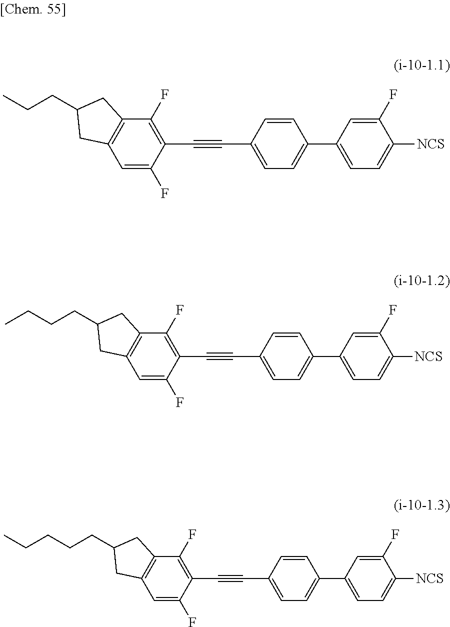

Specific examples of compounds represented by general formula (i-10-2) include the compounds represented by structural formula (i-10-2.1) to (i-10-2.4) below.

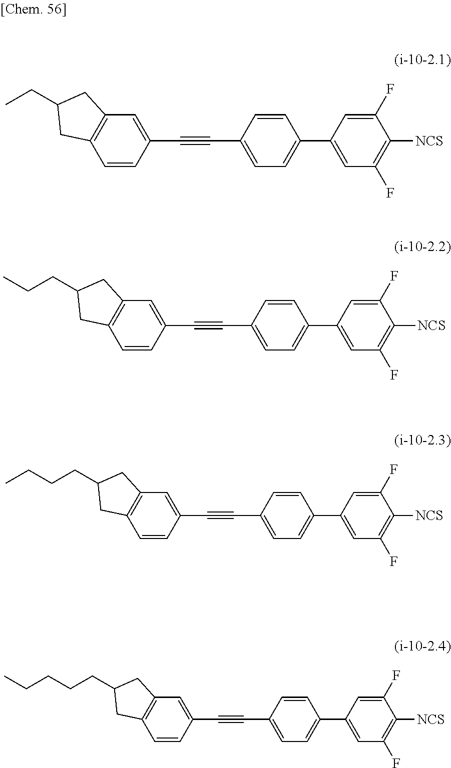

Specific examples of compounds represented by general formula (i-10-3) include the compounds represented by structural formula (i-10-3.1) to (i-10-3.3) below.

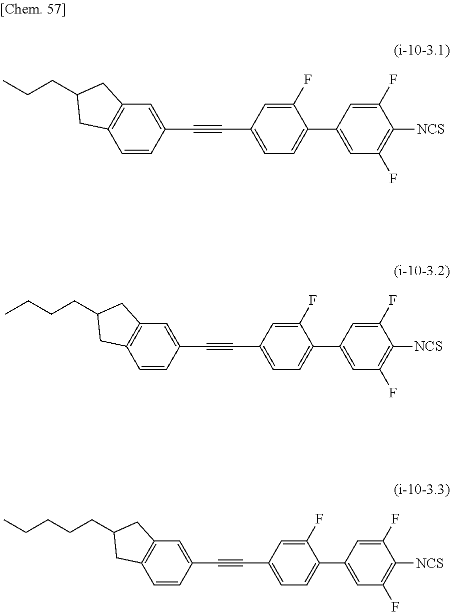

Compounds represented by general formula (i-11) are preferably compounds represented by general formula (i-11-1) to (i-11-6) below.

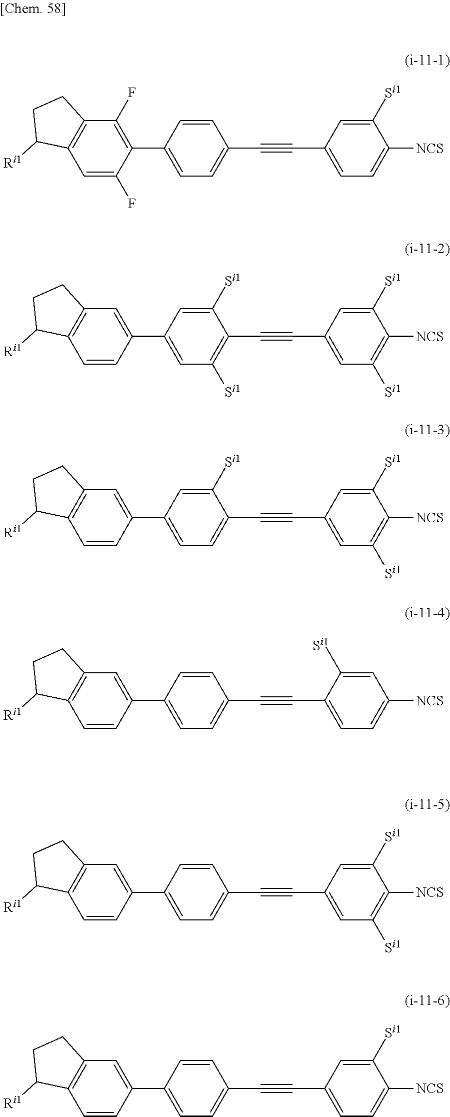

In general formula (i-11-1) to (i-11-6), Rⁱ¹ and Sⁱ¹ each independently have the same meanings as Rⁱ¹ and Sⁱ¹ in general formula (i) above.

Specific examples of compounds represented by general formula (i-11-1) include the compounds represented by structural formula (i-11-1.1) to (i-11-1.3) below.

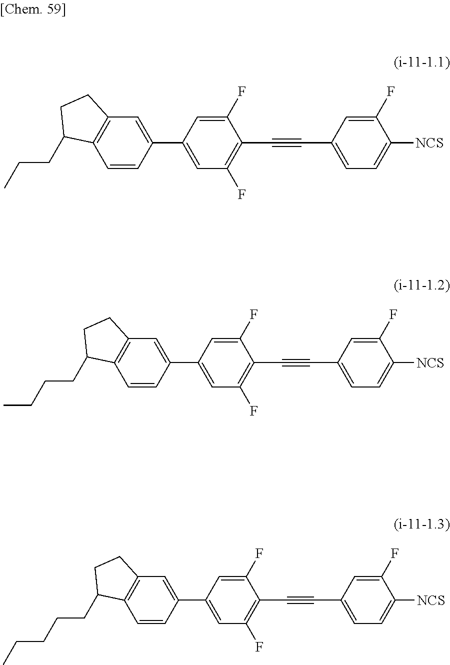

Specific examples of compounds represented by general formula (i-11-2) include the compounds represented by structural formula (i-11-2.1) to (i-11-2.3) below.

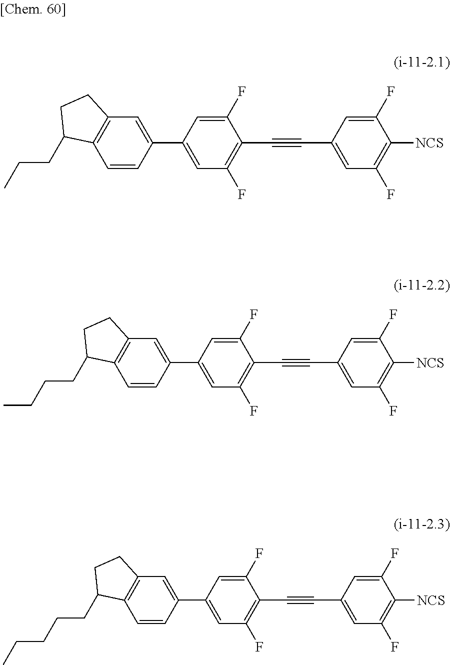

Specific examples of compounds represented by general formula (i-11-3) include the compounds represented by structural formula (i-11-3.1) and (i-11-3.2) below.

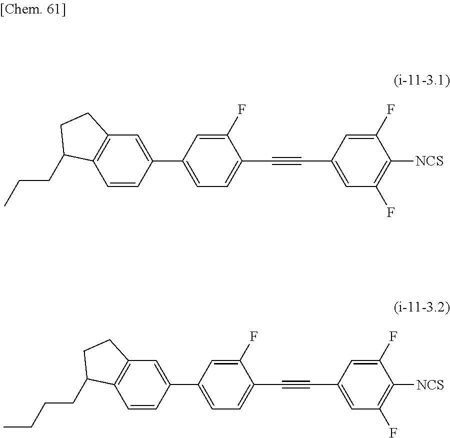

Specific examples of compounds represented by general formula (i-11-4) include the compounds represented by structural formula (i-11-4.1) to (i-11-4.3) below.

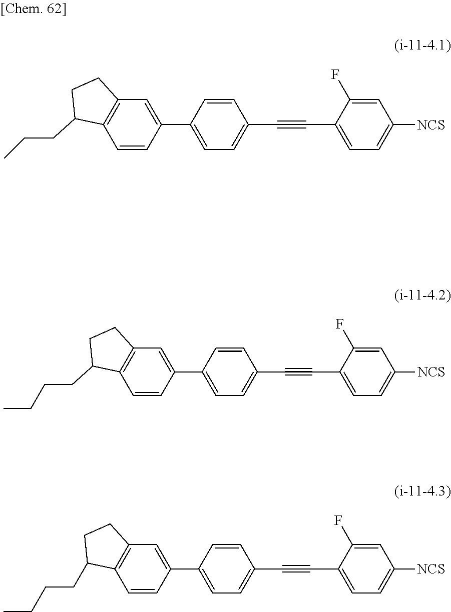

Specific examples of compounds represented by general formula (i-11-5) include the compounds represented by structural formula (i-11-5.1) to (i-11-5.3) below.

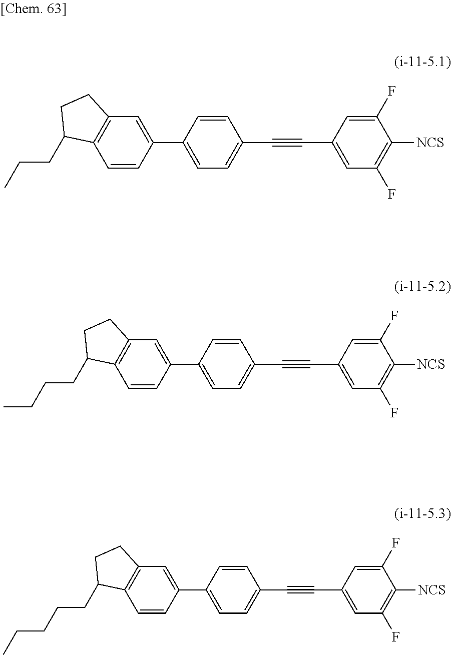

Specific examples of compounds represented by general formula (i-11-6) include the compounds represented by structural formula (i-11-6.1) and (i-11-6.2) below.

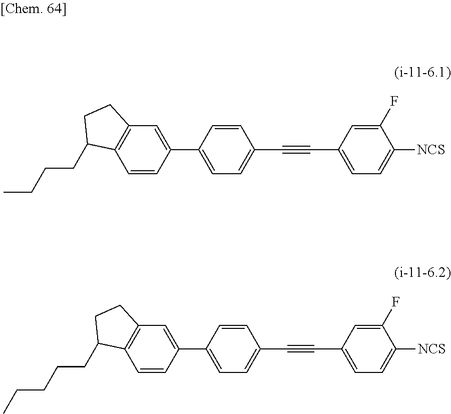

Compounds represented by general formula (i-12) are preferably compounds represented by general formula (i-12-1) to (i-12-4) below.

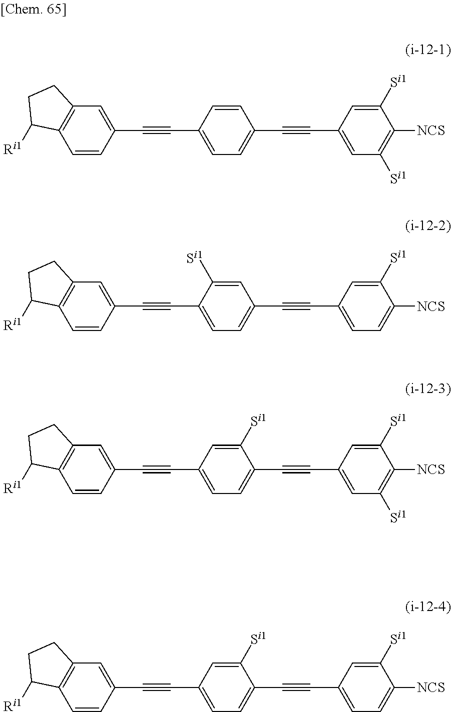

In general formula (i-12-1) to (i-12-4), Rⁱ¹ and Sⁱ¹ each independently have the same meanings as Rⁱ¹ and Sⁱ¹ in general formula (i) above.

Specific examples of compounds represented by general formula (i-12-1) include the compounds represented by structural formula (i-12-1.1) to (i-12-1.3) below.

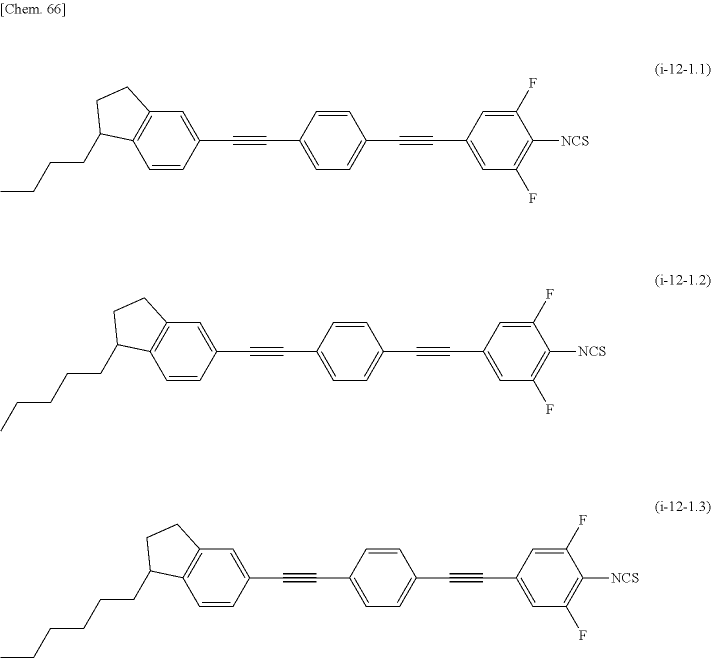

Specific examples of compounds represented by general formula (i-12-2) include the compounds represented by structural formula (i-12-2.1) and (i-12-2.2) below.

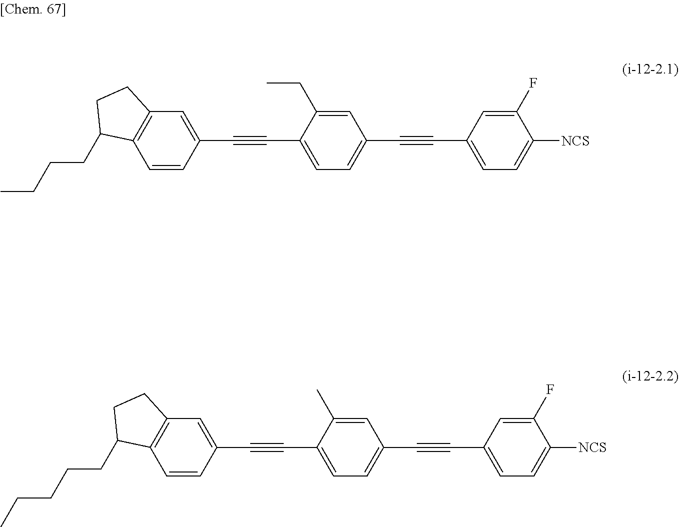

Specific examples of compounds represented by general formula (i-12-3) include the compounds represented by structural formula (i-12-3.1) and (i-12-3.2) below.

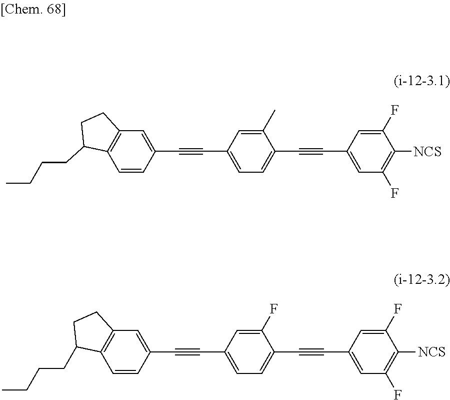

Specific examples of compounds represented by general formula (i-12-4) include the compounds represented by structural formula (i-12-4.1) and (i-12-4.2) below.

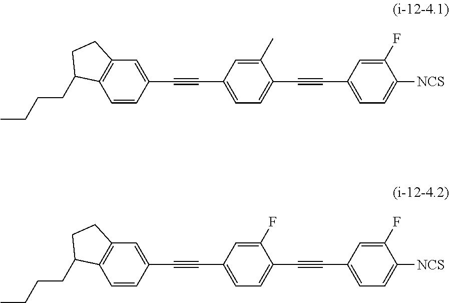

Compounds represented by general formula (i-13) are preferably compounds represented by general formula (i-13-1) below.

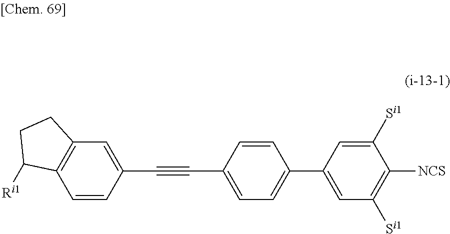

In general formula (i-13-1), Rⁱ¹ and Sⁱ¹ each independently have the same meanings as Rⁱ¹ and Sⁱ¹ in general formula (i) above.

Specific examples of compounds represented by general formula (i-13-1) include the compounds represented by structural formula (i-13-1.1) to (i-13-1.3) below.

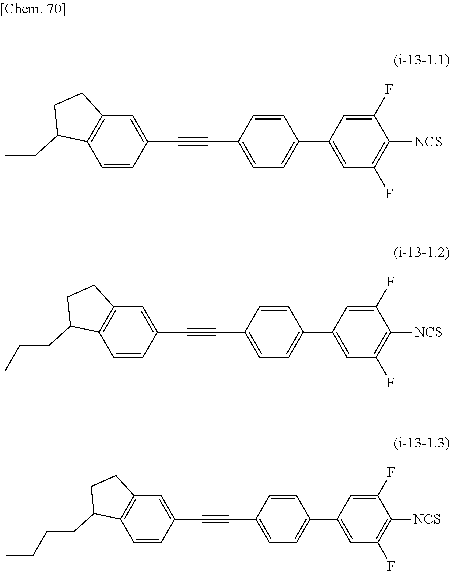

Compounds represented by general formula (i-14) are preferably compounds represented by general formula (i-14-1) below.

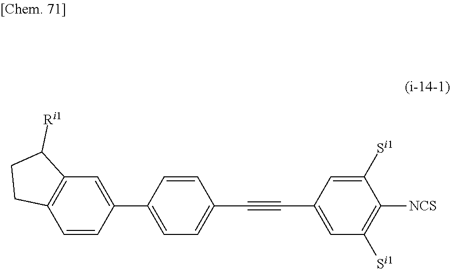

In general formula (i-14-1), Rⁱ¹ and Sⁱ¹ each independently have the same meanings as Rⁱ¹ and Sⁱ¹ in general formula (i) above.

Specific examples of compounds represented by general formula (i-14-1) include the compounds represented by structural formula (i-14-1.1) and (i-14-1.2) below.

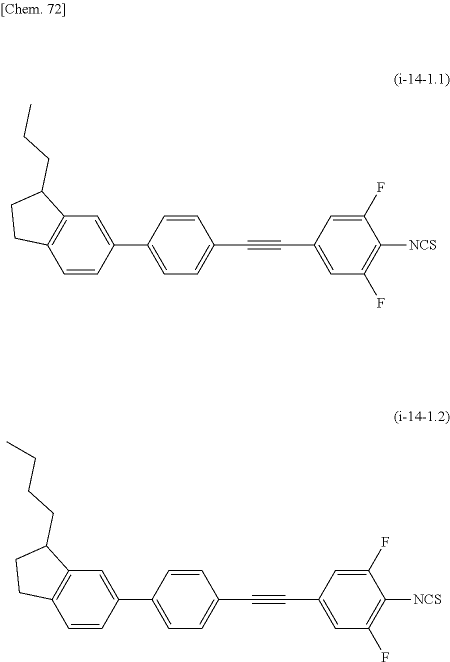

The number of types of compounds represented by general formula (i), general formula (i-1) to (i-14), general formula (i-1-1) and (i-1-2), general formula (i-2-1) to (i-2-4), general formula (i-3-1) to (i-3-3), general formula (i-4-1) to (i-4-6), general formula (i-5-1), general formula (i-6-1) and (i-6-2), general formula (i-7-1) and (i-7-2), general formula (i-8-1) to (i-8-5), general formula (i-9-1), general formula (i-10-1) to (i-10-3), general formula (i-11-1) to (i-11-6), general formula (i-12-1) to (i-12-4), general formula (i-13-1), general formula (i-14-1), structural formula (i-1-1.1) to (i-1-1.3), structural formula (i-1-2.1) to (i-1-2.3), structural formula (i-2-1.1) to (i-2-1.3), structural formula (i-2-2.1) to (i-2-2.3), structural formula (i-2-3.1) to (i-2-3.3), structural formula (i-2-4.1), structural formula (i-3-1.1) to (i-3-1.3), structural formula (i-3-2.1) to (i-3-2.3), structural formula (i-3-3.1) to (i-3-3.3), structural formula (i-4-1.1) to (i-4-1.3), structural formula (i-4-2.1) to (i-4-2.3), structural formula (i-4-3.1) to (i-4-3.3), structural formula (i-4-4.1) to (i-4-4.3), structural formula (i-4-5.1) to (i-4-5.4), structural formula (i-4-6.1) to (i-4-6.3), structural formula (i-5-1.1) and (i-5-1.2), structural formula (i-6-1.1) and (i-6-1.2), structural formula (i-6-2.1) and (i-6-2.2), structural formula (i-7-1.1), structural formula (i-7-2.1), structural formula (i-8-1.1) and (i-8-1.2), structural formula (i-8-2.1) and (i-8-2.2), structural formula (i-8-3.1) to (i-8-3.4), structural formula (i-8-4.1) and (i-8-4.2), structural formula (i-8-5.1) and (i-8-5.2), structural formula (1-9-1.1), structural formula (i-10-1.1) to (i-10-1.3), structural formula (i-10-2.1) to (i-10-2.4), structural formula (i-10-3.1) to (i-10-3.3), structural formula (i-11-1.1) to (i-11-1.3), structural formula (i-11-2.1) to (i-11-2.3), structural formula (i-11-3.1) and (i-11-3.2), structural formula (i-11-4.1) to (i-11-4.3), structural formula (i-11-5.1) to (i-11-5.3), structural formula (i-11-6.1) and (i-11-6.2), structural formula (i-12-1.1) to (i-12-1.3), structural formula (i-12-2.1) and (i-12-2.2), structural formula (i-12-3.1) and (i-12-3.2), structural formula (i-12-4.1) and (i-12-4.2), structural formula (i-13-1.1) to (i-13-1.3), or structural formula (i-14-1.1) and (i-14-1.2) used in the liquid crystal composition is one or two or more, preferably from one to ten, preferably from one to five, preferably from one to three.

The lower limit to the total amount of compounds represented by general formula (i), general formula (i-1) to (i-14), general formula (i-1-1) and (i-1-2), general formula (i-2-1) to (i-2-4), general formula (i-3-1) to (i-3-3), general formula (i-4-1) to (i-4-6), general formula (i-5-1), general formula (i-6-1) and (i-6-2), general formula (i-7-1) and (i-7-2), general formula (i-8-1) to (i-8-5), general formula (i-9-1), general formula (i-10-1) to (i-10-3),
general formula (i-11-1) to (i-11-6), general formula (i-12-1) to (i-12-4), general formula (i-13-1), general formula (i-14-1), structural formula (i-1-1.1) to (i-1-1.3), structural formula (i-1-2.1) to (i-1-2.3), structural formula (i-2-1.1) to (i-2-1.3), structural formula (i-2-2.1) to (i-2-2.3), structural formula (i-2-3.1) to (i-2-3.3), structural formula (i-2-4.1), structural formula (i-3-1.1) to (i-3-1.3), structural formula (i-3-2.1) to (i-3-2.3), structural formula (i-3-3.1) to (i-3-3.3), structural formula (i-4-1.1) to (i-4-1.3), structural formula (i-4-2.1) to (i-4-2.3), structural formula (i-4-3.1) to (i-4-3.3), structural formula (i-4-4.1) to (i-4-4.3), structural formula (i-4-5.1) to (i-4-5.4), structural formula (i-4-6.1) to (i-4-6.3), structural formula (i-5-1.1) and (i-5-1.2), structural formula (i-6-1.1) and (i-6-1.2), structural formula (i-6-2.1) and (i-6-2.2), structural formula (i-7-1.1), structural formula (i-7-2.1), structural formula (i-8-1.1) and (i-8-1.2), structural formula (i-8-2.1) and (i-8-2.2), structural formula (i-8-3.1) to (i-8-3.4), structural formula (i-8-4.1) and (i-8-4.2), structural formula (i-8-5.1) and (i-8-5.2), structural formula (1-9-1.1), structural formula (i-10-1.1) to (i-10-1.3), structural formula (i-10-2.1) to (i-10-2.4), structural formula (i-10-3.1) to (i-10-3.3), structural formula (i-11-1.1) to (i-11-1.3), structural formula (i-11-2.1) to (i-11-2.3), structural formula (i-11-3.1) and (i-11-3.2), structural formula (i-11-4.1) to (i-11-4.3), structural formula (i-11-5.1) to (i-11-5.3), structural formula (i-11-6.1) and (i-11-6.2), structural formula (i-12-1.1) to (i-12-1.3), structural formula (i-12-2.1) and (i-12-2.2), structural formula (i-12-3.1) and (i-12-3.2), structural formula (i-12-4.1) and (i-12-4.2), structural formula (i-13-1.1) to (i-13-1.3), or structural formula (i-14-1.1) and (i-14-1.2) in 100% by mass of the liquid crystal composition is preferably 0.5% by mass or more, preferably 1% by mass or more, preferably 3% by mass or more, preferably 5% by mass or more, preferably 10% by mass or more, preferably 15% by mass or more, preferably 20% by mass or more.

The upper limit to the total amount of compounds represented by general formula (i), general formula (i-1) to (i-14), general formula (i-1-1) and (i-1-2), general formula (i-2-1) to (i-2-4), general formula (i-3-1) to (i-3-3), general formula (i-4-1) to (i-4-6), general formula (i-5-1), general formula (i-6-1) and (i-6-2), general formula (i-7-1) and (i-7-2), general formula (i-8-1) to (i-8-5), general formula (i-9-1), general formula (i-10-1) to (i-10-3),
general formula (i-11-1) to (i-11-6), general formula (i-12-1) to (i-12-4), general formula (i-13-1), general formula (i-14-1), structural formula (i-1-1.1) to (i-1-1.3), structural formula (i-1-2.1) to (i-1-2.3), structural formula (i-2-1.1) to (i-2-1.3), structural formula (i-2-2.1) to (i-2-2.3), structural formula (i-2-3.1) to (i-2-3.3), structural formula (i-2-4.1), structural formula (i-3-1.1) to (i-3-1.3), structural formula (i-3-2.1) to (i-3-2.3), structural formula (i-3-3.1) to (i-3-3.3), structural formula (i-4-1.1) to (i-4-1.3), structural formula (i-4-2.1) to (i-4-2.3), structural formula (i-4-3.1) to (i-4-3.3), structural formula (i-4-4.1) to (i-4-4.3), structural formula (i-4-5.1) to (i-4-5.4), structural formula (i-4-6.1) to (i-4-6.3), structural formula (i-5-1.1) and (i-5-1.2), structural formula (i-6-1.1) and (i-6-1.2), structural formula (i-6-2.1) and (i-6-2.2), structural formula (i-7-1.1), structural formula (i-7-2.1), structural formula (i-8-1.1) and (i-8-1.2), structural formula (i-8-2.1) and (i-8-2.2), structural formula (i-8-3.1) to (i-8-3.4), structural formula (i-8-4.1) and (i-8-4.2), structural formula (i-8-5.1) and (i-8-5.2), structural formula (1-9-1.1), structural formula (i-10-1.1) to (i-10-1.3), structural formula (i-10-2.1) to (i-10-2.4), structural formula (i-10-3.1) to (i-10-3.3), structural formula (i-11-1.1) to (i-11-1.3), structural formula (i-11-2.1) to (i-11-2.3), structural formula (i-11-3.1) and (i-11-3.2), structural formula (i-11-4.1) to (i-11-4.3), structural formula (i-11-5.1) to (i-11-5.3), structural formula (i-11-6.1) and (i-11-6.2), structural formula (i-12-1.1) to (i-12-1.3), structural formula (i-12-2.1) and (i-12-2.2), structural formula (i-12-3.1) and (i-12-3.2), structural formula (i-12-4.1) and (i-12-4.2), structural formula (i-13-1.1) to (i-13-1.3), or structural formula (i-14-1.1) and (i-14-1.2) in 100% by mass of the liquid crystal composition is preferably 35% by mass or less, preferably 30% by mass or less, preferably 25% by mass or less, preferably 20% by mass or less, preferably 15% by mass or less, preferably 10% by mass or less, preferably 5% by mass or less.

From the viewpoint(s) of solubility, Δn, and/or Δεᵣ, it is preferred that the total amount of compounds represented by general formula (i), general formula (i-1) to (i-14), general formula (i-1-1) and (i-1-2), general formula (i-2-1) to (i-2-4), general formula (i-3-1) to (i-3-3), general formula (i-4-1) to (i-4-6), general formula (i-5-1), general formula (i-6-1) and (i-6-2), general formula (i-7-1) and (i-7-2), general formula (i-8-1) to (i-8-5), general formula (i-9-1), general formula (i-10-1) to (i-10-3),
general formula (i-11-1) to (i-11-6), general formula (i-12-1) to (i-12-4), general formula (i-13-1), general formula (i-14-1), structural formula (i-1-1.1) to (i-1-1.3), structural formula (i-1-2.1) to (i-1-2.3), structural formula (i-2-1.1) to (i-2-1.3), structural formula (i-2-2.1) to (i-2-2.3), structural formula (i-2-3.1) to (i-2-3.3), structural formula (i-2-4.1), structural formula (i-3-1.1) to (i-3-1.3), structural formula (i-3-2.1) to (i-3-2.3), structural formula (i-3-3.1) to (i-3-3.3), structural formula (i-4-1.1) to (i-4-1.3), structural formula (i-4-2.1) to (i-4-2.3), structural formula (i-4-3.1) to (i-4-3.3), structural formula (i-4-4.1) to (i-4-4.3), structural formula (i-4-5.1) to (i-4-5.4), structural formula (i-4-6.1) to (i-4-6.3), structural formula (i-5-1.1) and (i-5-1.2), structural formula (i-6-1.1) and (i-6-1.2), structural formula (i-6-2.1) and (i-6-2.2), structural formula (i-7-1.1), structural formula (i-7-2.1), structural formula (i-8-1.1) and (i-8-1.2), structural formula (i-8-2.1) and (i-8-2.2), structural formula (i-8-3.1) to (i-8-3.4), structural formula (i-8-4.1) and (i-8-4.2), structural formula (i-8-5.1) and (i-8-5.2), structural formula (1-9-1.1), structural formula (i-10-1.1) to (i-10-1.3), structural formula (i-10-2.1) to (i-10-2.4), structural formula (i-10-3.1) to (i-10-3.3), structural formula (i-11-1.1) to (i-11-1.3), structural formula (i-11-2.1) to (i-11-2.3), structural formula (i-11-3.1) and (i-11-3.2), structural formula (i-11-4.1) to (i-11-4.3), structural formula (i-11-5.1) to (i-11-5.3), structural formula (i-11-6.1) and (i-11-6.2), structural formula (i-12-1.1) to (i-12-1.3), structural formula (i-12-2.1) and (i-12-2.2), structural formula (i-12-3.1) and (i-12-3.2), structural formula (i-12-4.1) and (i-12-4.2), structural formula (i-13-1.1) to (i-13-1.3), or structural formula (i-14-1.1) and (i-14-1.2) in 100% by mass of the liquid crystal composition be from 0.5% to 35% by mass, preferably from 1% to 30% by mass, preferably from 3% to 25% by mass.

Compounds represented by general formula (i) (including subordinate concepts) can be synthesized using known synthetic methods. In the following, some of them will be presented by way of example.

### (Process 1) Production of a Compound Represented by Formula (s-6) Below

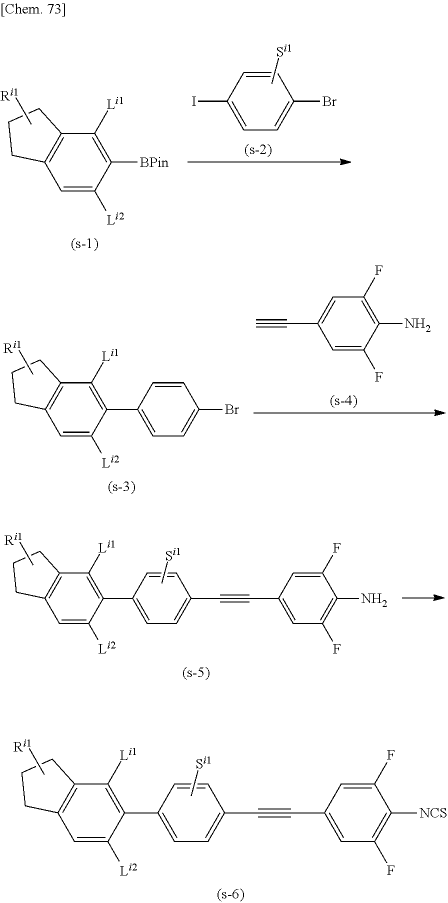

In general formula (s-i) to (s-6), Rⁱ¹, Lⁱ¹, Lⁱ², and Sⁱ¹ have the same meanings as Rⁱ¹, Lⁱ¹, L², and Sⁱ¹ in general formula (i) above.

First, a compound represented by general formula (s-i) is allowed to react with a compound represented by general formula (s-2), through which a compound represented by general formula (s-3) can be obtained.

An example of a reaction method is the Suzuki coupling reaction, which uses a metal catalyst and a base.

Specific examples of metal catalysts include [1,1′-bis(diphenylphosphino)ferrocene]palladium(II) dichloride, palladium(II) acetate, dichlorobis[di-tert-butyl(p-dimethylaminophenyl)phosphino]palladium(II), dichlorobis(triphenylphosphine)palladium(II), and tetrakis(triphenylphosphine)palladium(0).

When palladium(II) acetate is used as the metal catalyst, a ligand, such as triphenylphosphine or 2-dicyclohexylphosphino-2′,4′,6′-triisopropylbiphenyl, may be added.

Specific examples of bases include potassium carbonate, potassium phosphate, and cesium carbonate.

Then the compound represented by general formula (s-3) is allowed to react with a compound represented by general formula (s-4), through which a compound represented by general formula (s-5) can be obtained.

An example of a reaction method is the Sonogashira coupling reaction, which uses a palladium catalyst, a copper catalyst, and a base.

Specific examples of palladium catalysts include [1,1′-bis(diphenylphosphino)ferrocene]palladium(II) dichloride, palladium(II) acetate, dichlorobis[di-tert-butyl(p-dimethylaminophenyl)phosphino]palladium(II), dichlorobis(triphenylphosphine)palladium(II), and tetrakis(triphenylphosphine)palladium(0).

When palladium(II) acetate is used as the palladium catalyst, a ligand, such as triphenylphosphine or 2-dicyclohexylphosphino-2′,4′,6′-triisopropylbiphenyl, may be added.

A specific example of a copper catalyst is copper(I) iodide.

Specific examples of bases include triethylamine and diisopropylamine.

Finally, the compound represented by general formula (s-5) is allowed to react, for example with 1,1-thiocarbonyldiimidazole, 1,1-thiocarbonyldi-2(1H)-pyridone, or thiophosgene, through which a compound represented by general formula (s-6) as the target compound can be obtained.

### (Process 2) Production of a Compound Represented by Formula (s-14) Below

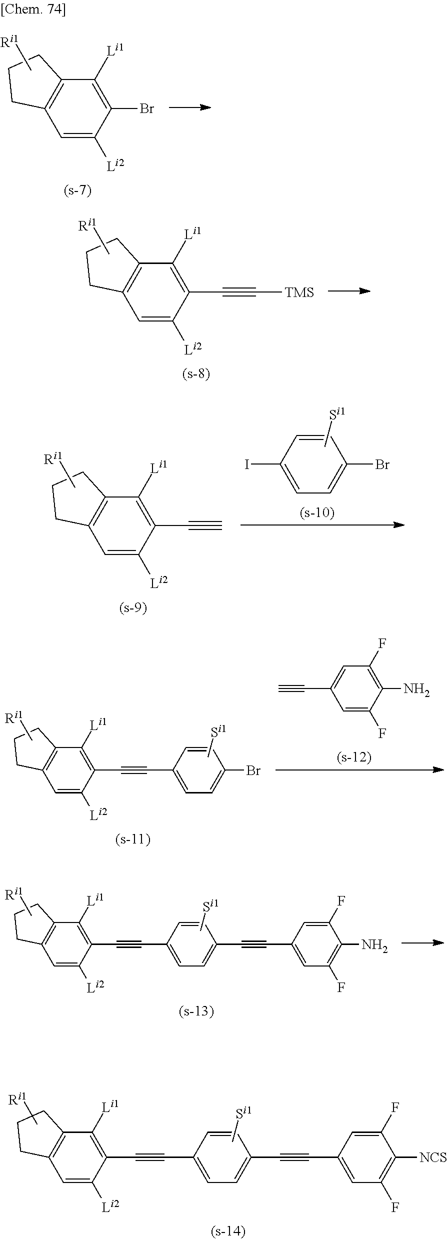

In general formula (s-7) to (s-14), Rⁱ¹, Lⁱ¹, Lⁱ², and Sⁱ¹ have the same meanings as Rⁱ¹, Lⁱ¹, Lⁱ², and Sⁱ¹ in general formula (i).

First, a compound represented by general formula (s-7) is allowed to react with trimethylsilylacetylene, through which a compound represented by general formula (s-8) can be obtained.

An example of a reaction method is the Sonogashira coupling reaction, which uses a palladium catalyst, a copper catalyst, and a base.

Specific examples of palladium catalysts, copper catalysts, and bases include the compounds listed in (Process 1).

Then the compound represented by general formula (s-8) is allowed to react with potassium carbonate in an alcohol solvent, such as methanol, through which a compound represented by general formula (s-9) can be obtained.

Then the compound represented by general formula (s-9) is allowed to react with a compound represented by general formula (s-10), through which a compound represented by general formula (s-11) can be obtained.

An example of a reaction method is the Sonogashira coupling reaction, which uses a palladium catalyst, a copper catalyst, and a base.

Specific examples of palladium catalysts, copper catalysts, and bases include the compounds listed in (Process 1).

Then the compound represented by general formula (s-11) is allowed to react with a compound represented by general formula (s-12), through which a compound represented by general formula (s-13) can be obtained.

An example of a reaction method is the Sonogashira coupling reaction, which uses a palladium catalyst, a copper catalyst, and a base.

Specific examples of palladium catalysts, copper catalysts, and bases include the compounds listed in (Process 1).

Finally, the compound represented by general formula (s-13) is allowed to react, for example with 1,1-thiocarbonyldiimidazole, 1,1-thiocarbonyldi-2(1H)-pyridone, or thiophosgene, through which a compound represented by general formula (s-14) as the target compound can be obtained.

### Compound(s) Represented by General Formula (ii)

The liquid crystal composition according to the present invention contains one or two or more types of compounds represented by general formula (ii) below, which have an isothiocyanate group (—NCS).

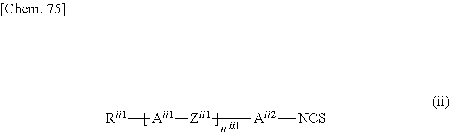

In general formula (ii), Rⁱⁱ¹ represents a C1 to C20 alkyl group.

The alkyl group is a linear-chain, branched, or cyclic alkyl group and preferably is a linear-chain alkyl group.

The number of carbon atoms in the alkyl group is preferably from two to ten, preferably from two to six.

One —CH₂— in the alkyl group, or each of two or more independently, may have been replaced with —O—, —S—, —CO—, and/or —CS—.

One or two or more —CH₂—CH₂-s in the alkyl group, furthermore, may be replaced with —CO—O—, —O—CO—, —CO—S—, —S—CO—, —CO—NH—, —NH—CO—, —CH═CH—, —CF═CF—, and/or —C≡C—.

Moreover, one or two or more —CH₂—CH₂—CH₂-s in the alkyl group may have been replaced with —O—CO—O—.

In addition, one hydrogen atom in the alkyl group, or each of two or more independently, may have been replaced by a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

For example, Rⁱⁱ¹ can represent a C1 to C19 alkoxy group as a result of the replacement of one —CH₂— in the alkyl group by —O—.

The alkoxy group is a linear-chain, branched, or cyclic alkoxy group and preferably is a linear-chain alkoxy group.

The number of carbon atoms in the alkoxy group is preferably from two to ten, preferably from two to six.

Rⁱ¹, furthermore, can represent a C1 to C19 alkylsulfanyl group (alkylthio group) as a result of the replacement of one —CH₂— in the alkyl group by —S—.

The alkylsulfanyl group is a linear-chain, branched, or cyclic alkylsulfanyl group and preferably is a linear-chain alkylsulfanyl group.

The number of carbon atoms in the alkylsulfanyl group is preferably from one to ten, preferably from one to six.

Moreover, Rⁱⁱ¹ can represent a C2 to C20 alkenyl group as a result of the replacement of one or two or more —CH₂—CH₂-s in the alkyl group by —CH═CH—.

The alkenyl group is a linear-chain, branched, or cyclic alkenyl group and preferably is a linear-chain alkenyl group.

The number of carbon atoms in the alkenyl group is preferably from two to ten, preferably from two to six.

Rⁱ¹, furthermore, can represent a C2 to C20 alkynyl group as a result of the replacement of one or two or more —CH₂—CH₂-s in the alkyl group by —C≡C—.

The alkynyl group is a linear-chain, branched, or cyclic alkynyl group and preferably is a linear-chain alkynyl group.

The number of carbon atoms in the alkynyl group is preferably from two to ten, preferably from two to six.

For an alkynyl group, an alkynyl group represented by formula (Rⁱⁱ¹-A) below is preferred from the viewpoints of ease of synthesis and the extension of the conjugated system.

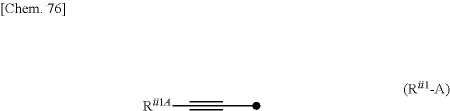

In formula (Rⁱⁱ¹-A), R^{ii1A} represents a C1 to C18 alkyl group.

The C1 to C18 alkyl group is a linear-chain, branched, or cyclic alkyl group and preferably is a linear-chain alkyl group.

The number of carbon atoms in the C1 to C18 alkyl group is preferably from one to eight.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CF—, and/or —C≡C—.

Moreover, one —CH₂—CH₂—CH₂— in the alkyl group, or each of two or more independently, may have been replaced with —O—CO—O—.

In addition, one hydrogen atom in the alkyl group, or each of two or more independently, may have been replaced with a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

In formula (Rⁱⁱ¹-A), furthermore, the black dot represents a bond to Aⁱⁱ¹.

Moreover, Rⁱⁱ¹ can represent a C2 to C19 alkenyloxy group as a result of the replacement of one —CH₂— in the alkyl group by —O— and the replacement of one or two or more —CH₂—CH₂-s by —CH═CH—.

The alkenyloxy group is a linear-chain, branched, or cyclic alkenyloxy group and preferably is a linear-chain alkenyloxy group.

The number of carbon atoms in the alkenyloxy group is preferably from two to ten, preferably from two to six.

Rⁱⁱ¹, furthermore, can represent a C1 to C20 halogenated alkyl group as a result of the replacement of one or two or more hydrogen atoms in the alkyl group by a halogen atom.

The halogenated alkyl group is a linear-chain, branched, or cyclic halogenated alkyl group and preferably is a linear-chain halogenated alkyl group.

The number of carbon atoms in the halogenated alkyl group is preferably from two to ten, preferably from two to six.

Moreover, Rⁱⁱ¹ can represent a C1 to C19 halogenated alkoxy group as a result of the replacement of one —CH₂— in the alkyl group by —O— and the replacement of one or two or more hydrogen atoms in the alkyl group by a halogen atom.

The halogenated alkoxy group is a linear-chain, branched, or cyclic halogenated alkoxy group and preferably is a linear-chain halogenated alkoxy group.

The number of carbon atoms in the halogenated alkoxy group is preferably from two to ten, preferably from two to six.

Specific examples of C1 to C20 alkyl groups (including substituted ones) at Rⁱⁱ¹ include the groups represented by formula (Rⁱⁱ¹-1) to (Rⁱⁱ¹-56).

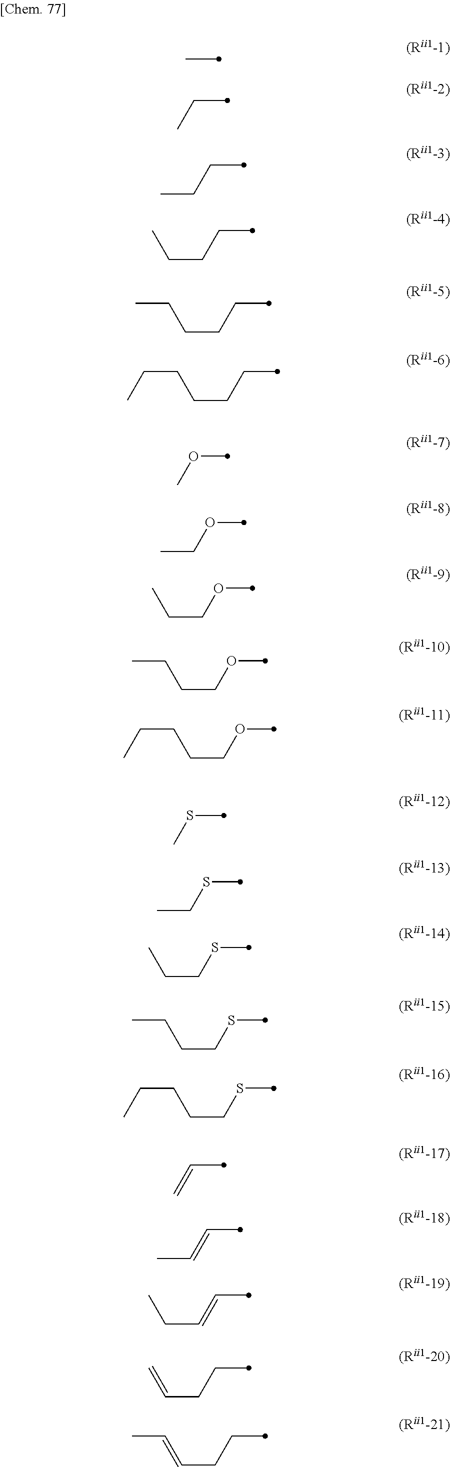

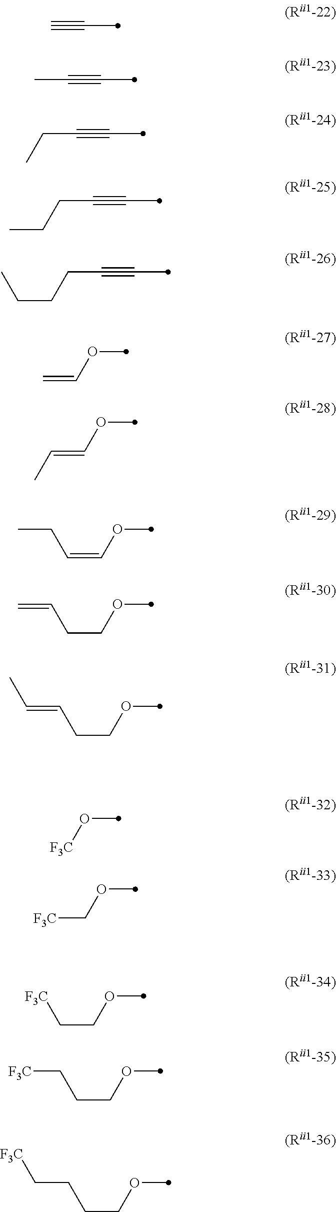

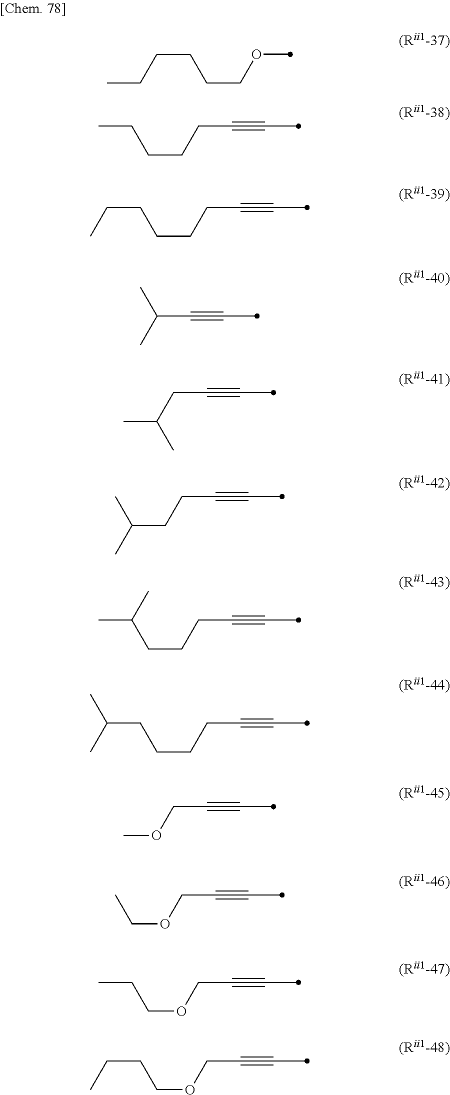

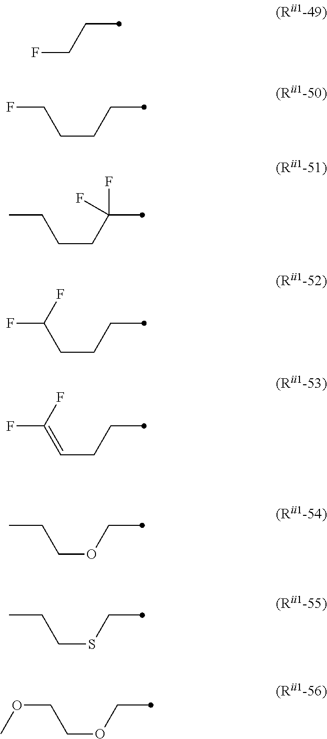

In formula (Rⁱⁱ¹-1) to (Rⁱⁱ¹-56), the black dot represents a bond to Aⁱⁱ¹.

When the ring structure to which Rⁱⁱ¹ is bound is a phenyl group (aromatic), linear-chain C1 to C5 alkyl groups, linear-chain C1 to C4 alkoxy groups, and C4 and C5 alkenyl groups are preferred. When the ring structure to which Rⁱⁱ¹ is bound is a saturated ring structure, such as cyclohexane, pyran, or dioxane, linear-chain C1 to C5 alkyl groups, linear-chain C1 to C4 alkoxy groups, and linear-chain C2 to C5 alkenyl groups are preferred.

For Rⁱⁱ¹, furthermore, it is preferred that the total number of carbon atoms and oxygen atoms, if any, be five or fewer, and it is preferred that Rⁱⁱ¹ be a linear-chain group, when the stabilization of the nematic phase is sought.

It should be noted that from the viewpoint of solubility, it is preferred that Rⁱⁱ¹ be a C2 to C8 linear-chain or branched alkyl group, a C2 to C8 linear-chain alkoxy group, a C1 to C8 linear-chain halogenated alkoxy group, a C2 to C8 linear-chain alkynyl group, or a C1 to C6 linear-chain alkylsulfanyl group.

In general formula (ii), Aⁱⁱ¹ and Aⁱⁱ² each independently represent a group selected from the group consisting of group (a), group (b), group (c), and group (d) below:
(a) a 1,4-cyclohexylene group (One —CH₂— or two or more nonadjacent —CH₂-s present in the group may be replaced by —O— and/or —S—.);
(b) a 1,4-phenylene group (One —CH═ or two or more —CH═s present in the group may be replaced by —N═.);
(c) a 1,4-cyclohexenylene group, a bicyclo[2.2.2]octan-1,4-diyl group, a naphthalen-2,6-diyl group, a naphthalen-1,4-diyl group, a 1,2,3,4-tetrahydronaphthalen-2,6-diyl group, a 5,6,7,8-tetrahydronaphthalen-1,4-diyl group, a decahydronaphthalen-2,6-diyl group, an anthracen-2,6-diyl group, an anthracen-1,4-diyl group, an anthracen-9,10-diyl group, or a phenanthren-2,7-diyl group (One —CH═ or two or more —CH═s present in a naphthalen-2,6-diyl group, naphthalen-1,4-diyl group, 1,2,3,4-tetrahydronaphthalen-2,6-diyl group, 5,6,7,8-tetrahydronaphthalen-1,4-diyl group, anthracen-2,6-diyl group, anthracen-1,4-diyl group, anthracen-9,10-diyl group, or phenanthren-2,7-diyl group may be replaced by —N═.);
(d) a thiophen-2,5-diyl group, a benzothiophen-2,5-diyl group, a benzothiophen-2,6-diyl group, a benzothiophen-3,7-diyl group, a dibenzothiophen-2,6-diyl group, a thieno[3,2-b]thiophen-2,5-diyl group, or a benzo[1,2-b:4,5-b′]dithiophen-2,6-diyl group (One —CH═ or two or more —CH═s present in the group may be replaced by —N═.)

One hydrogen atom in Aⁱⁱ¹ and Aⁱⁱ², or each of two or more independently, may have been replaced by a substituent Sⁱⁱ¹.

The substituent Sⁱⁱ¹ represents any of a halogen atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group.

Examples of halogen atoms include a fluorine atom, a chlorine atom, a bromine atom, and an iodine atom.

A C1 to C20 alkyl group is a linear-chain, branched, or cyclic alkyl group and preferably is a linear-chain alkyl group.

The number of carbon atoms in the C1 to C20 alkyl group is preferably from two to ten, preferably from two to six.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CF—, and/or —C≡C—.

One —CH₂—CH₂—CH₂— in the alkyl group, or each of two or more independently, may have been replaced with —O—CO—O—.

In addition, one hydrogen atom in the alkyl group, or each of two or more independently, may have been replaced by a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

The substituent Sⁱⁱ¹ is preferably a fluorine atom or chlorine atom.

It is, furthermore, preferred that Aⁱⁱ² or at least one of Aⁱⁱ¹ be substituted with at least one substituent Sⁱⁱ¹. Preferably, the group is substituted with a halogen atom, and preferably is substituted with a fluorine atom.

It should be noted that when there are multiple Sⁱⁱ¹s, they may be the same or may be different.

The position in Aⁱⁱ¹ at which it is substituted with a substituent or substituents Sⁱⁱ¹ is preferably any of formula (Aⁱⁱ¹-SP-1) to (Aⁱⁱ¹-SP-6) below.

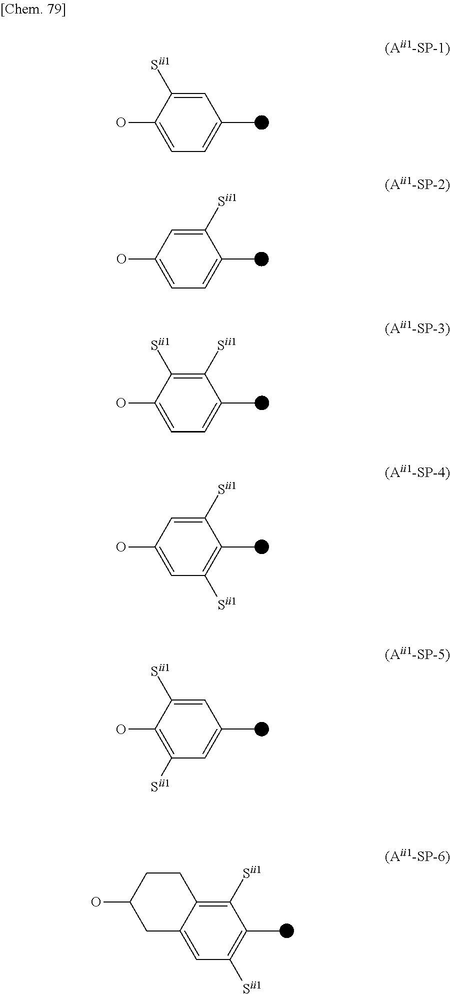

In formula (Aⁱⁱ¹-SP-1) to (Aⁱⁱ¹-SP-6), the white dot represents a bond to Rⁱⁱ¹ or Zⁱⁱ¹, and the black dot represents a bond to Zⁱⁱ¹.

The position in Aⁱⁱ² at which it is substituted with a substituent or substituents Sⁱⁱ¹ is preferably any of formula (Aⁱⁱ²-SP-1) to (Aⁱⁱ²-SP-8) below.

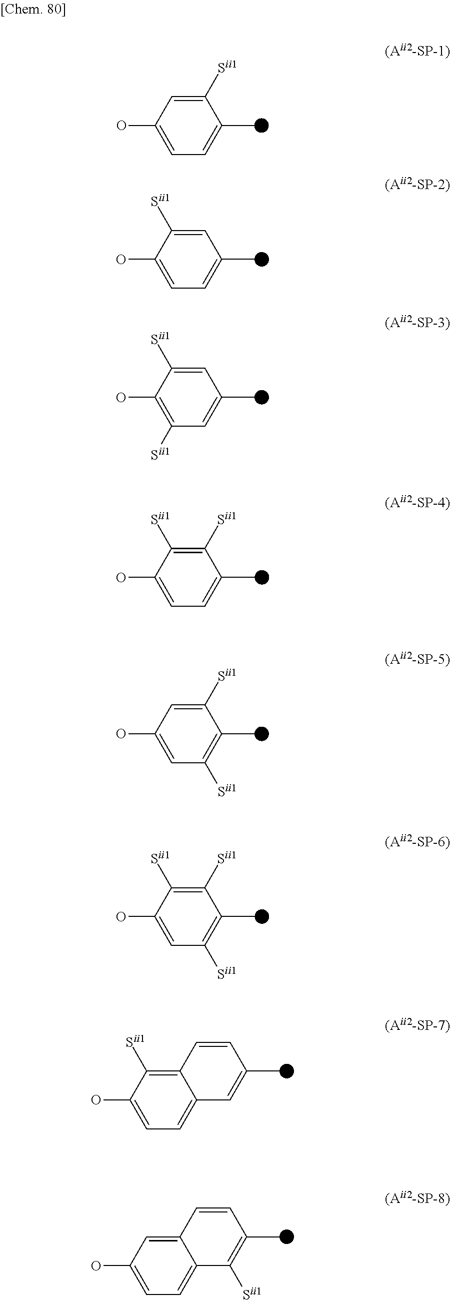

In formula (Aⁱⁱ²-SP-1) to (Aⁱⁱ²-SP-8), the white dot represents a bond to Zⁱⁱ¹, and the black dot represents a bond to the isothiocyanate group (—NCS).

More specifically, it is preferred that Aⁱⁱ¹ represent any of formula (Aⁱⁱ¹-1) to (Aⁱⁱ¹-25) below.

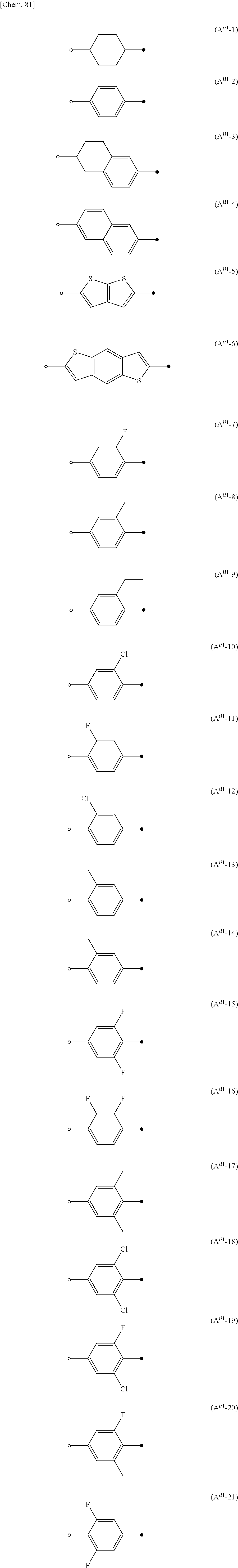

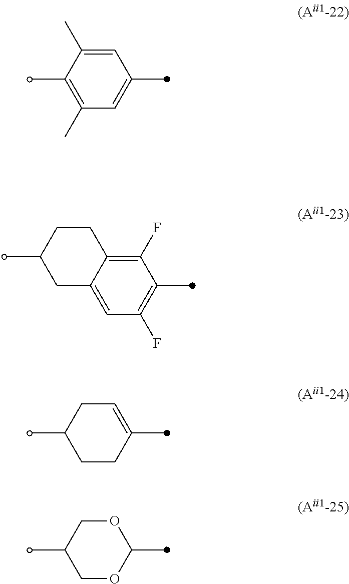

In formula (Aⁱⁱ¹-1) to (Aⁱⁱ¹-25), the white dot represents a bond to Rⁱⁱ¹ or Zⁱⁱ¹, and the black dot represents a bond to Zⁱⁱ¹.

More specifically, it is preferred that Aⁱⁱ² represent any of formula (Aⁱⁱ²-1) to (Aⁱⁱ²-8) below.

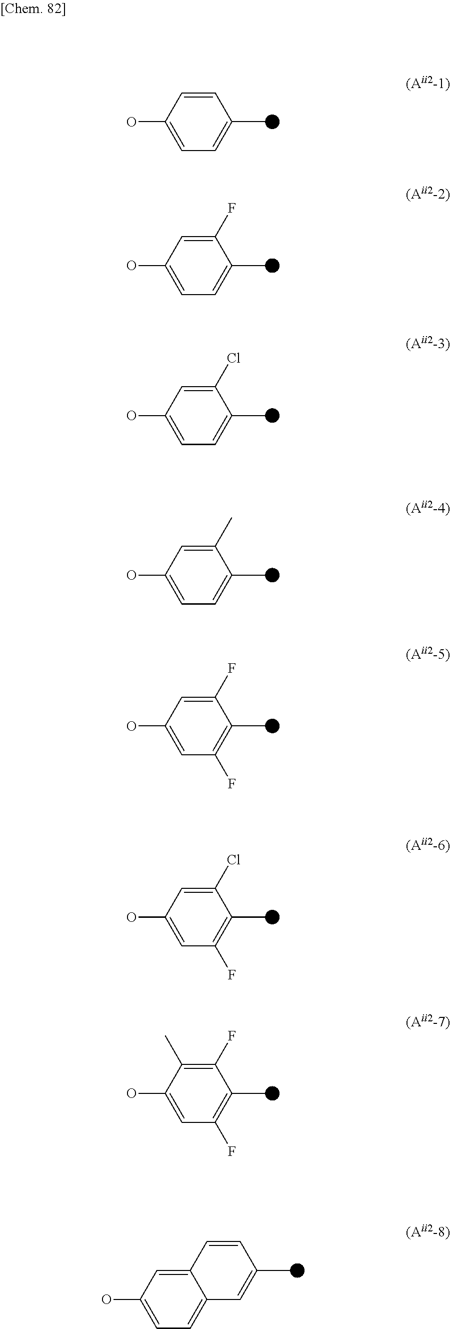

In formula (Aⁱⁱ²-1) to (Aⁱⁱ²-8), the white dot represents a bond to Zⁱⁱ¹, and the black dot represents a bond to the isothiocyanate group (—NCS).

In general formula (ii), Zⁱⁱ¹ represents any of a single bond or a C1 to C20 alkylene group.

One —CH₂— in the alkylene group, or each of two or more independently, may have been replaced with —O—, —CF₂—, and/or —CO—.

One —CH₂—CH₂— in the alkylene group, or each of two or more independently, furthermore, may be replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—.

Moreover, one —CH₂—CH₂—CH₂— in the alkylene group, or each of two or more independently, may have been replaced with —O—CO—O—.

When a C1 to C20 alkylene group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

Specific examples of C1 to C20 alkylene groups (including substituted ones) include the groups represented by formula (Zⁱⁱ¹-1) to (Zⁱ¹¹-24).

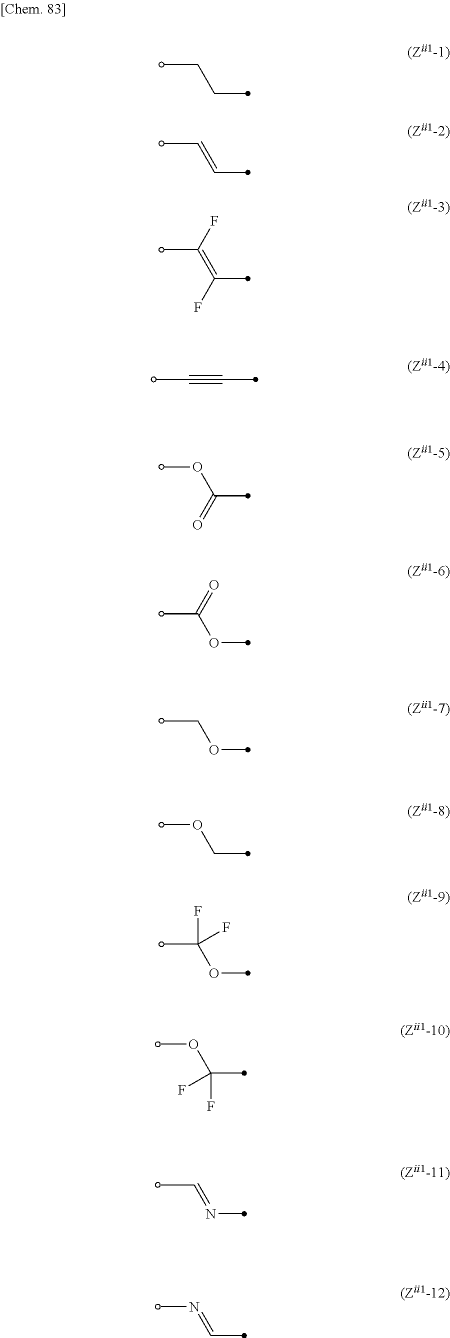

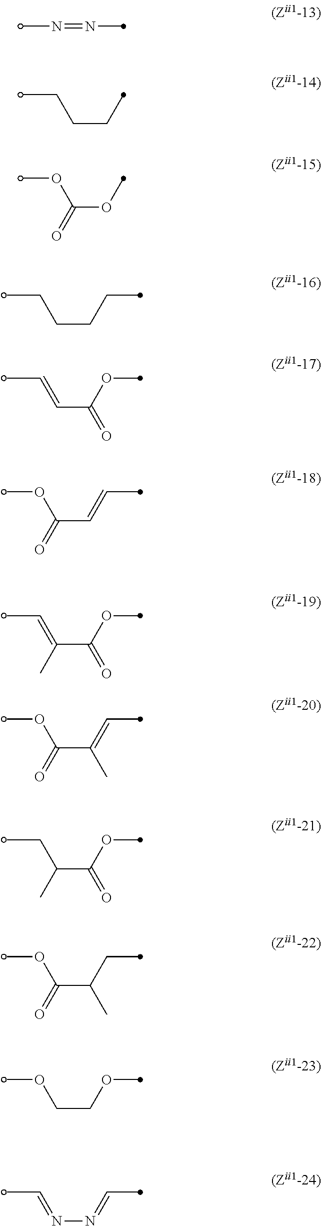

In formula (Zⁱⁱ¹-1) to (Zⁱⁱ¹-24), the white dot represents a bond to Aⁱⁱ¹, and the black dot represents a bond to Aⁱⁱ¹ or Aⁱⁱ².

In general formula (ii), nⁱⁱ¹ represents an integer of 1 to 4, preferably 1 or 2.

When nⁱⁱ¹ is 1, it is preferred that Zⁱⁱ¹ represent a single bond or —C≡C— from the viewpoint(s) of Δn and/or Δεᵣ.

When nⁱⁱ¹ is 2, furthermore, it is preferred that the Zⁱⁱ¹s represent single bonds or —C≡C-s from the viewpoint(s) of Δn and/or Δεᵣ.

It should be noted that when multiple Aⁱⁱ¹s and multiple Zⁱⁱ¹s are present in general formula (ii), the Aⁱⁱ¹s may be the same or may be different, and the Zⁱⁱ¹s may be the same or may be different.

For compounds represented by general formula (ii), however, compounds represented by general formula (i) (including subordinate concepts) are excluded.

The compound or compounds represented by general formula (ii) are preferably at least one compound represented by general formula (ii-1) to (ii-8) below.

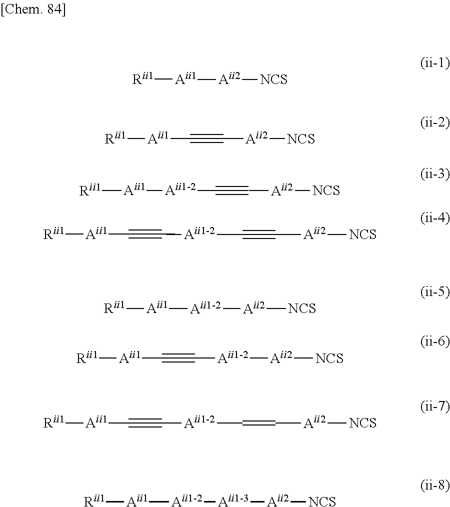

In general formula (ii-1) to (ii-8), Rⁱⁱ¹, Aⁱⁱ¹, and Aⁱⁱ² have the same meanings as Rⁱⁱ¹, Aⁱⁱ¹, and Aⁱⁱ², respectively, in general formula (ii) above.

In general formula (ii-3) to (ii-8), the definitions of Aⁱⁱ¹⁻² and Aⁱⁱ¹⁻³ are each independently the same as the definition of Aⁱⁱ¹ in general formula (ii) above.

Compounds represented by general formula (ii-1) are preferably compounds represented by general formula (ii-1-1) and (ii-1-2) below.

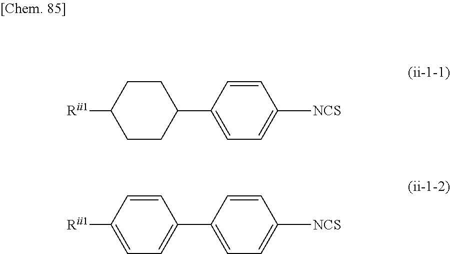

In general formula (ii-1-1) and (ii-1-2), Rⁱⁱ¹ has the same meaning as Rⁱⁱ¹, respectively, in general formula (ii) above, independently at each occurrence.

Specific examples of compounds represented by general formula (ii-1-1) include the compounds represented by structural formula (ii-1-1.1) to (ii-1-1.4) below.

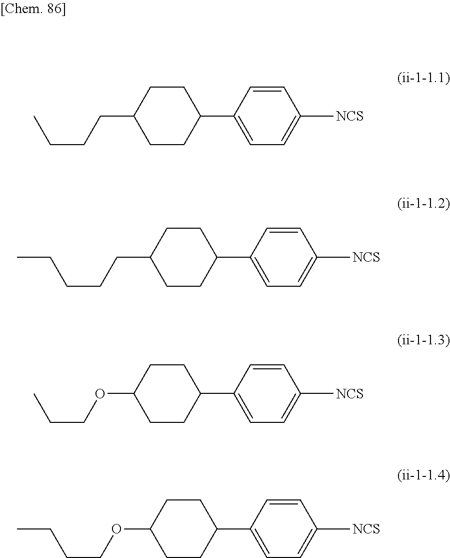

Specific examples of compounds represented by general formula (ii-1-2) include the compounds represented by structural formula (ii-1-2.1) to (ii-1-2.6) below.

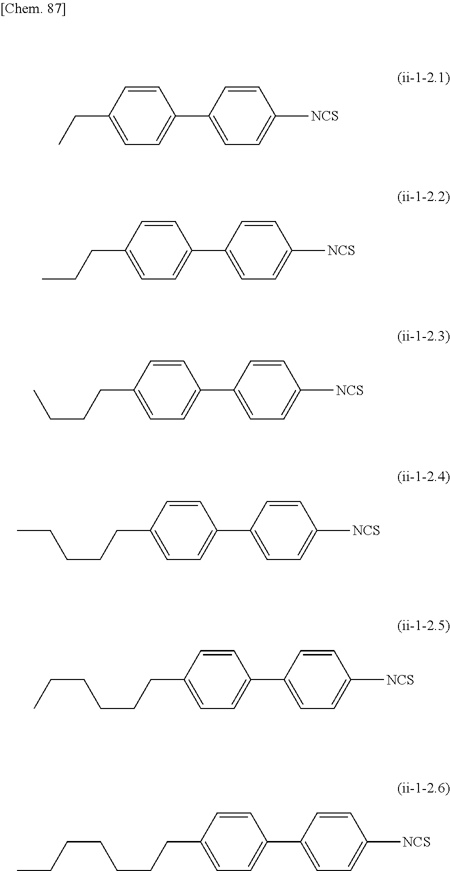

Compounds represented by general formula (ii-2) are preferably compounds represented by general formula (ii-2-1) to (ii-2-10) below.

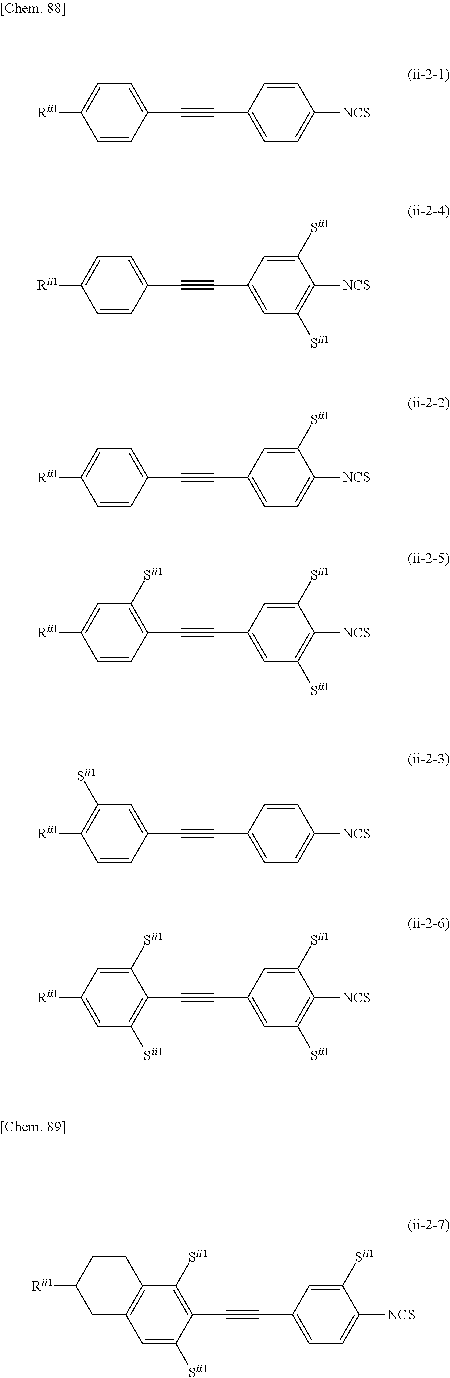

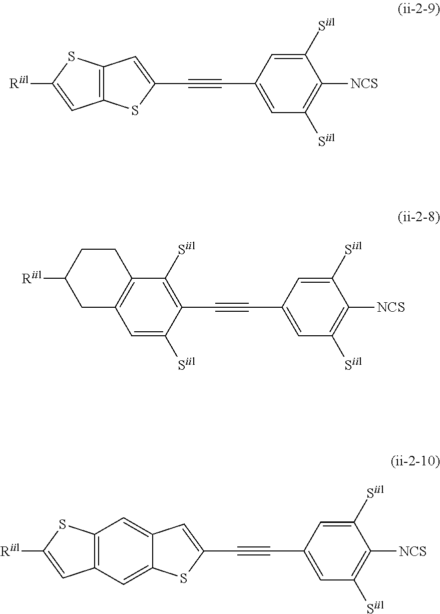

In general formula (ii-2-1) to (ii-2-10), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-2-1) include the compounds represented by structural formula (ii-2-1.1) to (ii-2-1.4) below.

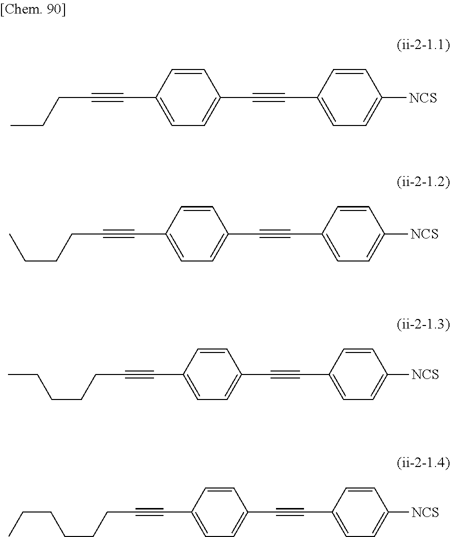

Specific examples of compounds represented by general formula (ii-2-2) include the compounds represented by structural formula (ii-2-2.1) to (ii-2-2.10) below.

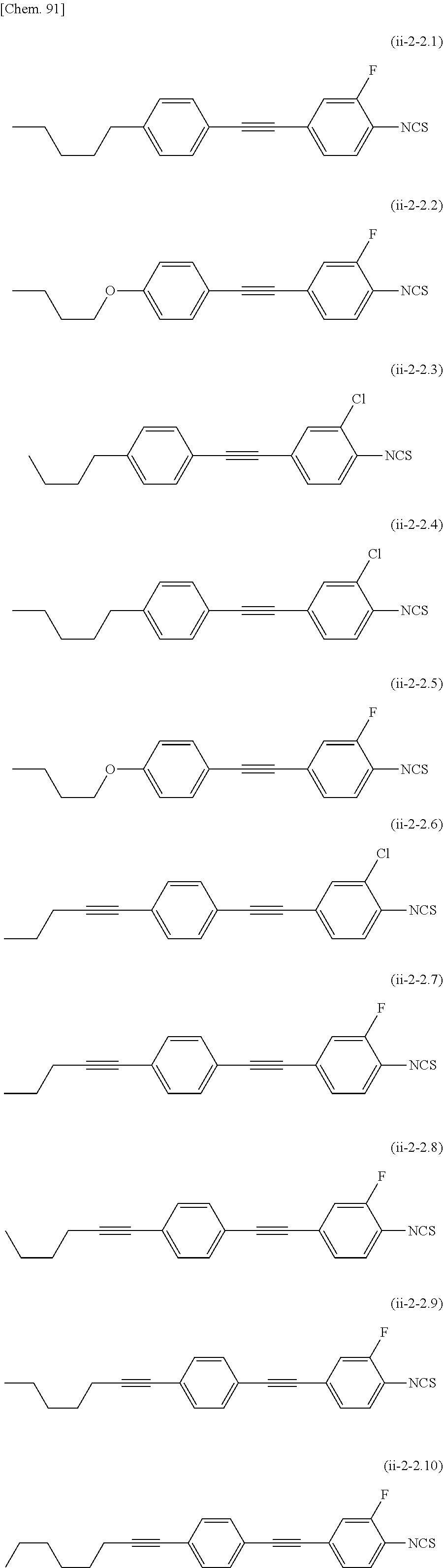

Specific examples of compounds represented by general formula (ii-2-3) include the compounds represented by structural formula (ii-2-3.1) to (ii-2-3.3) below.

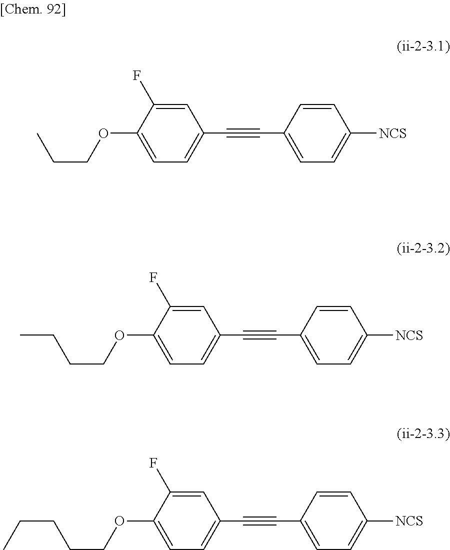

Specific examples of compounds represented by general formula (ii-2-4) include the compounds represented by structural formula (ii-2-4.1) to (ii-2-4.8) below.

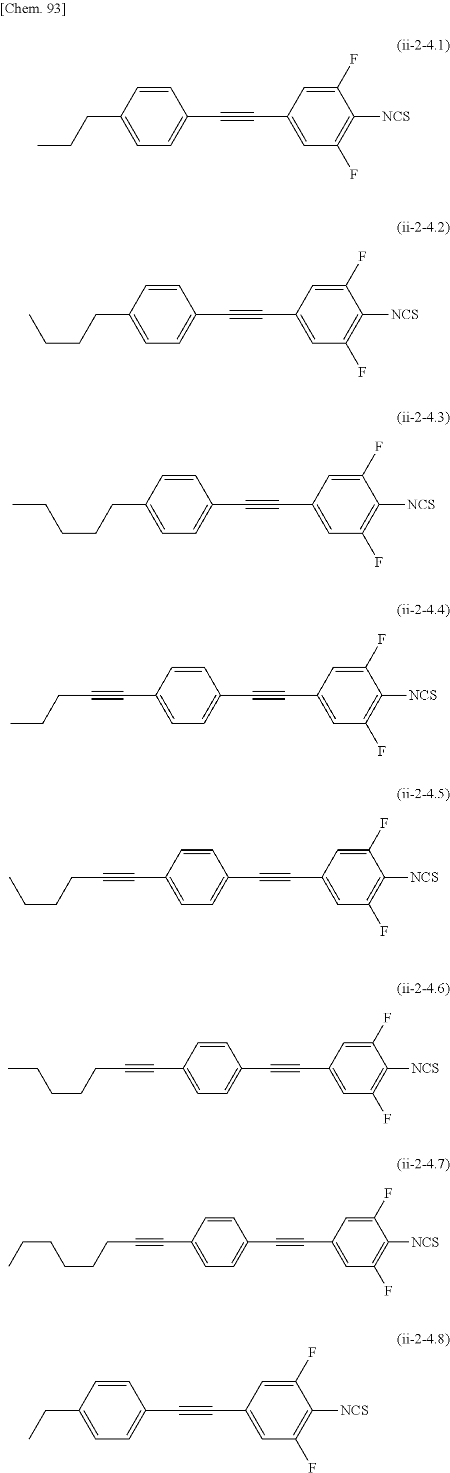

Specific examples of compounds represented by general formula (ii-2-5) include the compounds represented by structural formula (ii-2-5.1) to (ii-2-5.4) below.

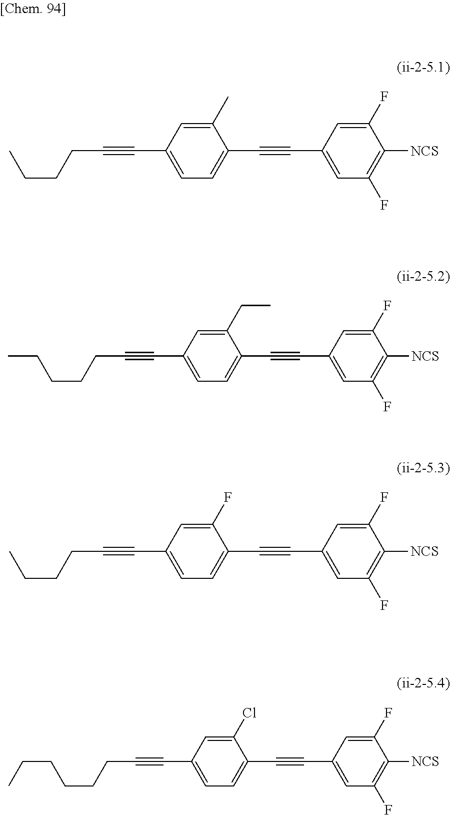

Specific examples of compounds represented by general formula (ii-2-6) include the compounds represented by structural formula (ii-2-6.1) to (ii-2-6.4) below.

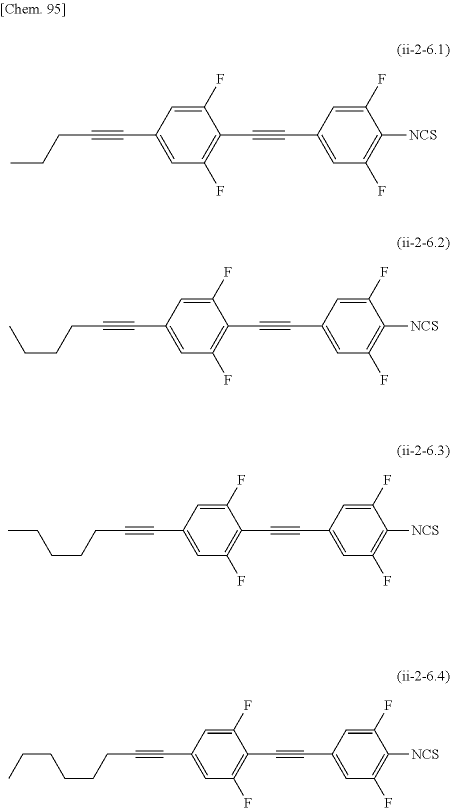

Specific examples of compounds represented by general formula (ii-2-7) include the compounds represented by structural formula (ii-2-7.1) to (ii-2-7.3) below.

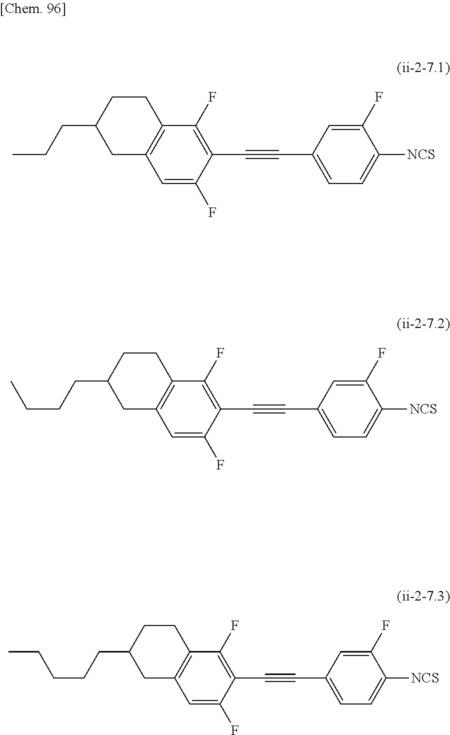

Specific examples of compounds represented by general formula (ii-2-8) include the compounds represented by structural formula (ii-2-8.1) to (ii-2-8.3) below.

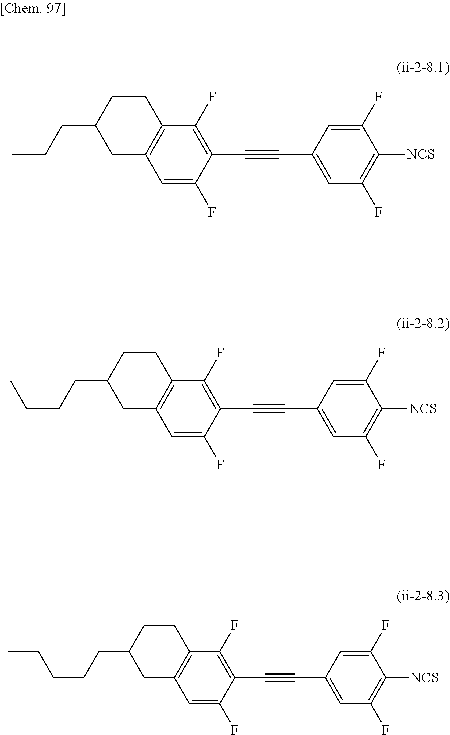

Specific examples of compounds represented by general formula (ii-2-9) include the compounds represented by structural formula (ii-2-9.1) to (ii-2-9.4) below.

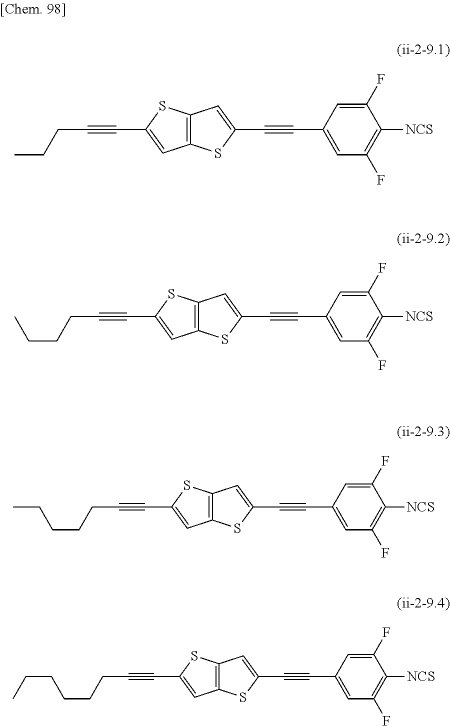

Specific examples of compounds represented by general formula (ii-2-10) include the compounds represented by structural formula (ii-2-10.1) to (ii-2-10.4) below.

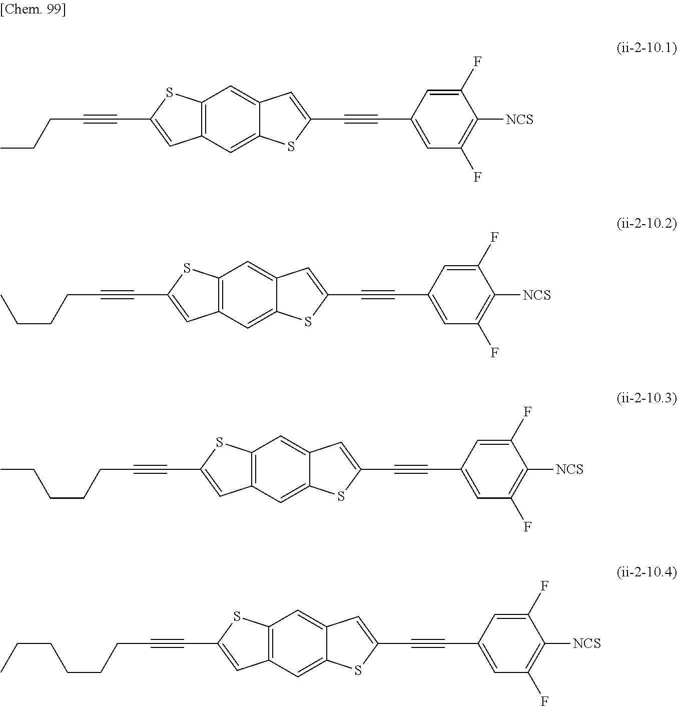

Compounds represented by general formula (ii-3) are preferably compounds represented by general formula (ii-3-1) to (ii-3-16) below.

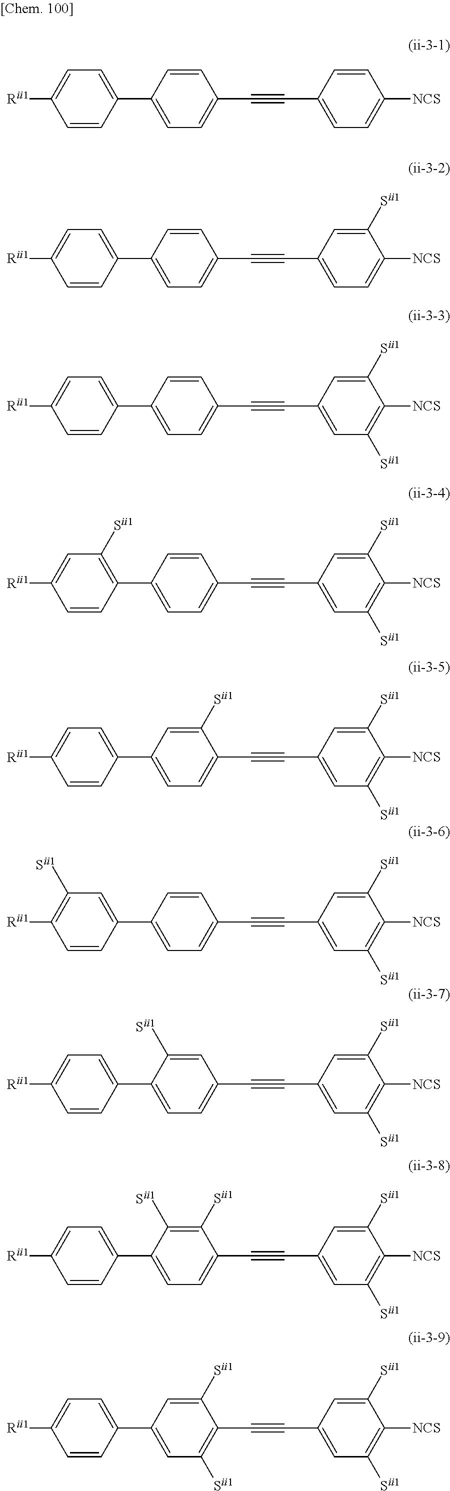

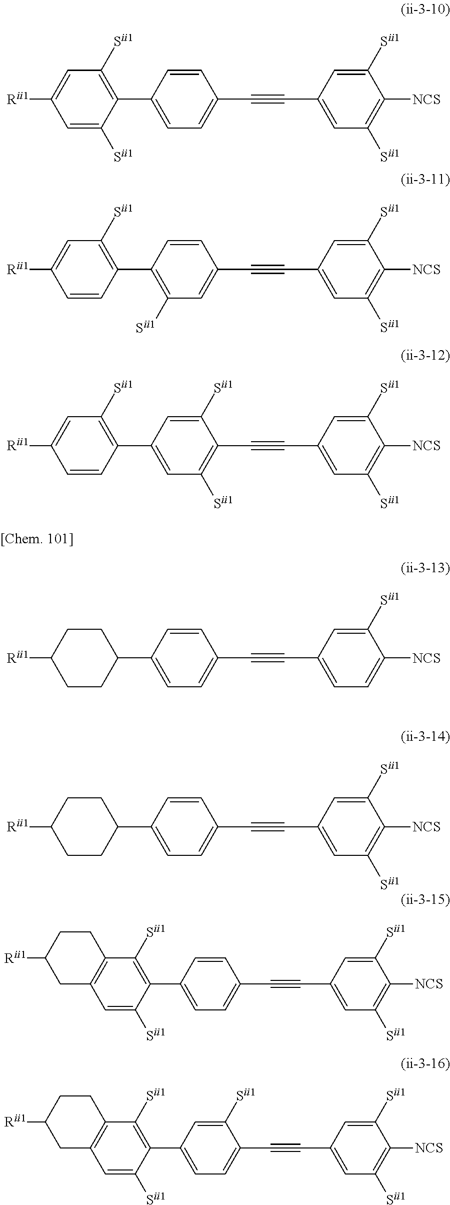

In general formula (ii-3-1) to (ii-3-16), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-3-1) include the compounds represented by structural formula (ii-3-1.1) to (ii-3-1.4) below.

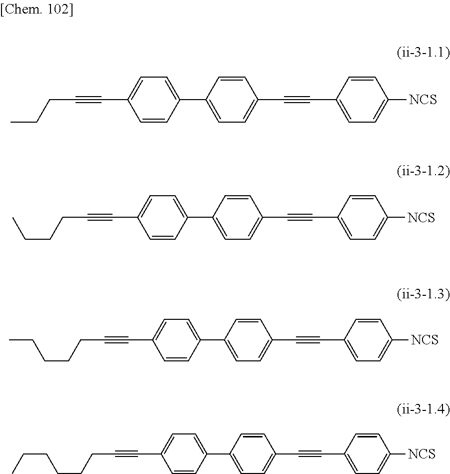

Specific examples of compounds represented by general formula (ii-3-2) include the compounds represented by structural formula (ii-3-2.1) to (ii-3-2.4) below.

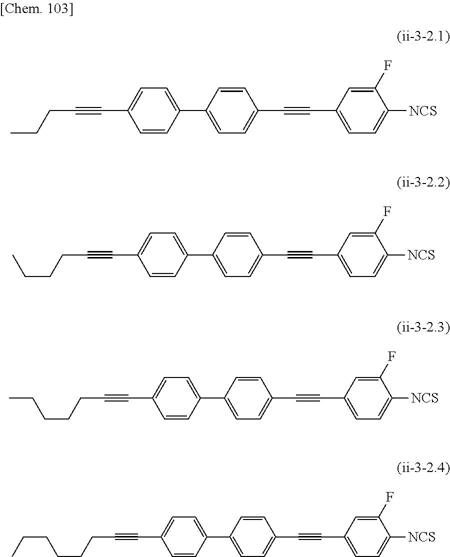

Specific examples of compounds represented by general formula (ii-3-3) include the compounds represented by structural formula (ii-3-3.1) to (ii-3-3.7) below.

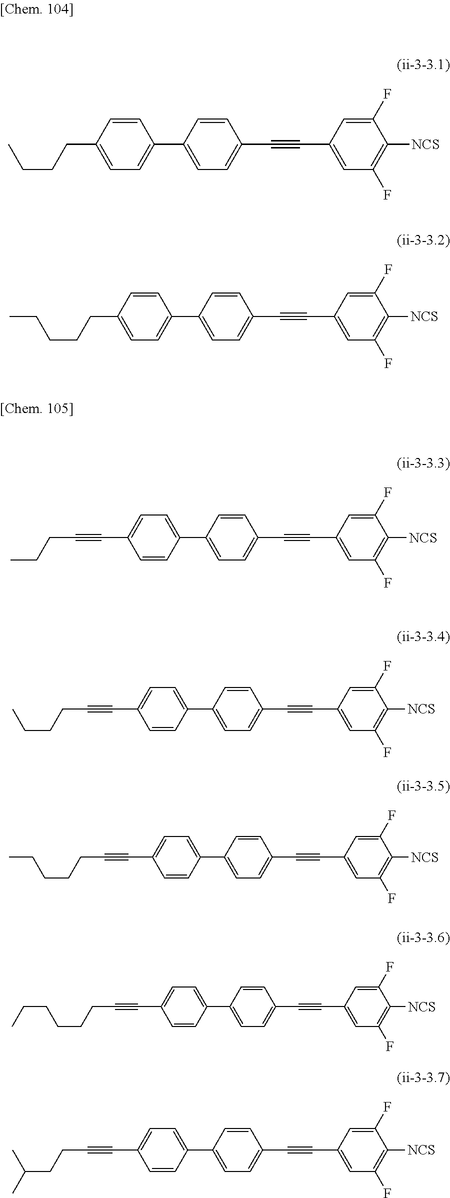

Specific examples of compounds represented by general formula (ii-3-4) include the compounds represented by structural formula (ii-3-4.1) to (ii-3-4.5) below.

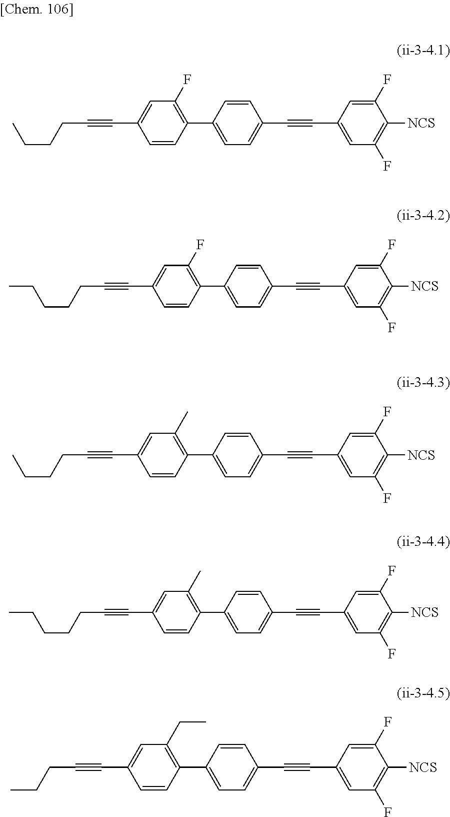

Specific examples of compounds represented by general formula (ii-3-5) include the compounds represented by structural formula (ii-3-5.1) to (i-3-5.7) below.

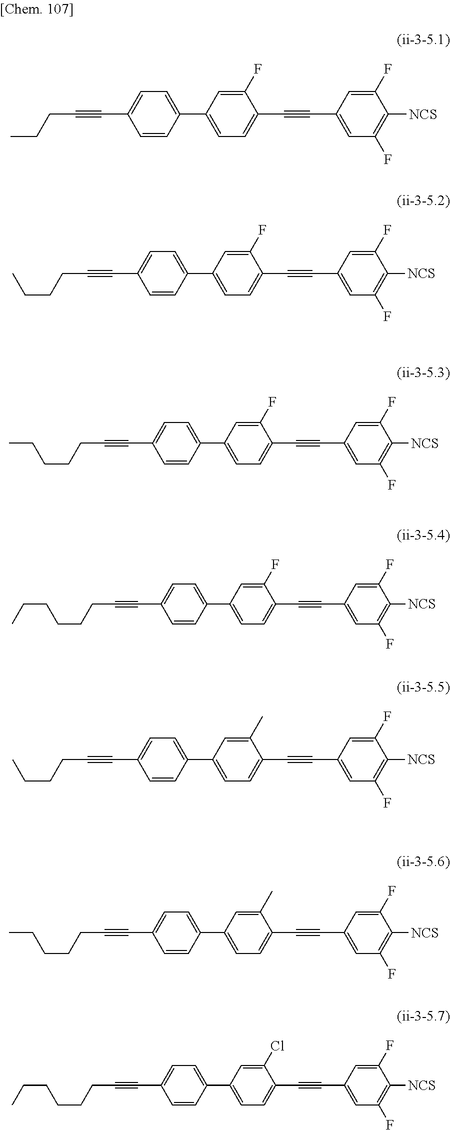

Specific examples of compounds represented by general formula (ii-3-6) include the compounds represented by structural formula (ii-3-6.1) to (ii-3-6.3) below.

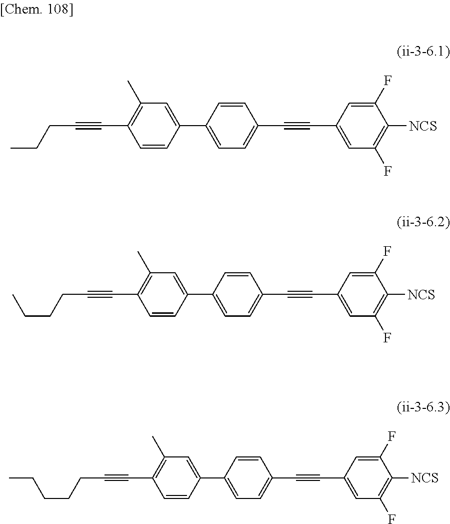

Specific examples of compounds represented by general formula (ii-3-7) include the compounds represented by structural formula (ii-3-7.1) to (ii-3-7.7) below.

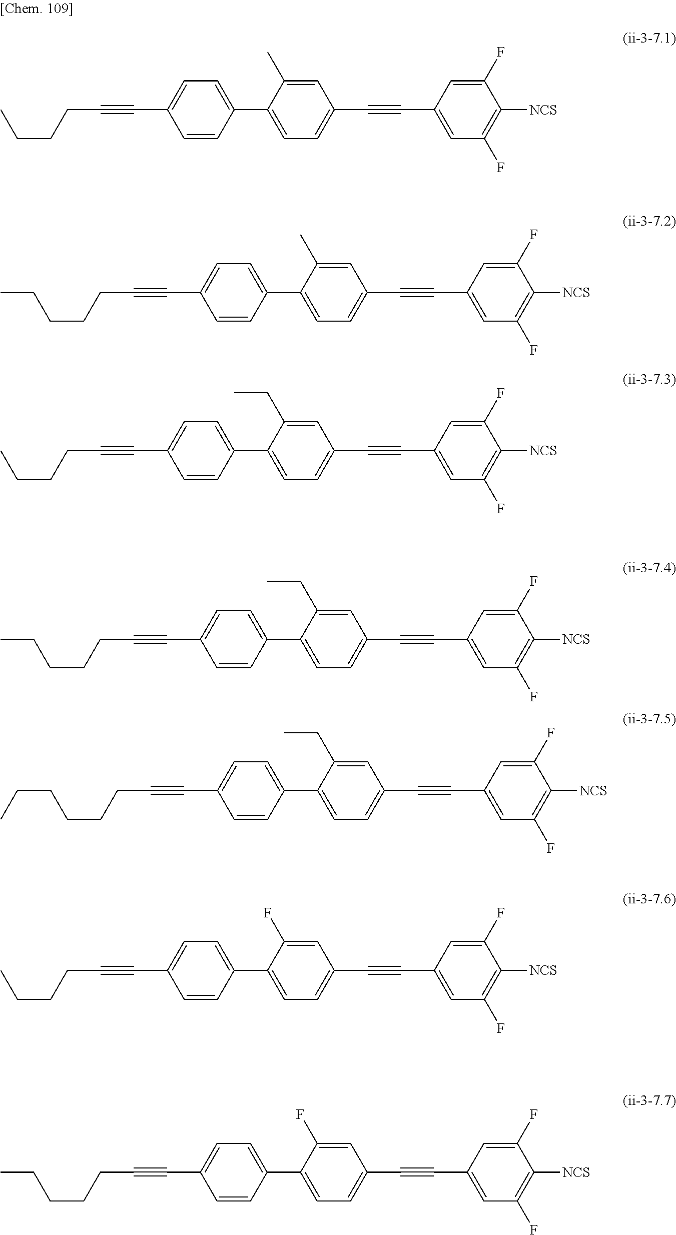

Specific examples of compounds represented by general formula (ii-3-8) include the compounds represented by structural formula (ii-3-8.1) to (ii-3-8.3) below.

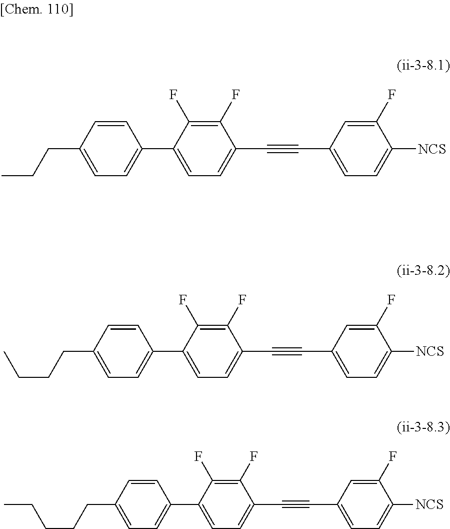

Specific examples of compounds represented by general formula (ii-3-9) include the compounds represented by structural formula (ii-3-9.1) to (ii-3-9.4) below.

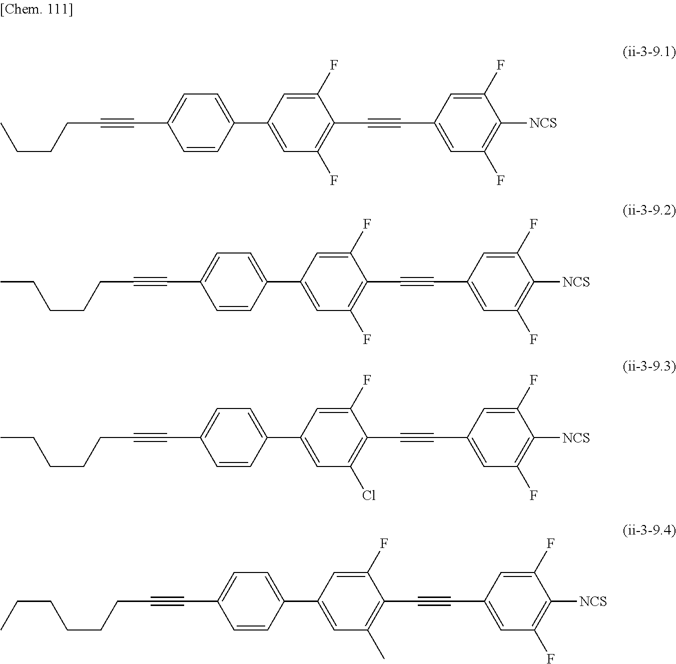

Specific examples of compounds represented by general formula (ii-3-10) include the compounds represented by structural formula (ii-3-10.1) to (ii-3-10.3) below.

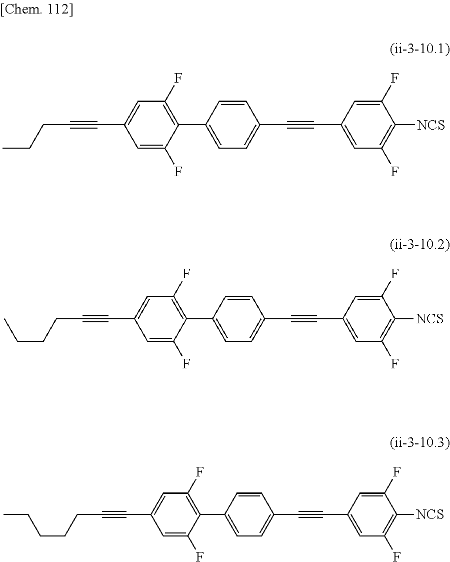

Specific examples of compounds represented by general formula (ii-3-11) include the compounds represented by structural formula (ii-3-11.1) to (ii-3-11.3) below.

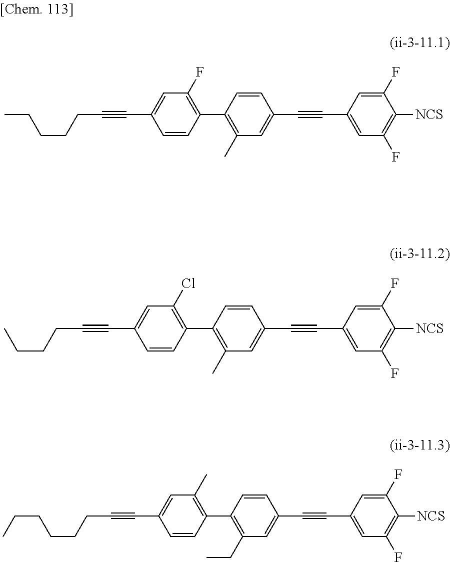

Specific examples of compounds represented by general formula (ii-3-12) include the compounds represented by structural formula (ii-3-12.1) to (ii-3-12.3) below.

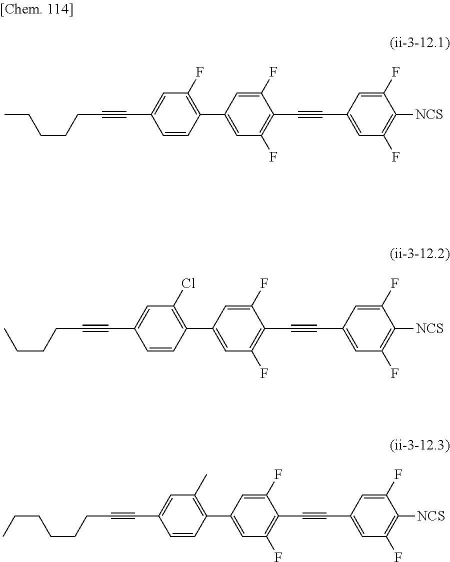

Specific examples of compounds represented by general formula (ii-3-13) include the compounds represented by structural formula (ii-3-13.1) to (ii-3-13.4) below.

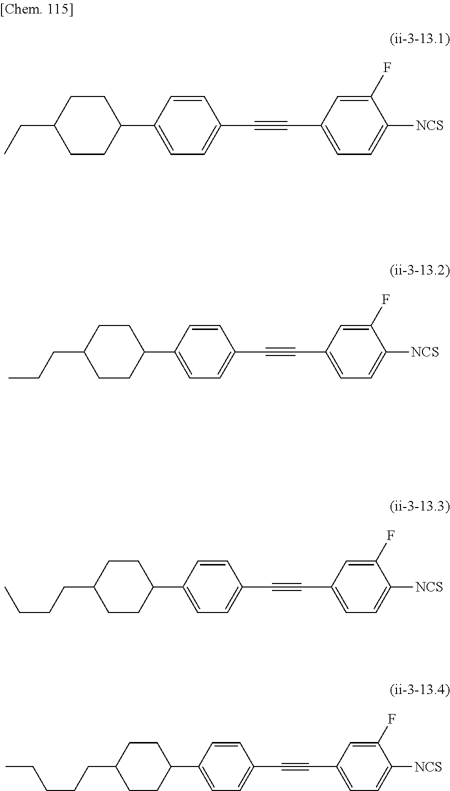

Specific examples of compounds represented by general formula (ii-3-14) include the compounds represented by structural formula (ii-3-14.1) to (ii-3-14.3) below.

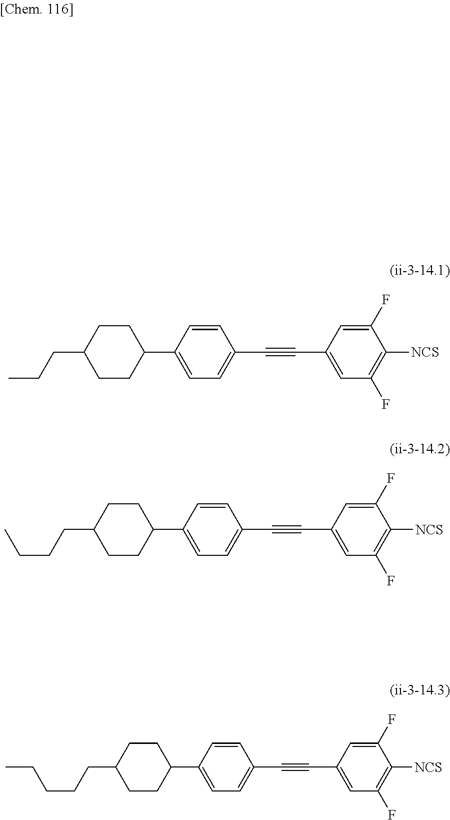

Specific examples of compounds represented by general formula (ii-3-15) include the compounds represented by structural formula (ii-3-15.1) to (ii-3-15.3) below.

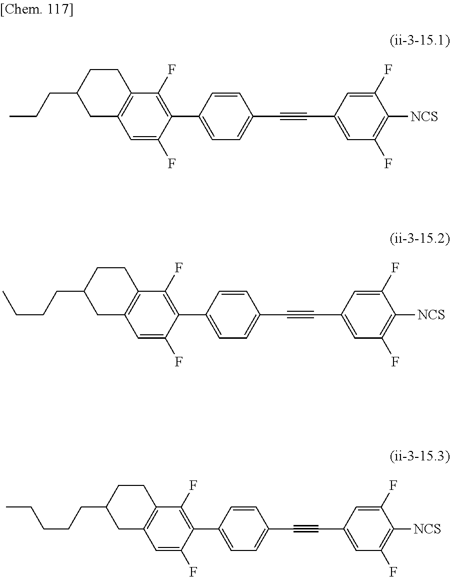

Specific examples of compounds represented by general formula (ii-3-16) include the compounds represented by structural formula (ii-3-16.1) to (ii-3-16.3) below.

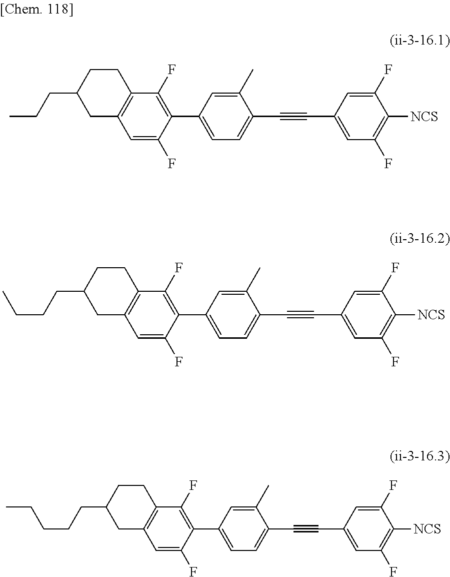

Compounds represented by general formula (ii-4) are preferably compounds represented by general formula (ii-4-1) to (ii-4-26) below.

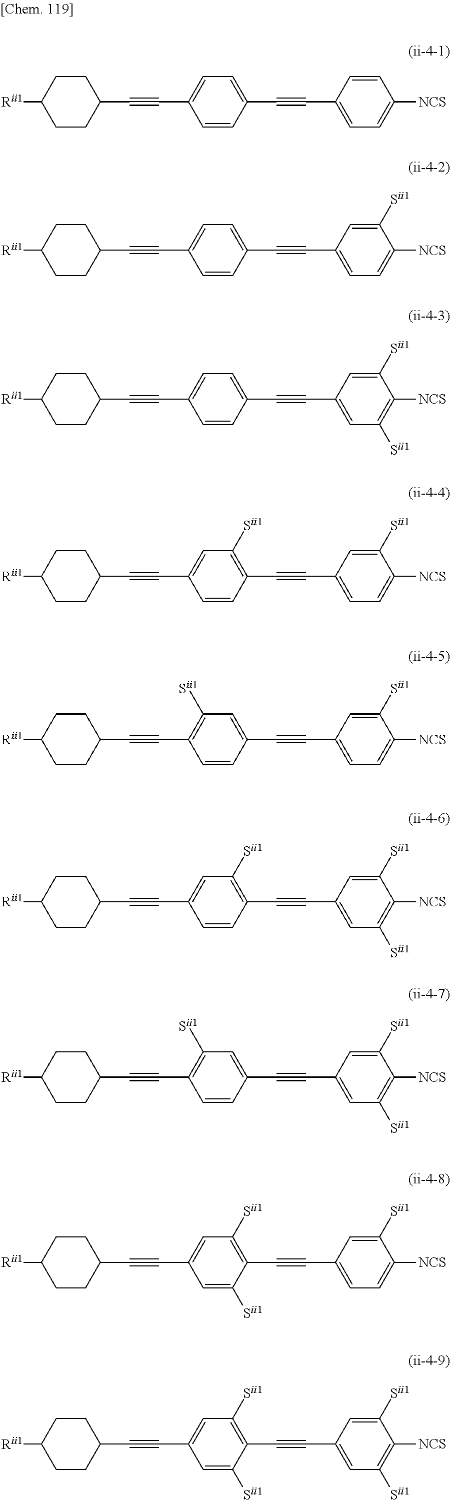

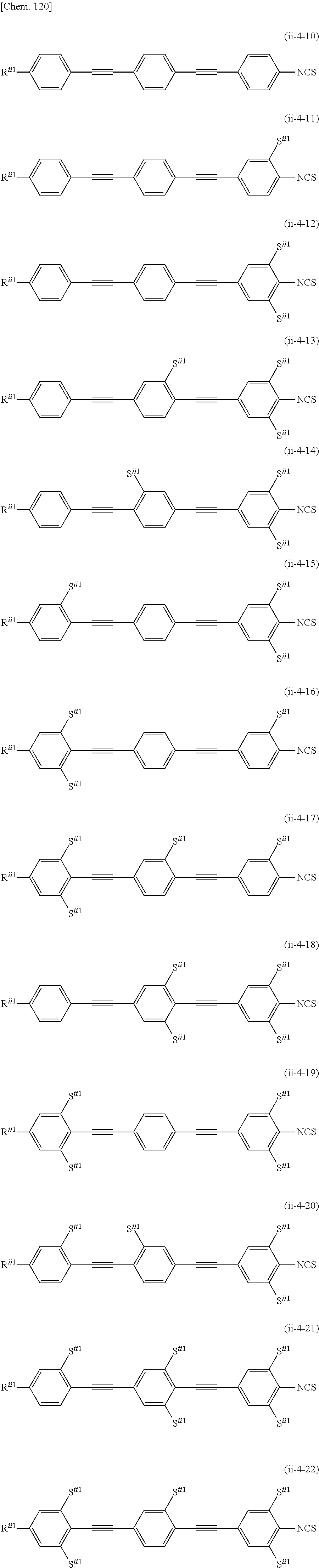

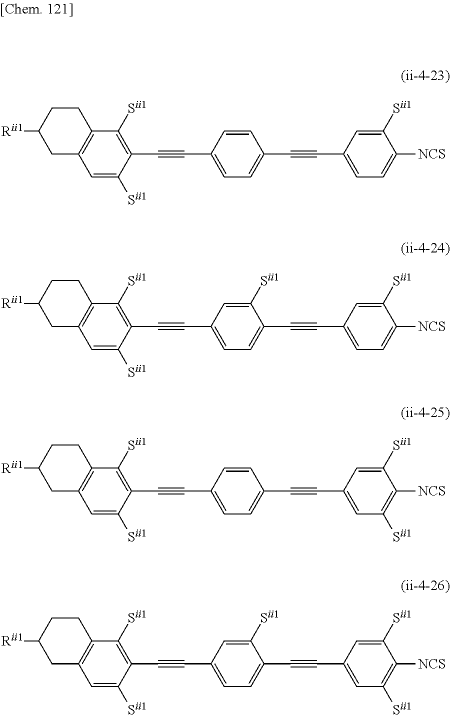

In general formula (ii-4-1) to (ii-4-26), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-4-1) include the compounds represented by structural formula (ii-4-1.1) to (ii-4-1.4) below.

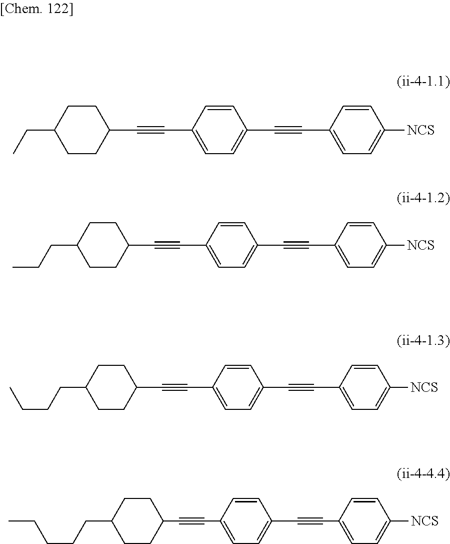

Specific examples of compounds represented by general formula (ii-4-2) include the compounds represented by structural formula (ii-4-2.1) to (ii-4-2.4) below.

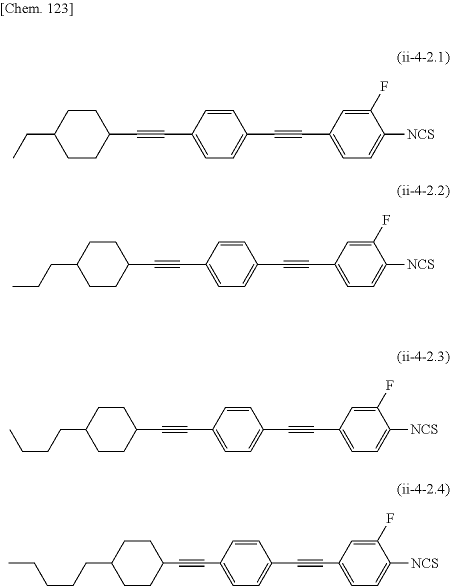

Specific examples of compounds represented by general formula (ii-4-3) include the compounds represented by structural formula (ii-4-3.1) to (ii-4-3.8) below.

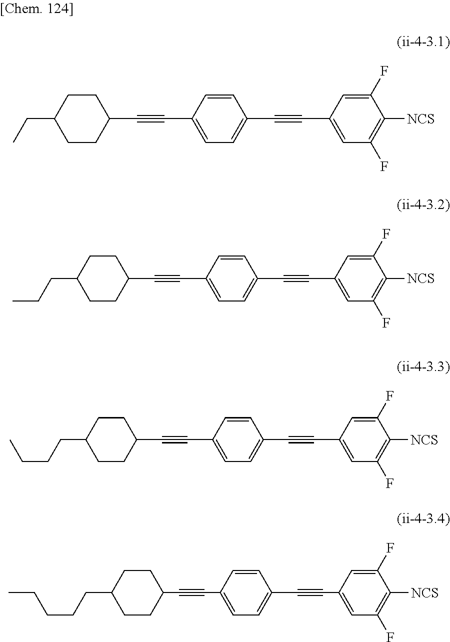

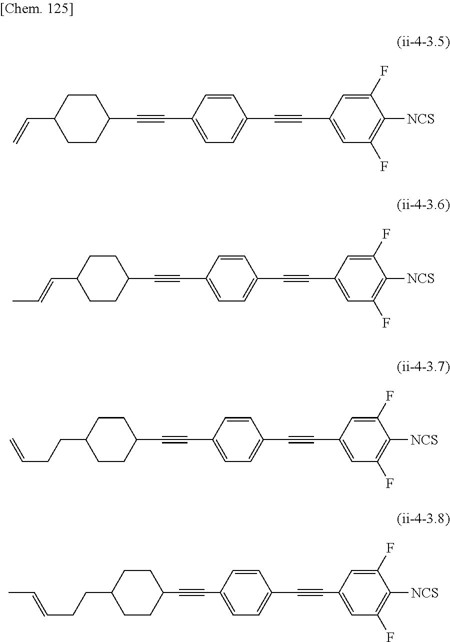

Specific examples of compounds represented by general formula (ii-4-4) include the compounds represented by structural formula (ii-4-4.1) to (ii-4-4.5) below.

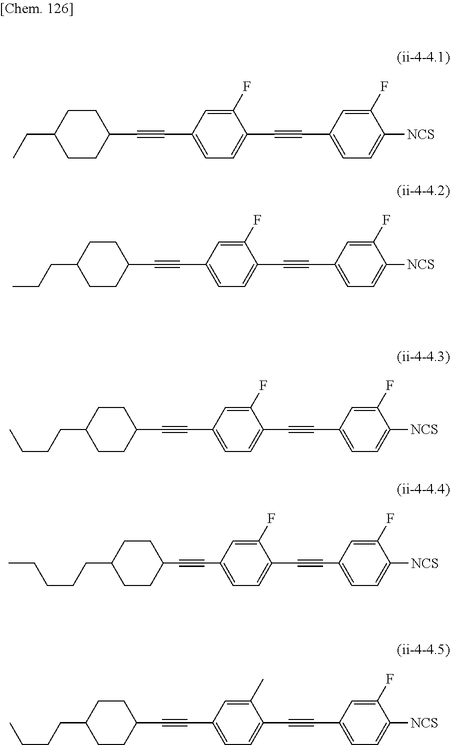

Specific examples of compounds represented by general formula (ii-4-5) include the compound represented by structural formula (ii-4-5.1) below.

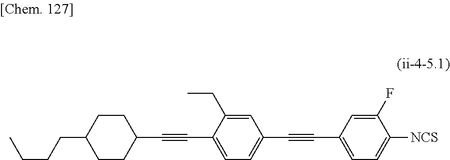

Specific examples of compounds represented by general formula (ii-4-6) include the compounds represented by structural formula (ii-4-6.1) to (ii-4-6.5) below.

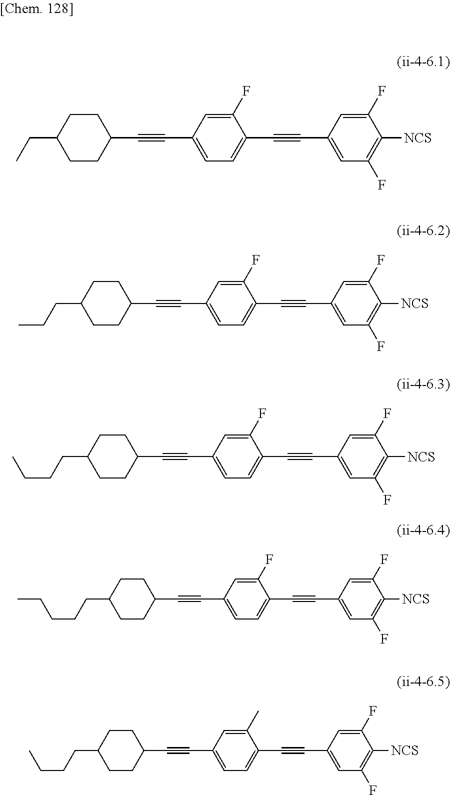

Specific examples of compounds represented by general formula (ii-4-7) include the compound represented by structural formula (ii-4-7.1) below.

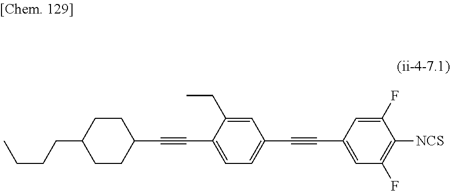

Specific examples of compounds represented by general formula (ii-4-8) include the compounds represented by structural formula (ii-4-8.1) to (ii-4-8.4) below.

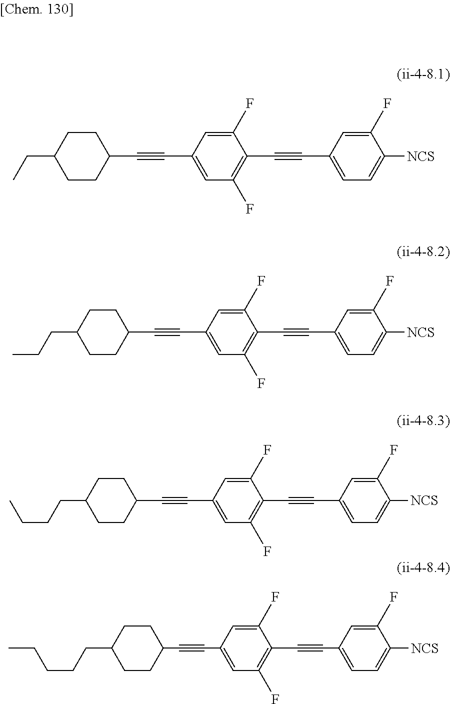

Specific examples of compounds represented by general formula (ii-4-9) include the compounds represented by structural formula (ii-4-9.1) to (ii-4-9.4) below.

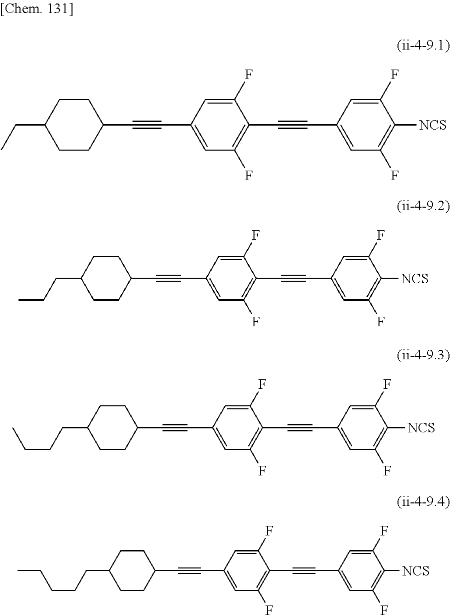

Specific examples of compounds represented by general formula (ii-4-10) include the compounds represented by structural formula (ii-4-10.1) to (ii-4-10.4) below.

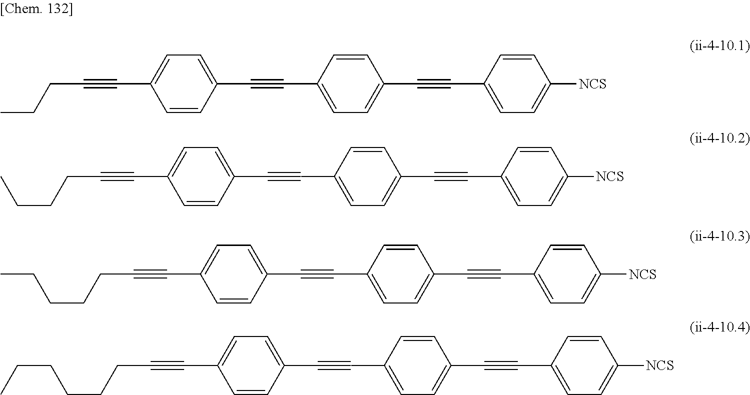

Specific examples of compounds represented by general formula (ii-4-11) include the compounds represented by structural formula (ii-4-11.1) to (i-4-11.5) below.

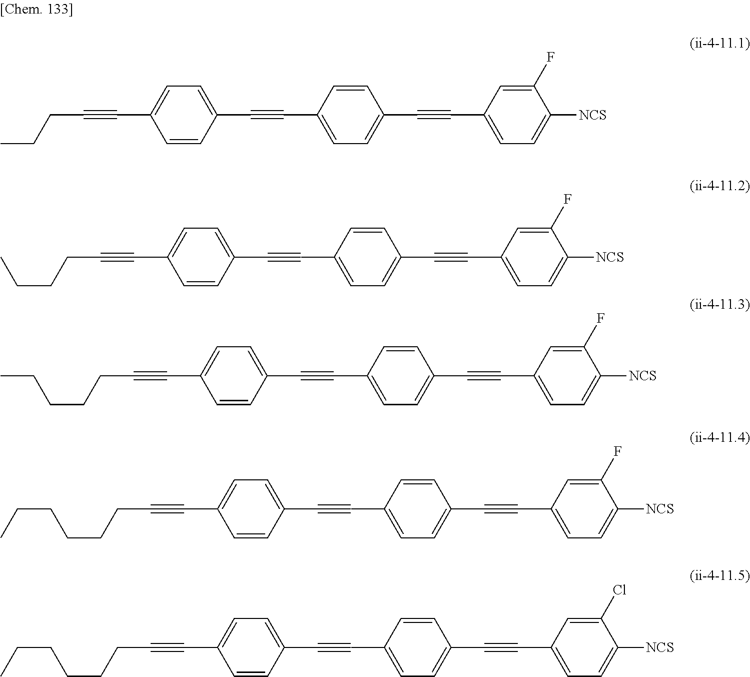

Specific examples of compounds represented by general formula (ii-4-12) include the compounds represented by structural formula (ii-4-12.1) to (ii-4-12.5) below.

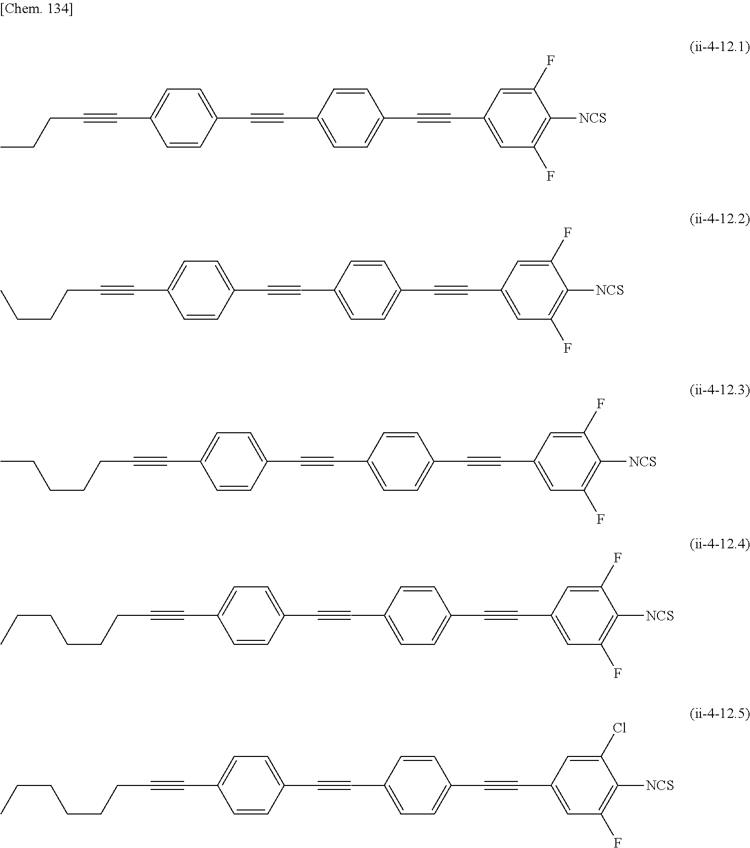

Specific examples of compounds represented by general formula (ii-4-13) include the compounds represented by structural formula (ii-4-13.1) to (ii-4-13.4) below.

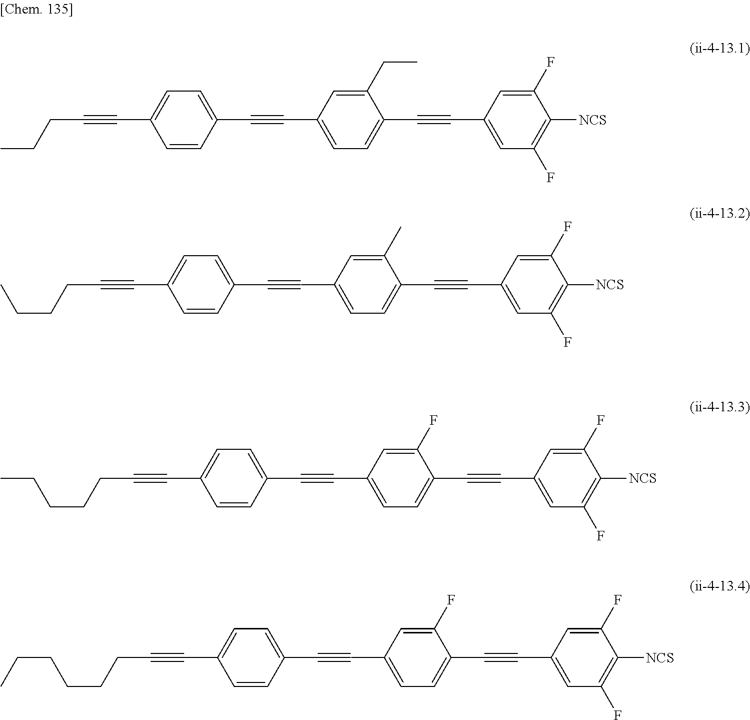

Specific examples of compounds represented by general formula (ii-4-14) include the compounds represented by structural formula (ii-4-14.1) to (ii-4-14.4) below.

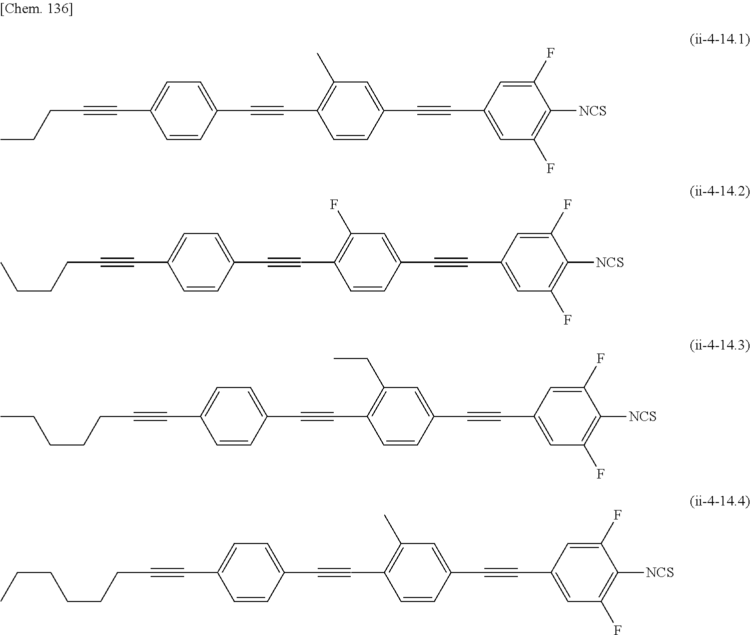

Specific examples of compounds represented by general formula (ii-4-15) include the compounds represented by structural formula (ii-4-15.1) to (ii-4-15.6) below.

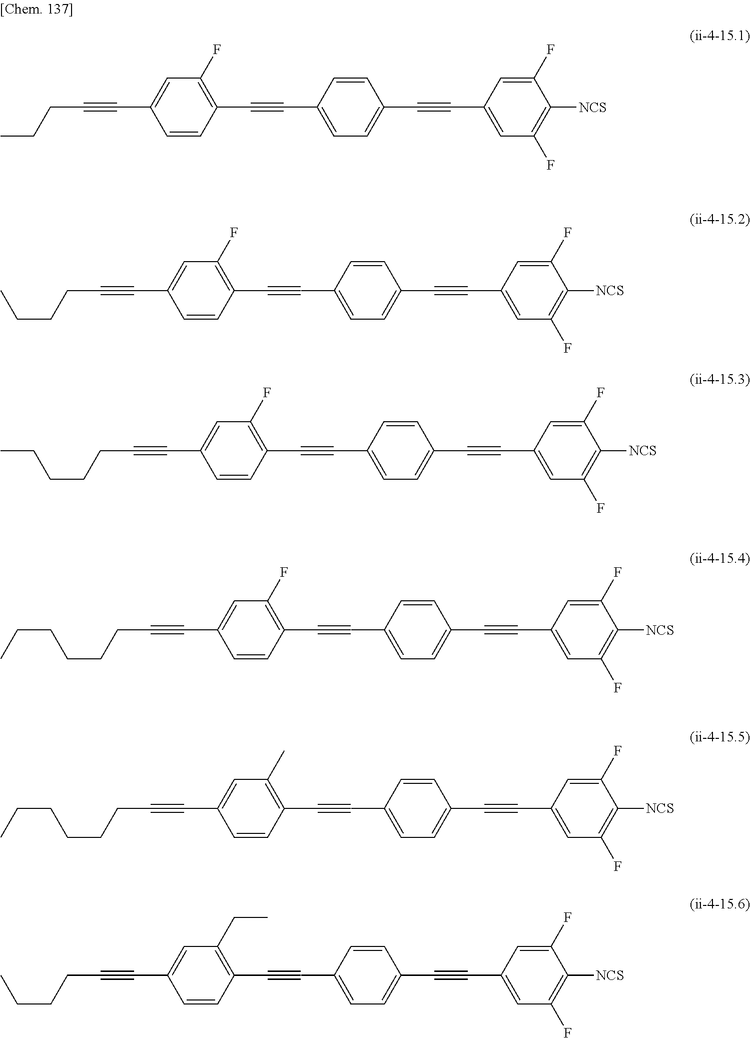

Specific examples of compounds represented by general formula (ii-4-16) include the compounds represented by structural formula (ii-4-16.1) to (ii-4-16.3) below.

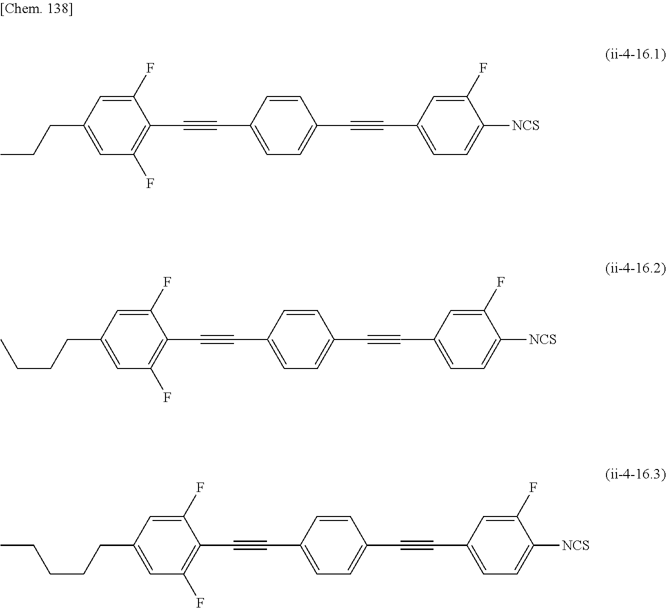

Specific examples of compounds represented by general formula (ii-4-17) include the compounds represented by structural formula (ii-4-17.1) to (ii-4-17.3) below.

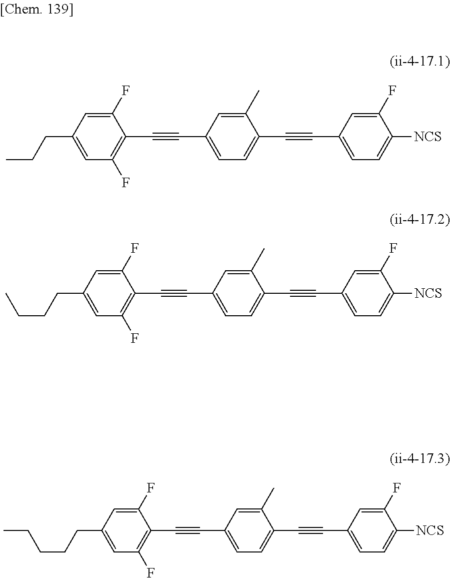

Specific examples of compounds represented by general formula (ii-4-18) include the compounds represented by structural formula (ii-4-18.1) to (ii-4-18.4) below.

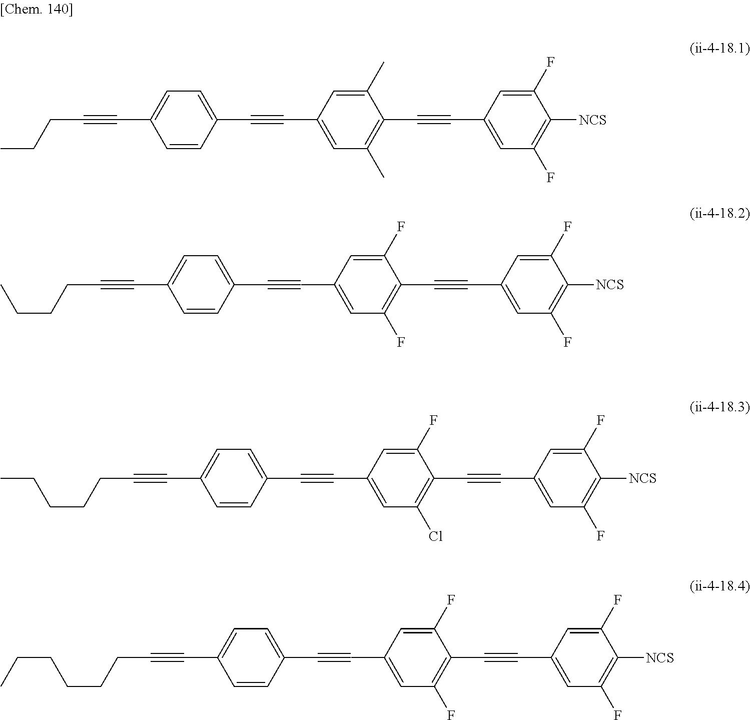

Specific examples of compounds represented by general formula (ii-4-19) include the compounds represented by structural formula (ii-4-19.1) to (ii-4-19.8) below.

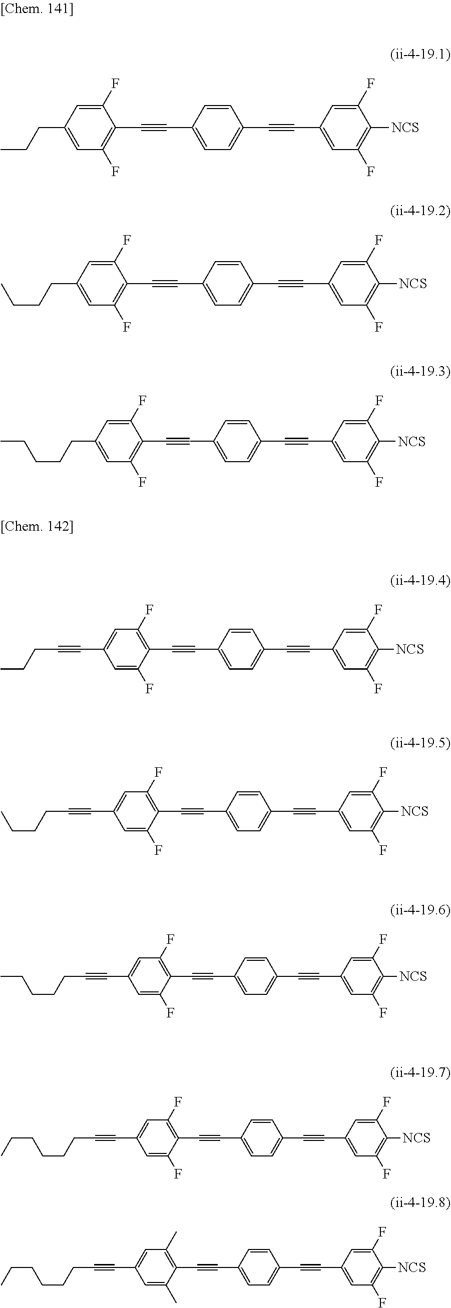

Specific examples of compounds represented by general formula (ii-4-20) include the compounds represented by structural formula (ii-4-20.1) to (ii-4-20.4) below.

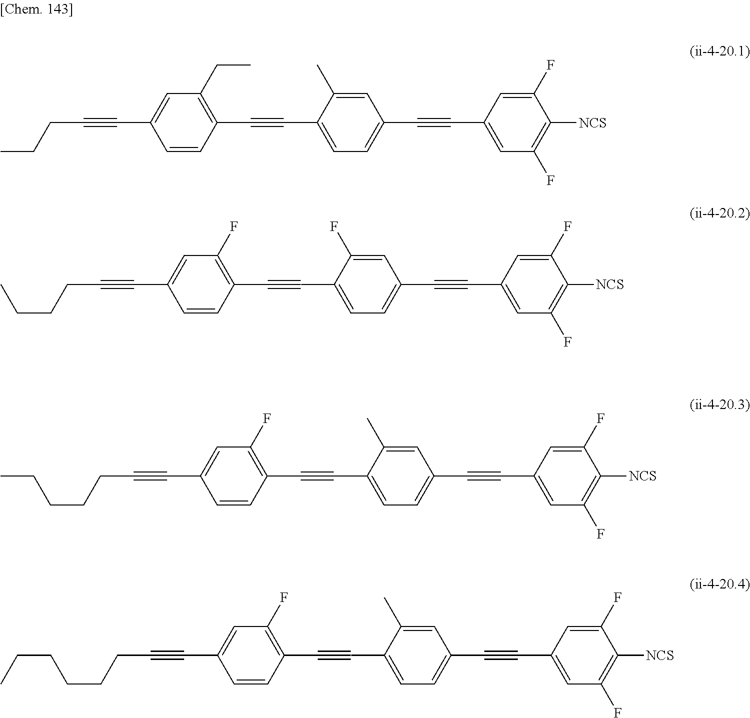

Specific examples of compounds represented by general formula (ii-4-21) include the compounds represented by structural formula (ii-4-21.1) to (ii-4-21.4) below.

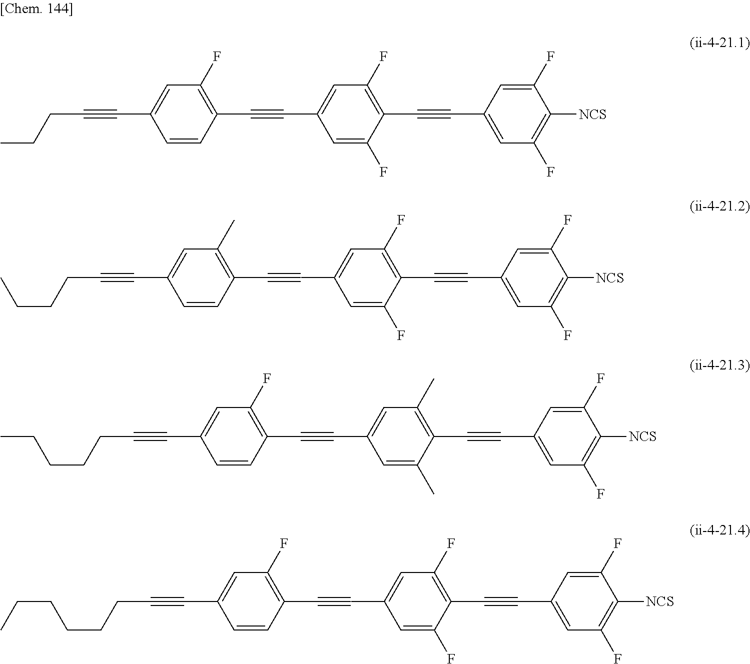

Specific examples of compounds represented by general formula (ii-4-22) include the compounds represented by structural formula (ii-4-22.1) to (ii-4-22.3) below.

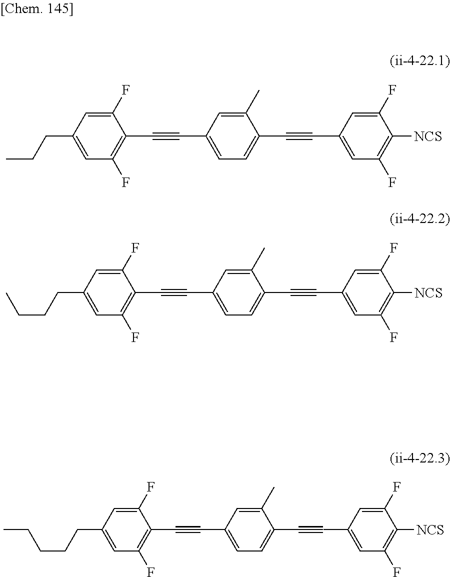

Specific examples of compounds represented by general formula (ii-4-23) include the compounds represented by structural formula (ii-4-23.1) to (ii-4-23.3) below.

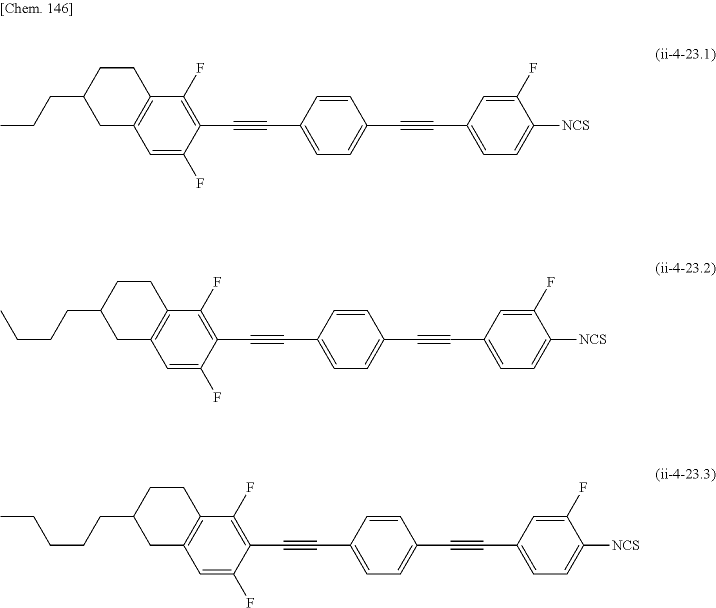

Specific examples of compounds represented by general formula (ii-4-24) include the compounds represented by structural formula (ii-4-24.1) to (ii-4-24.3) below.

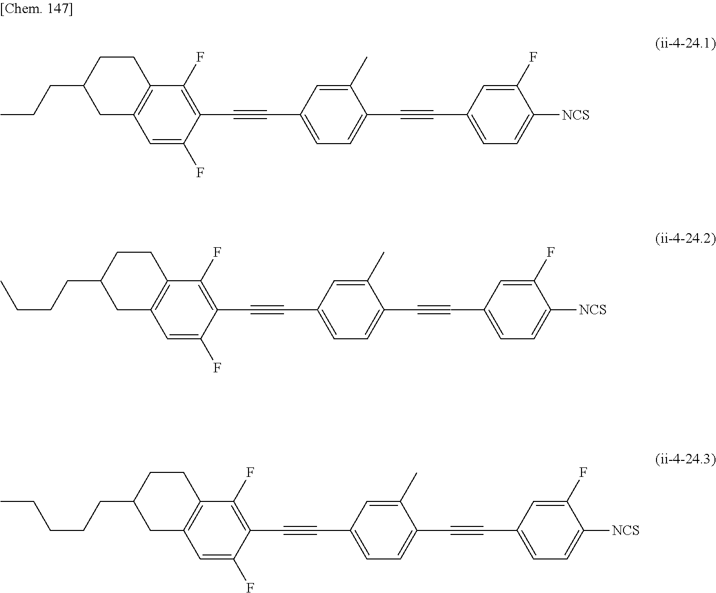

Specific examples of compounds represented by general formula (ii-4-25) include the compounds represented by structural formula (ii-4-25.1) to (ii-4-25.3) below.

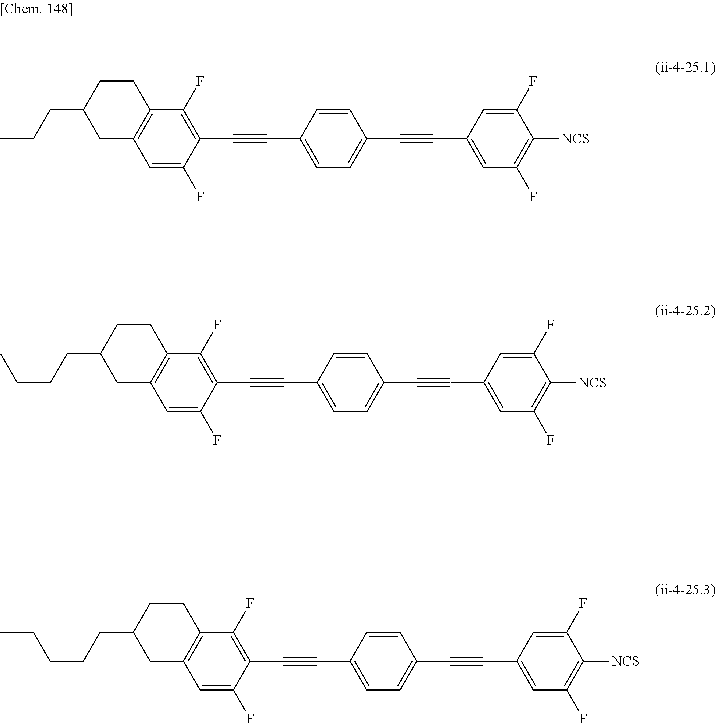

Specific examples of compounds represented by general formula (ii-4-26) include the compounds represented by structural formula (ii-4-26.1) to (ii-4-26.3) below.

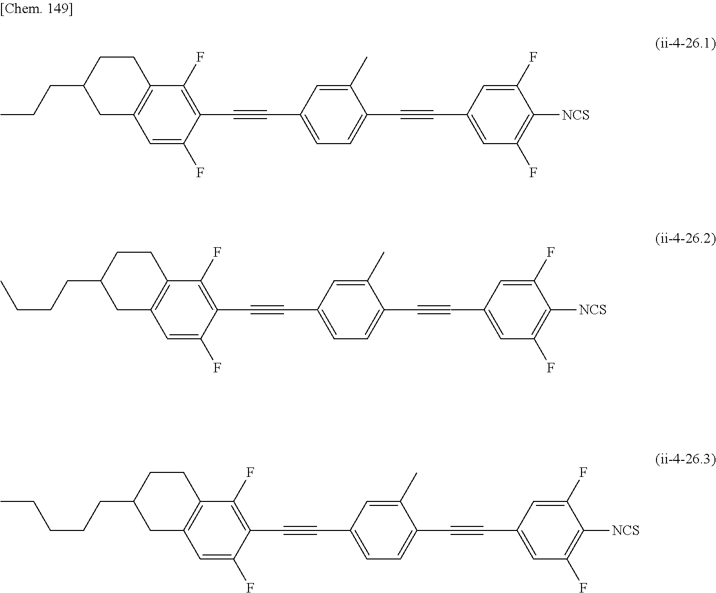

Compounds represented by general formula (ii-5) are preferably compounds represented by general formula (ii-5-1) to (ii-5-5) below.

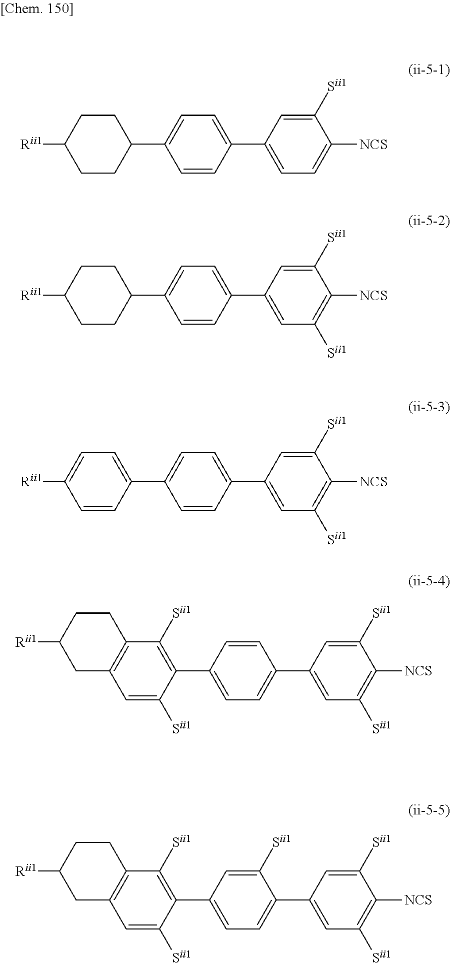

In general formula (ii-5-1) to (ii-5-5), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-5-1) include the compounds represented by structural formula (ii-5-1.1) to (ii-5-1.4) below.

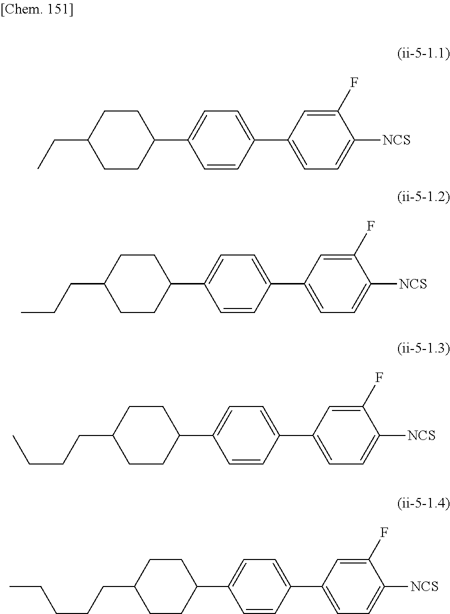

Specific examples of compounds represented by general formula (ii-5-2) include the compounds represented by structural formula (ii-5-2.1) to (ii-5-2.4) below.

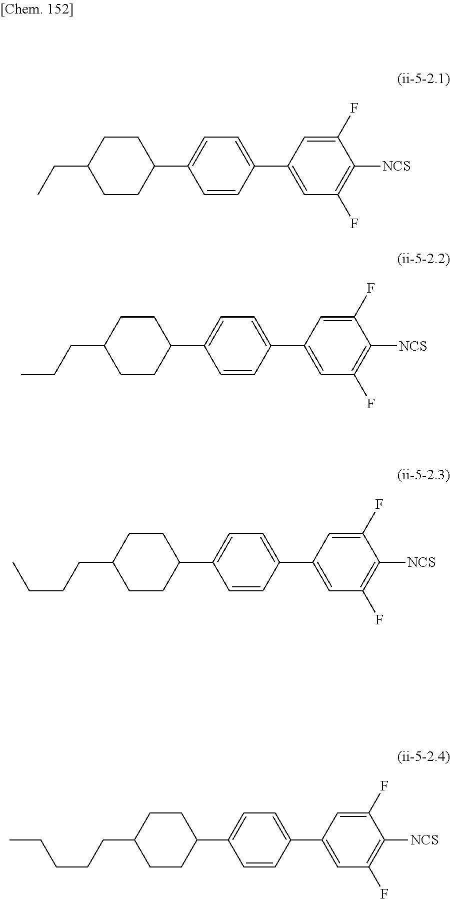

Specific examples of compounds represented by general formula (ii-5-3) include the compound represented by structural formula (ii-5-3.1) below.

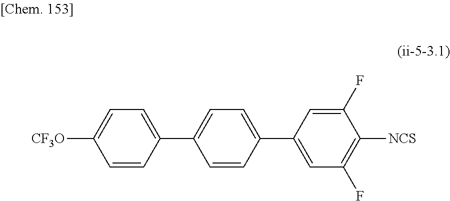

Specific examples of compounds represented by general formula (ii-5-4) include the compounds represented by structural formula (ii-5-4.1) to (ii-5-4.3) below.

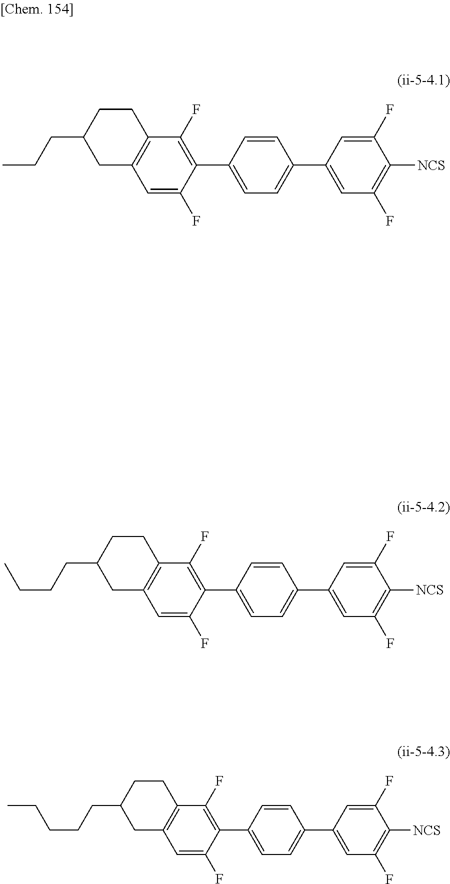

Specific examples of compounds represented by general formula (ii-5-5) include the compounds represented by structural formula (ii-5-5.1) to (ii-5-5.3) below.

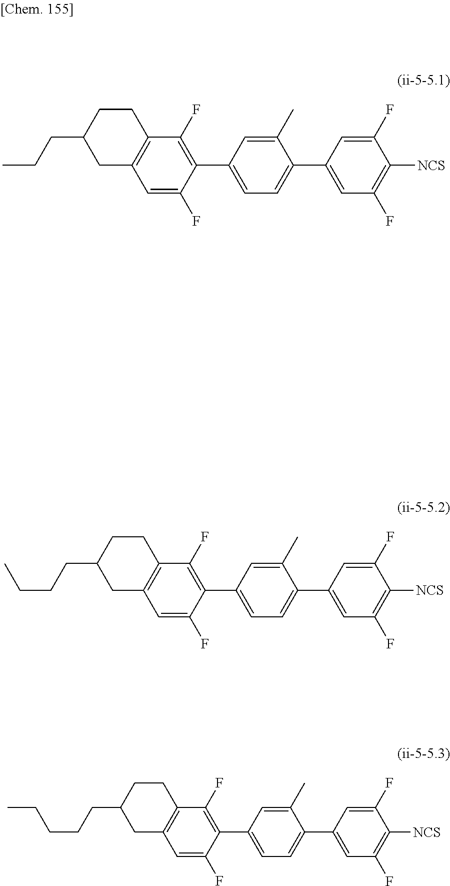

Compounds represented by general formula (ii-6) are preferably compounds represented by general formula (ii-6-1) to (ii-6-33) below.

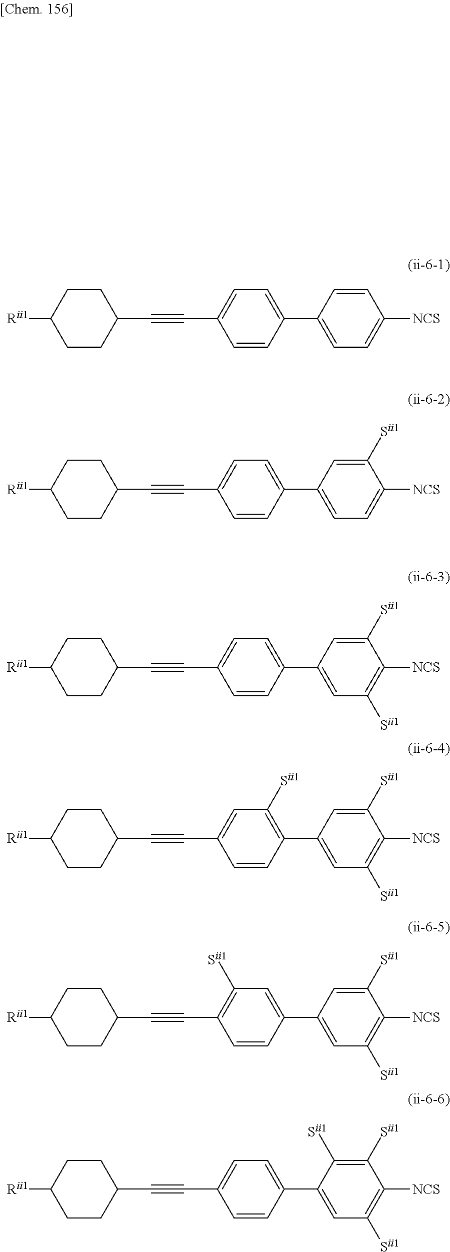

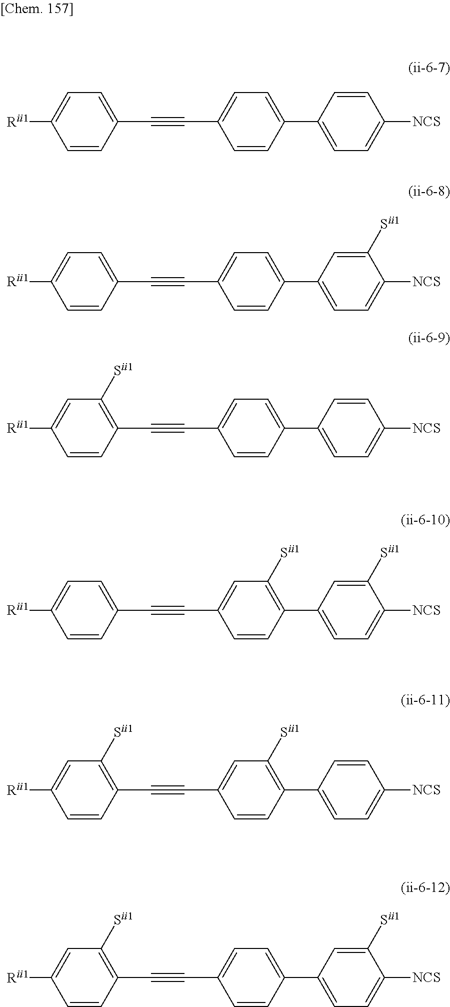

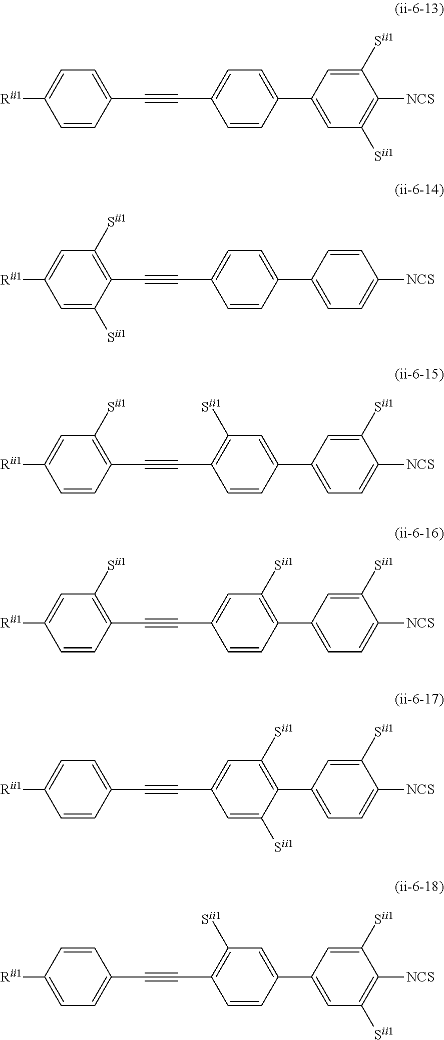

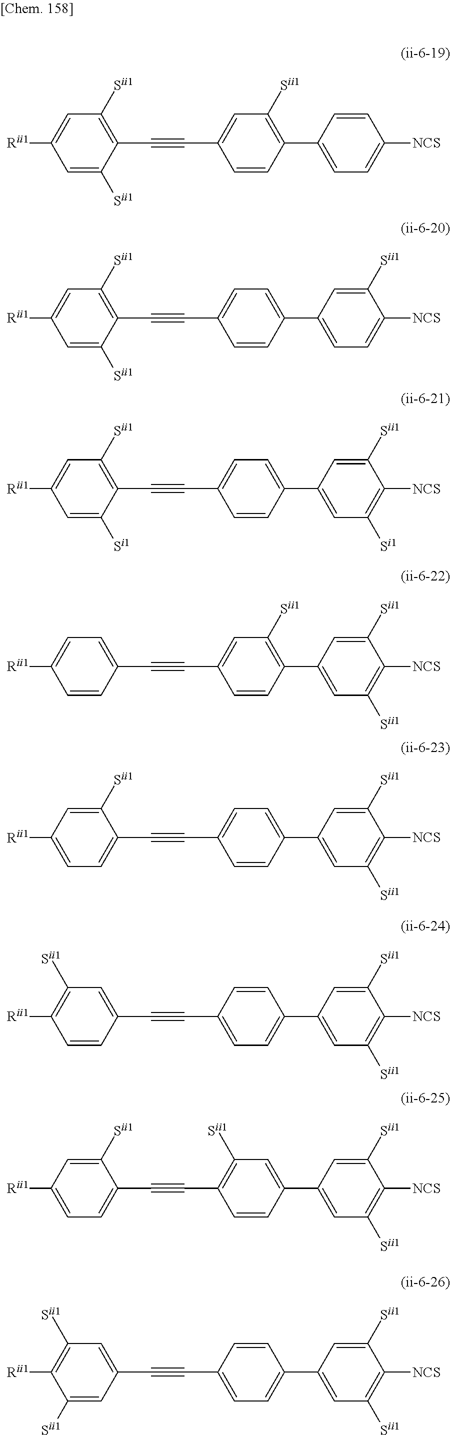

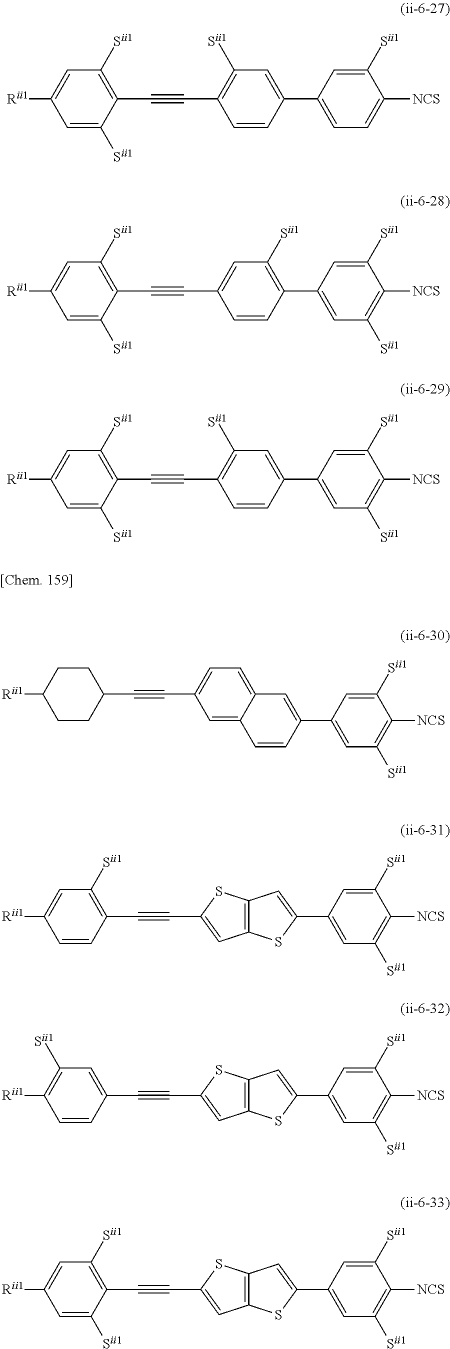

In general formula (ii-6-1) to (ii-6-33), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-6-1) include the compounds represented by structural formula (ii-6-1.1) to (ii-6-1.4) below.

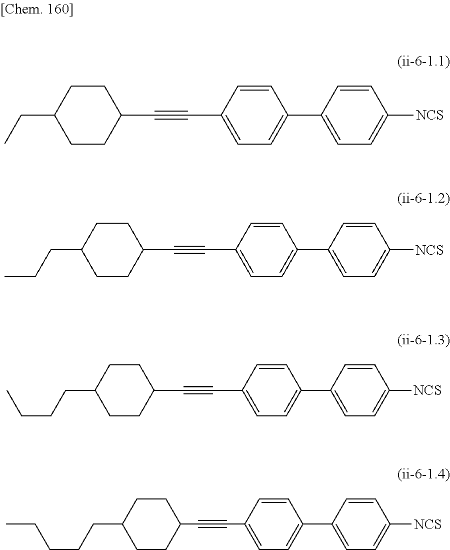

Specific examples of compounds represented by general formula (ii-6-2) include the compounds represented by structural formula (ii-6-2.1) to (ii-6-2.4) below.

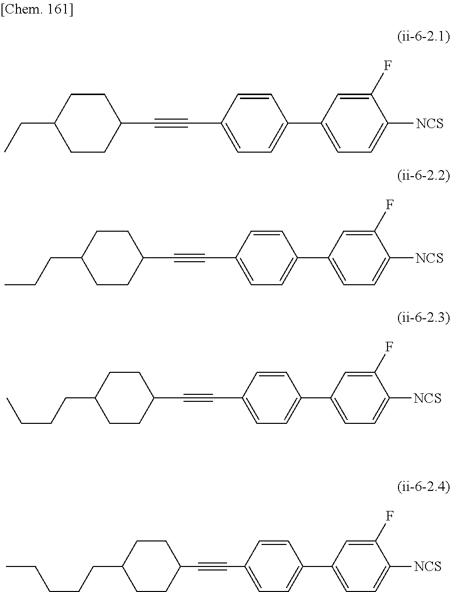

Specific examples of compounds represented by general formula (ii-6-3) include the compounds represented by structural formula (ii-6-3.1) to (ii-6-3.8) below.

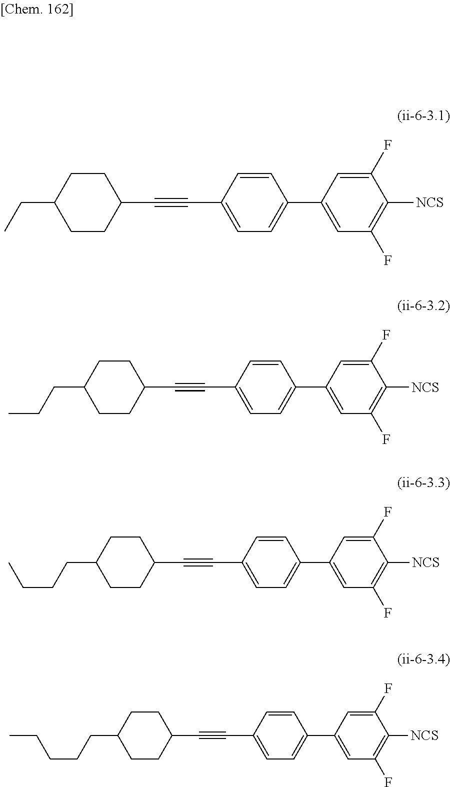

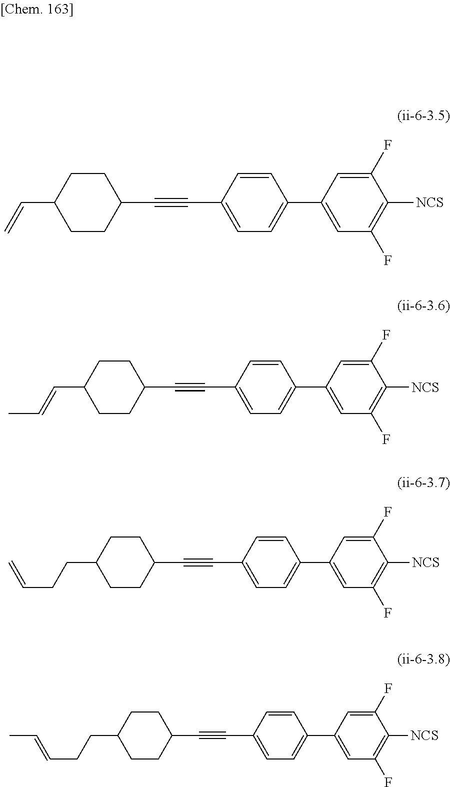

Specific examples of compounds represented by general formula (ii-6-4) include the compounds represented by structural formula (ii-6-4.1) to (ii-6-4.4) below.

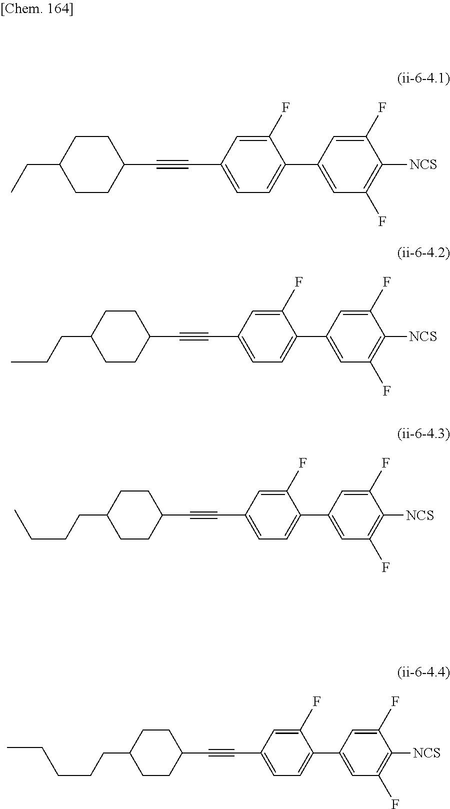

Specific examples of compounds represented by general formula (ii-6-5) include the compounds represented by structural formula (ii-6-5.1) to (ii-6-5.4) below.

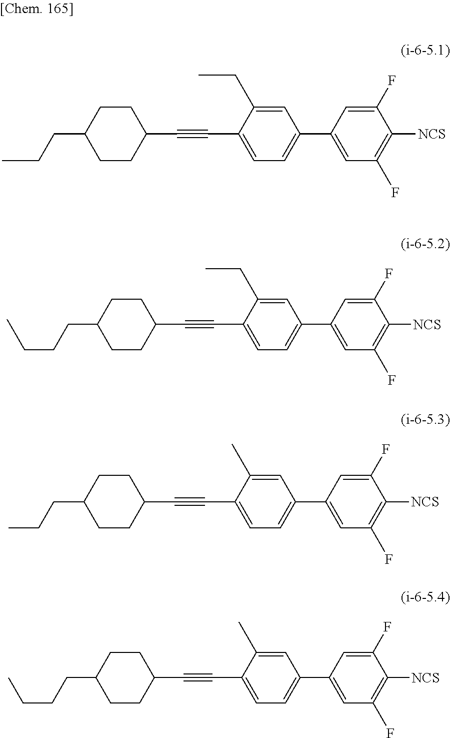

Specific examples of compounds represented by general formula (ii-6-6) include the compounds represented by structural formula (ii-6-6.1) and (ii-6-6.2) below.

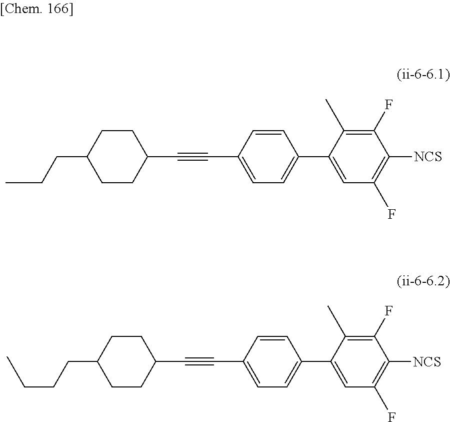

Specific examples of compounds represented by general formula (ii-6-7) include the compounds represented by structural formula (ii-6-7.1) to (ii-6-7.8) below.

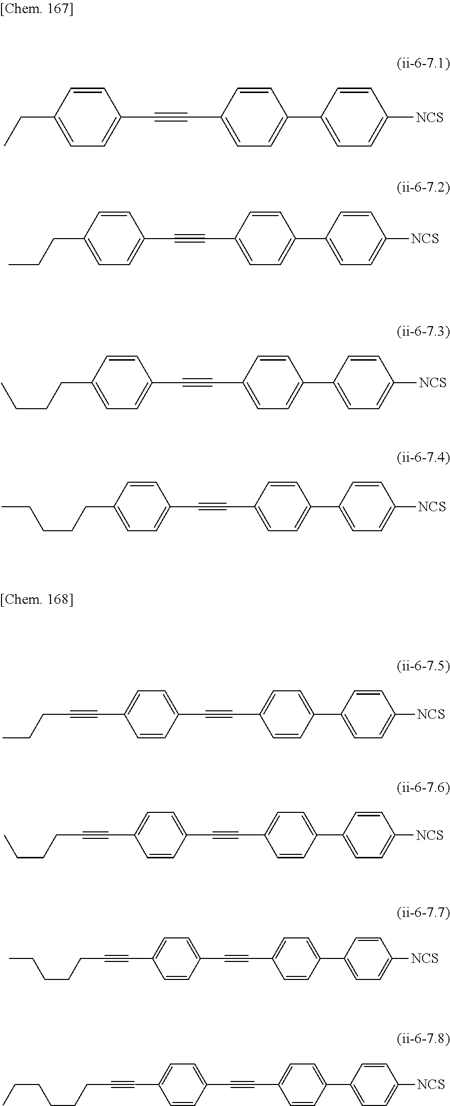

Specific examples of compounds represented by general formula (ii-6-8) include the compounds represented by structural formula (ii-6-8.1) to (ii-6-8.9) below.

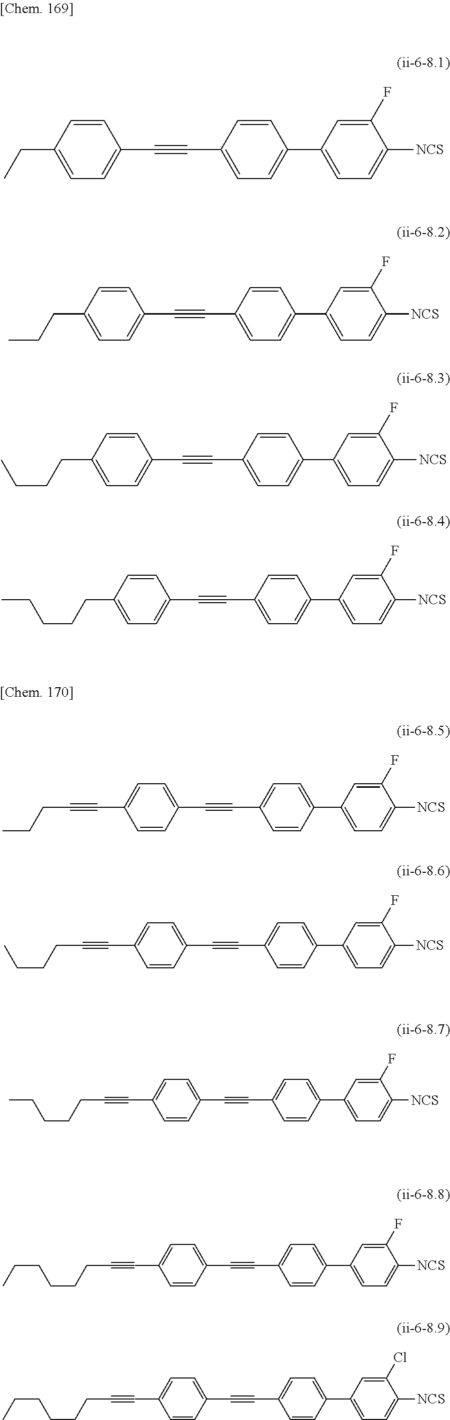

Specific examples of compounds represented by general formula (ii-6-9) include the compounds represented by structural formula (ii-6-9.1) to (ii-6-9.4) below.

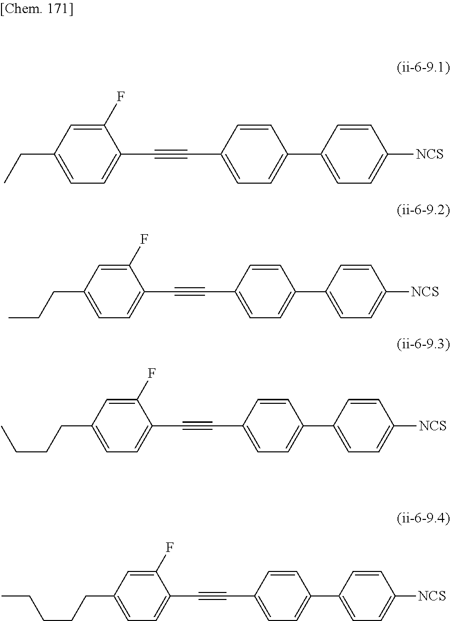

Specific examples of compounds represented by general formula (ii-6-10) include the compounds represented by structural formula (ii-6-10.1) to (ii-6-10.4) below.

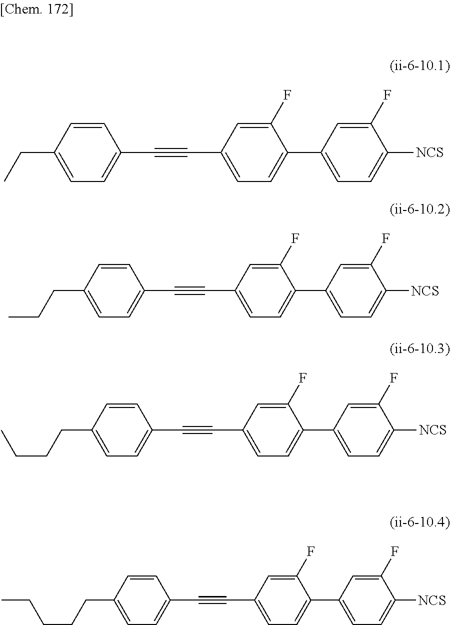

Specific examples of compounds represented by general formula (ii-6-11) include the compounds represented by structural formula (ii-6-11.1) to (ii-6-11.4) below.

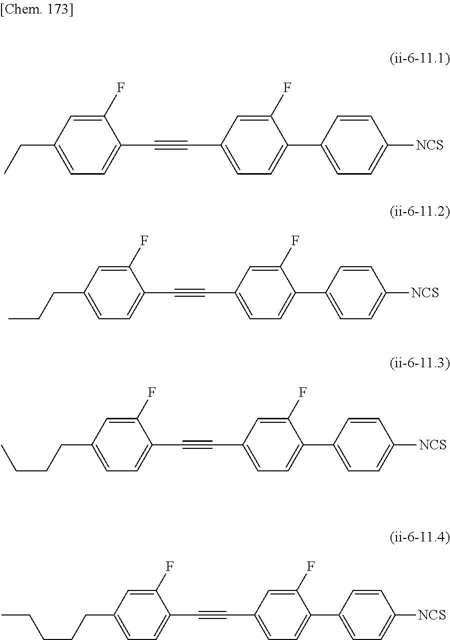

Specific examples of compounds represented by general formula (ii-6-12) include the compounds represented by structural formula (ii-6-12.1) to (ii-6-12.4) below.

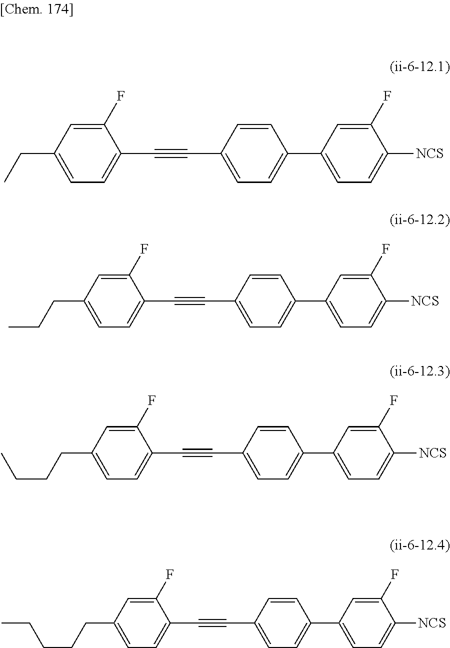

Specific examples of compounds represented by general formula (ii-6-13) include the compounds represented by structural formula (ii-6-13.1) to (ii-6-13.20) below.

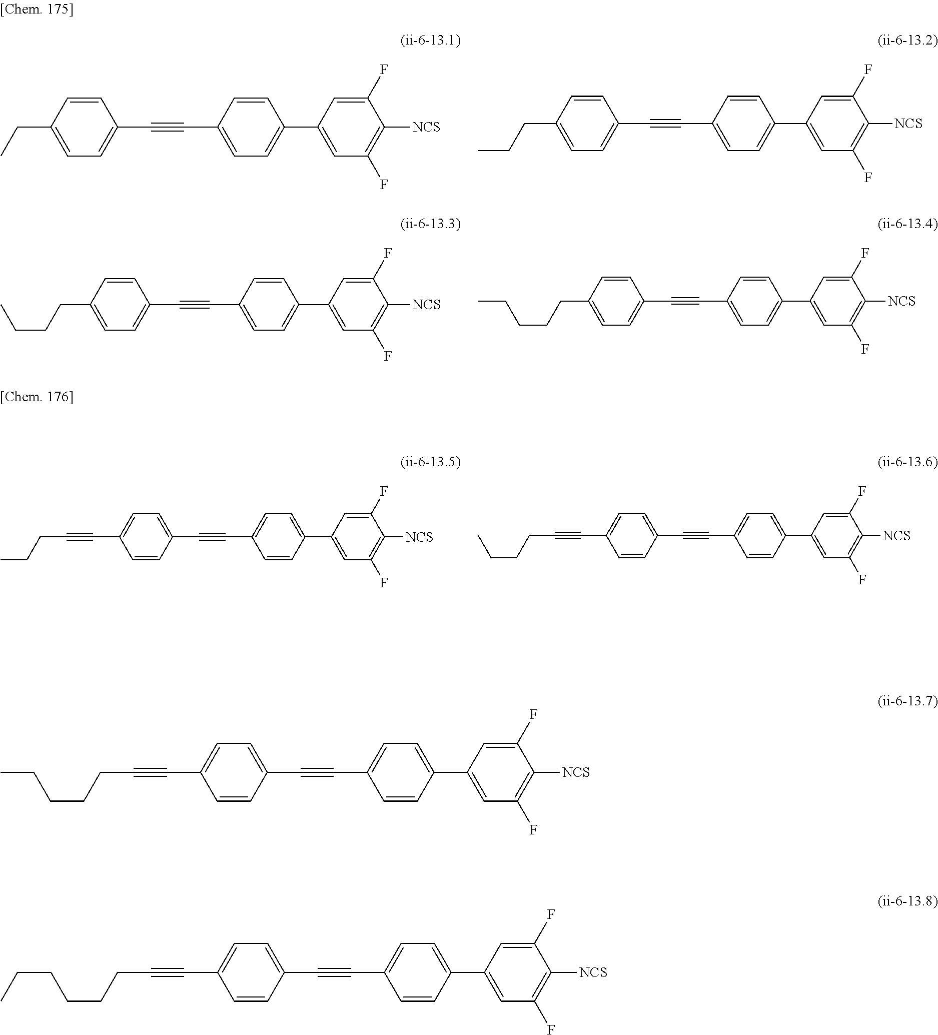

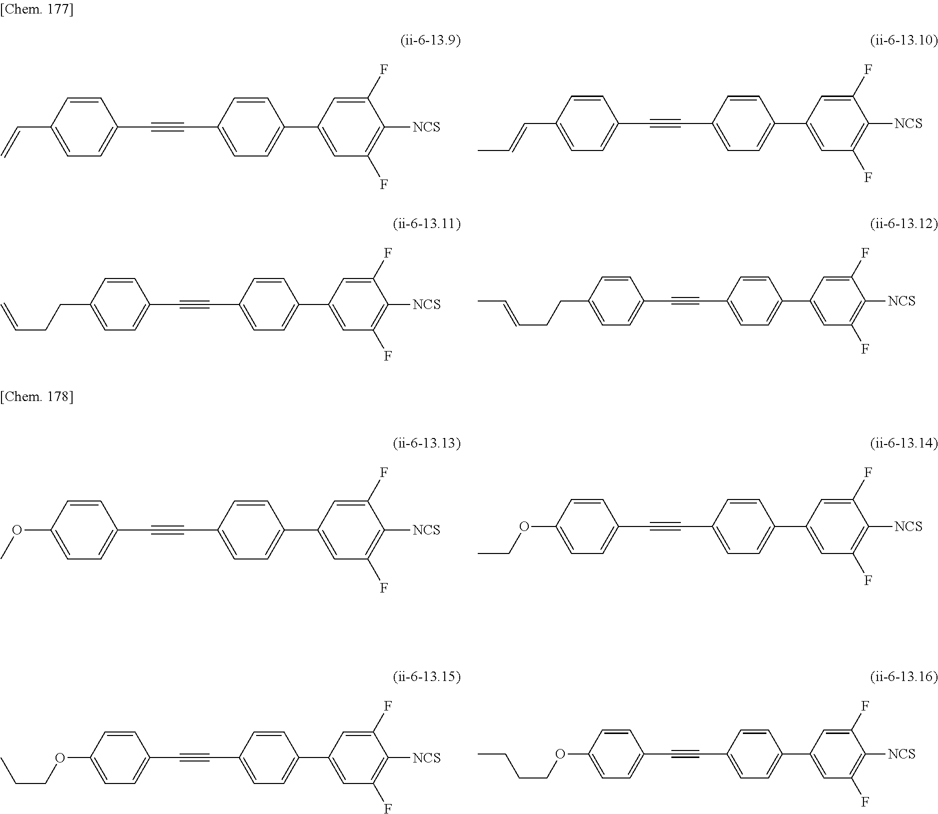

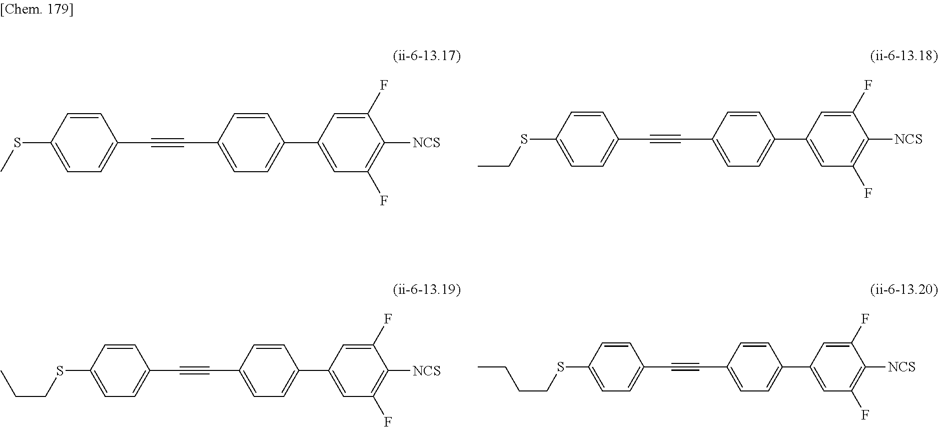

Specific examples of compounds represented by general formula (ii-6-14) include the compounds represented by structural formula (ii-6-14.1) to (ii-6-14.4) below.

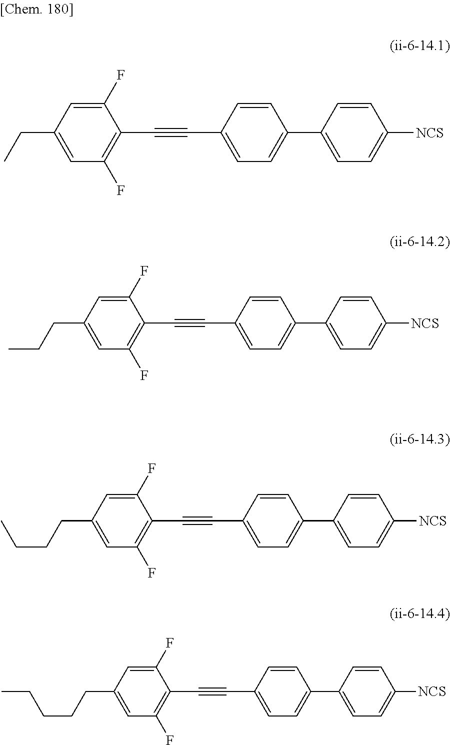

Specific examples of compounds represented by general formula (ii-6-15) include the compound represented by structural formula (ii-6-15.1) below.

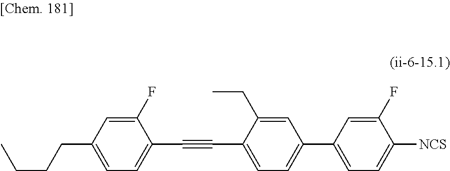

Specific examples of compounds represented by general formula (ii-6-16) include the compounds represented by structural formula (ii-6-16.1) to (ii-6-16.5) below.

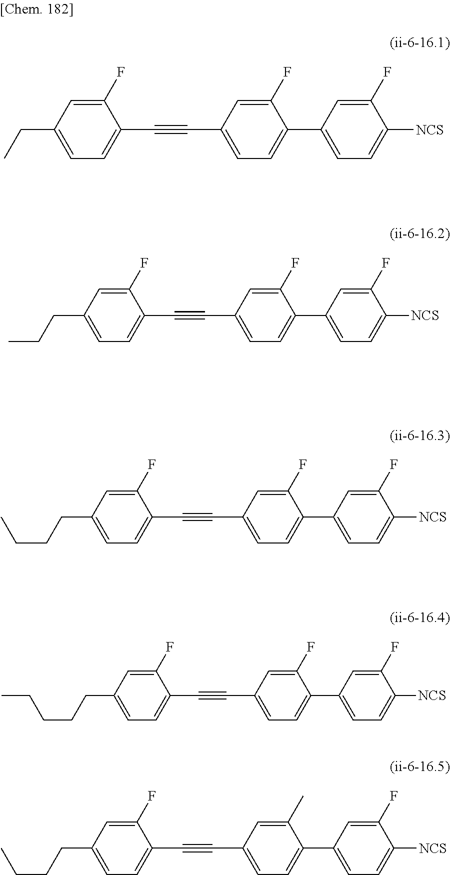

Specific examples of compounds represented by general formula (ii-6-17) include the compounds represented by structural formula (ii-6-17.1) to (ii-6-17.4) below.

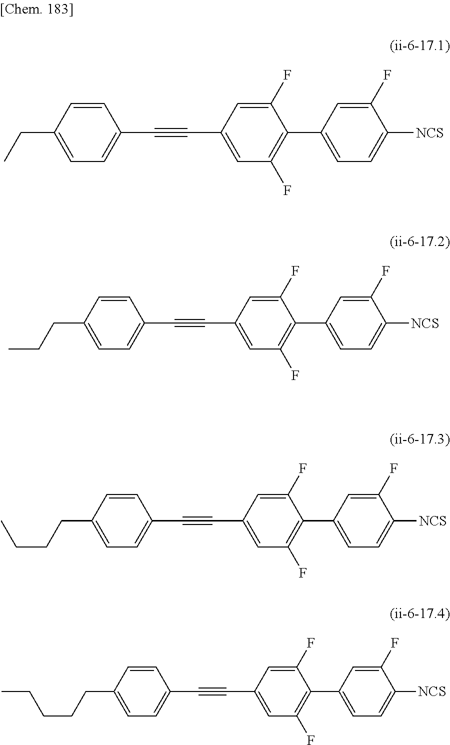

Specific examples of compounds represented by general formula (ii-6-18) include the compounds represented by structural formula (ii-6-18.1) to (ii-6-18.8) below.

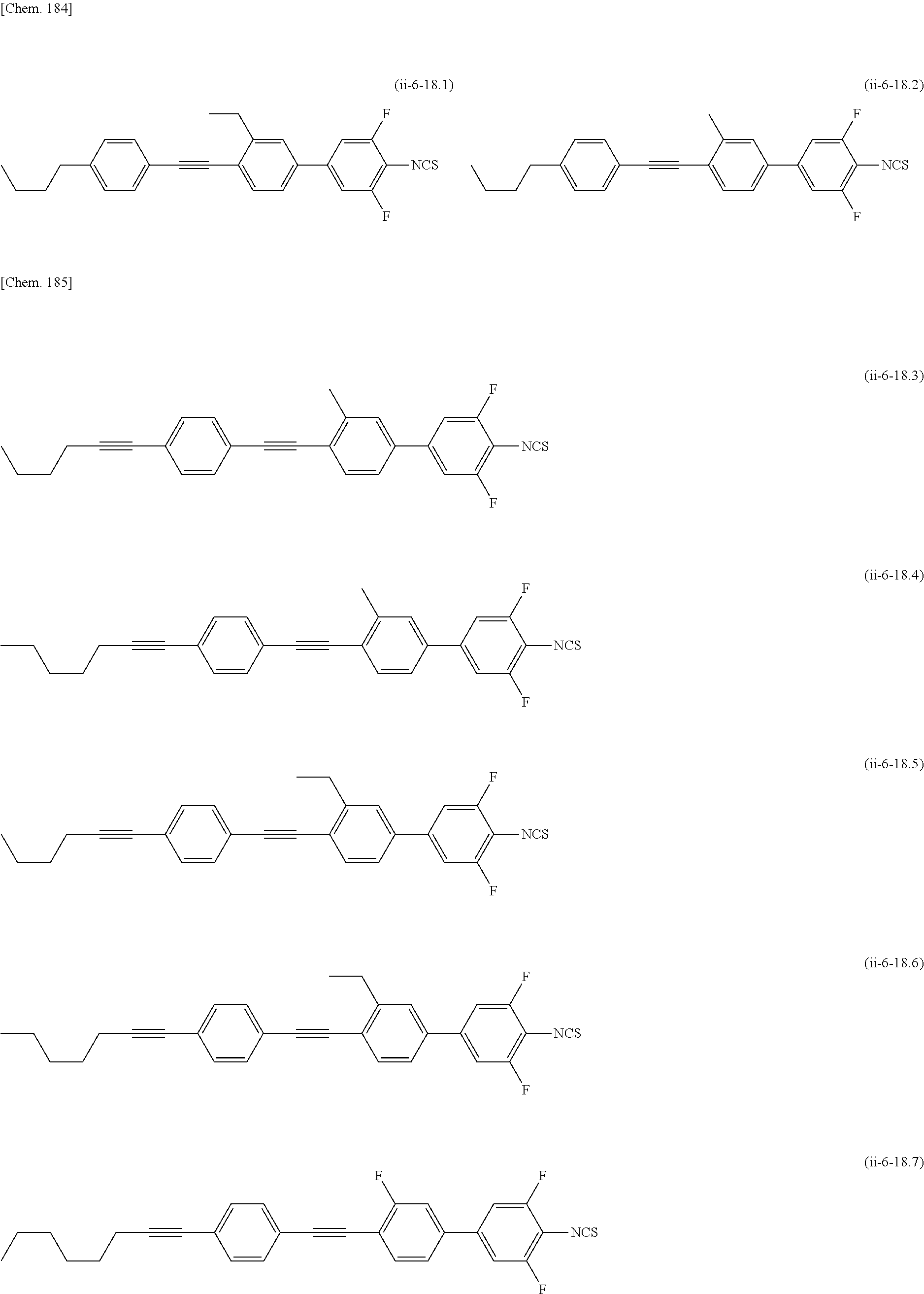

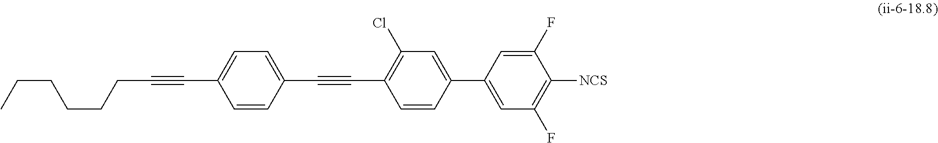

Specific examples of compounds represented by general formula (11-6-19) include the compounds represented by structural formula (ii-6-19.1) to (ii-6-19.4) below.

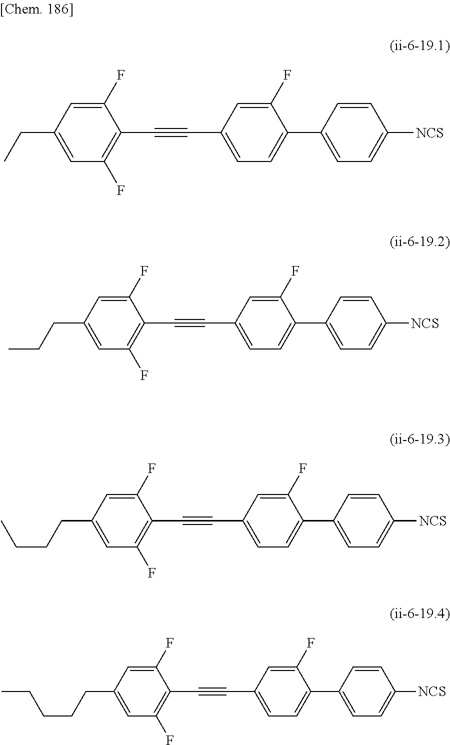

Specific examples of compounds represented by general formula (ii-6-20) include the compounds represented by structural formula (ii-6-20.1) to (ii-6-20.4) below.

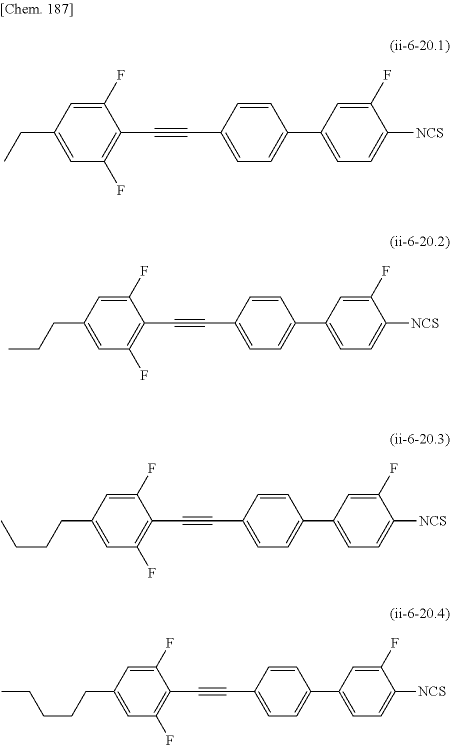

Specific examples of compounds represented by general formula (ii-6-21) include the compounds represented by structural formula (ii-6-21.1) to (ii-6-21.4) below.

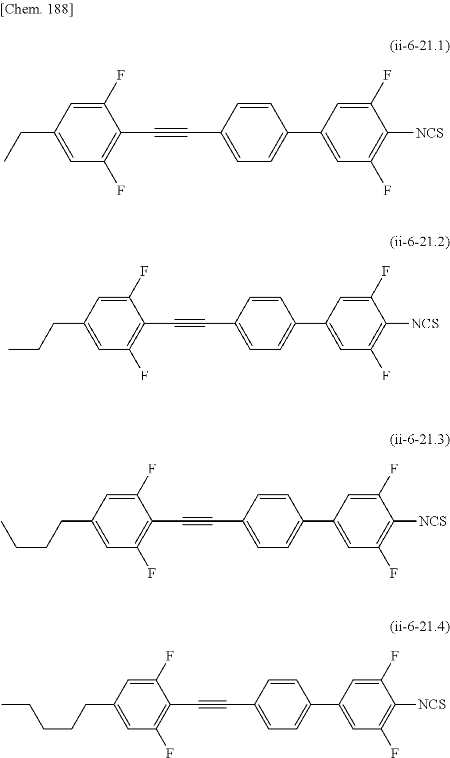

Specific examples of compounds represented by general formula (ii-6-22) include the compounds represented by structural formula (ii-6-22.1) to (ii-6-22.14) below.

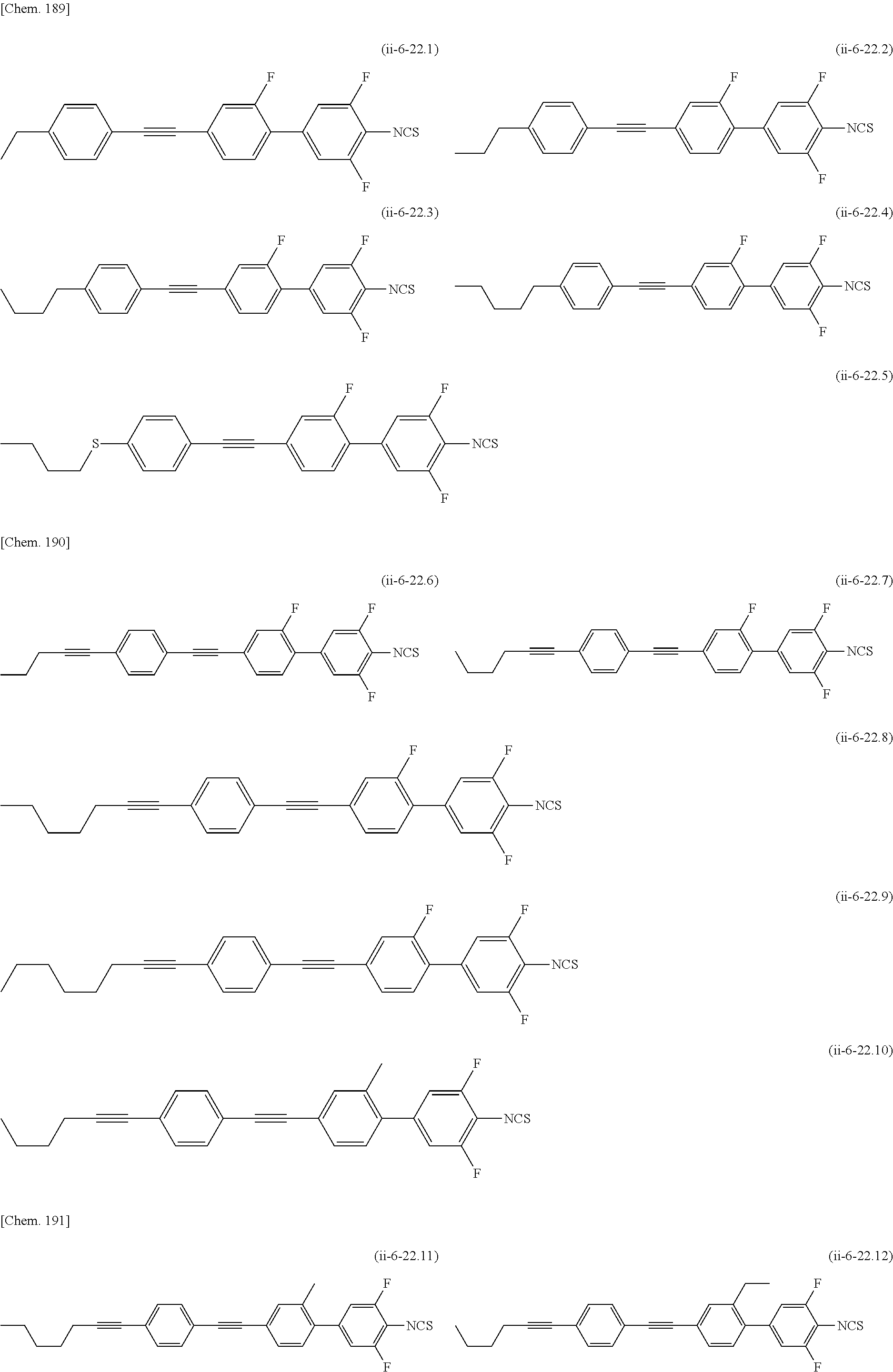

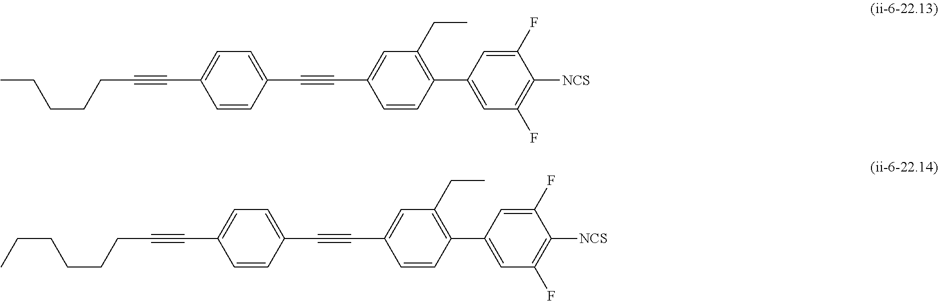

Specific examples of compounds represented by general formula (ii-6-23) include the compounds represented by structural formula (ii-6-23.1) to (ii-6-23.10) below.

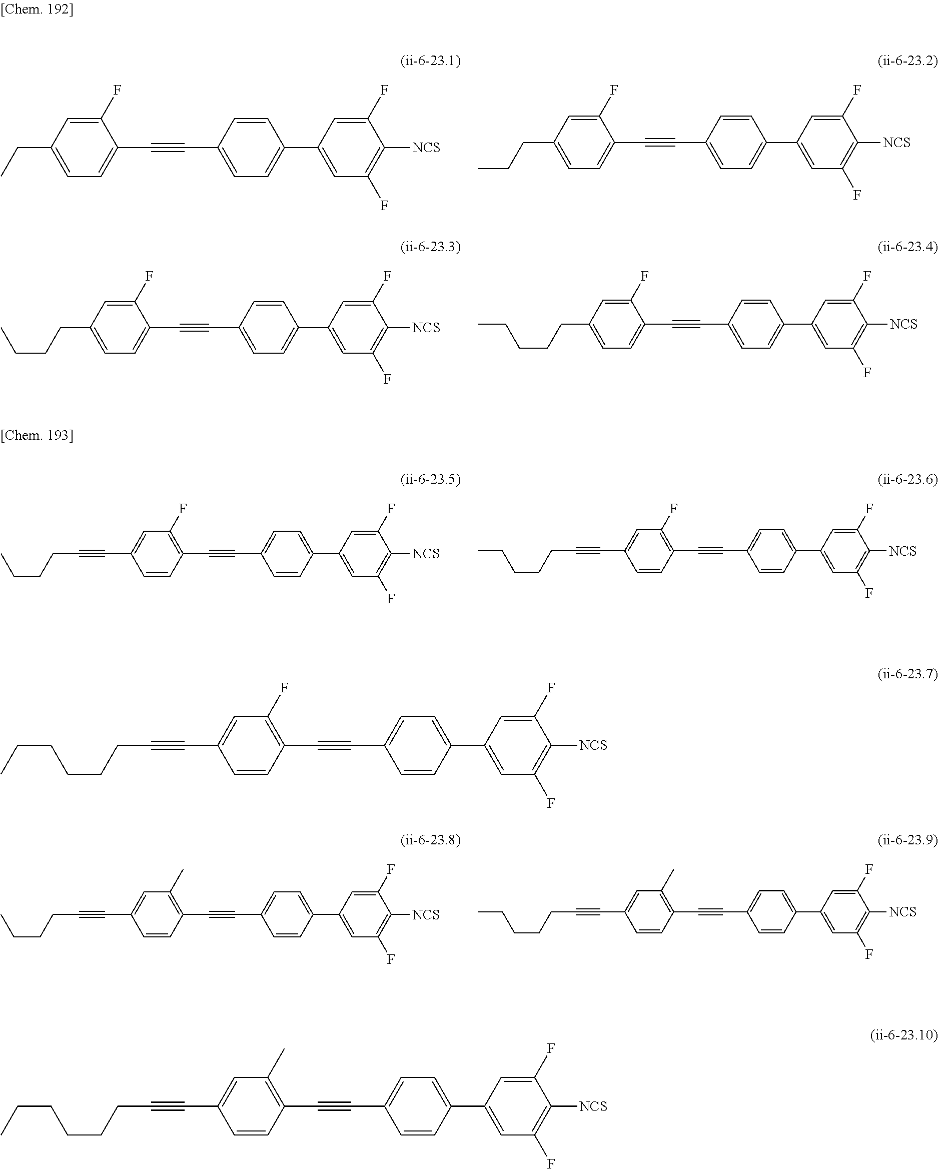

Specific examples of compounds represented by general formula (ii-6-24) include the compounds represented by structural formula (ii-6-24.1) and (ii-6-24.2) below.

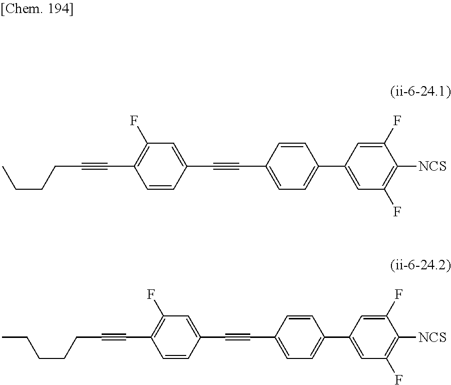

Specific examples of compounds represented by general formula (ii-6-25) include the compounds represented by structural formula (ii-6-25.1) to (ii-6-25.5) below.

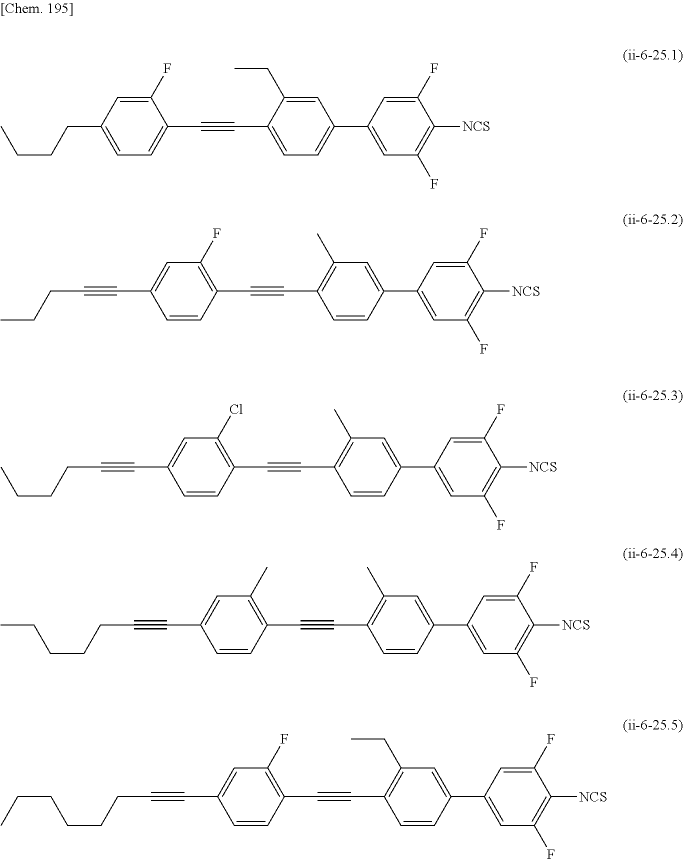

Specific examples of compounds represented by general formula (ii-6-26) include the compounds represented by structural formula (ii-6-26.1) and (ii-6-26.2) below.

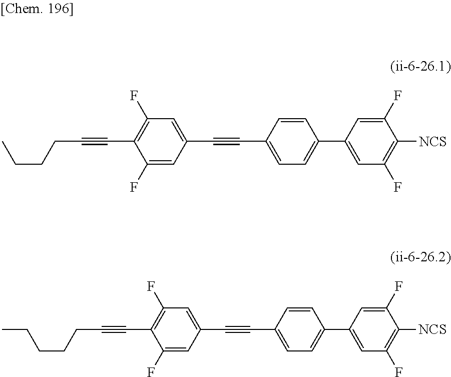

Specific examples of compounds represented by general formula (ii-6-27) include the compound represented by structural formula (ii-6-27.1) below.

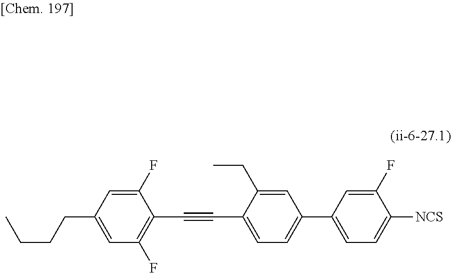

Specific examples of compounds represented by general formula (ii-6-28) include the compounds represented by structural formula (ii-6-28.1) to (ii-6-28.5) below.

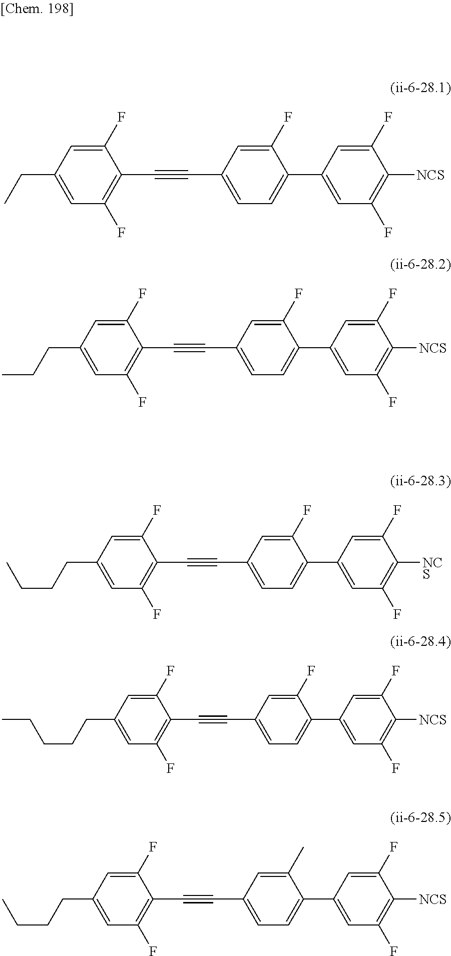

Specific examples of compounds represented by general formula (ii-6-29) include the compound represented by structural formula (ii-6-29.1) below.

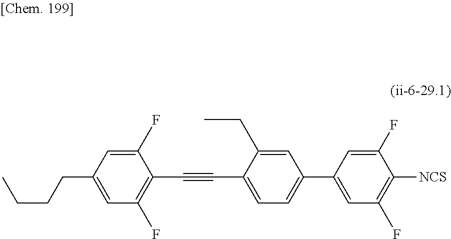

Specific examples of compounds represented by general formula (ii-6-30) include the compound represented by structural formula (ii-6-30.1) below.

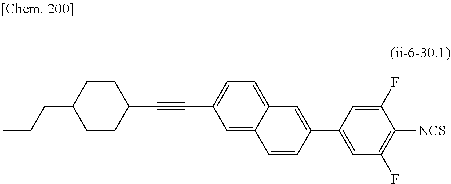

Specific examples of compounds represented by general formula (ii-6-31) include the compounds represented by structural formula (ii-6-31.1) to (ii-6-31.5) below.

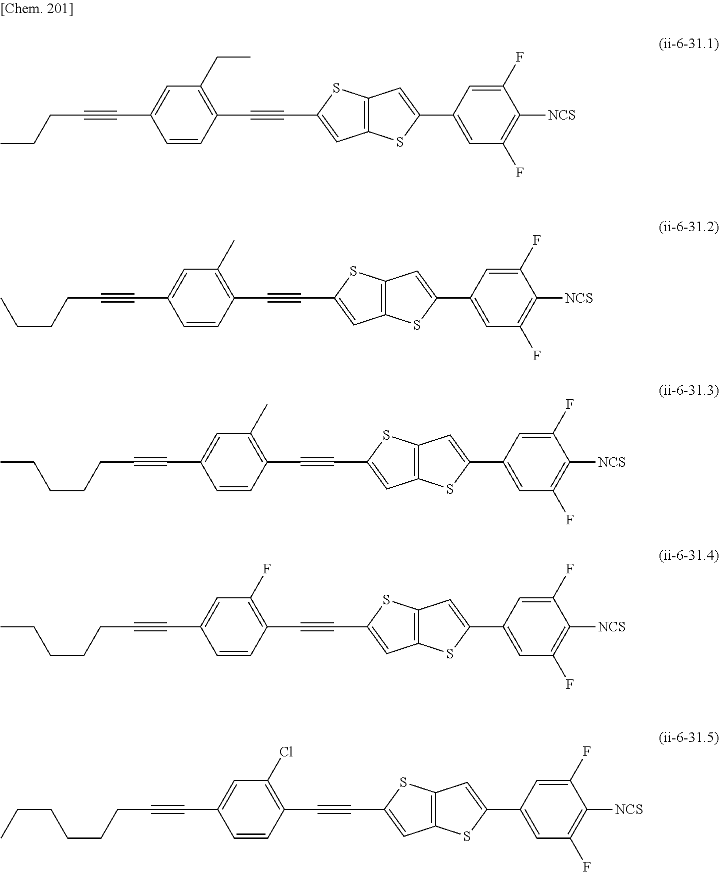

Specific examples of compounds represented by general formula (ii-6-32) include the compound represented by structural formula (ii-6-32.1) below.

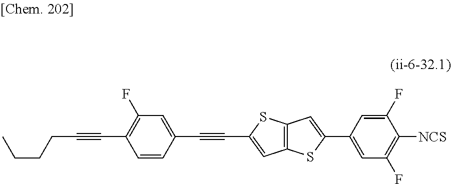

Specific examples of compounds represented by general formula (ii-6-33) include the compounds represented by structural formula (ii-6-33.1) to (ii-6-33.4) below.

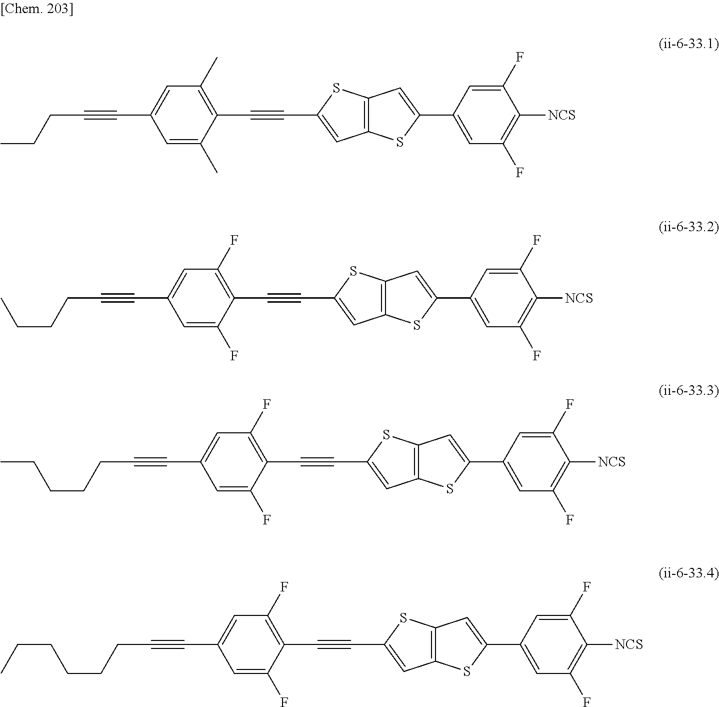

Compounds represented by general formula (ii-7) are preferably compounds represented by general formula (ii-7-1) below.

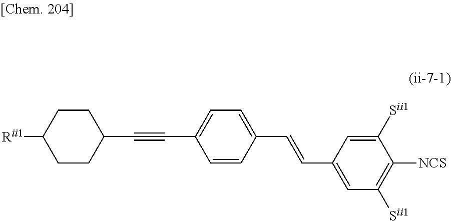

In general formula (ii-7-1), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-7-1) include the compound represented by structural formula (ii-7-1.1) below.

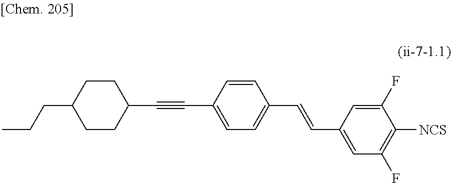

Compounds represented by general formula (ii-8) are preferably compounds represented by general formula (ii-8-1) and (ii-8-2) below.

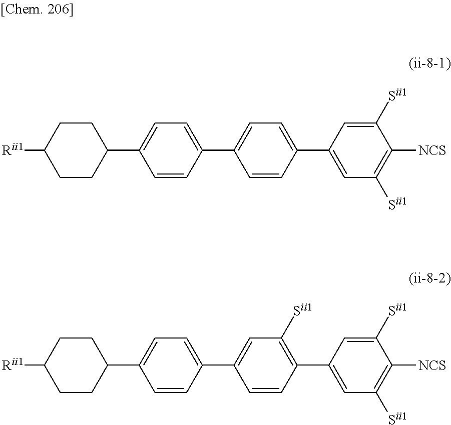

In general formula (ii-8-1) and (ii-8-2), Rⁱⁱ¹ and Sⁱⁱ¹ have the same meanings as Rⁱⁱ¹ and Sⁱⁱ¹, respectively, in general formula (ii) above, independently at each occurrence.

Specific examples of compounds represented by general formula (ii-8-1) include the compounds represented by structural formula (ii-8-1.1) to (ii-8-1.3) below.

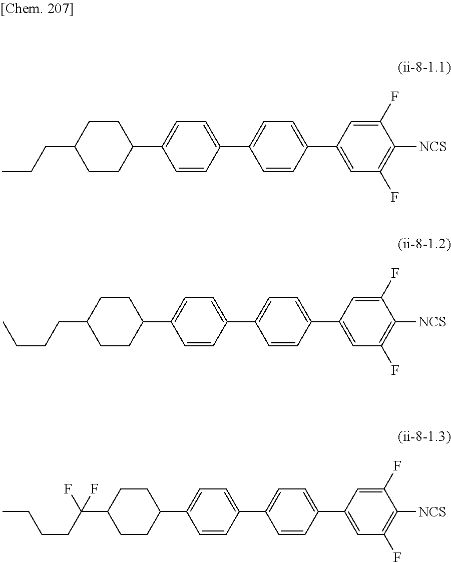

Specific examples of compounds represented by general formula (ii-8-2) include the compounds represented by structural formula (ii-8-2.1) to (ii-8-2.6) below.

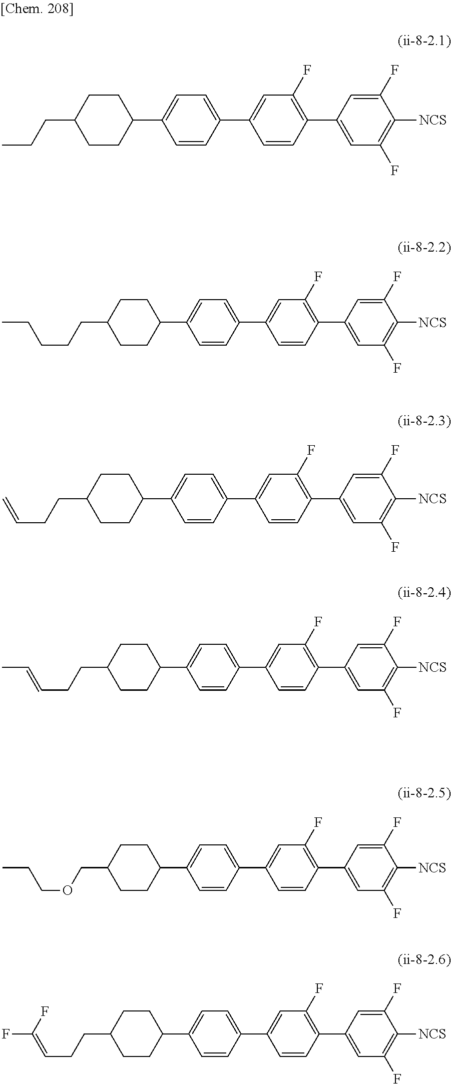

The number of types of compounds represented by general formula (ii), general formula (ii-1) to (ii-8), general formula (ii-1-1) and (ii-1-2), general formula (ii-2-1) to (ii-2-10), general formula (ii-3-1) to (ii-3-16), general formula (ii-4-1) to (ii-4-26), general formula (ii-5-1) to (ii-5-5), general formula (ii-6-1) to (ii-6-33), general formula (ii-7-1), general formula (ii-8-1) and (ii-8-2), structural formula (ii-1-1.1) to (ii-1-1.4), structural formula (ii-1-2.1) to (ii-1-2.6), structural formula (ii-2-1.1) to (ii-2-1.4), structural formula (ii-2-2.1) to (ii-2-2.10), structural formula (ii-2-3.1) to (ii-2-3.3), structural formula (ii-2-4.1) to (ii-2-4.8), structural formula (ii-2-5.1) to (ii-2-5.4), structural formula (ii-2-6.1) to (ii-2-6.4), structural formula (ii-2-7.1) to (ii-2-7.3), structural formula (ii-2-8.1) to (ii-2-8.3), structural formula (ii-2-9.1) to (ii-2-9.4), structural formula (ii-2-10.1) to (ii-2-10.4), structural formula (ii-3-1.1) to (ii-3-1.4), structural formula (ii-3-2.1) to (ii-3-2.4), structural formula (ii-3-3.1) to (ii-3-3.7), structural formula (ii-3-4.1) to (ii-3-4.5), structural formula (ii-3-5.1) to (ii-3-5.7), structural formula (ii-3-6.1) to (ii-3-6.3), structural formula (ii-3-7.1) to (ii-3-7.7), structural formula (ii-3-8.1) to (ii-3-8.3), structural formula (ii-3-9.1) to (ii-3-9.4), structural formula (ii-3-10.1) to (ii-3-10.3), structural formula (ii-3-11.1) to (ii-3-11.3), structural formula (ii-3-12.1) to (ii-3-12.3), structural formula (ii-3-13.1) to (ii-3-13.4), structural formula (ii-3-14.1) to (ii-3-14.3), structural formula (ii-3-15.1) to (ii-3-15.3), structural formula (ii-3-16.1) to (ii-3-16.3), structural formula (ii-4-1.1) to (ii-4-1.4), structural formula (ii-4-2.1) to (ii-4-2.4), structural formula (ii-4-3.1) to (ii-4-3.8), structural formula (ii-4-4.1) to (ii-4-4.5), structural formula (ii-4-5.1), structural formula (ii-4-6.1) to (ii-4-6.5), structural formula (ii-4-7.1), structural formula (ii-4-8.1) to (ii-4-8.4), structural formula (ii-4-9.1) to (ii-4-9.4), structural formula (ii-4-10.1) to (ii-4-10.4), structural formula (ii-4-11.1) to (ii-4-11.5), structural formula (ii-4-12.1) to (ii-4-12.5), structural formula (ii-4-13.1) to (ii-4-13.4), structural formula (ii-4-14.1) to (ii-4-14.4), structural formula (ii-4-15.1) to (ii-4-15.6), structural formula (ii-4-16.1) to (ii-4-16.3), structural formula (ii-4-17.1) to (ii-4-17.3), structural formula (ii-4-18.1) to (ii-4-18.4), structural formula (ii-4-19.1) to (ii-4-19.8), structural formula (ii-4-20.1) to (ii-4-20.4), structural formula (ii-4-21.1) to (ii-4-21.4), structural formula (ii-4-22.1) to (ii-4-22.3), structural formula (ii-4-23.1) to (ii-4-23.3), structural formula (ii-4-24.1) to (ii-4-24.3), structural formula (ii-4-25.1) to (ii-4-25.3), structural formula (ii-4-26.1) to (ii-4-26.3), structural formula (ii-5-1.1) to (ii-5-1.4), structural formula (ii-5-2.1) to (ii-5-2.4), structural formula (ii-5-3.1), structural formula (ii-5-4.1) to (ii-5-4.3), structural formula (ii-5-5.1) to (ii-5-5.3), structural formula (ii-6-1.1) to (ii-6-1.4), structural formula (ii-6-2.1) to (ii-6-2.4), structural formula (ii-6-3.1) to (ii-6-3.8), structural formula (ii-6-4.1) to (ii-6-4.4), structural formula (ii-6-5.1) to (ii-6-5.4), structural formula (ii-6-6.1) and (ii-6-6.2), structural formula (ii-6-7.1) to (ii-6-7.8), structural formula (ii-6-8.1) to (ii-6-8.9), structural formula (ii-6-9.1) to (ii-6-9.4), structural formula (ii-6-10.1) to (ii-6-10.4), structural formula (ii-6-11.1) to (ii-6-11.4), structural formula (ii-6-12.1) to (ii-6-12.4), structural formula (ii-6-13.1) to (ii-6-13.20), structural formula (ii-6-14.1) to (ii-6-14.4), structural formula (ii-6-15.1), structural formula (ii-6-16.1) to (ii-6-16.5), structural formula (ii-6-17.1) to (ii-6-17.4), structural formula (ii-6-18.1) to (ii-6-18.8), structural formula (ii-6-19.1) to (ii-6-19.4), structural formula (ii-6-20.1) to (ii-6-20.4), structural formula (ii-6-21.1) to (ii-6-21.4), structural formula (ii-6-22.1) to (ii-6-22.14), structural formula (ii-6-23.1) to (ii-6-23.10), structural formula (ii-6-24.1) and (ii-6-24.2), structural formula (ii-6-25.1) to (ii-6-25.5), structural formula (ii-6-26.1) and (ii-6-26.2), structural formula (ii-6-27.1), structural formula (ii-6-28.1) to (ii-6-28.5), structural formula (ii-6-29.1), structural formula (ii-6-30.1), structural formula (ii-6-31.1) to (ii-6-31.5), structural formula (ii-6-32.1), structural formula (ii-6-33.1) to (ii-6-33.4), structural formula (ii-7-1.1), structural formula (ii-8-1.1) to (ii-8-1.3), or structural formula (ii-8-2.1) to (ii-8-2.6) used in the liquid crystal composition is one or two or more, preferably from one to twenty, preferably from two to fifteen.

The lower limit to the total amount of compounds represented by general formula (ii), general formula (ii-1) to (ii-8), general formula (ii-1-1) and (ii-1-2), general formula (ii-2-1) to (ii-2-10), general formula (ii-3-1) to (ii-3-16), general formula (ii-4-1) to (ii-4-26), general formula (ii-5-1) to (ii-5-5), general formula (ii-6-1) to (ii-6-33), general formula (ii-7-1), general formula (ii-8-1) and (ii-8-2), structural formula (ii-1-1.1) to (ii-1-1.4), structural formula (ii-1-2.1) to (ii-1-2.6), structural formula (ii-2-1.1) to (ii-2-1.4), structural formula (ii-2-2.1) to (ii-2-2.10), structural formula (ii-2-3.1) to (ii-2-3.3), structural formula (ii-2-4.1) to (ii-2-4.8), structural formula (ii-2-5.1) to (ii-2-5.4), structural formula (ii-2-6.1) to (ii-2-6.4), structural formula (ii-2-7.1) to (ii-2-7.3), structural formula (ii-2-8.1) to (ii-2-8.3), structural formula (ii-2-9.1) to (ii-2-9.4), structural formula (ii-2-10.1) to (ii-2-10.4), structural formula (ii-3-1.1) to (ii-3-1.4), structural formula (ii-3-2.1) to (ii-3-2.4), structural formula (ii-3-3.1) to (ii-3-3.7), structural formula (ii-3-4.1) to (ii-3-4.5), structural formula (ii-3-5.1) to (ii-3-5.7), structural formula (ii-3-6.1) to (ii-3-6.3), structural formula (ii-3-7.1) to (ii-3-7.7), structural formula (ii-3-8.1) to (ii-3-8.3), structural formula (ii-3-9.1) to (ii-3-9.4), structural formula (ii-3-10.1) to (ii-3-10.3), structural formula (ii-3-11.1) to (ii-3-11.3), structural formula (ii-3-12.1) to (ii-3-12.3), structural formula (ii-3-13.1) to (ii-3-13.4), structural formula (ii-3-14.1) to (ii-3-14.3), structural formula (ii-3-15.1) to (ii-3-15.3), structural formula (ii-3-16.1) to (ii-3-16.3), structural formula (ii-4-1.1) to (ii-4-1.4), structural formula (ii-4-2.1) to (ii-4-2.4), structural formula (ii-4-3.1) to (ii-4-3.8), structural formula (ii-4-4.1) to (ii-4-4.5), structural formula (ii-4-5.1), structural formula (ii-4-6.1) to (ii-4-6.5), structural formula (ii-4-7.1), structural formula (ii-4-8.1) to (ii-4-8.4), structural formula (ii-4-9.1) to (ii-4-9.4), structural formula (ii-4-10.1) to (ii-4-10.4), structural formula (ii-4-11.1) to (ii-4-11.5), structural formula (ii-4-12.1) to (ii-4-12.5), structural formula (ii-4-13.1) to (ii-4-13.4), structural formula (ii-4-14.1) to (ii-4-14.4), structural formula (ii-4-15.1) to (ii-4-15.6), structural formula (ii-4-16.1) to (ii-4-16.3), structural formula (ii-4-17.1) to (ii-4-17.3), structural formula (ii-4-18.1) to (ii-4-18.4), structural formula (ii-4-19.1) to (ii-4-19.8), structural formula (ii-4-20.1) to (ii-4-20.4), structural formula (ii-4-21.1) to (ii-4-21.4), structural formula (ii-4-22.1) to (ii-4-22.3), structural formula (ii-4-23.1) to (ii-4-23.3), structural formula (ii-4-24.1) to (ii-4-24.3), structural formula (ii-4-25.1) to (ii-4-25.3), structural formula (ii-4-26.1) to (ii-4-26.3), structural formula (ii-5-1.1) to (ii-5-1.4), structural formula (ii-5-2.1) to (ii-5-2.4), structural formula (ii-5-3.1), structural formula (ii-5-4.1) to (ii-5-4.3), structural formula (ii-5-5.1) to (ii-5-5.3), structural formula (ii-6-1.1) to (ii-6-1.4), structural formula (ii-6-2.1) to (ii-6-2.4), structural formula (ii-6-3.1) to (ii-6-3.8), structural formula (ii-6-4.1) to (ii-6-4.4), structural formula (ii-6-5.1) to (ii-6-5.4), structural formula (ii-6-6.1) and (ii-6-6.2), structural formula (ii-6-7.1) to (ii-6-7.8), structural formula (ii-6-8.1) to (ii-6-8.9), structural formula (ii-6-9.1) to (ii-6-9.4), structural formula (ii-6-10.1) to (ii-6-10.4), structural formula (ii-6-11.1) to (ii-6-11.4), structural formula (ii-6-12.1) to (ii-6-12.4), structural formula (ii-6-13.1) to (ii-6-13.20), structural formula (ii-6-14.1) to (ii-6-14.4), structural formula (ii-6-15.1), structural formula (ii-6-16.1) to (ii-6-16.5), structural formula (ii-6-17.1) to (ii-6-17.4), structural formula (ii-6-18.1) to (ii-6-18.8), structural formula (ii-6-19.1) to (ii-6-19.4), structural formula (ii-6-20.1) to (ii-6-20.4), structural formula (ii-6-21.1) to (ii-6-21.4), structural formula (ii-6-22.1) to (ii-6-22.14), structural formula (ii-6-23.1) to (ii-6-23.10), structural formula (ii-6-24.1) and (ii-6-24.2), structural formula (ii-6-25.1) to (ii-6-25.5), structural formula (ii-6-26.1) and (ii-6-26.2), structural formula (ii-6-27.1), structural formula (ii-6-28.1) to (ii-6-28.5), structural formula (ii-6-29.1), structural formula (ii-6-30.1), structural formula (ii-6-31.1) to (ii-6-31.5), structural formula (ii-6-32.1), structural formula (ii-6-33.1) to (ii-6-33.4), structural formula (ii-7-1.1), structural formula (ii-8-1.1) to (ii-8-1.3), or structural formula (ii-8-2.1) to (ii-8-2.6) in 100% by mass of the liquid crystal composition is preferably 1% by mass or more, preferably 5% by mass or more, preferably 10% by mass or more, preferably 15% by mass or more, preferably 20% by mass or more, preferably 25% by mass or more, preferably 30% by mass or more, preferably 35% by mass or more, preferably 40% by mass or more, preferably 45% by mass or more, preferably 75% by mass or more, preferably 80% by mass or more, preferably 85% by mass or more.

The upper limit to the total amount of compounds represented by general formula (ii), general formula (ii-1) to (ii-8), general formula (ii-1-1) and (ii-1-2), general formula (ii-2-1) to (ii-2-10), general formula (ii-3-1) to (ii-3-16), general formula (ii-4-1) to (ii-4-26), general formula (ii-5-1) to (ii-5-5), general formula (ii-6-1) to (ii-6-33), general formula (ii-7-1), general formula (ii-8-1) and (ii-8-2), structural formula (ii-1-1.1) to (ii-1-1.4), structural formula (ii-1-2.1) to (ii-1-2.6), structural formula (ii-2-1.1) to (ii-2-1.4), structural formula (ii-2-2.1) to (ii-2-2.10), structural formula (ii-2-3.1) to (ii-2-3.3), structural formula (ii-2-4.1) to (ii-2-4.8), structural formula (ii-2-5.1) to (ii-2-5.4), structural formula (ii-2-6.1) to (ii-2-6.4), structural formula (ii-2-7.1) to (ii-2-7.3), structural formula (ii-2-8.1) to (ii-2-8.3), structural formula (ii-2-9.1) to (ii-2-9.4), structural formula (ii-2-10.1) to (ii-2-10.4), structural formula (ii-3-1.1) to (ii-3-1.4), structural formula (ii-3-2.1) to (ii-3-2.4), structural formula (ii-3-3.1) to (ii-3-3.7), structural formula (ii-3-4.1) to (ii-3-4.5), structural formula (ii-3-5.1) to (ii-3-5.7), structural formula (ii-3-6.1) to (ii-3-6.3), structural formula (ii-3-7.1) to (ii-3-7.7), structural formula (ii-3-8.1) to (ii-3-8.3), structural formula (ii-3-9.1) to (ii-3-9.4), structural formula (ii-3-10.1) to (ii-3-10.3), structural formula (ii-3-11.1) to (ii-3-11.3), structural formula (ii-3-12.1) to (ii-3-12.3), structural formula (ii-3-13.1) to (ii-3-13.4), structural formula (ii-3-14.1) to (ii-3-14.3), structural formula (ii-3-15.1) to (ii-3-15.3), structural formula (ii-3-16.1) to (ii-3-16.3), structural formula (ii-4-1.1) to (ii-4-1.4), structural formula (ii-4-2.1) to (ii-4-2.4), structural formula (ii-4-3.1) to (ii-4-3.8), structural formula (ii-4-4.1) to (ii-4-4.5), structural formula (ii-4-5.1), structural formula (ii-4-6.1) to (ii-4-6.5), structural formula (ii-4-7.1), structural formula (ii-4-8.1) to (ii-4-8.4), structural formula (ii-4-9.1) to (ii-4-9.4), structural formula (ii-4-10.1) to (ii-4-10.4), structural formula (ii-4-11.1) to (ii-4-11.5), structural formula (ii-4-12.1) to (ii-4-12.5), structural formula (ii-4-13.1) to (ii-4-13.4), structural formula (ii-4-14.1) to (ii-4-14.4), structural formula (ii-4-15.1) to (ii-4-15.6), structural formula (ii-4-16.1) to (ii-4-16.3), structural formula (ii-4-17.1) to (ii-4-17.3), structural formula (ii-4-18.1) to (ii-4-18.4), structural formula (ii-4-19.1) to (ii-4-19.8), structural formula (ii-4-20.1) to (ii-4-20.4), structural formula (ii-4-21.1) to (ii-4-21.4), structural formula (ii-4-22.1) to (ii-4-22.3), structural formula (ii-4-23.1) to (ii-4-23.3), structural formula (ii-4-24.1) to (ii-4-24.3), structural formula (ii-4-25.1) to (ii-4-25.3), structural formula (ii-4-26.1) to (ii-4-26.3), structural formula (ii-5-1.1) to (ii-5-1.4), structural formula (ii-5-2.1) to (ii-5-2.4), structural formula (ii-5-3.1), structural formula (ii-5-4.1) to (ii-5-4.3), structural formula (ii-5-5.1) to (ii-5-5.3), structural formula (ii-6-1.1) to (ii-6-1.4), structural formula (ii-6-2.1) to (ii-6-2.4), structural formula (ii-6-3.1) to (ii-6-3.8), structural formula (ii-6-4.1) to (ii-6-4.4), structural formula (ii-6-5.1) to (ii-6-5.4), structural formula (ii-6-6.1) and (ii-6-6.2), structural formula (ii-6-7.1) to (ii-6-7.8), structural formula (ii-6-8.1) to (ii-6-8.9), structural formula (ii-6-9.1) to (ii-6-9.4), structural formula (ii-6-10.1) to (ii-6-10.4), structural formula (ii-6-11.1) to (ii-6-11.4), structural formula (ii-6-12.1) to (ii-6-12.4), structural formula (ii-6-13.1) to (ii-6-13.20), structural formula (ii-6-14.1) to (ii-6-14.4), structural formula (ii-6-15.1), structural formula (ii-6-16.1) to (ii-6-16.5), structural formula (ii-6-17.1) to (ii-6-17.4), structural formula (ii-6-18.1) to (ii-6-18.8), structural formula (ii-6-19.1) to (ii-6-19.4), structural formula (ii-6-20.1) to (ii-6-20.4), structural formula (ii-6-21.1) to (ii-6-21.4), structural formula (ii-6-22.1) to (ii-6-22.14), structural formula (ii-6-23.1) to (ii-6-23.10), structural formula (ii-6-24.1) and (ii-6-24.2), structural formula (ii-6-25.1) to (ii-6-25.5), structural formula (ii-6-26.1) and (ii-6-26.2), structural formula (ii-6-27.1), structural formula (ii-6-28.1) to (ii-6-28.5), structural formula (ii-6-29.1), structural formula (ii-6-30.1), structural formula (ii-6-31.1) to (ii-6-31.5), structural formula (ii-6-32.1), structural formula (ii-6-33.1) to (ii-6-33.4), structural formula (ii-7-1.1), structural formula (ii-8-1.1) to (ii-8-1.3), or structural formula (ii-8-2.1) to (ii-8-2.6) in 100% by mass of the liquid crystal composition is preferably 95% by mass or less, preferably 90% by mass or less, preferably 55% by mass or less, preferably 50% by mass or less, preferably 45% by mass or less, preferably 40% by mass or less, preferably 35% by mass or less, preferably 25% by mass or less, preferably 15% by mass or less, preferably 5% by mass or less.

From the viewpoint(s) of solubility, Δn, and/or Δεᵣ, it is preferred that the total amount of compounds represented by general formula (ii), general formula (ii-1) to (ii-8), general formula (ii-1-1) and (ii-1-2), general formula (ii-2-1) to (ii-2-10), general formula (ii-3-1) to (ii-3-16), general formula (ii-4-1) to (ii-4-26), general formula (ii-5-1) to (ii-5-5), general formula (ii-6-1) to (ii-6-33), general formula (ii-7-1), general formula (ii-8-1) and (ii-8-2), structural formula (ii-1-1.1) to (ii-1-1.4), structural formula (ii-1-2.1) to (ii-1-2.6), structural formula (ii-2-1.1) to (ii-2-1.4), structural formula (ii-2-2.1) to (ii-2-2.10), structural formula (ii-2-3.1) to (ii-2-3.3), structural formula (ii-2-4.1) to (ii-2-4.8), structural formula (ii-2-5.1) to (ii-2-5.4), structural formula (ii-2-6.1) to (ii-2-6.4), structural formula (ii-2-7.1) to (ii-2-7.3), structural formula (ii-2-8.1) to (ii-2-8.3), structural formula (ii-2-9.1) to (ii-2-9.4), structural formula (ii-2-10.1) to (ii-2-10.4), structural formula (ii-3-1.1) to (ii-3-1.4), structural formula (ii-3-2.1) to (ii-3-2.4), structural formula (ii-3-3.1) to (ii-3-3.7), structural formula (ii-3-4.1) to (ii-3-4.5), structural formula (ii-3-5.1) to (ii-3-5.7), structural formula (ii-3-6.1) to (ii-3-6.3), structural formula (ii-3-7.1) to (ii-3-7.7), structural formula (ii-3-8.1) to (ii-3-8.3), structural formula (ii-3-9.1) to (ii-3-9.4), structural formula (ii-3-10.1) to (ii-3-10.3), structural formula (ii-3-11.1) to (ii-3-11.3), structural formula (ii-3-12.1) to (ii-3-12.3), structural formula (ii-3-13.1) to (ii-3-13.4), structural formula (ii-3-14.1) to (ii-3-14.3), structural formula (ii-3-15.1) to (ii-3-15.3), structural formula (ii-3-16.1) to (ii-3-16.3), structural formula (ii-4-1.1) to (ii-4-1.4), structural formula (ii-4-2.1) to (ii-4-2.4), structural formula (ii-4-3.1) to (ii-4-3.8), structural formula (ii-4-4.1) to (ii-4-4.5), structural formula (ii-4-5.1), structural formula (ii-4-6.1) to (ii-4-6.5), structural formula (ii-4-7.1), structural formula (ii-4-8.1) to (ii-4-8.4), structural formula (ii-4-9.1) to (ii-4-9.4), structural formula (ii-4-10.1) to (ii-4-10.4), structural formula (ii-4-11.1) to (ii-4-11.5), structural formula (ii-4-12.1) to (ii-4-12.5), structural formula (ii-4-13.1) to (ii-4-13.4), structural formula (ii-4-14.1) to (ii-4-14.4), structural formula (ii-4-15.1) to (ii-4-15.6), structural formula (ii-4-16.1) to (ii-4-16.3), structural formula (ii-4-17.1) to (ii-4-17.3), structural formula (ii-4-18.1) to (ii-4-18.4), structural formula (ii-4-19.1) to (ii-4-19.8), structural formula (ii-4-20.1) to (ii-4-20.4), structural formula (ii-4-21.1) to (ii-4-21.4), structural formula (ii-4-22.1) to (ii-4-22.3), structural formula (ii-4-23.1) to (ii-4-23.3), structural formula (ii-4-24.1) to (ii-4-24.3), structural formula (ii-4-25.1) to (ii-4-25.3), structural formula (ii-4-26.1) to (ii-4-26.3), structural formula (ii-5-1.1) to (ii-5-1.4), structural formula (ii-5-2.1) to (ii-5-2.4), structural formula (ii-5-3.1), structural formula (ii-5-4.1) to (ii-5-4.3), structural formula (ii-5-5.1) to (ii-5-5.3), structural formula (ii-6-1.1) to (ii-6-1.4), structural formula (ii-6-2.1) to (ii-6-2.4), structural formula (ii-6-3.1) to (ii-6-3.8), structural formula (ii-6-4.1) to (ii-6-4.4), structural formula (ii-6-5.1) to (ii-6-5.4), structural formula (ii-6-6.1) and (ii-6-6.2), structural formula (ii-6-7.1) to (ii-6-7.8), structural formula (ii-6-8.1) to (ii-6-8.9), structural formula (ii-6-9.1) to (ii-6-9.4), structural formula (ii-6-10.1) to (ii-6-10.4), structural formula (ii-6-11.1) to (ii-6-11.4), structural formula (ii-6-12.1) to (ii-6-12.4), structural formula (ii-6-13.1) to (ii-6-13.20), structural formula (ii-6-14.1) to (ii-6-14.4), structural formula (ii-6-15.1), structural formula (ii-6-16.1) to (ii-6-16.5), structural formula (ii-6-17.1) to (ii-6-17.4), structural formula (ii-6-18.1) to (ii-6-18.8), structural formula (ii-6-19.1) to (ii-6-19.4), structural formula (ii-6-20.1) to (ii-6-20.4), structural formula (ii-6-21.1) to (ii-6-21.4), structural formula (ii-6-22.1) to (ii-6-22.14), structural formula (ii-6-23.1) to (ii-6-23.10), structural formula (ii-6-24.1) and (ii-6-24.2), structural formula (ii-6-25.1) to (ii-6-25.5), structural formula (ii-6-26.1) and (ii-6-26.2), structural formula (ii-6-27.1), structural formula (ii-6-28.1) to (ii-6-28.5), structural formula (ii-6-29.1), structural formula (ii-6-30.1), structural formula (ii-6-31.1) to (ii-6-31.5), structural formula (ii-6-32.1), structural formula (ii-6-33.1) to (ii-6-33.4), structural formula (ii-7-1.1), structural formula (ii-8-1.1) to (ii-8-1.3), or structural formula (ii-8-2.1) to (ii-8-2.6) in 100% by mass of the liquid crystal composition be from 1% to 95% by mass, preferably from 5% to 90% by mass, preferably from 5% to 55% by mass, preferably from 10% to 45% by mass.

Compounds represented by general formula (ii) (including subordinate concepts) can be synthesized using known synthetic methods.

### Other Compounds

From the viewpoint(s) of Δn and/or Δεᵣ, the liquid crystal composition according to the present invention is allowed to further contain one or two or more types of compounds represented by general formula (vt) below, which has at least one —C≡C— as a linking group.

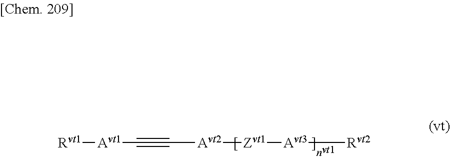

In general formula (vt), R^{vt} represents a hydrogen atom or a C1 to C20 alkyl group.

A C1 to C20 alkyl group is a linear-chain, branched, or cyclic alkyl group and preferably is a linear-chain alkyl group.

The number of carbon atoms in the C1 to C20 alkyl group is preferably from two to ten, preferably from two to six.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CF—, and/or —C≡C—.

Moreover, one —CH₂—CH₂—CH₂— in the alkyl group, or each of two or more independently, may have been replaced with —O—CO—O—.

In addition, one hydrogen atom in the alkyl group, or each of two or more independently, may have been replaced with a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

For example, R^{vt1} can represent a C1 to C19 alkoxy group as a result of the replacement of one —CH₂— in the alkyl group by —O—.

The alkoxy group is a linear-chain, branched, or cyclic alkoxy group and preferably is a linear-chain alkoxy group.

The number of carbon atoms in the alkoxy group is preferably from two to ten, preferably from two to six.

R^{vt1}, furthermore, can represent a C1 to C19 alkylsulfanyl group (alkylthio group) as a result of the replacement of one —CH₂— in the alkyl group by —S—.

The alkylsulfanyl group is a linear-chain, branched, or cyclic alkylsulfanyl group and preferably is a linear-chain alkylsulfanyl group.

The number of carbon atoms in the alkylsulfanyl group is preferably from one to ten, preferably from one to six.

Moreover, R^{vt1} can represent a C2 to C20 alkenyl group as a result of the replacement of one or two or more —CH₂—CH₂-s in the alkyl group by —CH═CH—.

The alkenyl group is a linear-chain, branched, or cyclic alkenyl group and preferably is a linear-chain alkenyl group.

The number of carbon atoms in the alkenyl group is preferably from two to ten, preferably from two to six.

R^{vt1}, furthermore, can represent a C2 to C20 alkynyl group as a result of the replacement of one or two or more —CH₂—CH₂-s in the alkyl group by —C≡C—.

The alkynyl group is a linear-chain, branched, or cyclic alkynyl group and preferably is a linear-chain alkynyl group.

The number of carbon atoms in the alkynyl group is preferably from two to ten, preferably from two to six.

Moreover, R^{vt1} can represent a C2 to C19 alkenyloxy group as a result of the replacement of one —CH₂— in the alkyl group by —O— and the replacement of one or two or more —CH₂—CH₂-s by —CH═CH—.

The alkenyloxy group is a linear-chain, branched, or cyclic alkenyloxy group and preferably is a linear-chain alkenyloxy group.

The number of carbon atoms in the alkenyloxy group is preferably from two to ten, preferably from two to six.

R^{vt1}, furthermore, can represent a C1 to C20 halogenated alkyl group as a result of the replacement of one or two or more hydrogen atoms in the alkyl group by a halogen atom.

The halogenated alkyl group is a linear-chain, branched, or cyclic halogenated alkyl group and preferably is a linear-chain halogenated alkyl group.

The number of carbon atoms in the halogenated alkyl group is preferably from two to ten, preferably from two to six.

Moreover, R^{vt1} can represent a C1 to C19 halogenated alkoxy group as a result of the replacement of one —CH₂— in the alkyl group by —O— and the replacement of one or two or more hydrogen atoms in the alkyl group by a halogen atom.

The halogenated alkoxy group is a linear-chain, branched, or cyclic halogenated alkoxy group and preferably is a linear-chain halogenated alkoxy group.

The number of carbon atoms in the halogenated alkoxy group is preferably from two to ten, preferably from two to six.

Specific examples of C1 to C20 alkyl groups (including substituted ones) at R^{vt1} include the groups represented by formula (R^{vt1}-1) to (R^{vt1}-36).

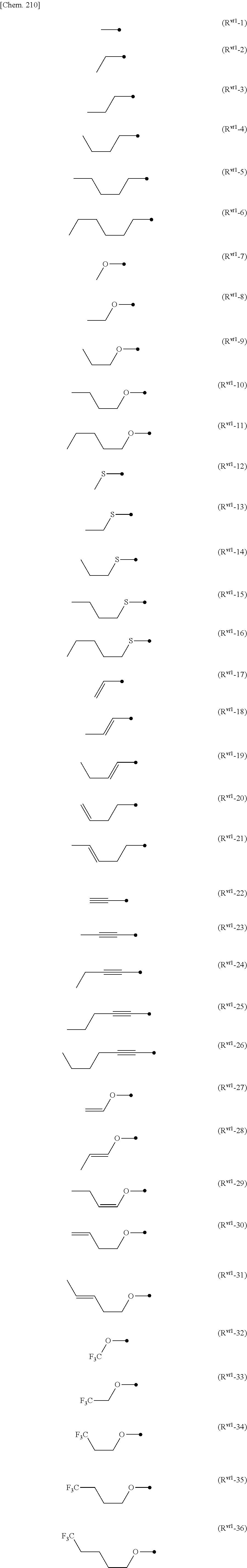

In formula (R^{vt1}-1) to (R^{vt1}-36), the black dot represents a bond to A^{vt1}.

R^{vt1} is preferably a C1 to C12 alkyl group when the overall reliability of the liquid crystal composition is a priority.

When the reduction of the overall viscosity of the liquid crystal composition is a priority, it is preferred that R^{vt1} be a C2 to C8 alkenyl group.

When the ring structure to which R^{vt1} is bound is a phenyl group (aromatic), linear-chain C1 to C5 alkyl groups, linear-chain C1 to C4 alkoxy groups, and C4 and C5 alkenyl groups are preferred. When the ring structure to which R^{vt1} is bound is a saturated ring structure, such as cyclohexane, pyran, or dioxane, linear-chain C1 to C5 alkyl groups, linear-chain C1 to C4 alkoxy groups, and linear-chain C2 to C5 alkenyl groups are preferred.

For R^{vt1}, furthermore, it is preferred that the total number of carbon atoms and oxygen atoms, if any, be five or fewer, and it is preferred that R^{vt1} be a linear-chain group, when the stabilization of the nematic phase is sought.

It should be noted that from the viewpoint of solubility, it is preferred that R^{vt1} be a C2 to C6 linear-chain alkyl group or C1 to C6 linear-chain alkylsulfanyl group.

In general formula (vt), R^{vt2} represents any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, or a C1 to C20 alkyl group.

A C1 to C20 alkyl group is a linear-chain, branched, or cyclic alkyl group and preferably is a linear-chain alkyl group.

The number of carbon atoms in the alkyl group is preferably from two to ten, preferably from two to six.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CF—, and/or —C≡C—.

Moreover, one —CH₂—CH₂—CH₂— in the alkyl group, or each of two or more independently, may have been replaced with —O—CO—O—.

In addition, one hydrogen atom in the alkyl group, or each of two or more independently, may have been replaced with a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

For example, R^{vt2} can represent a C1 to C19 alkoxy group as a result of the replacement of one —CH₂— in the alkyl group by —O—.

The alkoxy group is a linear-chain, branched, or cyclic alkoxy group and preferably is a linear-chain alkoxy group.

The number of carbon atoms in the alkoxy group is preferably from two to ten, preferably from two to six.

R^{vt2}, furthermore, can represent a C1 to C19 alkylsulfanyl group (alkylthio group) as a result of the replacement of one —CH₂— in the alkyl group by —S—.

The alkylsulfanyl group is a linear-chain, branched, or cyclic alkylsulfanyl group and preferably is a linear-chain alkylsulfanyl group.

The number of carbon atoms in the alkylsulfanyl group is preferably from one to ten, preferably from one to six.

Moreover, R^{vt2} can represent a C2 to C20 alkenyl group as a result of the replacement of one or two or more —CH₂—CH₂-s in the alkyl group by —CH═CH—.

The alkenyl group is a linear-chain, branched, or cyclic alkenyl group and preferably is a linear-chain alkenyl group.

The number of carbon atoms in the alkenyl group is preferably from two to ten, preferably from two to six.

R^{vt2}, furthermore, can represent a C2 to C20 alkynyl group as a result of the replacement of one or two or more —CH₂—CH₂-s in the alkyl group by —C≡C—.

The alkynyl group is a linear-chain, branched, or cyclic alkynyl group and preferably is a linear-chain alkynyl group.

The number of carbon atoms in the alkynyl group is preferably from two to ten, preferably from two to six.

Moreover, R^{vt2} can represent a C2 to C19 alkenyloxy group as a result of the replacement of one —CH₂— in the alkyl group by —O— and the replacement of one or two or more —CH₂—CH₂-s by —CH═CH—.

The alkenyloxy group is a linear-chain, branched, or cyclic alkenyloxy group and preferably is a linear-chain alkenyloxy group.

The number of carbon atoms in the alkenyloxy group is preferably from two to ten, preferably from two to six.

R^{vt2}, furthermore, can represent a C1 to C20 halogenated alkyl group as a result of the replacement of one or two or more hydrogen atoms in the alkyl group by a halogen atom.

The halogenated alkyl group is a linear-chain, branched, or cyclic halogenated alkyl group and preferably is a linear-chain halogenated alkyl group.

The number of carbon atoms in the halogenated alkyl group is preferably from two to ten, preferably from two to six.

Moreover, R^{vt2} can represent a C1 to C19 halogenated alkoxy group as a result of the replacement of one —CH₂— in the alkyl group by —O— and the replacement of one or two or more hydrogen atoms in the alkyl group by a halogen atom.

The halogenated alkoxy group is a linear-chain, branched, or cyclic halogenated alkoxy group and preferably is a linear-chain halogenated alkoxy group.

The number of carbon atoms in the halogenated alkoxy group is preferably from two to ten, preferably from two to six.

Specific examples of C1 to C20 alkyl groups (including substituted ones) at R^{vt2} include the groups represented by formula (R^{vt2}-1) to (R^{vt2}-36).

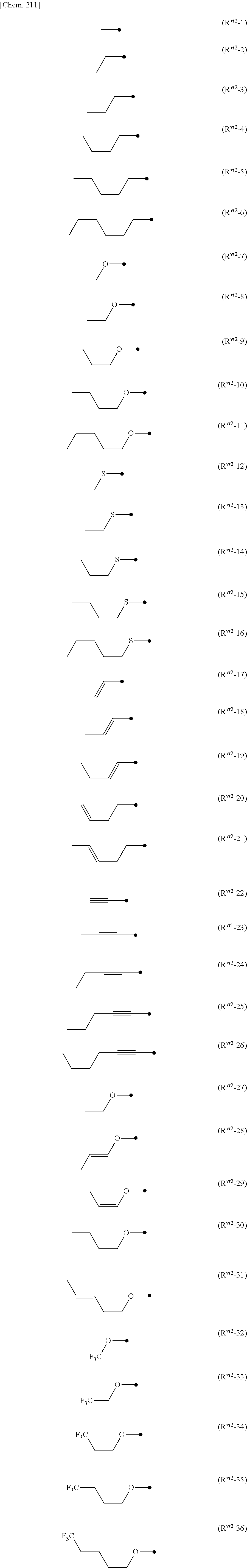

In formula (R^{vt2}-1) to (R^{vt2}-36), the black dot represents a bond to A^{vt3}.

When the ring structure to which R^{vt2} is bound is a phenyl group (aromatic), linear-chain C1 to C5 alkyl groups, linear-chain C1 to C4 alkoxy groups, and C4 and C5 alkenyl groups are preferred. When the ring structure to which R^{vt2} is bound is a saturated ring structure, such as cyclohexane, pyran, or dioxane, linear-chain C1 to C5 alkyl groups, linear-chain C1 to C4 alkoxy groups, and linear-chain C2 to C5 alkenyl groups are preferred.

For R^{vt2}, furthermore, it is preferred that the total number of carbon atoms and oxygen atoms, if any, be five or fewer, and it is preferred that R^{vt2} be a linear-chain group, when the stabilization of the nematic phase is sought.

It should be noted that from the viewpoint(s) of solubility, Δn, and/or Δεᵣ, it is preferred that R^{vt2} be a fluorine atom, a cyano group, a C2 to C6 linear-chain alkyl group, a C1 to C6 linear-chain alkoxy group, or a C1 to C6 linear-chain alkylsulfanyl group.

In general formula (vt), A^{vt1}, A^{vt2}, and A^{vt3} each independently represent any of a C3 to C16 hydrocarbon ring or a C3 to C16 heterocycle.

More specifically, it is preferred that the C3 to C16 hydrocarbon ring or C3 to C16 heterocycle represent a group selected from the group consisting of group (a), group (b), group (c), and group (d) below:
(a) a 1,4-cyclohexylene group (One —CH₂—, or two or more —CH₂-s not adjacent to each other, present in the group may be replaced by —O— or —S—.);
(b) a 1,4-phenylene group (One —CH═ or two or more —CH═s present in the group may be replaced by —N═.);
(c) a 1,4-cyclohexenylene group, a bicyclo[2.2.2]octan-1,4-diyl group, a naphthalen-2,6-diyl group, a naphthalen-1,4-diyl group, a 1,2,3,4-tetrahydronaphthalen-2,6-diyl group, a 5,6,7,8-tetrahydronaphthalen-1,4-diyl group, a decahydronaphthalen-2,6-diyl group, an anthracen-2,6-diyl group, an anthracen-1,4-diyl group, an anthracen-9,10-diyl group, or a phenanthren-2,7-diyl group (One —CH═ or two or more —CH═s present in a naphthalen-2,6-diyl group, naphthalen-1,4-diyl group, 1,2,3,4-tetrahydronaphthalen-2,6-diyl group, 5,6,7,8-tetrahydronaphthalen-1,4-diyl group, anthracen-2,6-diyl group, anthracen-1,4-diyl group, anthracen-9,10-diyl group, or phenanthren-2,7-diyl group may be replaced by —N═.);
(d) a thiophen-2,5-diyl group, a benzothiophen-2,5-diyl group, a benzothiophen-2,6-diyl group, a benzothiophen-3,7-diyl group, a dibenzothiophen-2,6-diyl group, or a thieno[3,2-b]thiophen-2,5-diyl group (One —CH═ or two or more —CH═s present in the group may be replaced by —N═.)

One hydrogen atom in A^{vt1}, A^{vt2}, and A^{vt3}, or each of two or more independently, may have been replaced by a substituent S^{vt1}.

The substituent S^{vt1} represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group.

The alkyl group is a linear-chain, branched, or cyclic alkyl group and preferably is a linear-chain alkyl group.

The number of carbon atoms in the alkyl group is preferably from two to ten, preferably from three to six.

One —CH₂— in the alkyl group, or each of two or more independently, may have been replaced with —O—, —S—, and/or —CO—.

One —CH₂—CH₂— in the alkyl group, or each of two or more independently, furthermore, may be replaced with —CH═CH—, —CF═CF—, —C≡C—, —CO—O—, —O—CO—, —CO—S—, —S—CO—, —CO—NH—, and/or —NH—CO—.

Moreover, one or two or more —CH₂—CH₂—CH₂-s in the alkyl group may be replaced with —O—CO—O—.

One hydrogen atom in the alkyl group, or each of two or more independently, may have been replaced with a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

The substituent S^{vt1} is preferably a fluorine atom or C1 to C3 linear-chain alkyl group.

It is, furthermore, preferred that at least one of A^{vt1}, A^{vt2}, or A^{vt3} be substituted with at least one substituent S^{vt1}.

Moreover, A^{vt1} is preferably substituted with at least one substituent S^{vt1}.

It should be noted that when there are multiple S^{vt1}s, they may be the same or may be different.

The position in A^{vt1} at which it is substituted with a substituent or substituents S^{vt1} is preferably any of formula (A^{vt1}-SP-1) to (A^{vt1}-SP-3) below.

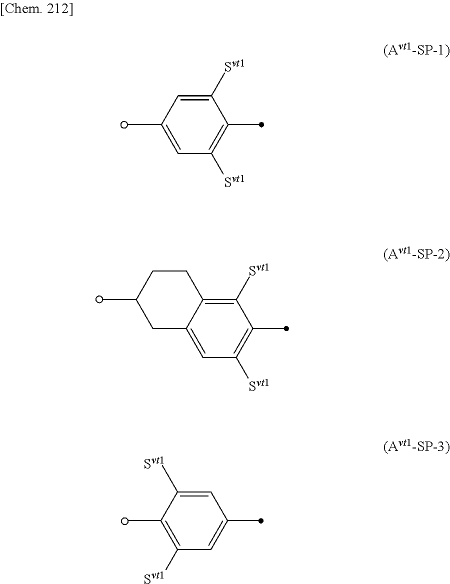

In formula (A^{vt1}-SP-1) to (A^{vt1}-SP-3), the white dot represents a bond to R^{vt1}, and the black dot represents a bond to —C≡C—.

The position in A^{vt2} at which it is substituted with a substituent or substituents S^{vt1} is preferably any of formula (A^{vt2}-SP-1) to (A^{vt2}-SP-7) below. From the viewpoint of compatibility with other liquid crystal compounds, it is preferred that A^{vt2} represent any of formula (A^{vt2}-SP-1) to (A^{vt2}-SP-7) below.

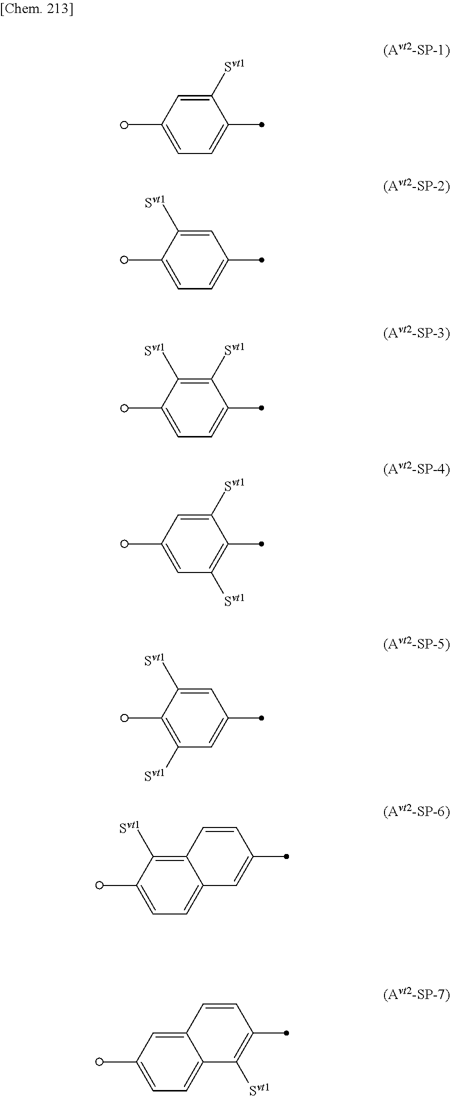

In formula (A^{vt2}-SP-1) to (A^{vt2}-SP-7), the white dot represents a bond to —C≡C—, and the black dot represents a bond to Z^{vt1}.

The position in A^{vt3} at which it is substituted with a substituent or substituents S^{vt1} is preferably any of formula (A^{vt3}-SP-1) to (A^{vt3}-SP-8) below. From the viewpoint of solubility, it is preferred that A^{vt3} represent any of formula (A^{vt3}-SP-1) to (A^{vt3}-SP-5) below.

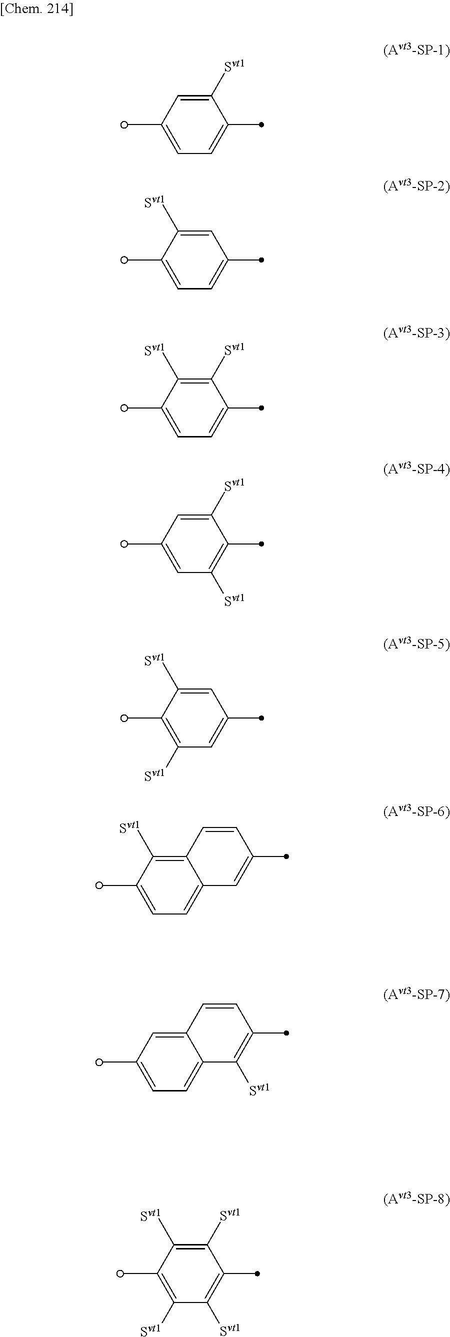

In formula (A^{vt3}-SP-1) to (A^{vt3}-SP-8), the white dot represents a bond to Z^{vt1}, and the black dot represents a bond to Z^{vt1} or R^{vt2}.

More specifically, it is preferred that A^{vt1} represent any of formula (A^{vt1}-1) to (A^{vt1}-5) below.

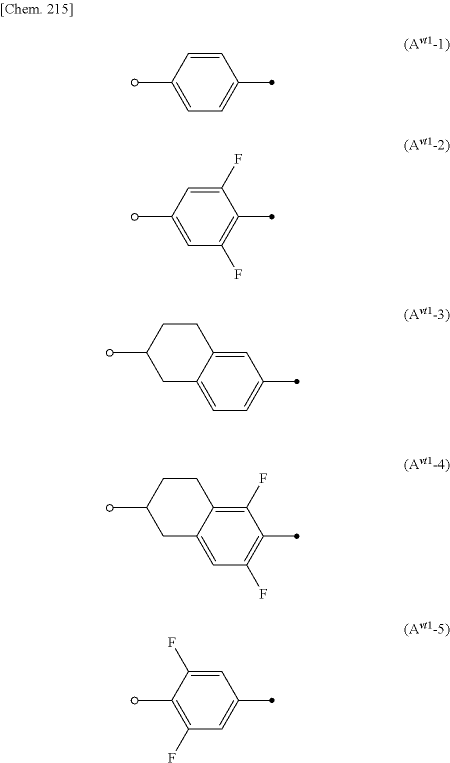

In formula (A^{vt1}-1) to (A^{vt1}-5), the white dot represents a bond to R^{vt1}, and the black dot represents a bond to —C≡C—.

More specifically, it is preferred that A^{vt2} represent any of formula (A^{vt2}-1) to (A^{vt2}-6) below.

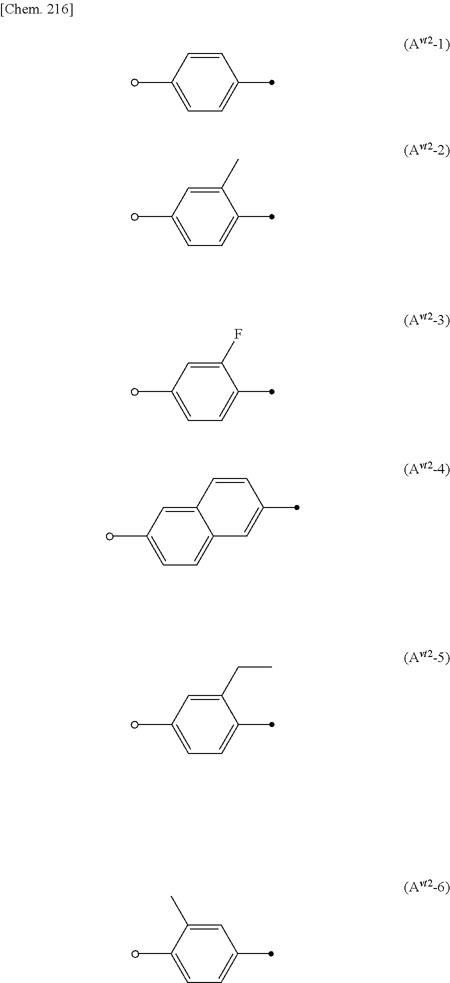

In formula (A^{vt2}-1) to (A^{vt2}-6), the white dot represents a bond to —C≡C—, and the black dot represents a bond to Z^{vt1}.

More specifically, it is preferred that A^{vt3} represent any of formula (A^{vt3}-1) to (A^{vt3}-5) below.

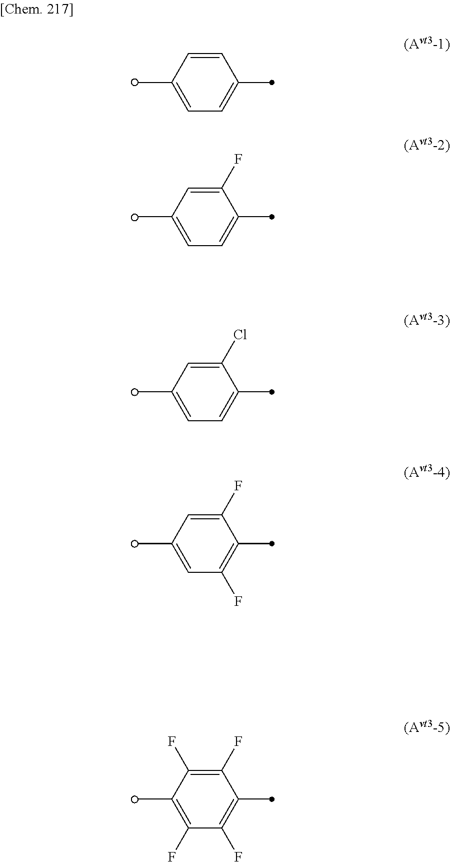

In formula (A^{vt3}-1) to (A^{vt3}-5), the white dot represents a bond to Z^{vt1}, and the black dot represents a bond to Z^{vt1} or R^{vt2}.

In general formula (vt), Z^{vt1} represents any of a single bond or a C1 to C20 alkylene group, independently at each occurrence.

The alkylene group is a linear-chain, branched, or cyclic alkylene group and preferably is a linear-chain alkylene group.

The number of carbon atoms in the alkylene group is preferably from two to ten, preferably from two to six.

One —CH₂— in the alkylene group, or each of two or more independently, may have been replaced with —O—, —CF₂—, and/or —CO—.

One —CH₂—CH₂— in the alkylene group, or each of two or more independently, furthermore, may be replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—.

Moreover, one —CH₂—CH₂—CH₂— in the alkylene group, or each of two or more independently, may have been replaced with —O—CO—O—.

When the alkylene group is substituted with a predetermined group or groups, however, an oxygen atom and an oxygen atom are not directly bound together.

Specific examples of C1 to C20 alkylene groups (including substituted ones) include the groups represented by formula (Z^{vt1}-1) to (Z^{vt1}-24).

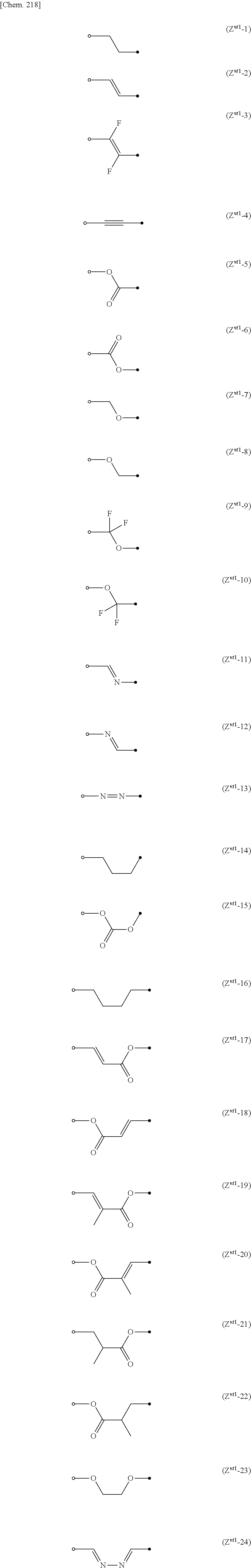

In formula (Z^{vt}-18) to (Z^{vt}1-24), the white dot represents a bond to A^{vt2} or A^{vt3}, and the black dot represents a bond to A^{vt3}.

In general formula (vt), n^{vt1} represents an integer of 1 to 3, preferably an integer of 1 or 2.

When n^{vt1} is 1, it is preferred that Z^{vt1} represent —C≡C— from the viewpoint(s) of Δn and/or Δεᵣ.

When n^{vt1} is 2 or 3, furthermore, it is preferred that at least one of the Z^{vt1}s represent —C≡C— from the viewpoint(s) of Δn and/or Δεᵣ.

It should be noted that when multiple A^{vt3}s and multiple Z^{vt1}s are present in general formula (vt), the A^{vt}3s may be the same or may be different, and the Z^{vt1}s may be the same or may be different.

The compound or compounds represented by general formula (vt) are preferably at least one compound represented by general formula (vt-1) below.

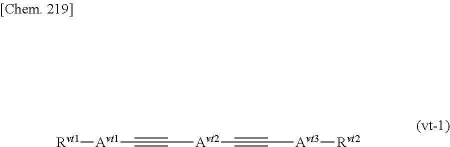

In general formula (vt-1), R^{vt1}, R^{vt2}, A^{vt1}, A^{vt2}, and A^{vt3} have the same meanings as R^{vt1}, R^{vt2}, A^{vt1}, A^{vt2}, and A^{vt3}, respectively, in general formula (vt) above.

Compounds represented by general formula (vt-1) are preferably compounds represented by general formula (vt-1-1) to (vt-1-3) below.

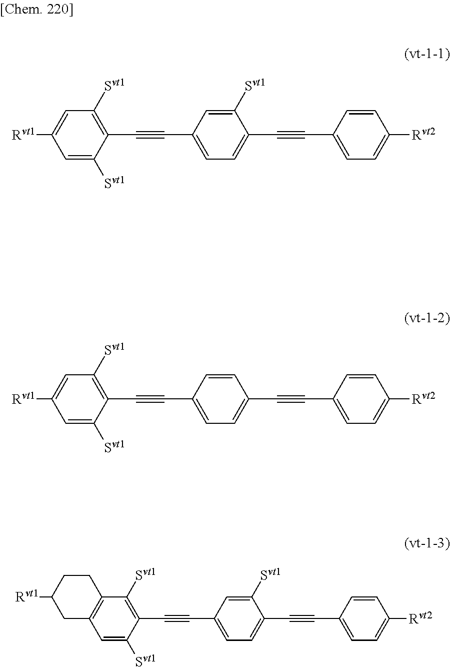

In general formula (vt-1-1) to (vt-1-3), R^{vt1}, R^{vt2}, and S^{vt1} have the same meanings as R^{vt1}, R^{vt2}, and S^{vt1}, respectively, in general formula (vt) above, independently at each occurrence.

Specific examples of compounds represented by general formula (vt-1-1) include the compounds represented by structural formula (vt-1-1.1) to (vt-1-1.24) below.

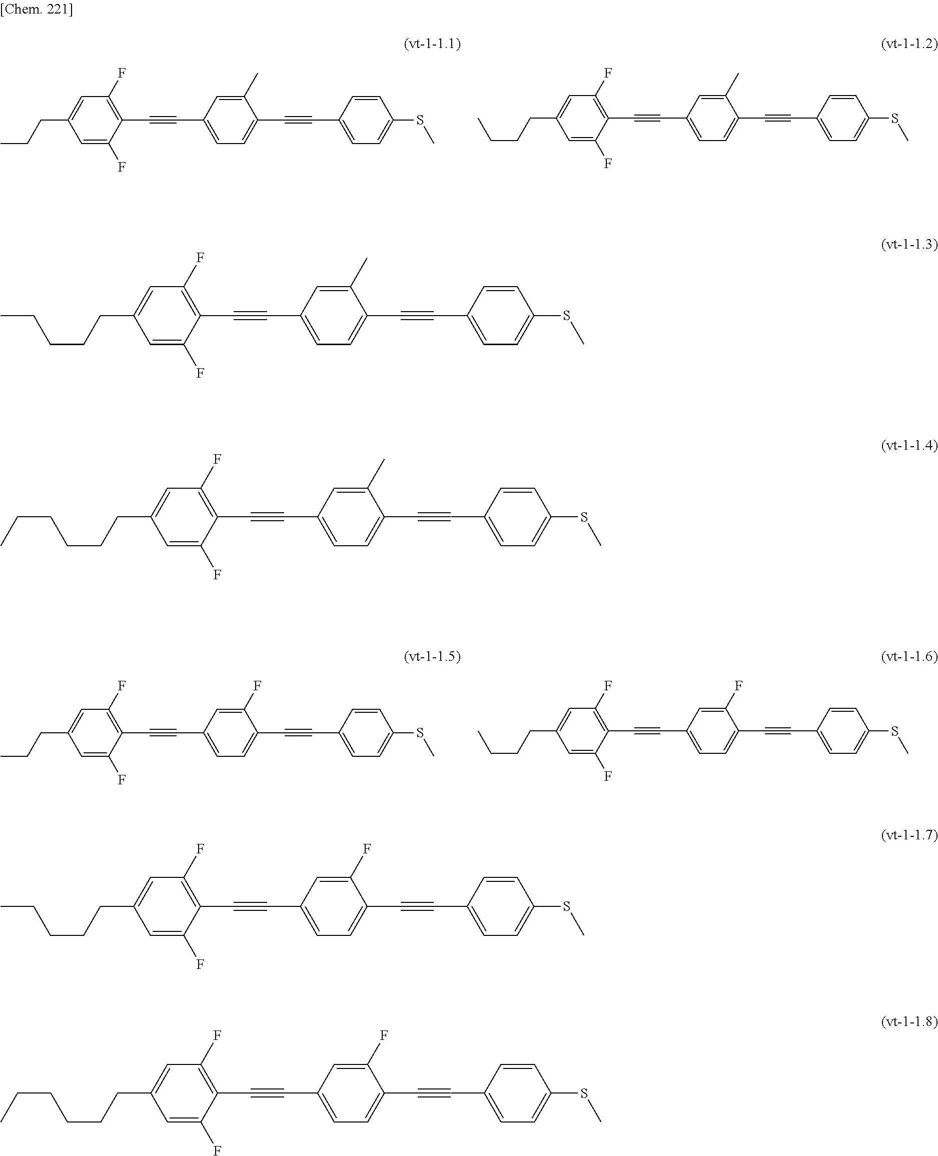

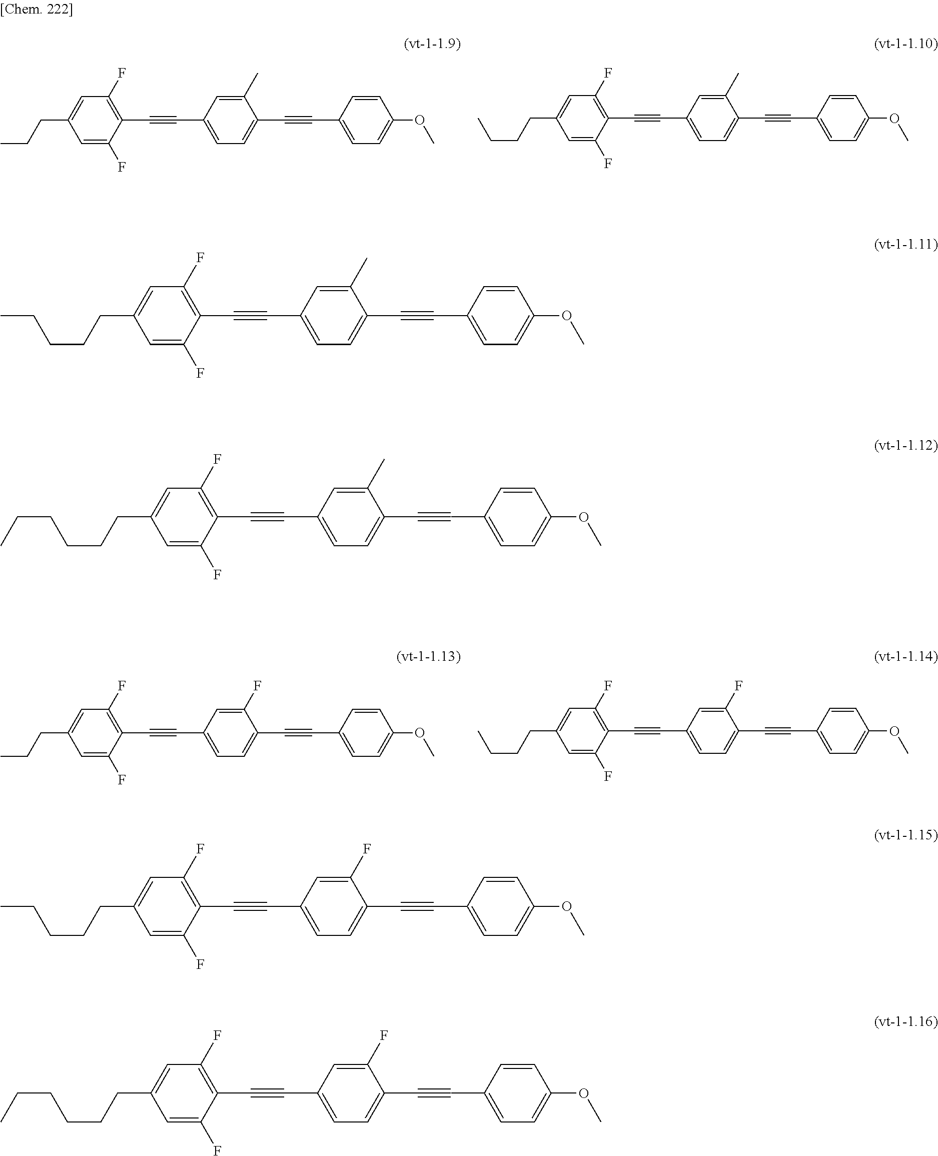

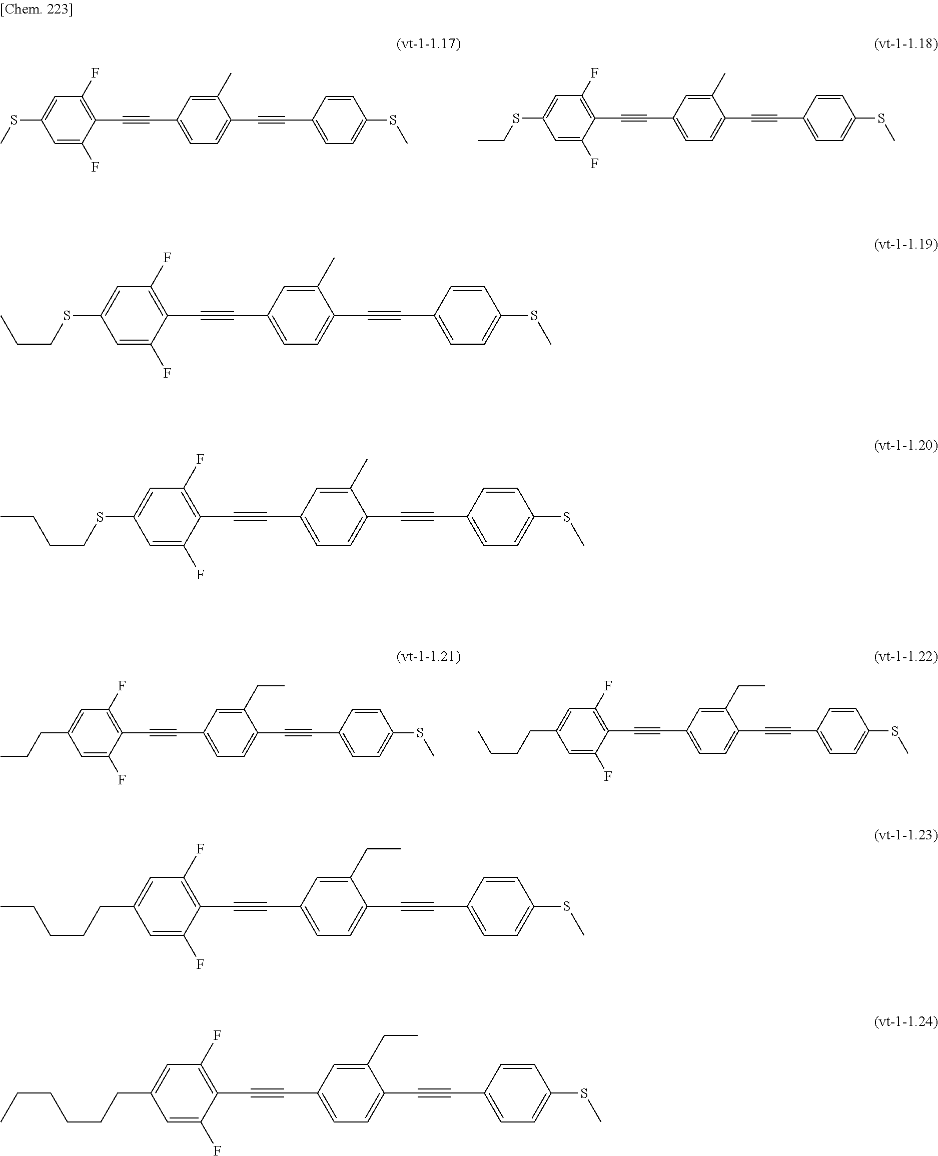

Specific examples of compounds represented by general formula (vt-1-2) include the compounds represented by structural formula (vt-1-2.1) to (vt-1-2.8) below.

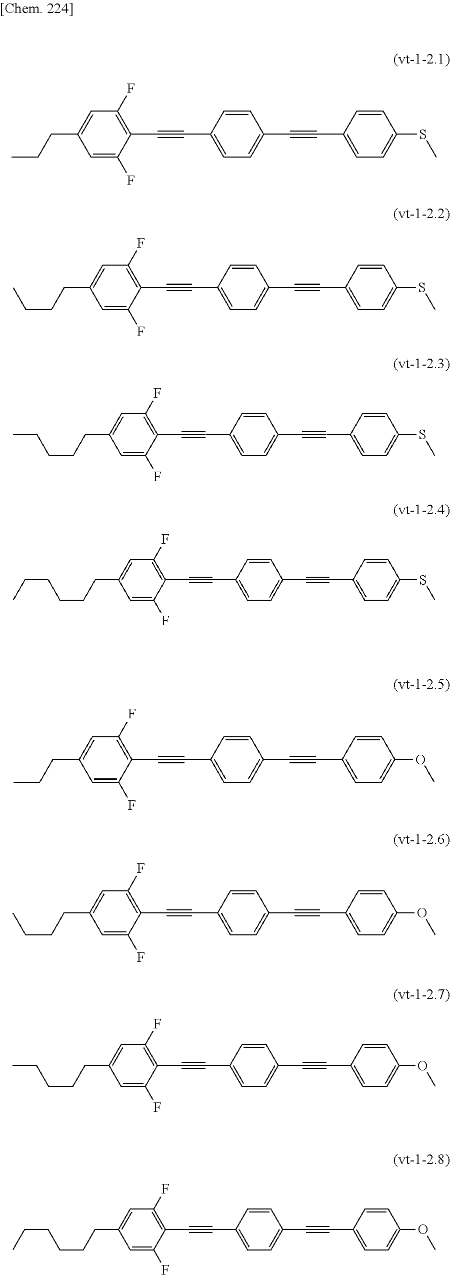

Specific examples of compounds represented by general formula (vt-1-3) include the compounds represented by structural formula (vt-1-3.1) to (vt-1-3.4) below.

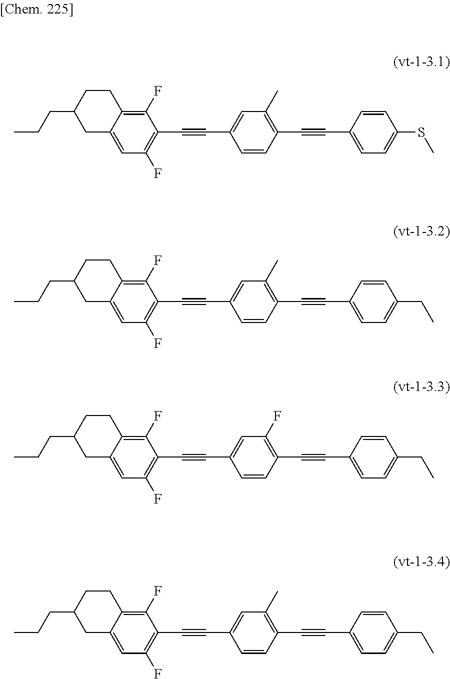

The number of types of compounds represented by general formula (vt), general formula (vt-1), general formula (vt-1-1) to (vt-1-3),
structural formula (vt-1-1.1) to (vt-1-1.24), structural formula (vt-1-2.1) to (vt-1-2.8), or structural formula (vt-1-3.1) to (vt-1-3.4) used in the liquid crystal composition is one or two or more, preferably from one to five, preferably from one to four, preferably from one to three, preferably one or two, preferably one.

The lower limit to the total amount of compounds represented by general formula (vt), general formula (vt-1), general formula (vt-1-1) to (vt-1-3),
structural formula (vt-1-1.1) to (vt-1-1.24), structural formula (vt-1-2.1) to (vt-1-2.8), or structural formula (vt-1-3.1) to (vt-1-3.4) in 100% by mass of the liquid crystal composition is preferably 0.1% by mass or more, preferably 0.5% by mass or more, preferably 1% by mass or more.

The upper limit to the total amount of compounds represented by general formula (vt), general formula (vt-1), general formula (vt-1-1) to (vt-1-3),
structural formula (vt-1-1.1) to (vt-1-1.24), structural formula (vt-1-2.1) to (vt-1-2.8), or structural formula (vt-1-3.1) to (vt-1-3.4) in 100% by mass of the liquid crystal composition is preferably 25% by mass or less, preferably 20% by mass or less, preferably 15% by mass or less.

From the viewpoint(s) of solubility, Δn, and/or Δεᵣ, it is preferred that the total amount of compounds represented by general formula (vt), general formula (vt-1), general formula (vt-1-1) to (vt-1-3),
structural formula (vt-1-1.1) to (vt-1-1.24), structural formula (vt-1-2.1) to (vt-1-2.8), or structural formula (vt-1-3.1) to (vt-1-3.4) in 100% by mass of the liquid crystal composition be from 0.1% to 25% by mass, preferably from 0.5% to 20% by mass, preferably from 1% to 15% by mass.

Compounds represented by general formula (vt) (including subordinate concepts) can be synthesized using known synthetic methods.

From the viewpoint of solubility, the liquid crystal composition according to the present invention is allowed to further contain one or two or more types of compounds represented by general formula (np-1) to (np-3) below.

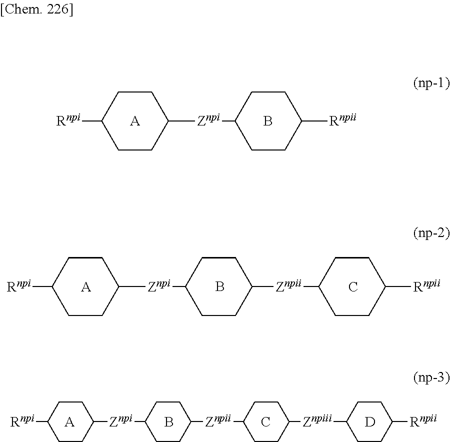

In general formula (np-1) to (np-3), R^{npi} and R^{npii} each independently represent any of a C1 to C20 alkyl group or a halogen atom.

A C1 to C20 alkyl group is a linear-chain, branched, or cyclic alkyl group and preferably is a linear-chain alkyl group.

The number of carbon atoms in the C1 to C20 alkyl group is preferably from two to ten, preferably from two to six.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CH—, —CH═CF—, —CF═CF—, and/or —C≡C—.

Moreover, one —CH₂—CH₂—CH₂— in the alkyl group, or each of two or more independently, may have been replaced with —O—CO—O—.

In addition, one hydrogen atom in the alkyl group, or each of two or more independently, may have been replaced with a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

For example, R^{npi} and R^{npii} can represent C1 to C19 alkoxy groups as a result of the replacement of one —CH₂— in the alkyl groups by —O—.

The alkoxy groups are linear-chain, branched, or cyclic alkoxy groups and preferably are linear-chain alkoxy groups.

The number of carbon atoms in the alkoxy groups is preferably from two to ten, preferably from two to six.

R^{npi} and R^{npii} furthermore, can represent C1 to C19 alkylsulfanyl groups (thioalkyl groups) as a result of the replacement of one —CH₂— in the alkyl groups by —S—.

The alkylsulfanyl groups are linear-chain, branched, or cyclic alkylsulfanyl groups and preferably are linear-chain alkylsulfanyl groups.

The number of carbon atoms in the alkylsulfanyl groups is preferably from two to ten, preferably from two to six.

Moreover, R^{npi} and R^{npii} can represent C2 to C20 alkenyl groups as a result of the replacement of one or two or more —CH₂—CH₂-s in the alkyl groups by —CH═CH—.

The alkenyl groups are linear-chain, branched, or cyclic alkenyl groups and preferably are linear-chain alkenyl groups.

The number of carbon atoms in the alkenyl groups is preferably from two to ten, preferably from two to six.

R^{npi} and R^{npii}, furthermore, can represent C2 to C20 alkynyl groups as a result of the replacement of one or two or more —CH₂—CH₂-s in the alkyl groups by —C≡C—.

The alkynyl group are linear-chain, branched, or cyclic alkynyl groups and preferably are linear-chain alkynyl groups.

The number of carbon atoms in the alkynyl groups is preferably from two to ten, preferably from two to six.

Moreover, R^{npi} and R^{npii} can represent C2 to C19 alkenyloxy groups as a result of the replacement of one —CH₂— in the alkyl groups by —O— and the replacement of one or two or more —CH₂—CH₂-s by —CH═CH—.

The alkenyloxy groups are linear-chain, branched, or cyclic alkenyloxy groups and preferably are linear-chain alkenyloxy groups.

The number of carbon atoms in the alkenyloxy groups is preferably from two to ten, preferably from two to six.

R^{npi} and R^{npii}, furthermore, can represent C1 to C20 halogenated alkyl groups as a result of the replacement of one or two or more hydrogen atoms in the alkyl groups by a halogen atom.

The halogenated alkyl groups are linear-chain, branched, or cyclic halogenated alkyl groups and preferably are linear-chain halogenated alkyl groups.

The number of carbon atoms in the halogenated alkyl groups is preferably from two to ten, preferably from two to six.

R^{npi} and R^{npii} can represent C1 to C19 halogenated alkoxy groups as a result of the replacement of one —CH₂— in the alkyl groups by —O— and the replacement of one or two or more hydrogen atoms in the alkyl groups by a halogen atom.

The halogenated alkoxy groups are linear-chain, branched, or cyclic halogenated alkoxy groups and preferably are linear-chain halogenated alkoxy groups.

The number of carbon atoms in the halogenated alkoxy groups is preferably from two to ten, preferably from two to six.

Specific examples of C1 to C20 alkyl groups (including substituted ones) at R^{npi} and R^{npii} include the groups represented by formula (R^{npi/ii}-1) to (R^{npi/ii}-36).

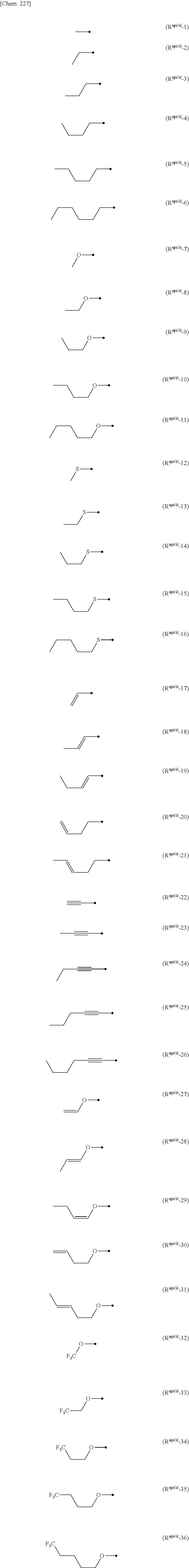

In formula (R^{npi}/ii-1) to (R^{npi}/ii-36), the black dot represents a bond to ring A, ring B, ring C, or ring D.

Examples of halogen atoms at R^{npi} and R^{npii} include fluorine atoms, chlorine atoms, bromine atoms, and iodine atoms.

In general formula (np-1) to (np-3), ring A, ring B, ring C, and ring D each independently represent a group selected from the group consisting of group (a), group (b), group (c), and group (d) below:
(a) a 1,4-cyclohexylene group (One —CH₂— or two or more nonadjacent —CH₂-s present in the group may be replaced by —O—.);
(b) a 1,4-phenylene group (One —CH═ or two or more —CH═s present in the group may be replaced by —N═.);
(c) a naphthalen-2,6-diyl group, a 1,2,3,4-tetrahydronaphthalen-2,6-diyl group, or a decahydronaphthalen-2,6-diyl group (One —CH═ or two or more —CH═s present in a naphthalen-2,6-diyl group or 1,2,3,4-tetrahydronaphthalen-2,6-diyl group may have been replaced by —N═.);
(d) a 1,4-cyclohexenylene group, a 1,3-dioxan-trans-2,5-diyl group, a pyrimidin-2,5-diyl group, or a pyridin-2,5-diyl group.

One hydrogen atom in ring A, ring B, ring C, and ring D, or each of two or more independently, may have been replaced by a substituent S^{npi1}.

The substituent S^{npi1} represents any of a halogen atom, a cyano group, or a C1 to C20 alkyl group.

Examples of halogen atoms include a fluorine atom, a chlorine atom, a bromine atom, and an iodine atom. From the viewpoints of stability and safety, a fluorine atom is preferred.

A C1 to C20 alkyl group is a linear-chain, branched, or cyclic alkyl group and preferably is a linear-chain alkyl group.

The number of carbon atoms in the C1 to C20 alkyl group is preferably from two to ten, preferably from two to six.

One —CH₂— in the alkyl group, or each of two or more independently, may have been replaced with —O—, —S—, —CO—, and/or —CS—.

One —CH₂—CH₂— in the alkyl group, or each of two or more independently, furthermore, may have been replaced with —CO—O—, —O—CO—, —CO—S—, —S—CO—, —CO—NH—, —NH—CO—, —CH═CH—, —CF═CF—, and/or —C≡C—.

One —CH₂—CH₂—CH₂— in the alkyl group, or each of two or more independently, may have been replaced with —O—CO—O—.

In addition, one hydrogen atom in the alkyl group, or each of two or more independently, may have been replaced by a halogen atom.

Examples of halogen atoms include a fluorine atom, a chlorine atom, a bromine atom, and an iodine atom.

When the alkyl group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

From the viewpoint of Vₜₕ, it is preferred that the substituent S^{npi1} be a halogen atom. Preferably, S^{npi1} is a fluorine atom.

It should be noted that when there are multiple S^{npi1}s, they may be the same or may be different.

The position in ring A at which it is substituted with a substituent or substituents S^{npi1} is preferably formula (A-SP-1) below.

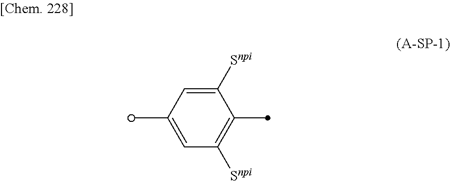

In formula (A-SP-1), the white dot represents a bond to R^{npi}, and the black dot represents a bond to Z^{npi}.

More specifically, it is preferred that ring A represent any of formula (A-1) to (A-3) below.

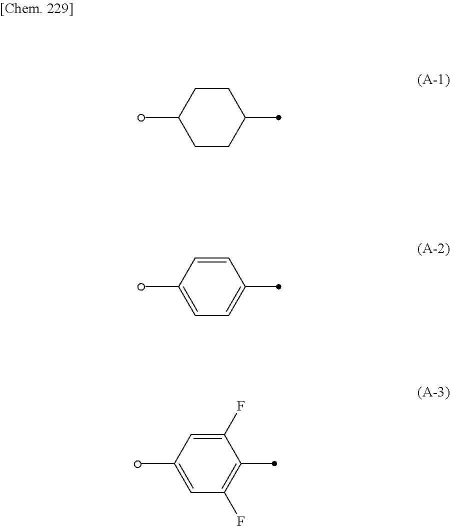

In formula (A-1) to (A-3), the white dot represents a bond to R^{npi}, and the black dot represents a bond to Z^{npi}.

More specifically, it is preferred that ring B represent any of formula (B-1) or (B-2) below.

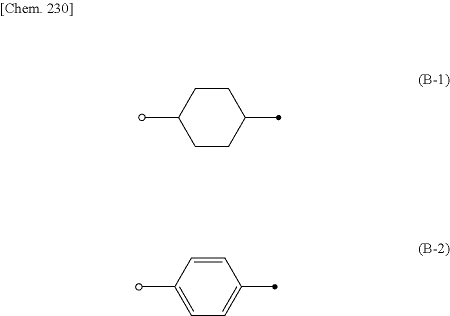

In formula (B-1) and (B-2), the white dot represents a bond to Z^{npii}, and the black dot represents a bond to R^{npii} or Z^{npii}.

More specifically, it is preferred that ring C represent any of formula (C-1) or (C-2) below.

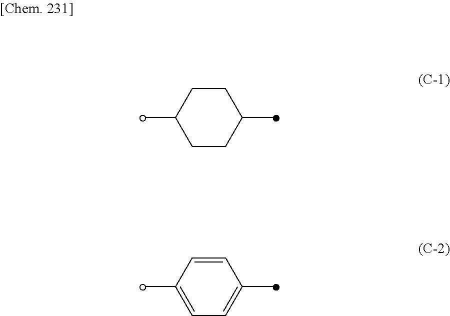

In formula (C-1) and (C-2), the white dot represents a bond to Z^{npii}, and the black dot represents a bond to R^{npii} or Z^{npiii}.

In general formula (np-1) to (np-3), Z^{npi}, Z^{npii}, and Z^{npiii} each independently represent any of a single bond or a C1 to C20 alkylene group.

One —CH₂— in the alkylene group, or each of two or more independently, may have been replaced with —O—, —CF₂—, and/or —CO—.

One —CH₂—CH₂— in the alkylene group, or each of two or more independently, furthermore, may be replaced with —CH₂—CH(CH₃)—, —CH (CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—.

Moreover, one —CH₂—CH₂—CH₂— in the alkylene group, or each of two or more independently, may have been replaced with —O—CO—O—.

When a C1 to C20 alkylene group is substituted with a predetermined group or groups, however, an oxygen atom and an oxygen atom are not directly bound together.

From the viewpoint of the stability of the compound, furthermore, it is preferred that a sulfur atom and a sulfur atom and/or an oxygen atom and a sulfur atom be not directly bound together.

Specific examples of C1 to C20 alkylene groups (including substituted ones) include the groups represented by formula (Z^{npi/ii/iii}-1) to (Z^{npi/ii/iii}-24).

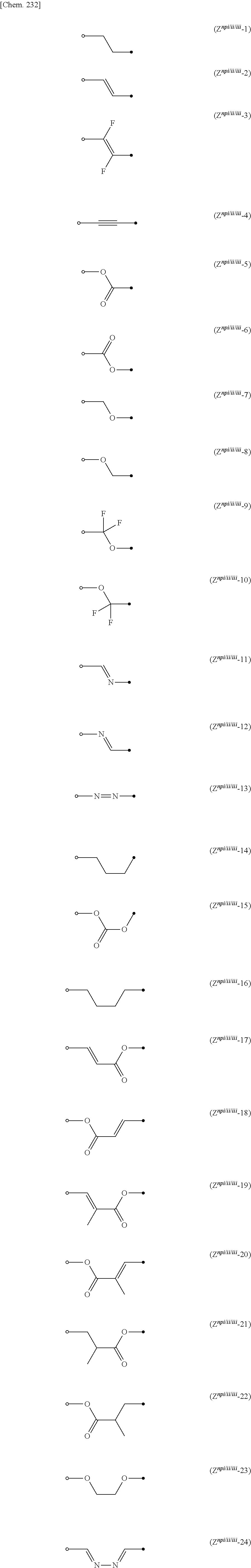

In formula (Z^{npi/ii/iii}-1) to (Z^{npi/ii/iii}-24), the white dot represents a bond to ring A, ring B, or ring C, and the black dot represents a bond to ring B, ring C, or ring D.

From the viewpoint(s) of Δn and/or Δεᵣ, it is preferred that Z^{npi}, Z^{npii}, and Z^{npiii} each independently represent any of a single bond, —C≡C—, or —CO—O—.

For compounds represented by general formula (np-1) to (np-3), however, compounds represented by general formula (vt) (including subordinate concepts) are excluded.

Compounds represented by general formula (np-2) are preferably compounds represented by general formula (np-2-1) to (np-2-3) below.

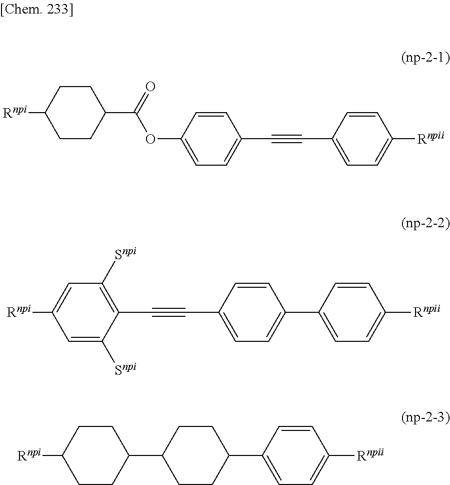

In general formula (np-2-1) to (np-2-3), R^{npi}, R^{npii} and S^{npi} have the same meanings as R^{npi}, R^{P}ii, and S^{npi}, respectively, in general formula (np-1) to (np-3) above.

Specific examples of compounds represented by general formula (np-2-1) include the compound represented by structural formula (np-2-1.1) below.

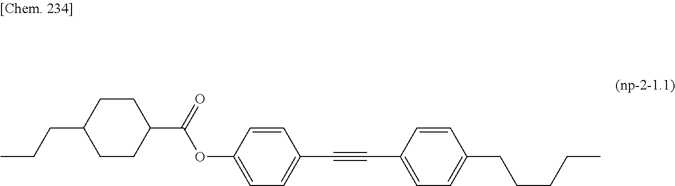

Specific examples of compounds represented by general formula (np-2-2) include the compounds represented by structural formula (np-2-2.1) to (np-2-2.5) below.

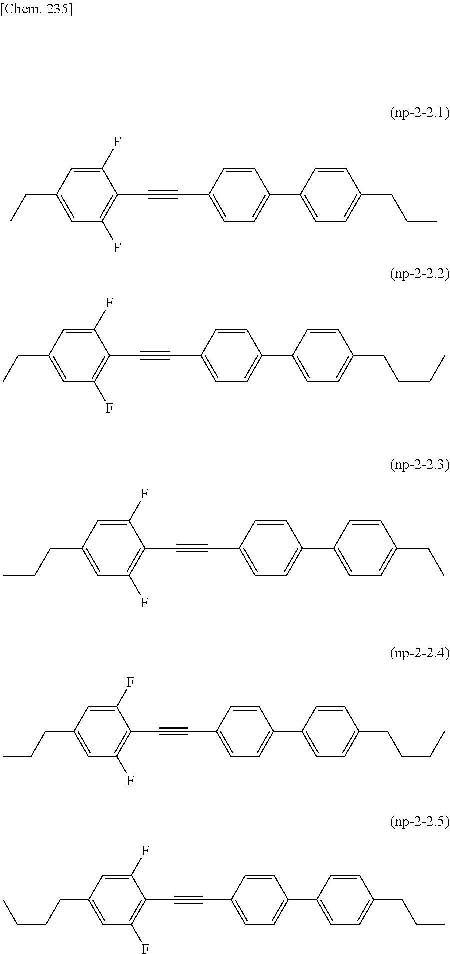

Specific examples of compounds represented by general formula (np-2-3) include the compounds represented by structural formula (np-2-3.1) to (np-2-3.3) below.

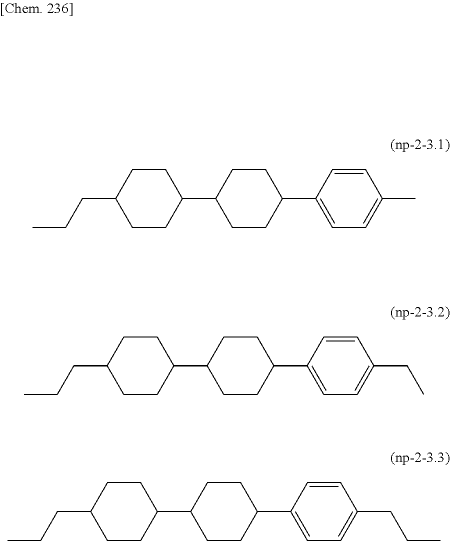

The number of types of compounds represented by general formula (np-1) to (np-3), general formula (np-2-1) to (np-2-3), structural formula (np-2-1.1), structural formula (np-2-2.1) to (np-2-2.5), or structural formula (np-2-3.1) to (np-2-3.3) used in the liquid crystal composition is one or two or more, preferably from one to ten, preferably from one to eight, preferably from one to six, preferably from one to four, preferably one or two.

The lower limit to the total amount of compounds represented by general formula (np-1) to (np-3), general formula (np-2-1) to (np-2-3), structural formula (np-2-1.1), structural formula (np-2-2.1) to (np-2-2.5), or structural formula (np-2-3.1) to (np-2-3.3) in 100% by mass of the liquid crystal composition is preferably 0.1% by mass, preferably 0.5% by mass, preferably 1% by mass.

The upper limit to the total amount of compounds represented by general formula (np-1) to (np-3), general formula (np-2-1) to (np-2-3), structural formula (np-2-1.1), structural formula (np-2-2.1) to (np-2-2.5), or structural formula (np-2-3.1) to (np-2-3.3) in 100% by mass of the liquid crystal composition is preferably 30% by mass, preferably 20% by mass, preferably 15% by mass.

From the viewpoint(s) of solubility, Δn, and/or Δεᵣ, it is preferred that the total amount of compounds represented by general formula (np-1) to (np-3), general formula (np-2-1) to (np-2-3), structural formula (np-2-1.1), structural formula (np-2-2.1) to (np-2-2.5), or structural formula (np-2-3.1) to (np-2-3.3) in 100% by mass of the liquid crystal composition be from 0.1% to 30% by mass, preferably from 0.5% to 20% by mass, preferably from 1% to 15% by mass.

Compounds represented by general formula (np-1) to (np-3) (including subordinate concepts) can be synthesized using known synthetic methods.

### (Liquid Crystal Composition)

A liquid crystal composition according to the present invention can be produced by, for example, mixing at least one compound represented by general formula (i) and at least one compound represented by general formula (ii) as described above, optionally with other compounds as described above and additives.

Examples of additives include stabilizers, colorant compounds, polymerizable compounds, and azotolane compounds.

Examples of stabilizers include hydroquinones, hydroquinone monoalkyl ethers, tertiary butylcatechols, pyrogallols, thiophenols, nitro compounds, R-naphthylamines, R-naphthols, nitroso compounds, hindered phenols, and hindered amines.

Examples of hindered phenols include the hindered phenolic antioxidants represented by structural formula (XX-1) to (XX-3) below.

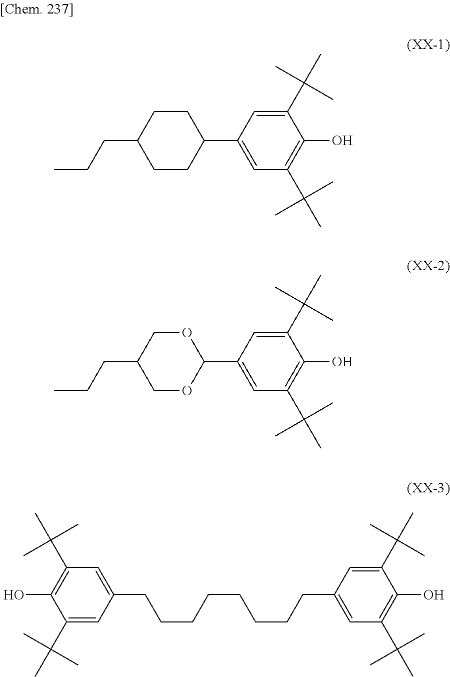 [SMILES: CCCC1CCC(c2cc(C(C)(C)C)c(O)c(C(C)(C)C)c2)CC1 ; CCCC1COC(c2cc(C(C)(C)C)c(O)c(C(C)(C)C)c2)OC1 ; CC(C)(C)c1cc(CCCCCCCCc2cc(C(C)(C)C)c(O)c(C(C)(C)C)c2)cc(C(C)(C)C)c1O]

Examples of hindered amines include the hindered amine photostabilizers represented by structural formula (YY-1) and (YY-2) below.

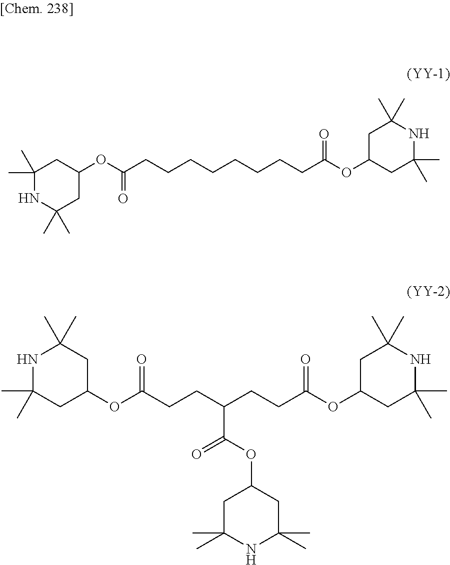

The number of types of stabilizers in the liquid crystal composition when a stabilizer or stabilizers are used is one or two or more, preferably from one to ten, preferably from one to eight, preferably from one to six, preferably from one to four, preferably one or two.

The total amount of stabilizers in 100% by mass of the liquid crystal composition when a stabilizer or stabilizers are used is preferably from 0.005% to 1% by mass, preferably from 0.02% to 0.50% by mass, preferably from 0.03% to 0.35% by mass.

As for the combination of compounds used in the liquid crystal composition, furthermore, the following combinations are preferred from the viewpoint(s) of solubility, Δn, and/or Δεᵣ:
1. A combination of a compound represented by general formula (i)
(or a subordinate concept) and a compound represented by general formula (ii) (or a subordinate concept);
2. A combination of a compound represented by general formula (i)
(or a subordinate concept), a compound represented by general formula (ii-2) (or a subordinate concept), and a compound
represented by general formula (ii-3) (or a subordinate concept);
3. A combination of a compound represented by general formula (i-4)
(or a subordinate concept), a compound represented by general formula (ii-2) (or a subordinate concept), a compound
represented by general formula (ii-3) (or a subordinate concept), a compound represented by general formula (ii-4) (or a subordinate concept), and a compound represented by general formula (ii-6) (or a subordinate concept);
4. A combination of a compound represented by general formula (i-4)
(or a subordinate concept), a compound represented by general formula (ii-1) (or a subordinate concept), a compound
represented by general formula (ii-2) (or a subordinate concept), a compound represented by general formula (ii-3) (or a subordinate
concept), and a compound represented by general formula (ii-5) (or a subordinate concept);
5. A combination of a compound represented by general formula (i-4)
(or a subordinate concept), a compound represented by general formula (ii-2) (or a subordinate concept), a compound
represented by general formula (ii-3) (or a subordinate concept), and a compound represented by general formula (ii-6) (or a
subordinate concept);
6. A combination of a compound represented by general formula (i-10)
(or a subordinate concept), a compound represented by general formula (ii-2) (or a subordinate concept), a compound
represented by general formula (ii-3) (or a subordinate concept), a compound represented by general formula (ii-4) (or a subordinate
concept), and a compound represented by general formula (ii-6) (or a subordinate concept);
7. A combination of a compound represented by general formula (i-4)
(or a subordinate concept), a compound represented by general formula (i-11) (or a subordinate concept), a compound
represented by general formula (ii-2) (or a subordinate concept), a compound represented by general formula (ii-3) (or a subordinate
concept), a compound represented by general formula (ii-4) (or a subordinate concept), and a compound represented
by general formula (ii-6) (or a subordinate concept);
8. A combination of a compound represented by general formula (i-4)
(or a subordinate concept), a compound represented by general formula (i-8) (or a subordinate concept), a compound represented
by general formula (ii-2) (or a subordinate concept), a compound represented by general formula (ii-3) (or a subordinate
concept), a compound represented by general formula (ii-4) (or a subordinate concept), and a compound represented
by general formula (ii-6) (or a subordinate concept).

### <Characteristic Parameters of the Liquid Crystal Composition>

The liquid crystal phase upper limit temperature (Tₙᵢ) is the temperature at which a liquid crystal composition undergoes a phase transition from the nematic phase to the isotropic phase.

Tₙᵢ is measured by making a preparation, which is obtained by sandwiching the liquid crystal composition between a glass slide and a coverslip, and observing it under a polarization microscope while heating it on a hot stage.

Alternatively, Tₙᵢ can also be measured by differential scanning calorimetry (DSC).

The unit used is “° C.”

A higher Tₙᵢ results in a higher temperature at which the nematic phase can be maintained, allowing for a broader driving temperature range.

The liquid crystal phase upper limit temperature (Tₙᵢ) of the liquid crystal composition according to the present invention can be set as appropriate according to the situation, such as when the liquid crystal display element is used indoors, in an automobile, or in other situations in which its external temperature can be controlled, or when it is used outdoors. From the viewpoint of the driving temperature range, however, it is preferred that Tₙᵢ be 100° C. or above, preferably from 100° C. to 200° C., preferably from 110° C. to 180° C.

The liquid crystal phase lower limit temperature (T_{→n}) is the temperature at which a liquid crystal composition undergoes a phase transition to the nematic phase from another phase (the glass phase, smectic phase, or crystalline phase).

T_{→n} is measured by loading the liquid crystal composition into a glass capillary, immersing the capillary in a refrigerant at −70° C. to induce a phase transition of the liquid crystal composition to another phase, and observing the composition while increasing the temperature.

Alternatively, T_{→n} can also be measured by differential scanning calorimetry (DSC).

The unit used is “° C.”

A lower T_{→n} results in a lower temperature at which the nematic phase can be maintained, allowing for a broader driving temperature range.

From the viewpoint of driving temperatures, it is preferred that the liquid crystal phase lower limit temperature (T_{→n}) of the liquid crystal composition according to the present invention be 10° C. or below, preferably from −70° C. to 0° C., preferably from −40° C. to −5° C.

Δn (refractive index anisotropy) is correlated with Δn in the near-infrared region used with optical sensors, which will be described later herein.

A greater Δn results in greater phase modulation power for light at the target wavelength, making the liquid crystal composition particularly suitable for optical sensors.

Δn at 25° C. and 589 nm is determined from the difference (nₑ-nₒ) between the extraordinary refractive index (nₑ) and the ordinary refractive index (nₒ) of the liquid crystal composition using an Abbe refractometer.

Alternatively, Δn can also be determined from a retardation meter.

Between the retardation Re, the thickness of the liquid crystal layer d, and Δn, the relationship Δn=Re/d holds.

The liquid crystal composition is poured into a glass cell having a cell gap (d) of approximately 3.0 μm with a polyimide alignment film that has undergone antiparallel rubbing treatment, and the in-plane Re is measured using RETS-100 retardation film-optical material inspection system (manufactured by Otsuka Electronics Co., Ltd.).

The measurement is performed under the conditions of a temperature of 25° C. and 589 nm, and the result is unitless.

From the viewpoint of phase modulation power for light at a wavelength, it is preferred that the Δn at 25° C. and 589 nm of the liquid crystal composition according to the present invention be 0.38 or greater, preferably from 0.38 to 0.60, preferably from 0.40 to 0.55, preferably from 0.40 to 0.50.

Rotational viscosity (γ₁) is a coefficient of viscosity related to the rotation of liquid crystal molecules. γ₁ can be measured by loading the liquid crystal composition into a glass cell with a cell gap of approximately 10 μm, applying a voltage of 50 V, and measuring it using LCM-2 (manufactured by TOYO Corporation).

For a liquid crystal composition with positive dielectric anisotropy, a planar cell is used. For a liquid crystal composition with negative dielectric anisotropy, a homeotropic cell is used.

The measurement is performed at a temperature of 25° C., and the unit used is mPa-s.

A smaller γ₁ results in a shorter response time of the liquid crystal composition, making the composition suitable for all types of liquid crystal display elements.

From the viewpoint of response time, it is preferred that the rotational viscosity (γ₁) of the liquid crystal composition at 25° C. of the liquid crystal composition according to the present invention be from 150 to 2000 mPa s, preferably from 200 to 1500 mPa-s, preferably from 250 to 1000 mPa-s.

The threshold voltage (Vₜₕ) is correlated with the driving voltage of a liquid crystal composition.

Vₜₕ can be determined by loading the liquid crystal composition into a TN cell with a gap of 8.3 μm and determining it from the transmittance when a voltage is applied.

The measurement is performed at 25° C., and the unit used is “V”.

A lower Vₜh results in a lower voltage at which the liquid crystal composition can be driven.

From the viewpoint of driving voltage, it is preferred that the Vₜₕ at 25° C. of the liquid crystal composition according to the present invention be 3.0 V or lower, preferably from 0.3 to 3.0 V, preferably from 0.5 to 2.7 V, preferably from 0.7 to 2.5 V, preferably from 0.9 to 2.3 V, preferably from 1.1 to 2.1 V, preferably from 1.3 to 2.1 V.

As for the dielectric anisotropy in the radiofrequency region, higher dielectric anisotropy results in greater phase modulation power for radio waves within the target frequency band, making the liquid crystal composition particularly suitable for antenna applications.

In antenna applications, furthermore, a smaller dielectric tangent in the radiofrequency region is advantageous because it leads to a smaller energy loss within the target frequency band.

For the liquid crystal composition according to the present invention, the dielectric anisotropy Δεᵣ and the mean dielectric tangent tan δᵢₛₒ at 10 GHz were measured as typical characteristics in the radiofrequency region.

Δεᵣ=(εᵣ∥−ϵᵣ⊥), and tan δᵢₛₒ=(2εᵣ⊥ tan δ⊥+εᵣ∥tan δ∥)/(2εr⊥+εr∥).

In this context, “εᵣ” is the dielectric constant, “tan δ” is the dielectric tangent, the subscript “∥” means that the component is in the direction parallel to the direction of orientation of the liquid crystal, and the subscript “⊥” means that the component is in the direction perpendicular to the direction of orientation of the liquid crystal.

Δεᵣ and tan δᵢₛₒ can be measured by the following method.

First, the liquid crystal composition is introduced into a capillary tube made of polytetrafluoroethylene (PTFE).

The capillary tube used here has an inner radius of 0.80 mm and an outer radius of 0.835 mm, with the effective length being 4.0 cm.

The capillary tube with the sealed liquid crystal composition is introduced into the center of a cavity resonator having a resonant frequency of 10 GHz (manufactured by EM labs, Inc.).

This cavity resonator has external dimensions of a diameter of 30 mm and a width of 26 mm.

Then a signal is input, and the resulting output signal is recorded using a network analyzer (manufactured by Keysight Technologies, Inc.).

Using the differences between the resonant frequency and other parameters of the PTFE capillary tube without the sealed liquid crystal composition and the resonant frequency and other parameters of the PTFE capillary tube with the sealed liquid crystal composition, the dielectric constant (εᵣ) and the loss angle (δ) at 10 GHz are determined.

The tangent of the 5 obtained, furthermore, is the dielectric tangent (tan δ).

It should be noted that the resonant frequency and other parameters obtained using the PTFE capillary tube with the sealed liquid crystal composition are determined as the values of characteristic components perpendicular to the direction of orientation of the liquid crystal molecules and the values of characteristic components parallel to the direction of orientation of the liquid crystal molecules through the control of the orientation of the liquid crystal molecules.

To align the liquid crystal molecules in the direction perpendicular to the PTFE capillary tube (perpendicular to the direction of the effective length) or in the direction parallel to the tube (parallel to the direction of the effective length) a magnetic field from a permanent magnet or electromagnet is used.

The magnetic field has, for example, a gap width of 45 mm, with the strength of the magnetic field near its center being 0.23 tesla.

By rotating the PTFE capillary tube with the sealed liquid crystal composition parallel or perpendicular to the magnetic field, the desired characteristic components are obtained.

The measurement is performed at a temperature of 25° C., and both Δεᵣ and tan δᵢₛₒ are unitless.

For the Δεᵣ at 25° C. of the liquid crystal composition according to the present invention, a greater value is preferred. From the viewpoint of phase modulation power in the GHz band, however, it is preferred that Δεᵣ be 0.90 or greater, preferably from 0.90 to 1.40, preferably from 0.95 to 1.40, preferably from 1.00 to 1.35.

For the tan δᵢₛₒ at 25° C. of the liquid crystal composition according to the present invention, a smaller value is preferred. From the viewpoint of loss in the GHz band, however, it is preferred that tan δᵢₛₒ be 0.025 or less, preferably from 0.001 to 0.025, preferably from 0.003 to 0.020, preferably from 0.005 to 0.017, preferably from 0.007 to 0.015, preferably from 0.008 to 0.013, preferably from 0.009 to 0.012.

### (Liquid Crystal Display Element, Sensor, Liquid Crystal Lens, Optical Communication Equipment, and Antenna)

A liquid crystal display element, a sensor, a liquid crystal lens, optical communication equipment, and an antenna made using a liquid crystal composition according to the present invention will now be described.

A liquid crystal display element according to the present invention is characterized by the use of a liquid crystal composition as described above, and preferably operates using the active matrix scheme or the passive matrix scheme

The liquid crystal display element according to the present invention, furthermore, is preferably a liquid crystal display element that reversibly switches the dielectric constant by reversibly change the direction of orientation of liquid crystal molecules in a liquid crystal composition as described above.

A sensor according to the present invention is characterized by the use of a liquid crystal composition as described above. Examples of its forms include distance measurement sensors that utilize electromagnetic waves, visible light, or infrared light, infrared sensors that utilize temperature changes, temperature sensors that utilize changes in the wavelength of reflected light caused by changes in the pitch of cholesteric liquid crystals, pressure sensors that utilize changes in the wavelength of reflected light, ultraviolet light sensors that utilize changes in the wavelength of reflected light caused by composition changes, electrical sensors that utilize temperature changes caused by voltage or current, radiation sensors that utilize temperature changes associated with the trajectory of radiation particles, ultrasonic sensors that utilizes changes in the arrangement of liquid crystal molecules caused by mechanical vibration of ultrasonic waves, and electromagnetic field sensors that utilize changes in the wavelength of reflected light caused by temperature changes or changes in the arrangement of liquid crystal molecules caused by an electric field.

Distance measurement sensors are preferably those for LiDAR (Light Detection And Ranging), which uses a light source.

The LiDAR is preferably that for satellites, aircraft, unmanned aerial vehicles (drones), automobiles, railroads, or ships.

Sensors for automobiles are preferably those for self-guided automobiles in particular.

The light source is preferably an LED or laser, preferably a laser.

The light used for LiDAR is preferably infrared light, and its wavelength is preferably from 800 to 2000 nm.

In particular, an infrared laser with a wavelength of 905 nm or 1550 nm is preferred.

When the cost of the light detector used or all-weather sensitivity is a priority, a 905-nm infrared laser is preferred. When safety concerning human vision is a priority, a 1550-nm infrared laser is preferred.

With a liquid crystal composition according to the present invention, which exhibits a high Δn, a sensor can be provided that achieves great phase modulation power in the visible-light, infrared-light, and electromagnetic-wave regions and is superior in detection sensitivity.

A liquid crystal lens according to the present invention is characterized by the use of a liquid crystal composition as described above. For example, one of its forms includes a first transparent electrode layer, a second transparent electrode layer, a liquid crystal layer disposed between the first transparent electrode layer and the second transparent electrode layer and containing the liquid crystal composition as described above, an insulating layer disposed between the second transparent electrode layer and the liquid crystal layer, and a high-resistance layer disposed between the insulating layer and the liquid crystal layer.

The liquid crystal lens according to the present invention is utilized as, for example, a 2D/3D switching lens or a lens for camera focusing.

Optical communication equipment according to the present invention is characterized by the use of a liquid crystal composition as described above. One example of its forms is LCOS (Liquid crystal on silicon), which is configured with a reflective layer (electrode) and a liquid crystal layer on top of it, with liquid crystals constituting individual ones of multiple pixels arranged in a two-dimensional array.

The optical communication equipment according to the present invention is used as, for example, a spatial phase modulator.

An antenna according to the present invention is characterized by the use of a liquid crystal composition as described above.

More specifically, the antenna according to the present invention includes a first substrate having multiple slots, a second substrate facing the first substrate and provided with a power feed section, a first dielectric layer disposed between the first substrate and the second substrate, multiple patch electrodes positioned corresponding to the multiple slots, a third substrate provided with the patch electrodes, and a liquid crystal layer disposed between the first substrate and the third substrate. The liquid crystal layer contains the liquid crystal composition as described above.

The liquid crystal composition used is a liquid crystal composition containing one or two or more types of compounds represented by general formula (i) (including subordinate concepts), which have an indane structure and an isothiocyanate group (—NCS), and one or two or more types of compounds represented by general formula (ii), which have an isothiocyanate group (—NCS). By using it, an antenna can be provided that has high reliability against external stimuli, such as heat, by virtue of a high Tₙᵢ, a large Δn, a low Vₜₕ, a large Δεᵣ, a small tan δiso, and good storability at low temperatures.

As a result of this, an antenna can be provided that allows for greater phase control of microwave or millimeter-wave electromagnetic waves.

The antenna according to the present invention preferably operates at Ka-band frequencies or K-band frequencies or Ku-band frequencies, which are used in satellite communications.

The antenna according to the present invention preferably has a configuration in which a radial line slot array and a patch antenna array are combined.

Regarding the structure of the antenna according to the present invention, information such as the matters described in International Publication No. 2021/157189 and other publications can be considered and applied.

### EXAMPLES

The present invention will now be described in further detail by providing examples. The present invention, however, is by no means limited to the examples below.

The compositions in the examples and comparative examples below contained the compounds in the percentages specified in the tables, and the amounts are given in “% by mass.”

In the description of the compounds, furthermore, the abbreviations below are used. It should be noted that a compound that can exist in both the cis and trans forms represents its trans form unless specified otherwise.

### <Ring Structures>

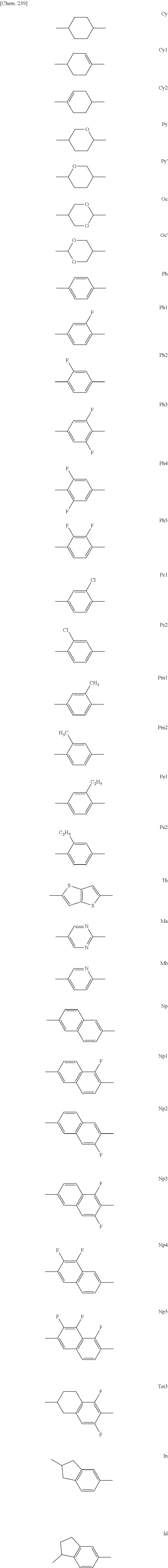

### <Terminal Structures>

TABLE 1

| Abbreviation | Chemical structure |
|---|---|
| -n | —CₙH₂ₙ₊₁ |
| n- | CₙH₂ₙ₊₁— |
| —On | —O—CₙH₂ₙ₊₁ |
| nO— | CₙH₂ₙ₊₁—O— |
| —Sn | —S—CₙH₂ₙ₊₁ |
| nS— | CₙH₂ₙ₊₁—S— |
| —V | —CH═CH₂ |
| V— | CH₂═CH— |
| —V1 | —CH═CH—CH₃ |
| 1V— | CH₃—CH═CH— |
| —2V | —CH₂—CH₂—CH═CH₂ |
| V2— | CH₂═CH—CH₂—CH₂— |
| —2V1 | —CH₂—CH₂—CH═CH—CH₃ |
| 1V2— | CH₃—CH═CH—CH₂—CH₂— |
| —OCF3 | —O—CF₃ |
| CF3O— | CF₃—O— |
| —H | —H |
| H— | H— |
| —CN | —CN |
| CN— | CN— |
| —NCS | —NCS |
| NCS— | NCS— |
| -(1)4 | —CH₂CH₂CH(CH₃)CH₃ |
| 4(1)- | CH₃CH(CH₃)CH₂CH₂— |

(Note that n in the table is a natural number.)

### <Linking Structures>

TABLE 2

| Abbreviation | Chemical structure |
|---|---|
| -n- | —CₙH₂ₙ— |
| -nO— | —CₙH₂ₙ—O— |
| —On- | —O—CₙH₂ₙ— |
| —COO— | —C(═O)—O— |
| —OCO— | —O—C(═O)— |
| —V— | —CH═CH— |
| -nV— | —CₙH₂ₙ—CH═CH— |
| —Vn- | —CH═CH—CₙH₂ₙ— |
| -T- | —C≡C— |
| —CF2O— | —CF₂—O— |
| —OCF2— | —O—CF₂— |
| -Az- | —N═N— |

(Note that n in the table is a natural number.)

### (Hindered Phenolic Antioxidants)

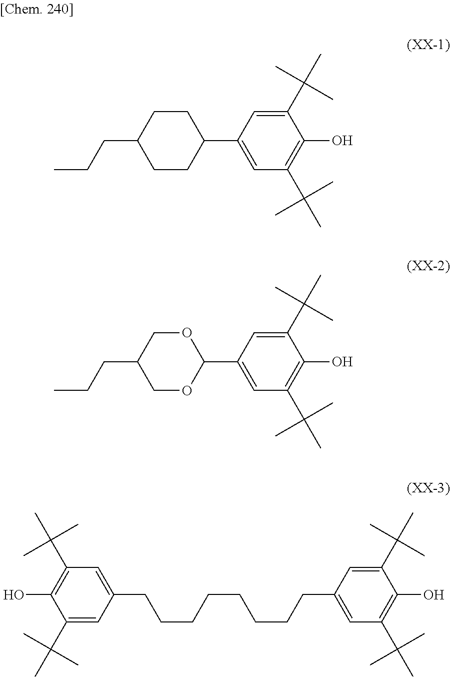 [SMILES: CCCC1CCC(c2cc(C(C)(C)C)c(O)c(C(C)(C)C)c2)CC1 ; CCCC1COC(c2cc(C(C)(C)C)c(O)c(C(C)(C)C)c2)OC1 ; CC(C)(C)c1cc(CCCCCCCCc2cc(C(C)(C)C)c(O)c(C(C)(C)C)c2)cc(C(C)(C)C)c1O]

### (Hindered Amine Photostabilizers)

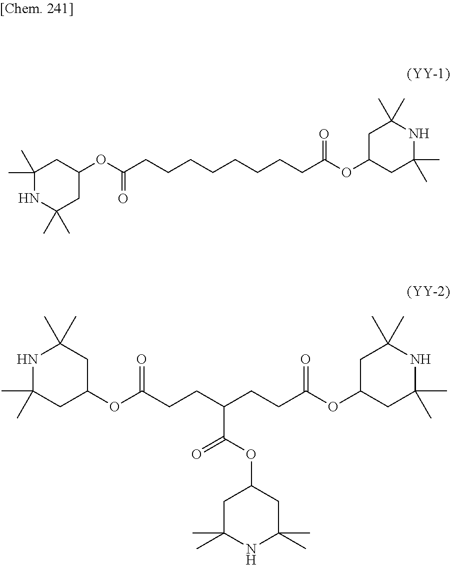

### Preparation of Liquid Crystal Compositions

LC-A and —B and LC-01 to -06 specified in Table 3 were prepared.

TABLE 3

| | LC-A | LC-B | LC-01 | LC-02 | LC-03 | LC-04 | LC-05 | LC-06 |
|---|---|---|---|---|---|---|---|---|
| 3-In-Ph-T-Ph3-NCS | | | 10 | 10 | 15 | | 6 | 5 |
| 4-In-Ph-T-Ph3-NCS | | | | 10 | | | | |
| 3-In-T-Ph-Ph3-NCS | | | | | | 10 | | |
| 3-Id-Ph-T-Ph3-NCS | | | | | | | 2 | |
| 4-Id-Ph-T-Ph3-NCS | | | | | | | 2 | |
| 4-In-T-Ph1-T-Ph3-NCS | | | | | | | | 5 |
| 4-T-Ph-T-Ph-Ph3-NCS | | | 3 | | | 5 | 3 | 5 |
| 5-T-Ph-T-Ph-Ph3-NCS | | | 3 | | 3 | | 3 | |
| 4-T-Ph-T-Ph1-Ph3-NCS | | | 4 | | 4 | 5 | 4 | 5 |
| 5-T-Ph-T-Ph1-Ph3-NCS | | | 7 | | 7 | 5 | 7 | 5 |
| 4-T-Pm2-Ph-T-Ph3-NCS | | | 4 | | | 10 | 4 | 10 |
| 4-T-Ph2-T-Ph-Ph3-NCS | | | 3 | | 3 | 5 | 3 | 5 |
| 4-T-Ph1-Ph-T-Ph3-NCS | | | 4 | | 3 | | 4 | |
| 5-T-Ph1-Ph-T-Ph3-NCS | | | 7 | | 7 | | 7 | |
| 4(1)-T-Ph-Ph-T-Ph3-NCS | | | | | 3 | | | |
| 5-Ph3-T-Ph-Ph3-NCS | | | 9 | | 10 | 10 | 9 | 10 |
| 3-Cy-T-Ph-Ph3-NCS | | | | | 5 | 3 | | 10 |
| 4-Cy-T-Ph-Ph3-NCS | | | | | 5 | 3 | | 6 |
| 3-Cy-T-Ph-T-Ph3-NCS | | | 6 | | | 6 | 6 | 6 |
| 4-Cy-T-Ph-T-Ph3-NCS | | | 5 | | | 6 | 5 | 6 |
| 5-Cy-Ph-NCS | 6 | | | 3 | | | | |
| 4-Ph-T-Pc1-NCS | 11 | | | 6 | | | | |
| 4O-Ph2-T-Ph-NCS | 5 | | | 5 | | | | |
| 5O-Ph2-T-Ph-NCS | 5 | | | 5 | | | | |
| 5-Ph-T-Ph1-NCS | 5 | | | 5 | | | | |
| 3-Ph-T-Ph3-NCS | | 13 | 17 | | 17 | 17 | 17 | 12 |
| 5-Ph-T-Ph3-NCS | | 11 | 18 | | 18 | 15 | 18 | 10 |
| 2-Cy-Ph-Ph3-NCS | 12 | | | 6 | | | | |
| 4-Cy-Ph-Ph3-NCS | 12 | | | 6 | | | | |
| 4-Cy-Ph-T-Ph1-NCS | 16 | | | 16 | | | | |
| 5-Cy-Ph-T-Ph1-NCS | 13 | | | 13 | | | | |
| 4-Cy-Ph-T-Ph3-NCS | | 14 | | | | | | |
| 5-Cy-Ph-T-Ph3-NCS | | 20 | | | | | | |
| CF3O-Ph-Ph-Ph3-NCS | | 24 | | | | | | |
| 4-Ph-Ph-T-Ph3-NCS | | 6 | | | | | | |
| 5-Ph-Ph-T-Ph3-NCS | | 12 | | | | | | |
| 5-Ph-Ph5-T-Ph1-NCS | 15 | | | 15 | | | | |
| Total [% by mass] | 100 | 100 | 100 | 100 | 100 | 100 | 100 | 100 |

### Examples 1 to 36 and Comparative Examples 1 and 2

The liquid crystal compositions specified in Tables 4 to 9 were prepared using LC-A and —B and LC-01 to -06, hindered phenolic antioxidants (XX-1) to (XX-3), and hindered amine photostabilizers (YY-1) and (YY-2), their characteristic parameters were measured, and a <Storability Test> was performed. The results are presented in Tables 4 to 9. It should be noted that in Comparative Example 2, the measurement of radiofrequency characteristics (Δεᵣ and tan δᵢₛₒ was not performed because the composition crystallized at room temperature.

### <Storability Test>

A 0.5-g portion of the liquid crystal composition was weighed into a 1-mL sample vial (manufactured by Maruemu Corporation), and defoaming by degassing at 150 to 250 Pa for 10 minutes was conducted. Then the vial was purged using dry nitrogen and sealed with the accompanying cap. This vial was stored for 2 weeks inside a temperature-controlled chamber (manufactured by ESPEC Corporation; SH-241) at −20° C., and the occurrence of the crystallization of the liquid crystal composition was visually checked every week.

TABLE 4

| | Comparative | Comparative | | | | | | |
|---|---|---|---|---|---|---|---|---|
| | Example 1 | Example 2 | Example 1 | Example 2 | Example 3 | Example 4 | Example 5 | Example 6 |
| Liquid | LC-A | LC-B | LC -01 | LC-02 | LC-03 | LC-04 | LC-05 | LC-06 |
| crystal | | | | | | | | |
| composition | | | | | | | | |
| Tₙᵢ[° C.] | 150 | 156 | 129 | 165 | 129 | 135 | 125 | 151 |
| Δn | 0.368 | 0.413 | 0.451 | 0.402 | 0.449 | 0.448 | 0.450 | 0.453 |
| Vₜₕ[V] | 2.05 | 2.00 | 1.71 | 2.00 | 1.72 | 1.70 | 1.70 | 1.69 |
| Δεᵣ | 1.091 | — | 1.214 | 1.099 | 1.225 | 1.220 | 1.213 | 1.227 |
| tanδᵢₛₒ | 0.019 | — | 0.013 | 0.018 | 0.012 | 0.010 | 0.014 | 0.013 |
| Storability | 2 weeks | Crystallized | 2 weeks | 2 weeks | 2 weeks | 2 weeks | 2 weeks | 2 weeks |
| (−20° C.) | without | at room | without | without | without | without | without | without |
| | crystallization | temperature | crystallization | crystallization | crystallization | crystallization | crystallization | crystallization |

TABLE 5

| | | Example 7 | Example 8 | Example 9 | Example 10 | Example 11 | Example 12 |
|---|---|---|---|---|---|---|---|
| Liquid | LC-01 | 99.70 | 99.80 | 99.80 | 99.80 | 99.75 | 99.75 |
| crystal | | | | | | | |
| composition | | | | | | | |
| [% by mass] | | | | | | | |
| Additives | XX-1 | | | 0.20 | | | |
| [% by mass] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| Total [% by mass] | 100.00 | 100.00 | 100.00 | 100.00 | 100.00 | 100.00 |
|---|---|---|---|---|---|---|
| Tₙᵢ[° C.] | 128 | 128 | 128 | 128 | 128 | 128 |
| Δn | 0.450 | 0.450 | 0.450 | 0.450 | 0.450 | 0.450 |
| Vₜₕ[V] | 1.71 | 1.71 | 1.71 | 1.71 | 1.71 | 1.71 |
| Δεᵣ | 1.214 | 1.214 | 1.214 | 1.214 | 1.214 | 1.214 |
| tanδᵢₛₒ | 0.013 | 0.013 | 0.013 | 0.013 | 0.013 | 0.013 |
| Storability | 2 weeks | 2 weeks | 2 weeks | 2 weeks | 2 weeks | 2 weeks |
| (−20° C.) | without | without | without | without | without | without |
| | crystallization | crystallization | crystallization | crystallization | crystallization | crystallization |

TABLE 6

| | | Example 13 | Example 14 | Example 15 | Example 16 | Example 17 | Example 18 |
|---|---|---|---|---|---|---|---|
| Liquid | LC-02 | 99.70 | 99.80 | 99.80 | 99.80 | 99.75 | 99.75 |
| crystal | | | | | | | |
| composition | | | | | | | |
| [% by mass] | | | | | | | |
| Additives | XX-1 | | | 0.20 | | | |
| [% by mass] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| Total [% by mass] | 100.00 | 100.00 | 100.00 | 100.00 | 100.00 | 100.00 |
|---|---|---|---|---|---|---|
| Tₙᵢ[° C.] | 164 | 164 | 164 | 164 | 164 | 164 |
| Δn | 0.401 | 0.401 | 0.401 | 0.401 | 0.401 | 0.401 |
| Vₜₕ[V] | 2.00 | 2.00 | 2.00 | 2.00 | 2.00 | 2.00 |
| Δεᵣ | 1.099 | 1.099 | 1.099 | 1.099 | 1.099 | 1.099 |
| tanδᵢₛₒ | 0.018 | 0.018 | 0.018 | 0.018 | 0.018 | 0.018 |
| Storability | 2 weeks | 2 weeks | 2 weeks | 2 weeks | 2 weeks | 2 weeks |
| (−20° C.) | without | without | without | without | without | without |
| | crystallization | crystallization | crystallization | crystallization | crystallization | crystallization |

TABLE 7

| | | Example 19 | Example 20 | Example 21 | Example 22 | Example 23 | Example 24 |
|---|---|---|---|---|---|---|---|
| Liquid | LC-03 | 99.70 | 99.80 | 99.80 | 99.80 | 99.75 | 99.75 |
| crystal | | | | | | | |
| composition | | | | | | | |
| [% by mass] | | | | | | | |
| Additives | XX-1 | | | 0.20 | | | |
| [% by mass] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [% by mass] | 100.00 | 100.00 | 100.00 | 100.00 | 100.00 | 100.00 |
| Tₙᵢ[° C.] | 128 | 128 | 128 | 128 | 128 | 128 |
| Δn | 0.448 | 0.448 | 0.448 | 0.448 | 0.448 | 0.448 |
| Vₜₕ[V] | 1.72 | 1.72 | 1.72 | 1.72 | 1.72 | 1.72 |
| Δεᵣ | 1.225 | 1.225 | 1.225 | 1.225 | 1.225 | 1.225 |
| tanδᵢₛₒ | 0.012 | 0.012 | 0.012 | 0.012 | 0.012 | 0.012 |
| Storability | 2 weeks | 2 weeks | 2 weeks | 2 weeks | 2 weeks | 2 weeks |
| (−20° C.) | without | without | without | without | without | without |
| | crystallization | crystallization | crystallization | crystallization | crystallization | crystallization |

TABLE 8

| | | Example 25 | Example 26 | Example 27 | Example 28 | Example 29 | Example 30 |
|---|---|---|---|---|---|---|---|
| Liquid | LC-04 | 99.70 | 99.80 | 99.80 | 99.80 | 99.75 | 99.75 |
| crystal | | | | | | | |
| composition | | | | | | | |
| [% by mass] | | | | | | | |
| Additives | XX-1 | | | 0.20 | | | |
| [% by mass] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| Total [% by mass] | 100.00 | 100.00 | 100.00 | 100.00 | 100.00 | 100.00 |
|---|---|---|---|---|---|---|
| Tₙᵢ[° C.] | 134 | 134 | 134 | 134 | 134 | 134 |
| Δn | 0.447 | 0.447 | 0.447 | 0.447 | 0.447 | 0.447 |
| Vₜₕ[V] | 1.70 | 1.70 | 1.70 | 1.70 | 1.70 | 1.70 |
| Δεᵣ | 1.220 | 1.220 | 1.220 | 1.220 | 1.220 | 1.220 |
| tanδᵢₛₒ | 0.010 | 0.010 | 0.010 | 0.010 | 0.010 | 0.010 |
| Storability | 2 weeks | 2 weeks | 2 weeks | 2 weeks | 2 weeks | 2 weeks |
| (−20° C.) | without | without | without | without | without | without |
| | crystallization | crystallization | crystallization | crystallization | crystallization | crystallization |

TABLE 9

| | | Example 31 | Example 32 | Example 33 | Example 34 | Example 35 | Example 36 |
|---|---|---|---|---|---|---|---|
| Liquid | LC-06 | 99.70 | 99.80 | 99.80 | 99.80 | 99.75 | 99.75 |
| crystal | | | | | | | |
| composition | | | | | | | |
| [% by mass] | | | | | | | |
| Additives | XX-1 | | | 0.20 | | | |
| [% by mass] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| Total [% by mass] | 100.00 | 100.00 | 100.00 | 100.00 | 100.00 | 100.00 |
|---|---|---|---|---|---|---|
| Tₙᵢ[° C.] | 150 | 150 | 150 | 150 | 150 | 150 |
| Δn | 0.452 | 0.452 | 0.452 | 0.452 | 0.452 | 0.452 |
| Vₜₕ[V] | 1.69 | 1.69 | 1.69 | 1.69 | 1.69 | 1.69 |
| Δεᵣ | 1.227 | 1.227 | 1.227 | 1.227 | 1.227 | 1.227 |
| tanδᵢₛₒ | 0.013 | 0.013 | 0.013 | 0.013 | 0.013 | 0.013 |
| Storability | 2 weeks | 2 weeks | 2 weeks | 2 weeks | 2 weeks | 2 weeks |
| (−20° C.) | without | without | without | without | without | without |
| | crystallization | crystallization | crystallization | crystallization | crystallization | crystallization |

From Examples 1 through 6, liquid crystal compositions made using at least one compound represented by general formula (i) and at least one compound represented by general formula (ii) were liquid crystal compositions having a high Tₙᵢ, a large Δn, a low Vₜₕ, a large Δεᵣ, and a small tan δᵢₛₒ and good storability at low temperatures.

In particular, Examples 1, 5, and 6 resulted in especially high Δn and Δεᵣ.

From Comparative Examples 1 and 2, by contrast, liquid crystal compositions made without a compound represented by general formula (i) exhibited a Δn lower than 0.38 or were found to have crystallized at room temperature.

From Examples 7 through 36, furthermore, even when a hindered phenolic antioxidant and/or a hindered amine photostabilizer was used in combination with them, the liquid crystal compositions were found to have a high Tₙᵢ, a large Δn, a low Vₜₕ, a large Δεᵣ, and a small tan δᵢₛₒ and good storability at low temperatures.

Moreover, from Examples 37 through 43, similar advantages were successfully observed even with compositions with modified chemical makeups. The results are presented in Tables 10 to 12.

TABLE 10

| | LC-07 |
|---|---|
| 3-In-Ph-T-Ph3-NCS | 10 |
| 5-T-Ph-T-Ph-Ph3-NCS | 3 |
| 4-T-Ph-T-Ph1-Ph3-NCS | 5 |
| 5-T-Ph-T-Ph1-Ph3-NCS | 7 |
| 4-T-Pm2-Ph-T-Ph3-NCS | 3 |
| 4-T-Ph2-T-Ph-Ph3-NCS | 5 |
| 4-T-Ph-Ph2-T-Ph3-NCS | 5 |
| 4-T-Ph1-Ph-T-Ph3-NCS | 5 |
| 5-T-Ph1-Ph-T-Ph3-NCS | 7 |
| 5-Ph3-T-Ph-Ph3-NCS | 5 |
| 3-Cy-T-Ph-T-Ph3-NCS | 5 |
| 4-Cy-T-Ph-T-Ph3-NCS | 5 |
| 2-Ph-T-Ph3-NCS | 8 |
| 3-Ph-T-Ph3-NCS | 17 |
| 5-Ph-T-Ph3-NCS | 10 |
| Total [% by mass] | 100 |

TABLE 11

| | Example 37 |
|---|---|
| Liquid crystal composition | LC-07 |
| Tₙᵢ [° C.] | 126 |
| Δn | 0.451 |
| Vₜₕ [V] | 1.70 |
| Δεᵣ | 1.226 |
| tanδᵢₛₒ | 0.013 |
| Storability (−20° C.) | 2 weeks without crystallization |

TABLE 12

| | | Example 38 | Example 39 | Example 40 | Example 41 | Example 42 | Example 43 |
|---|---|---|---|---|---|---|---|
| Liquid | LC-07 | 99.70 | 99.80 | 99.80 | 99.80 | 99.75 | 99.75 |
| crystal | | | | | | | |
| composition | | | | | | | |
| [% by mass] | | | | | | | |
| Additives | XX-1 | | | 0.20 | | | |
| [% by mass] | XX-2 | | | | 0.20 | 0.20 | |
| | XX-3 | 0.30 | 0.15 | | | | 0.20 |
| | YY-1 | | | | | 0.05 | 0.05 |
| | YY-2 | | 0.05 | | | | |

| | | | | | | |
|---|---|---|---|---|---|---|
| Total [% by mass] | 100.00 | 100.00 | 100.00 | 100.00 | 100.00 | 100.00 |
| Tₙᵢ[° C.] | 125 | 125 | 125 | 125 | 125 | 125 |
| Δn | 0.450 | 0.450 | 0.450 | 0.450 | 0.450 | 0.450 |
| Vₜₕ[V] | 1.70 | 1.70 | 1.70 | 1.70 | 1.70 | 1.70 |
| Δεᵣ | 1.226 | 1.226 | 1.226 | 1.226 | 1.226 | 1.226 |
| tanδᵢₛₒ | 0.013 | 0.013 | 0.013 | 0.013 | 0.013 | 0.013 |
| Storability | 2 weeks | 2 weeks | 2 weeks | 2 weeks | 2 weeks | 2 weeks |
| (−20° C.) | without | without | without | without | without | without |
| | crystallization | crystallization | crystallization | crystallization | crystallization | crystallization |

In the following, the synthesis of the compounds represented by general formula (i) will be described.

### (Synthesis Example 1) Production of the Compound Represented by Formula (I-1)

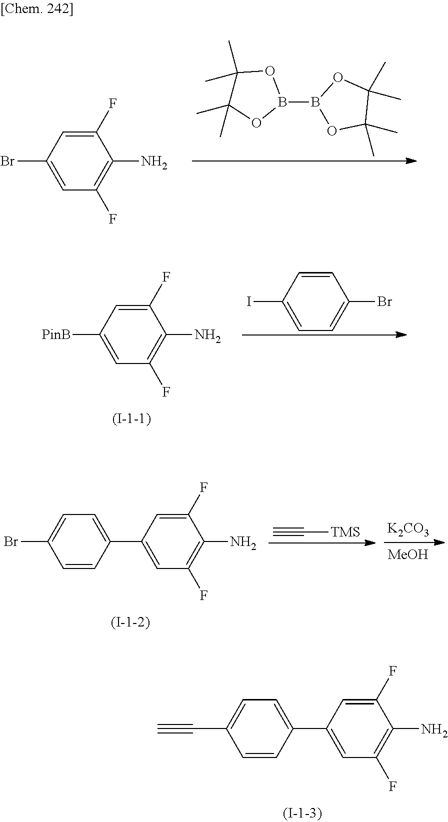

First, in a nitrogen atmosphere, 50 g of 4-bromo-2,6-difluoroaniline, 65 g of bis(pinacolato)diboron, 70 g of potassium acetate, and 500 mL of N,N-dimethylformamide were added to a reaction vessel at room temperature. Then, at room temperature, stirring was performed with added 4 g of bis(diphenylphosphino)ferrocenepalladium(II) dichloride. Subsequently, the mixture was heated to 90° C. and allowed to react for 4 hours. After the end of the reaction, 300 mL of a saturated aqueous solution of ammonium chloride was poured into the reaction solution, and extraction with 700 mL of ethyl acetate was performed. After the organic layer was washed with a saturated saline solution, purification was performed by column chromatography (silica gel, ethyl acetate/hexane=1/5 to 1/3), through which 49 g of the compound represented by formula (I-1-1) was obtained.

Then, in a nitrogen atmosphere, 20.0 g of the compound represented by formula (I-1-1), 21 g of 1-bromo-4-iodobenzene, and 200 mL of tetrahydrofuran were added to a reaction vessel at room temperature. Then 0.5 g of the bis(triphenylphosphine)palladium(II) dichloride dichloromethane complex was added, and the resulting mixture was heated to 60° C. Then, with stirring at 60° C., 80 mL of a solution in which 16 g of sodium carbonate had been dissolved in 80 mL of water was slowly added dropwise, and the resulting mixture was stirred for 2 hours at 65° C. After the end of the reaction, 100 mL of a saturated solution of ammonium chloride was poured into the reaction solution, and extraction with 200 mL of ethyl acetate was performed. After the organic layer was washed with a saturated saline solution, purification was performed by column chromatography (silica gel, ethyl acetate/hexane=1/5 to 1/3), through which 22 g of the compound represented by formula (I-1-2) was obtained.

Then, in a nitrogen atmosphere, 22 g of the compound represented by formula (I-1-2), 0.5 g of copper(I) iodide, 1.8 g of tetrakis(triphenylphosphine)palladium(0), 50 mL of triethylamine, and 50 mL of N,N-dimethylformamide were added to a reaction vessel at room temperature. Then, with heating at 75° C., 20 mL of a solution in which 10 g of trimethylsilylacetylene had been dissolved in 20 mL of N,N-dimethylformamide was added dropwise, and the resulting mixture was stirred for 2 hours at 75° C. After the end of the reaction, a saturated aqueous solution of ammonium chloride was poured into the reaction solution, and extraction with hexane was performed. After the organic layer was washed with a saturated saline solution, purification was performed by column chromatography (silica gel, ethyl acetate/hexane=1/5 to 1/3), and the solvent was distilled away to give a concentrate. Then the concentrate, 100 mL of methanol, and 1 g of potassium carbonate were added to a reaction vessel at room temperature, and stirring was performed at room temperature. After the end of the reaction, purification was performed by column chromatography (silica gel, ethyl acetate/hexane=1/5 to 1/3), giving 15 g of the compound represented by formula (I-1-3).

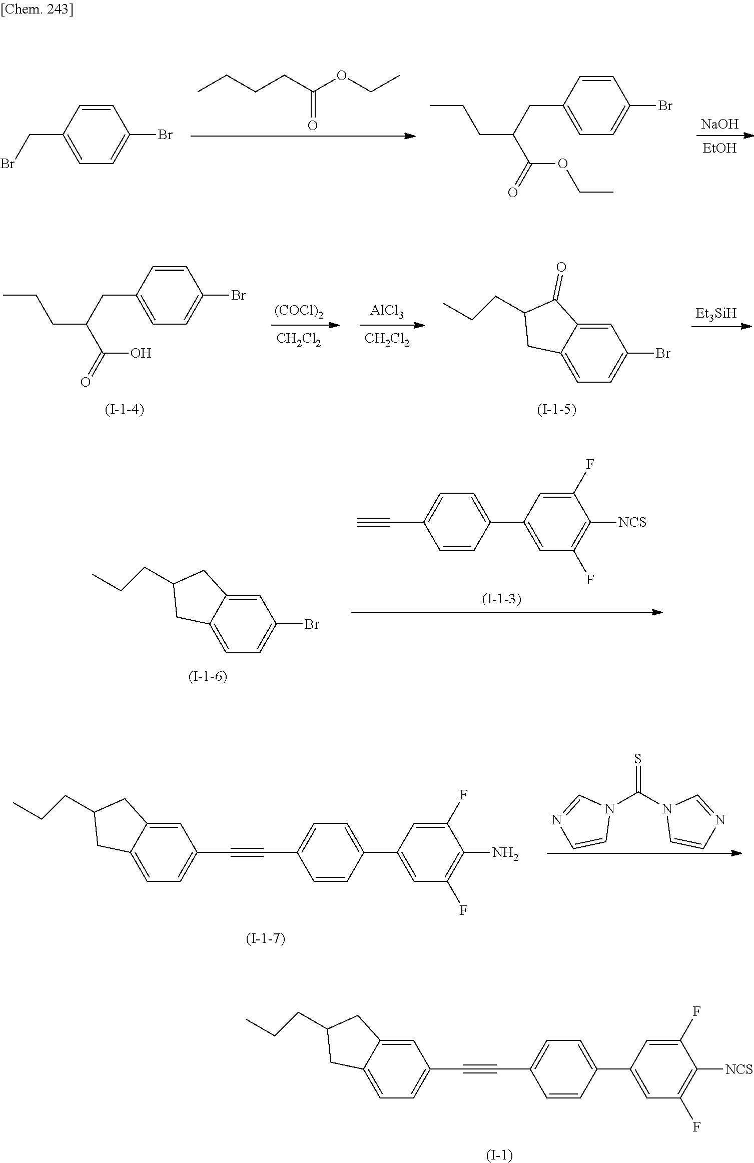

Then in a nitrogen atmosphere, 11 g of diisopropylamine and 150 mL of tetrahydrofuran were added to a reaction vessel at room temperature. Then the resulting mixture was cooled to −78° C., 46 mL of a n-hexane solution (2.4 mol/L) of n-butyllithium was added dropwise, and then the mixture was stirred for 30 minutes at −10° C. Then the mixture was cooled to −78° C., and 40 mL of a solution in which 14 g of ethyl valerate had been dissolved in 40 mL of tetrahydrofuran was added dropwise. After the end of addition, the resulting mixture was stirred for 60 minutes at −78° C., followed by slow dropwise addition of 100 mL of a solution in which 25 g of 4-bromobenzyl bromide had been dissolved in 100 mL of tetrahydrofuran. After the end of addition, the resulting mixture was stirred for 1 hour at −78° C., then warmed to room temperature, and stirred for another 3 hours. After the end of the reaction, 10% by mass hydrochloric acid was added, and extraction with 200 mL of toluene was performed. Subsequently, the organic layer was washed with a saturated saline solution, and the solvent was distilled away.

Then, in a nitrogen atmosphere, the resulting concentrate, 100 mL of ethanol, and 30 mL of a 10% by mass aqueous solution of sodium hydroxide were added to a reaction vessel, and the resulting mixture was allowed to react for 3 hours with thermal recirculation. After the end of the reaction, the resulting mixture was neutralized by adding 10% by mass hydrochloric acid, and extraction with 300 mL of ethyl acetate was performed. After the organic layer was washed with a saturated saline solution, recrystallization was performed using hexane, giving 16 g of the compound represented by formula (I-1-4).

Then, in a nitrogen atmosphere, 16 g of the compound represented by formula (I-1-4) and 100 mL of dichloromethane were added to a reaction vessel at room temperature, and the resulting mixture was cooled to 10° C. or below. Then 20 mL of a solution in which 7.4 g of oxalic acid dichloride had been dissolved in 20 mL of dichloromethane was slowly added dropwise, and, after the end of addition, the resulting mixture was allowed to react for 1 hour at room temperature. After the end of the reaction, the solvent and an excess of oxalic acid dichloride were distilled away, giving a concentrate.

Then, in a nitrogen atmosphere, 10 g of anhydrous aluminum trichloride and 100 mL of dichloromethane were added to a reaction vessel at room temperature, and the resulting mixture was cooled to 0° C. Then 20 mL of a solution in which the concentrate had been dissolved in 20 mL of dichloromethane was slowly added dropwise. After the end of addition, the resulting mixture was allowed to react for 2 hours at room temperature. After 10% by mass hydrochloric acid was added, extraction was performed with added 300 mL of dichloromethane. After the organic layer was washed with a saturated saline solution, purification was performed by column chromatography (silica gel, toluene/hexane=1/1), giving 12 g of the compound represented by formula (I-1-5).

Thereafter, in a nitrogen atmosphere, 12 g of the compound represented by formula (I-1-5), 30 mL of triethylsilane, and 50 mL of trifluoroacetic acid were added to a reaction vessel at room temperature, and the resulting mixture was allowed to react for 24 hours. After the end of the reaction, the reaction solution was put into an aqueous solution of sodium carbonate, the pH value was adjusted to 7 to 8, and extraction with 200 mL of toluene was performed. After the organic layer was washed with a saturated saline solution, purification was performed by column chromatography (silica gel, toluene/hexane=1/1), through which 10 g of the compound represented by formula (I-1-6) was obtained.

Then in a nitrogen atmosphere, 10 g of the compound represented by formula (I-1-6), 0.3 g of copper(I) iodide, 1.0 g of tetrakis(triphenylphosphine)palladium(0), 50 mL of triethylamine, and 50 mL of N,N-dimethylformamide were added to a reaction vessel at room temperature. Then, with heating at 85° C., a solution in which 11 g of the compound represented by formula (I-1-3) had been dissolved in 20 mL of N,N-dimethylformamide was added dropwise, and the resulting mixture was stirred for 2 hours at 85° C. After the end of the reaction, a saturated aqueous solution of ammonium chloride was poured into the reaction solution, and extraction with ethyl acetate was performed. After the organic layer was washed with a saturated saline solution, purification was performed by column chromatography (silica gel, ethyl acetate/toluene=0/10 to 1/5) to give 12 g of the compound represented by formula (I-1-7).

Then, in a nitrogen atmosphere, 12 g of the compound represented by formula (I-1-7), 80 mL of dichloromethane, and 6 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 11 g of the compound represented by formula (I-1) was obtained.

MS (EI): m/z=429

### (Synthesis Example 2) Production of the Compound Represented by Formula (I-2)

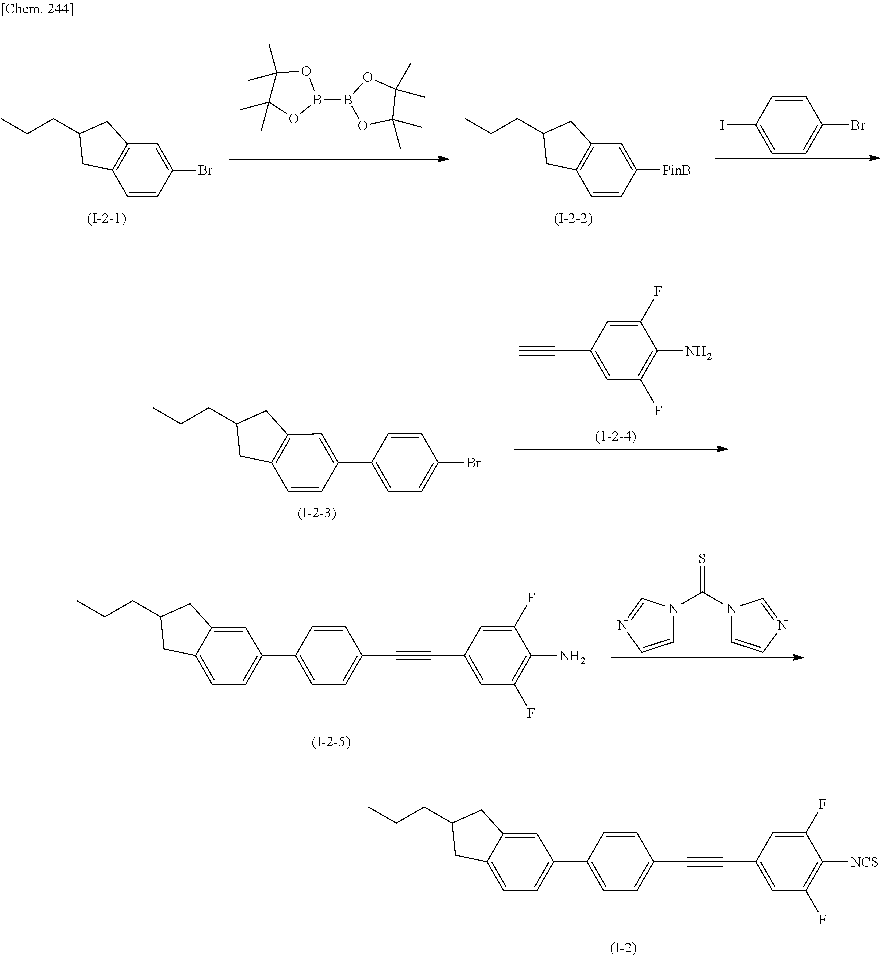

In a nitrogen atmosphere, 10 g of the compound represented by formula (I-2-1), 12 g of bis(pinacolato)diboron, 15 g of potassium acetate, and 100 mL of N,N-dimethylformamide were added to a reaction vessel at room temperature. Then, at room temperature, stirring was performed with added 1 g of bis(diphenylphosphino)ferrocenepalladium(II) dichloride. Then the mixture was heated to 90° C. and allowed to react for 4 hours. After the end of the reaction, 100 mL of a saturated aqueous solution of ammonium chloride was poured into the reaction solution, and extraction with 200 mL of ethyl acetate was performed. After the organic layer was washed with a saturated saline solution, purification was performed by column chromatography (silica gel, hexane/toluene=2/1), through which 11 g of the compound represented by formula (I-2-2) was obtained.

Then, in a nitrogen atmosphere, 11 g of the compound represented by formula (I-2-2), 11 g of 1-bromo-4-iodobenzene, and 120 mL of tetrahydrofuran were added to a reaction vessel at room temperature. Then 0.2 g of the bis(triphenylphosphine)palladium(II) dichloride dichloromethane complex was added, and the resulting vessel was heated to 60° C. Then, with stirring at 60° C., 40 mL of a solution in which 8 g of sodium carbonate had been dissolved in 40 mL of water was slowly added dropwise, and the resulting mixture was stirred for 2 hours at 65° C. After the end of the reaction, 100 mL of a saturated solution of ammonium chloride was poured into the reaction solution, and extraction with 200 mL of ethyl acetate was performed. After the organic layer was washed with a saturated saline solution, purification was performed by column chromatography (silica gel, hexane/toluene=1/3 to 1/2), through which 9 g of the compound represented by formula (I-2-3) was obtained.

In a nitrogen atmosphere, 9 g of the compound represented by formula (I-2-3), 0.25 g of copper(I) iodide, 0.8 g of tetrakis(triphenylphosphine)palladium(0), 25 mL of triethylamine, and 50 mL of N,N-dimethylformamide were added to a reaction vessel at room temperature. Then the resulting mixture was heated to 85° C., 10 mL of a solution in which 5 g of the compound represented by formula (I-2-4) had been dissolved in 10 mL of N,N-dimethylformamide was added dropwise, and the mixture was stirred for 2 hours at 85° C. After the end of the reaction, a saturated aqueous solution of ammonium chloride was poured into the reaction solution, and extraction with ethyl acetate was performed. After the organic layer was washed with a saturated saline solution, purification was performed by column chromatography (silica gel, ethyl acetate/toluene=1/5 to 1/3), giving 10.5 g of the compound represented by formula (I-2-5).

Then, in a nitrogen atmosphere, 10.5 g of the compound represented by formula (I-2-5), 80 mL of dichloromethane, and 7.7 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 9.5 g of the compound represented by formula (I-2) was obtained.

MS (EI): m/z=429

### (Synthesis Example 3) Production of the Compound Represented by Formula (I-3)

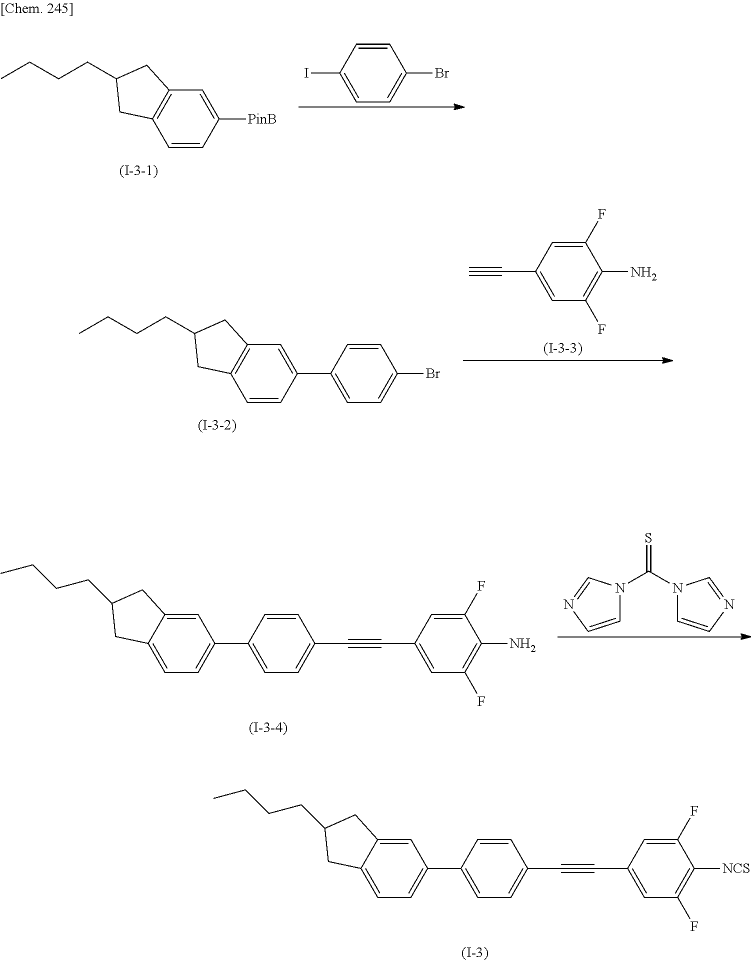

In a nitrogen atmosphere, 10 g of the compound represented by formula (I-3-1), synthesized by the same method as in Synthesis Example 2, 10 g of 1-bromo-4-iodobenzene, and 120 mL of tetrahydrofuran were added to a reaction vessel at room temperature. Then 0.2 g of the bis(triphenylphosphine)palladium(II) dichloride dichloromethane complex was added, and the resulting mixture was heated to 60° C. Then, with stirring at 60° C., 40 mL of a solution in which 7.5 g of sodium carbonate had been dissolved in 40 mL of water was slowly added dropwise, and the resulting mixture was stirred for 2 hours at 65° C. After the end of the reaction, 100 mL of a saturated solution of ammonium chloride was poured into the reaction solution, and extraction with 200 mL of ethyl acetate was performed. After the organic layer was washed with a saturated saline solution, purification was performed by column chromatography (silica gel, hexane/toluene=1/3 to 1/2), through which 8 g of the compound represented by formula (I-3-2) was obtained.

In a nitrogen atmosphere, 8 g of the compound represented by formula (I-3-2), 0.23 g of copper(I) iodide, 0.7 g of tetrakis(triphenylphosphine)palladium(0), 25 mL of triethylamine, and 50 mL of N,N-dimethylformamide were added to a reaction vessel at room temperature. Then the resulting mixture was heated to 85° C., a solution in which 4.5 g of the compound represented by formula (1-3-3) had been dissolved in 10 mL of N,N-dimethylformamide was added dropwise, and the mixture was stirred for 2 hours at 85° C. After the end of the reaction, a saturated aqueous solution of ammonium chloride was poured into the reaction solution, and extraction with ethyl acetate was performed. After the organic layer was washed with a saturated saline solution, purification was performed by column chromatography (silica gel, ethyl acetate/toluene=1/5 to 1/3), giving 8.5 g of the compound represented by formula (I-3-4).

Then, in a nitrogen atmosphere, 8.5 g of the compound represented by formula (I-3-4), 80 mL of dichloromethane, and 4.5 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 8 g of the compound represented by formula (I-3) was obtained.

MS (EI): m/z=443

### (Synthesis Example 4) Production of the Compound Represented by Formula (I-4)

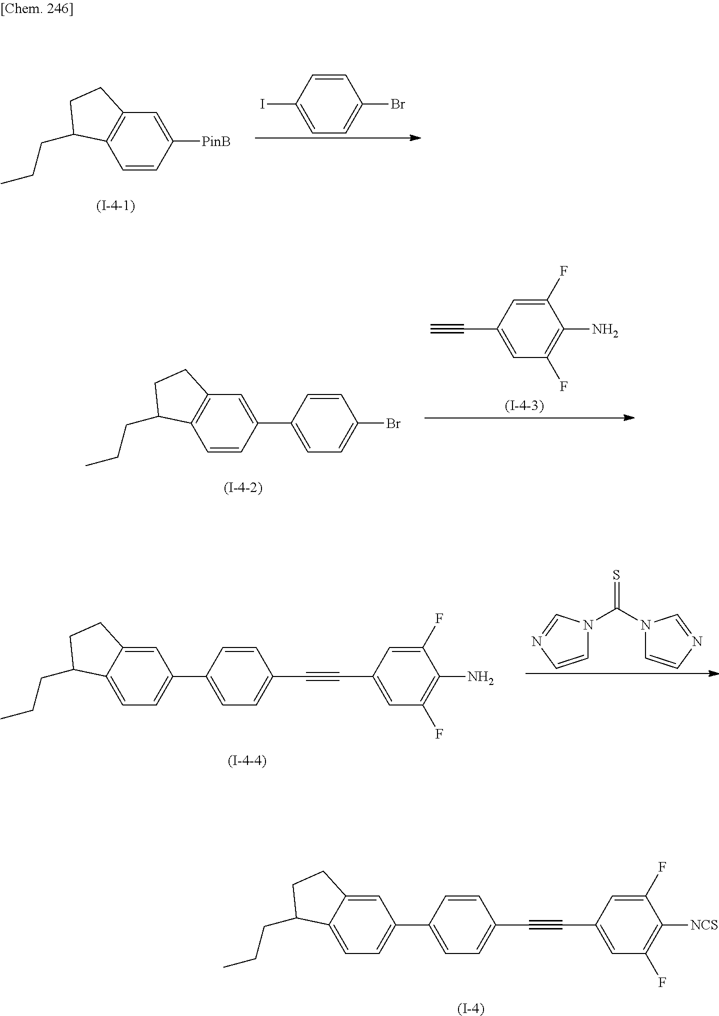

In a nitrogen atmosphere, 11 g of the compound represented by formula (I-4-1), 11 g of 1-bromo-4-iodobenzene, and 120 mL of tetrahydrofuran were added to a reaction vessel at room temperature. Then 0.2 g of the bis(triphenylphosphine)palladium(II) dichloride dichloromethane complex was added, and the resulting mixture was heated to 60° C. Then, with stirring at 60° C., 40 mL of a solution in which 8 g of sodium carbonate had been dissolved in 40 mL of water was slowly added dropwise, and the resulting mixture was stirred for 2 hours at 65° C. After the end of the reaction, 100 mL of a saturated solution of ammonium chloride was poured into the reaction solution, and extraction with 200 mL of ethyl acetate was performed. After the organic layer was washed with a saturated saline solution, purification was performed by column chromatography (silica gel, hexane/toluene=1/3 to 1/2), through which 9 g of the compound represented by formula (I-4-2) was obtained.

In a nitrogen atmosphere, 9 g of the compound represented by formula (I-4-2), 0.25 g of copper(I) iodide, 0.8 g of tetrakis(triphenylphosphine)palladium(0), 25 mL of triethylamine, and 50 mL of N,N-dimethylformamide were added to a reaction vessel at room temperature. Then the resulting mixture was heated to 85° C., 10 mL of a solution in which 5 g of the compound represented by formula (1-4-3) had been dissolved in 10 mL of N,N-dimethylformamide was added dropwise, and the mixture was stirred for 2 hours at 85° C. After the end of the reaction, a saturated aqueous solution of ammonium chloride was poured into the reaction solution, and extraction with ethyl acetate was performed. After the organic layer was washed with a saturated saline solution, purification was performed by column chromatography (silica gel, ethyl acetate/toluene=1/5 to 1/3), giving 10 g of the compound represented by formula (I-4-4).

Then, in a nitrogen atmosphere, 10 g of the compound represented by formula (I-4-4), 80 mL of dichloromethane, and 7.6 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 8.5 g of the compound represented by formula (I-4) was obtained.

MS (EI): m/z=429

### (Synthesis Example 5) Production of the Compound Represented by Formula (I-5)

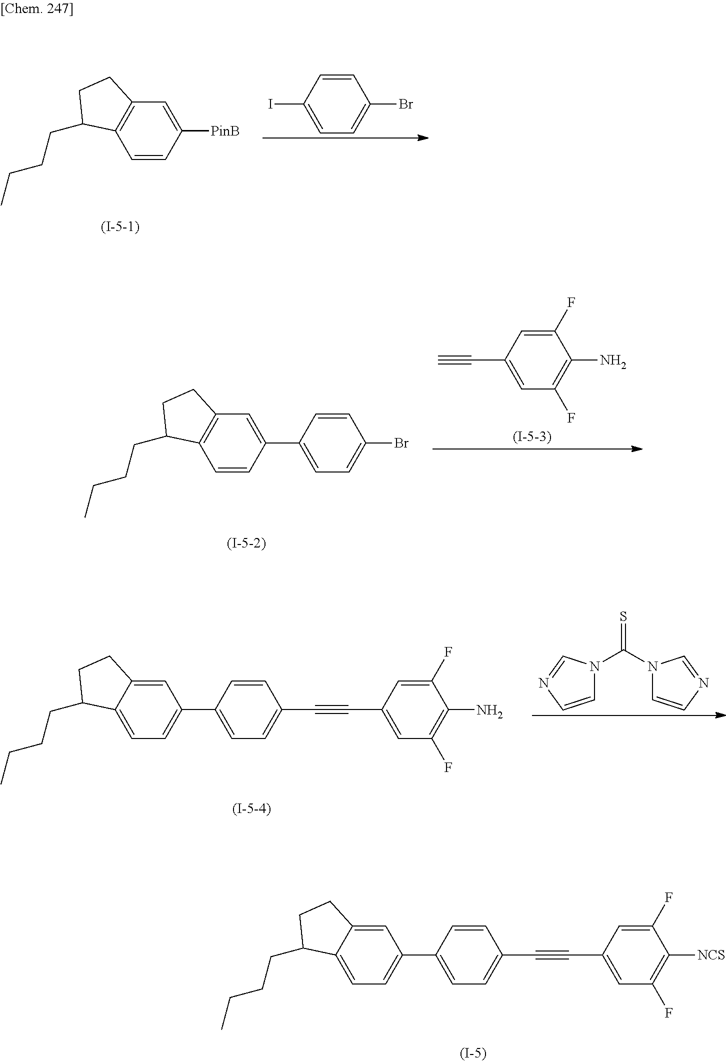

In a nitrogen atmosphere, 10 g of the compound represented by formula (I-5-1), synthesized by the same method as in Synthesis Example 2, 10 g of 1-bromo-4-iodobenzene, and 120 mL of tetrahydrofuran were added to a reaction vessel at room temperature. Then 0.2 g of the bis(triphenylphosphine)palladium(II) dichloride dichloromethane complex was added, and the resulting mixture was heated to 60° C. Then, with stirring at 60° C., 40 mL of a solution in which 7.5 g of sodium carbonate had been dissolved in 40 mL of water was slowly added dropwise, and the resulting mixture was stirred for 2 hours at 65° C. After the end of the reaction, 100 mL of a saturated solution of ammonium chloride was poured into the reaction solution, and extraction with 200 mL of ethyl acetate was performed. After the organic layer was washed with a saturated saline solution, purification was performed by column chromatography (silica gel, hexane/toluene=1/3 to 1/2), through which 8 g of the compound represented by formula (I-5-2) was obtained.

Then in a nitrogen atmosphere, 8 g of the compound represented by formula (I-5-2), 0.23 g of copper(I) iodide, 0.7 g of tetrakis(triphenylphosphine)palladium(0), 25 mL of triethylamine, and 50 mL of N,N-dimethylformamide were added to a reaction vessel at room temperature. Then the resulting mixture was heated to 85° C., 10 mL of a solution in which 4.5 g of the compound represented by formula (1-5-3) had been dissolved in 10 mL of N,N-dimethylformamide was added dropwise, and the mixture was stirred for 2 hours at 85° C. After the end of the reaction, a saturated aqueous solution of ammonium chloride was poured into the reaction solution, and extraction with ethyl acetate was performed. After the organic layer was washed with a saturated saline solution, purification was performed by column chromatography (silica gel, ethyl acetate/toluene=1/5 to 1/3), giving 8.2 g of the compound represented by formula (I-5-4).

Then, in a nitrogen atmosphere, 8.2 g of the compound represented by formula (I-5-4), 80 mL of dichloromethane, and 4.4 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 7.8 g of the compound represented by formula (I-5) was obtained.

MS (EI): m/z=443

### (Synthesis Example 6) Production of the Compound Represented by Formula (I-6)

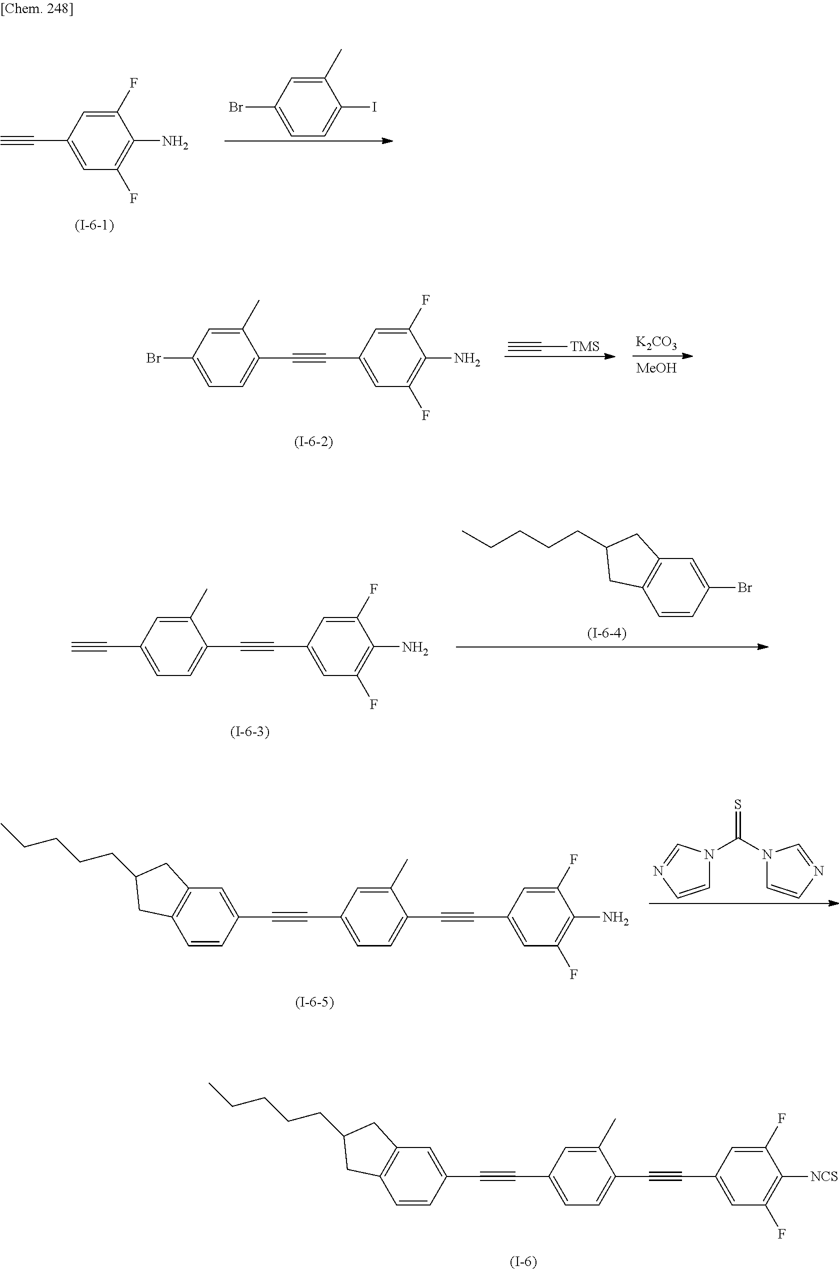

In a nitrogen atmosphere, 15 g of 1-bromo-4-iodo-3-methylbenzene, 0.3 g of copper(I) iodide, 1.8 g of tetrakis(triphenylphosphine)palladium(0), 50 mL of triethylamine, and 50 mL of N,N-dimethylformamide were added to a reaction vessel at room temperature. Then the resulting mixture was heated to 85° C., a solution in which 8.5 g of the compound represented by formula (1-6-1) had been dissolved in 20 mL of N,N-dimethylformamide was added dropwise, and the mixture was stirred for 2 hours at 85° C. After the end of the reaction, 100 mL of a saturated aqueous solution of ammonium chloride was poured into the reaction solution, and extraction with 200 mL of ethyl acetate was performed. After the organic layer was washed with a saturated saline solution, purification was performed by column chromatography (silica gel, ethyl acetate/toluene=1/5 to 1/3), through which 13.6 g of the compound represented by formula (I-6-2) was obtained.

Then in a nitrogen atmosphere, 13.6 g of the compound represented by formula (I-6-2), 0.5 g of copper(I) iodide, 1 g of tetrakis(triphenylphosphine)palladium(0), 50 mL of triethylamine, and 50 mL of N,N-dimethylformamide were added to a reaction vessel at room temperature. Then the resulting mixture was heated to 75° C., 10 mL of a solution in which 5.5 g of trimethylsilylacetylene had been dissolved in 10 mL of N,N-dimethylformamide was added dropwise, and the mixture was stirred for 2 hours at 75° C. After the end of the reaction, a saturated aqueous solution of ammonium chloride was poured into the reaction solution, and extraction with 200 mL of ethyl acetate was performed. After the organic layer was washed with a saturated saline solution, purification was performed by column chromatography (silica gel, ethyl acetate/hexane=1/5 to 1/3), and the product was concentrated by distilling the solvent away, giving a concentrate. Then the concentrate, 100 mL of methanol, and 2.5 g of potassium carbonate were added to a reaction vessel at room temperature, and stirring was performed at room temperature. After the end of the reaction, purification was performed by column chromatography (silica gel, ethyl acetate/hexane=1/5 to 1/3), giving 9.5 g of the compound represented by formula (I-6-3).

Then in a nitrogen atmosphere, 9 g of the compound represented by formula (1-6-4), 0.3 g of copper(I) iodide, 1.8 g of tetrakis(triphenylphosphine)palladium(0), 50 mL of triethylamine, and 50 mL of N,N-dimethylformamide were added to a reaction vessel at room temperature. Then the resulting mixture was heated to 85° C., 20 mL of a solution in which 9.5 g of the compound represented by formula (I-6-3) had been dissolved in 20 mL of N,N-dimethylformamide was added dropwise, and the mixture was stirred for 2 hours at 85° C. After the end of the reaction, 100 mL of a saturated aqueous solution of ammonium chloride was poured into the reaction solution, and extraction with 200 mL of ethyl acetate was performed. After the organic layer was washed with a saturated saline solution, purification was performed by column chromatography (silica gel, ethyl acetate/toluene=1/5 to 1/3), through which 13 g of the compound represented by formula (I-6-5) was obtained.

Then, in a nitrogen atmosphere, 13 g of the compound represented by formula (I-6-5), 90 mL of dichloromethane, and 5.5 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 12 g of the compound represented by formula (I-6) was obtained.

MS (EI): m/z=495

### (Synthesis Example 7) Production of the Compound Represented by Formula (I-7)

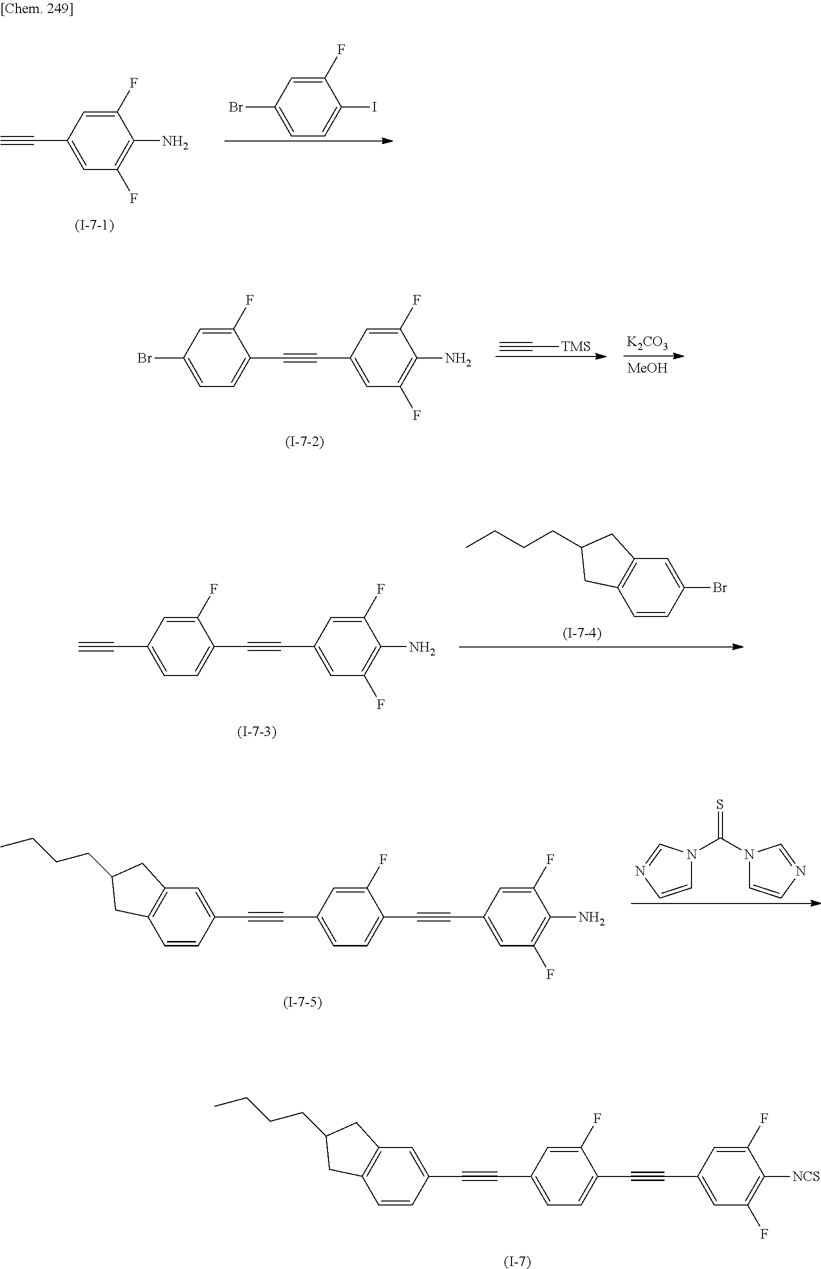

In a nitrogen atmosphere, 15 g of 1-bromo-3-fluoro-4-iodobenzene, 0.3 g of copper(I) iodide, 1.9 g of tetrakis(triphenylphosphine)palladium(0), 50 mL of triethylamine, and 50 mL of N,N-dimethylformamide were added to a reaction vessel at room temperature. Then the resulting mixture was heated to 85° C., 20 mL of a solution in which 8.3 g of the compound represented by formula (1-7-1) had been dissolved in 20 mL of N,N-dimethylformamide was added dropwise, and the mixture was stirred for 2 hours at 85° C. After the end of the reaction, 100 mL of a saturated aqueous solution of ammonium chloride was poured into the reaction solution, and extraction with 200 mL of ethyl acetate was performed. After the organic layer was washed with a saturated saline solution, purification was performed by column chromatography (silica gel, ethyl acetate/toluene=1/5 to 1/3), through which 13 g of the compound represented by formula (I-7-2) was obtained.

Then in a nitrogen atmosphere, 13 g of the compound represented by formula (I-7-2), 0.4 g of copper(I) iodide, 0.9 g of tetrakis(triphenylphosphine)palladium(0), 50 mL of triethylamine, and 50 mL of N,N-dimethylformamide were added to a reaction vessel at room temperature. Then the resulting mixture was heated to 75° C., 10 mL of a solution in which 5.2 g of trimethylsilylacetylene had been dissolved in 10 mL of N,N-dimethylformamide was added dropwise, and the mixture was stirred for 2 hours at 75° C. After the end of the reaction, a saturated aqueous solution of ammonium chloride was poured into the reaction solution, and extraction with 200 mL of ethyl acetate was performed. After the organic layer was washed with a saturated saline solution, purification was performed by column chromatography (silica gel, ethyl acetate/hexane=1/5 to 1/3), and the product was concentrated by distilling the solvent away, giving a concentrate. Then the concentrate, 100 mL of methanol, and 2.5 g of potassium carbonate were added to a reaction vessel at room temperature, and stirring was performed at room temperature. After the end of the reaction, purification was performed by column chromatography (silica gel, ethyl acetate/hexane=1/5 to 1/3), giving 9 g of the compound represented by formula (I-7-3).

Then in a nitrogen atmosphere, 8.4 g of the compound represented by formula (I-7-4), 0.2 g of copper(I) iodide, 1.5 g of tetrakis(triphenylphosphine)palladium(0), 50 mL of triethylamine, and 50 mL of N,N-dimethylformamide were added to a reaction vessel at room temperature. Then the resulting mixture was heated to 85° C., 20 mL of a solution in which 9 g of the compound represented by formula (1-7-3) had been dissolved in 20 mL of N,N-dimethylformamide was added dropwise, and the mixture was stirred for 2 hours at 85° C. After the end of the reaction, 100 mL of a saturated aqueous solution of ammonium chloride was poured into the reaction solution, and extraction with 200 mL of ethyl acetate was performed. After the organic layer was washed with a saturated saline solution, purification was performed by column chromatography (silica gel, ethyl acetate/toluene=1/5 to 1/3), through which 12 g of the compound represented by formula (I-7-5) was obtained.

Then, in a nitrogen atmosphere, 12 g of the compound represented by formula (I-7-5), 90 mL of dichloromethane, and 5 g of 1,1-thiocarbonyldiimidazole were added to a reaction vessel at room temperature, and stirring was performed at room temperature. After the end of the reaction, the organic layer was washed with a saturated saline solution, and then purification was performed by column chromatography (silica gel, toluene) and subsequent recrystallization (toluene/hexane=1/1), through which 11 g of the compound represented by formula (I-7) was obtained.

$$MS ( EI ) : m / z = 485$$

### INDUSTRIAL APPLICABILITY

The compound and liquid crystal composition according to the present invention can be utilized for liquid crystal display elements, sensors, liquid crystal lenses, optical communication equipment, and antennas.

1. A liquid crystal composition comprising one or two or more types of compounds represented by general formula (i) below 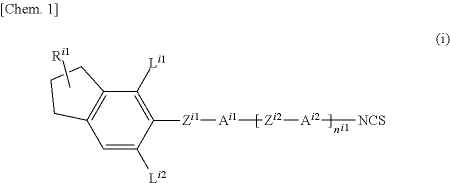
(In general formula (i),
Rⁱ¹ represents a hydrogen atom or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, has optionally been replaced with —CH═CH—CO—O—, —CH═CH—O—CO—, —CO—O—CH═CH—, or —O—CO—CH═CH—, and
one hydrogen atom in the alkyl group, or each of two or more independently, has optionally been replaced with a halogen atom, with the proviso that
an oxygen atom and an oxygen atom are not directly bound together,
Aⁱ¹ represents formula (Aⁱ¹-1), (Aⁱ¹-8), (Aⁱ¹-SP-1), (Aⁱ¹-SP-2) or (Aⁱ¹-SP-3) below, 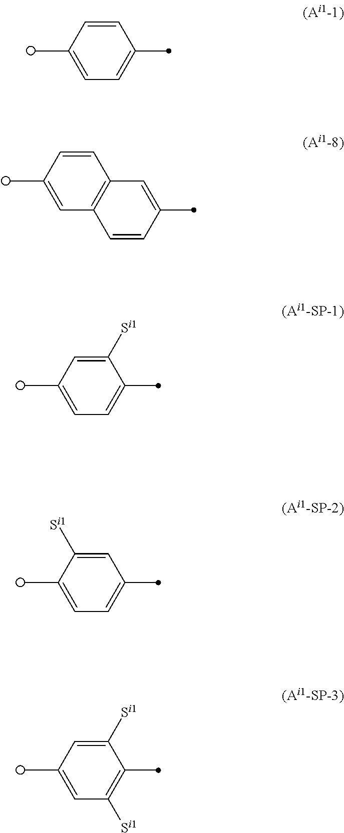
In formula (Aⁱ¹-1), (Aⁱ¹-8), (Aⁱ¹-SP-1), (Aⁱ¹-SP-2) or (Aⁱ¹-SP-3), the white dot represents a bond to Zⁱ¹, and the black dot represents a bond to Zⁱ²,
Aⁱ² represents any of a C3 to C16 hydrocarbon ring or a C3 to C16 heterocycle, where
one hydrogen atom in the Aⁱ², or each of two or more independently, has optionally been replaced by a substituent Sⁱ¹,
the substituent Sⁱ¹ represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, and/or —CO—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, is optionally replaced with —CH═CH—CO—O—, —CH═CH—O—CO—, —CO—O—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together, and
when there are a plurality of substituents Sⁱ¹, the substituents may be the same or may be different, Lⁱ¹ and Lⁱ² each independently represent any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, is optionally replaced with —O—CO—O—,
one —CH₂—CH₂—CH₂—CH₂— in the alkyl group, or each of two or more independently, has optionally been replaced with —CH═CH—CO—O—, —CH═CH—O—CO—, —CO—O—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together,
Zⁱ¹ and Zⁱ² each independently represent any of a single bond or a C1 to C20 alkylene group, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—, and
one —CH₂—CH₂—CH₂—CH₂— in the alkylene group, or each of two or more independently, has optionally been replaced with —CH═N—N═CH—, with the proviso that
an oxygen atom and an oxygen atom are not directly bound together, and
nⁱ¹ represents an integer of 0 to 3, with the proviso that
when a plurality of Aⁱ²s or Zⁱ²s are present, the Aⁱ²s may be the same or may be different, and the Zⁱ²s may be the same or may be different.) and
one or two or more types of compounds represented by general formula (ii) below 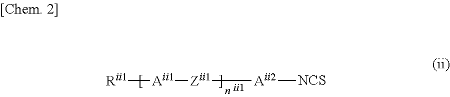
(In general formula (ii),
Rⁱⁱ¹ represents a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, is optionally replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced by a halogen atom, with the proviso that
an oxygen atom and an oxygen atom are not directly bound together,
Aⁱⁱ¹ and Aⁱⁱ² each independently represent a group selected from the group consisting of group (a), group (b), group (c), and group (d) below:
(a) a 1,4-cyclohexylene group (One —CH₂— or two or more nonadjacent —CH₂-s present in the group are optionally replaced by —O— and/or —S—.);
(b) a 1,4-phenylene group (One —CH═ or two or more —CH═s present in the group are optionally replaced by —N═.);
(c) a 1,4-cyclohexenylene group, a bicyclo[2.2.2]octan-1,4-diyl group, a naphthalen-2,6-diyl group, a naphthalen-1,4-diyl group, a 1,2,3,4-tetrahydronaphthalen-2,6-diyl group, a 5,6,7,8-tetrahydronaphthalen-1,4-diyl group, a decahydronaphthalen-2,6-diyl group, an anthracen-2,6-diyl group, an anthracen-1,4-diyl group, an anthracen-9,10-diyl group, or a phenanthren-2,7-diyl group (One —CH═ or two or more —CH═s present in the naphthalen-2,6-diyl group, naphthalen-1,4-diyl group, 1,2,3,4-tetrahydronaphthalen-2,6-diyl group, 5,6,7,8-tetrahydronaphthalen-1,4-diyl group, anthracen-2,6-diyl group, anthracen-1,4-diyl group, anthracen-9,10-diyl group, or phenanthren-2,7-diyl group are optionally replaced by —N═.);
(d) a thiophen-2,5-diyl group, a benzothiophen-2,5-diyl group, a benzothiophen-2,6-diyl group, a benzothiophen-3,7-diyl group, a dibenzothiophen-2,6-diyl group, a thieno[3,2-b]thiophen-2,5-diyl group, or a benzo[1,2-b:4,5-b′]dithiophen-2,6-diyl group (One —CH═ or two or more —CH═s present in the group are optionally replaced by —N═.), where
one hydrogen atom in the Aⁱ¹ and Aⁱ², or each of two or more independently, has optionally been replaced by a substituent Sⁱⁱ¹,
the substituent Sⁱ¹ represents any of a halogen atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced by a halogen atom, with the proviso that
an oxygen atom and an oxygen atom are not directly bound together, and
when there are a plurality of substituents Sⁱ¹, the substituents may be the same or may be different,
Zⁱⁱ¹ represents any of a single bond or a C1 to C20 alkylene group, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—,
one —CH₂—CH₂—CH₂— in the alkylene group, or each of two or more independently, has optionally been replaced with —O—CO—O—, and
an oxygen atom and an oxygen atom are not directly bound together,
nⁱⁱ¹ represents an integer of 1 to 4, and
when a plurality of Aⁱⁱ¹s and a plurality of Zⁱⁱ¹s are present, the Aⁱⁱ¹s may be the same or may be different, and the Zⁱⁱ¹s may be the same or may be different.
Compounds represented by general formula (i), however, are excluded.).

2. The liquid crystal composition according to claim 1, wherein the compound or compounds represented by general formula (i) are selected from the group consisting of compounds represented by general formula (i-1) to (i-14) below 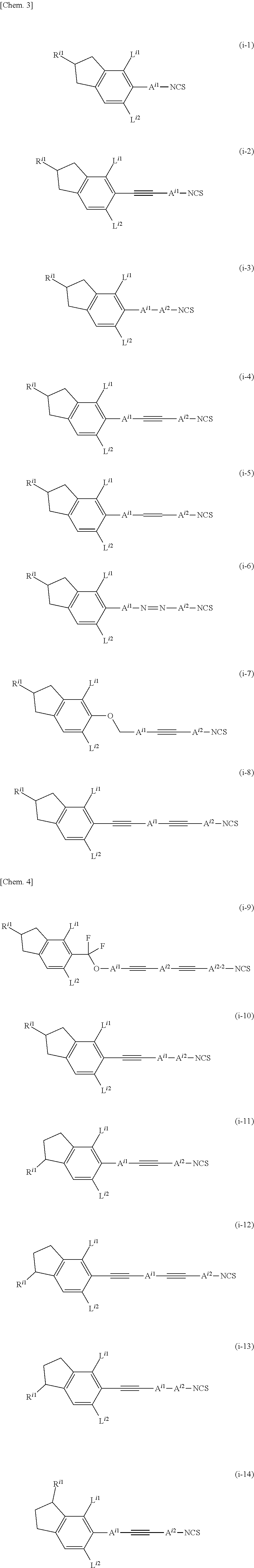
(In general formula (i-1) to (i-14),
Rⁱ¹, Aⁱ¹, Aⁱ², Lⁱ¹, and Lⁱ² have the same meanings as Rⁱ¹, Aⁱ¹, Aⁱ², Lⁱ¹, and Lⁱ², respectively, in general formula (i) above, and
in general formula (i-9), a definition of Aⁱ²⁻² is the same as a definition of Aⁱ² in general formula (i) above.)

3. The liquid crystal composition according to claim 1, wherein the compound or compounds represented by general formula (ii) are selected from the group consisting of compounds represented by general formula (ii-1) to (ii-8) below 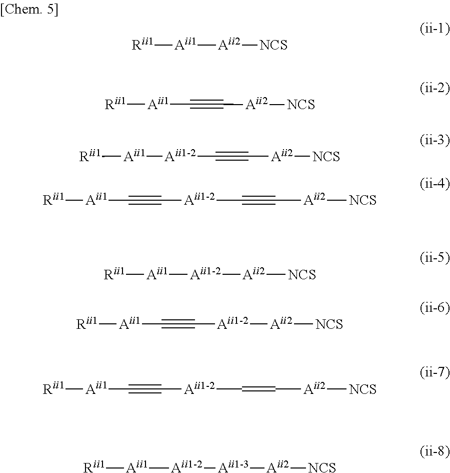
(In general formula (ii-1) to (ii-8),
Rⁱⁱ¹, Aⁱⁱ¹, and Aⁱⁱ² have the same meanings as Rⁱⁱ¹, Aⁱⁱ¹, and Aⁱⁱ², respectively, in general formula (ii) above, and
in general formula (ii-3) to (ii-9), definitions of Aⁱ¹⁻² and Aⁱ¹⁻³ are each independently the same as a definition of Aⁱⁱ¹ in general formula (ii) above.)

4. The liquid crystal composition according to claim 1, further comprising one or two or more types of compounds represented by general formula (vt) below 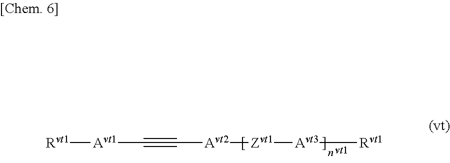
(In general formula (vt),
R^{vt1} represents a hydrogen atom or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced with a halogen atom, with the proviso that
an oxygen atom and an oxygen atom are not directly bound together,
R^{vt2} represents any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—, and
one hydrogen atom in the alkyl group, or each of two or more independently, has optionally been replaced with a halogen atom, with the proviso that
an oxygen atom and an oxygen atom are not directly bound together,
A^{vt1}, A^{vt2}, and A^{vt3} each independently represent any of a C3 to C16 hydrocarbon ring or a C3 to C16 heterocycle, where
one hydrogen atom in the A^{vt1}, A^{vt2}, and A^{vt3}, or each of two or more independently, has optionally been replaced by a substituent S^{vt1},
the substituent S^{vt1} represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, and/or —CO—,
one —CH₂—CH₂— in the alkyl group, or each of two or more independently, is optionally replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, is optionally replaced with —O—CO—O—, and
one hydrogen atom in the alkyl group, or each of two or more independently, has optionally been replaced with a halogen atom, with the proviso that
an oxygen atom and an oxygen atom are not directly bound together, and
when there are a plurality of substituents S^{vt1}, the substituents may be the same or may be different,
Z^{vt1} represents any of a single bond or a C1 to C20 alkylene group, independently at each occurrence, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—, and
one —CH₂—CH₂—CH₂— in the alkylene group, or each of two or more independently, has optionally been replaced with —O—CO—O—, with the proviso that
an oxygen atom and an oxygen atom are not directly bound together, and
n^{vt} represents an integer of 1 to 3, with the proviso that
when a plurality of A^{vt3}s and a plurality of Z^{vt1}s are present, the A^{vt3}s may be the same or may be different, and the Z^{vt1}s may be the same or may be different.)

5. The liquid crystal composition according to claim 1, wherein Δn at 25° C. and 589 nm is 0.38 or greater.

6. A liquid crystal display element comprising the liquid crystal composition according to claim 1.

7. The liquid crystal display element according to claim 6, wherein the element operates using an active matrix scheme or a passive matrix scheme.

8. A liquid crystal display element that reversibly switches a dielectric constant by reversibly change a direction of orientation of liquid crystal molecules in the liquid crystal composition according to claim 1.

9. A sensor comprising the liquid crystal composition according to claim 1.

10. A liquid crystal lens comprising the liquid crystal composition according to claim 1.

11. Optical communication equipment comprising the liquid crystal composition according to claim 1.

12. An antenna comprising the liquid crystal composition according to claim 1.

13. The antenna according to claim 12, wherein:
the antenna includes a first substrate having a plurality of slots,
a second substrate facing the first substrate and provided with a power feed section,
a first dielectric layer disposed between the first substrate and the second substrate,
a plurality of patch electrodes positioned corresponding to the plurality of slots,
a third substrate provided with the patch electrodes, and
a liquid crystal layer disposed between the first substrate and the third substrate; and
the liquid crystal layer contains the liquid crystal composition.

14. A compound represented by general formula (i) below 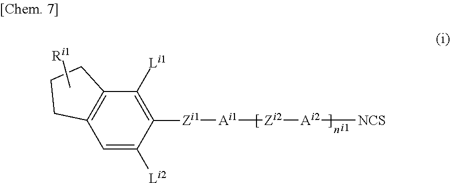
(In general formula (i),
Rⁱ¹ represents a hydrogen atom or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CO—O—, —O—CO—, —CO—S—, —S—CO—, —CO—NH—, —NH—CO—, —CH═CH—, —CF═CF—, and/or —C≡C—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, has optionally been replaced with —CH═CH—CO—O—, —CH═CH—O—CO—, —CO—O—CH═CH—, or —O—CO—CH═CH—, and
one hydrogen atom in the alkyl group, or each of two or more independently, has optionally been replaced with a halogen atom, with the proviso that
an oxygen atom and an oxygen atom are not directly bound together,
Aⁱ¹ and Aⁱ² each independently represent any of a C3 to C16 hydrocarbon ring or a C3 to C16 heterocycle, where
one hydrogen atom in the Aⁱ¹ and Aⁱ², or each of two or more independently, has optionally been replaced by a substituent S¹,
the substituent Sⁱ¹ represents any of a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, and/or —CO—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, has optionally been replaced with —O—CO—O—,
one —CH₂—CH₂—CH₂—CH₂— in the alkyl group, or each of two or more independently, is optionally replaced with —CH═CH—CO—O—, —CH═CH—O—CO—, —CO—O—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together, and
when there are a plurality of substituents Sⁱ¹, the substituents may be the same or may be different, Lⁱ¹ and Lⁱ² each independently represent any of a hydrogen atom, a fluorine atom, a chlorine atom, a bromine atom, an iodine atom, a pentafluorosulfanyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a C1 to C20 alkyl group, where
one —CH₂— in the alkyl group, or each of two or more independently, has optionally been replaced with —O—, —S—, —CO—, and/or —CS—,
one —CH₂—CH₂— in the alkyl group, or each of two or more independently, has optionally been replaced with —CH═CH—, —CF═CF—, —C≡C—, —CO—O—, —O—CO—, —CO—S—, —S—CO—, —CO—NH—, and/or —NH—CO—,
one —CH₂—CH₂—CH₂— in the alkyl group, or each of two or more independently, is optionally replaced with —O—CO—O—,
one —CH₂—CH₂—CH₂—CH₂— in the alkyl group, or each of two or more independently, has optionally been replaced with —CH═CH—CO—O—, —CH═CH—O—CO—, —CO—O—CH═CH—, and/or —O—CO—CH═CH—, and
one hydrogen atom in the alkyl group, or each of two or more independently, has optionally been replaced with a halogen atom, with the proviso that
an oxygen atom and an oxygen atom are not directly bound together,
Zⁱ¹ and Zⁱ² each independently represent any of a single bond or a C1 to C20 alkylene group, where
one —CH₂— in the alkylene group, or each of two or more independently, has optionally been replaced with —O—, —CF₂—, and/or —CO—,
one —CH₂—CH₂— in the alkylene group, or each of two or more independently, is optionally replaced with —CH₂—CH(CH₃)—, —CH(CH₃)—CH₂—, —CH═CH—, —CF═CF—, —CH═C(CH₃)—, —C(CH₃)=CH—, —CH═N—, —N═CH—, —N═N—, —C≡C—, —CO—O—, and/or —O—CO—, and
one —CH₂—CH₂—CH₂—CH₂— in the alkylene group, or each of two or more independently, has optionally been replaced with —CH═N—N═CH—, with the proviso that
an oxygen atom and an oxygen atom are not directly bound together, and
nⁱ¹ represents an integer of 0 to 3, with the proviso that
when a plurality of Aⁱ²s or Zⁱ²s are present, the Aⁱ²s may be the same or may be different, and the Zⁱ²s may be the same or may be different.)
